SEQUENCE LISTING (295 sequences)

SEQ ID NO: 1 - DNA, 92 bp, Canis familiaris
  misc_feature (31)..(31): n is a, c, g, or t
ccacaggaccgcagtgtaagtccgggctaanggaggcggggatatctatgcttaagctttgttttaactgcagtgtaagatgcgtgtattta

SEQ ID NO: 2 - DNA, 564 bp, Canis familiaris
gaaactagagttcatcctggcagctcaccgacctgcctgcaagatccctgatgacctgggcttccccgaagagatgtccgtggcttccctagatctgagcgggggcctgcccgaagctgccaccccggagtccgaggaggctttcaccctgcccctcctcaatgatcctgagcccaagccctccgtggagcccgtcaagagcatcggcagcatggagctgaaggccgagccctttgatgacttcctgtttccagcatcatccaggcccagcggctcggagaccgcccgctccgtgccagacatggacctgtctggttccttctatgcagcagactgggagcccctgcatggtggctccctggggatggggcccatggccacagagcccgagcctctgtgcacccccgtagtcacctgtactcctagctgcactacctatacgtcttccttcctcacccacgctgctggccctgtgagcaggcagaggggaggcggcaggcaccctagggtgctactgcccaagttggtgcattacagagaggagaaacacgtcttccctcgagg

SEQ ID NO: 3 - DNA, 337 bp, Canis familiaris
atctgctgaagggaaaggagaaaccagggaaaagagaaagaaacacaagagacttaagagacaggaggaggagatggccacaggagggggttcctctaggtgagatggaggttctcagagccaaatcctccccctctactccaccccagggctggtgtggaaggtctgttggcctgcgatcctttctgcccacttgcccttcctctgcagttcctactgcctgtgacttcagctgcctgaaacagccatgtccaagttcttcacctctatccaaagaacttgatttgcatggattttggatatatcatttcagtatcatctccatcgtatgcctgac

SEQ ID NO: 4 - DNA, 119 bp, Canis familiaris
cttcgcctgcgacatctgtgggagaaagtttgccaggagcgatgagcgcaagaggcataccaagatccacttaaggcaaaaggacaaaaaagcagacaaaggtgttgtggcctcctcag

SEQ ID NO: 5 - DNA, 391 bp, Canis familiaris
agcaataatctcttctgtaacttttattaatagtaatattattgtagtcctattgtactcactttttaaaagatttctagcatcatgaatgtcctacttcagtaagacccatttaaatgctgttgatattttagcagggatctttagtgcaacatatacatgttttagagaattgttagctagctgtacatgtttttaaaacgtttagctagctataaggctgtaattggaaacttgtactttttatttacaacaaaacatttattctgttaatccagtttgctaccaaaatattttttagatataagtgtgtgtctgtttagaagttagaaactttaaatactggtctgaggttccatttggattcattattacattgtcttgttaccaa

SEQ ID NO: 6 - DNA, 380 bp, Canis familiaris
  misc_feature (29)..(29): n is a, c, g, or t
ggagtgtgtattgttcccagtgacacatnctgagagctggtagttagtagcatgttgagccaggcctgggtctgtgtctcttatctctttctctttagtcttctcatagcattaactaatctattgggttcattattggaattaacctggtgctggatattttcgaattgtatctagtgcagctgattttaacaataactactgtgttcccggcaatagtgtgttctgattagcaatgnccaatattnaactaagaaaagatatgnctttattttctagtagatagaaataaatagctctatccatgtactgtagttttttcttcaacatcaatgttcattgtaacgttactgatcatgcattgttgaggtggtctgaat

SEQ ID NO: 7 - DNA, 203 bp, Canis familiaris
ctcccctcaaatcttagggtccatttgagtgttcacattagggaaaaactatttaagtgtgaagagtgtggtaagggcttcagtcacagtttacgtcttcaagcccatcagagagtccacactggagaaaaaccatacaaatgtgatatatgtggtaaggacttcaatcaccattcacgtcttaaataccatcagaaagtcca

SEQ ID NO: 8 - DNA, 596 bp, Canis familiaris
  misc_feature (345)..(345): n is a, c, g, or t
ttcctgtccttgcaaggatgcaaaccagcacatacacacccattgtggccactgtgatctttgggattattgcaggaatcatggtattattctttggatttgccaatctcttggacctcatgtcagttggggccctgctagtttactccctgctggcttttagtgttctcatcctcagacctgcagcagagaagctgactctacagggactattttttccaggcagccccacccccactccactctctggtcgggttgtctatgtttgctcctcactgcttgctctgctgctgactcttctctgcctggtgctggcccggtggccagatctgctttctggagacncagtgtggatcacagtggttgtgctgctcctggtgctcatcactggagtcactggagtcatctggagacagccacagaactccactccccttcacttcaaggtacctggtctgcctctcctcccactcctgagcatctttctcaatgtttatcttatggtacagatgacagctggcacctgggccctatttggtgtctggatgctgattgggtttgctatctaccttgcttatgtgatccagcagcacctg

SEQ ID NO: 9 - DNA, 149 bp, Canis familiaris
gatgaatggttgattgtcccatcctgagaccagttggtgggagcagaaatctatgctgcctggggacctgaatgttgccctgtgcaagaggtgcccttggctttggagggaaagataagtcgataactagctgctacccagaaatggtg

SEQ ID NO: 10 - DNA, 108 bp, Canis familiaris
tggactgccagggacacgtggtactgacggactttggcctctgcaaggagggtgtagagcctgaggagaccacatccacgttctgtggcacccctgagtacttggccc

SEQ ID NO: 11 - DNA, 303 bp, Canis familiaris
  misc_feature (257)..(257): n is a, c, g, or t
tacggaattccgaaagataccagcttgcaggaggtgtctagatatcgagcccggctggtgaaaggacgaagaattagcctttgaagtagccgcctgctggagctctttgagagaaaaccaaaaggaatcaagatgcctacggaacatgttttattaatgagctgtcctaatctttcctgccacttgttatgaagatcacaacgcacttacagggaactttatgggatttggttagctctgggtgaggggctgttgcnttaatggatgaacagatgtaacactggcctattactgtcgtcagga

SEQ ID NO: 12 - DNA, 246 bp, Canis familiaris
gtgctacctgaagcttagagaatacaccaaagctattgagtgctgtgacaaggcgcttggactagacagtgccaacgagaagggcttgtacaggaggggtgaagcccagctgctcatgaacgagtttgagtcggccaagggcgactttgagaaagtcctggaagtgaatccccagaataaggctgcacgactgcagatctccatgtgccaaaaaaaggctaaggagcacaatgagcgagaccgcag

SEQ ID NO: 13 - DNA, 298 bp, Canis familiaris
atcttatcactattgatctgatgcaaaattcatctcagaaagctcagaatgatgtggacatagctgatgtggcttattattttgaaaaagatgttaaagacgaatccttgttccattccagtaaaatggacctaagagtaaacggggaacaattggatctggatcctggtcgaactgcaatttactatgttgatgaaaaaccacctgaattttcaatgcagggtctacaagctggtattatcgctgtcattgtggttgtgacactagcagttattgctggaatcgttgtgctggttat

SEQ ID NO: 14 - DNA, 376 bp, Canis familiaris
tgagtgcagttcctgaaggttccactgcttccaaaccaaggaggggacaaagaacttatctgagcacctaccacatggaaggctctgttgggtcctgctaaggatgtcattagaaagttgaagacaattgctaaagtcctttcatctcccacctgcaacctccacagtgtgctgtgccatttctcctgccaggagtgcccatcctccgcatacccatctcttaagctccaactcataaaagcctgcttgaaacagcgccagccaaatctgattcctctcccttctgaatgcccggcacattcagtctgtacctttcctcgcagcacagcgtgtggcctgtctcatattctagtgatctgtttactaatccagctct

SEQ ID NO: 15 - DNA, 502 bp, Canis familiaris
ggaaaatttccaggccagccaattcacattgtatatgtaccttctcaccttcatcatatgctctttgaactattcaagaatgcaatgagggcaacagttgaacaccaggaaaactggccttcccttacaccaattgaagtgattgttgtcttgggaaaagaagatcttacaattaagatttcagacagaggaggtggtgttcccctgaggatcattgatcgcctctttagttacacatactccacggcaccaacgcctgtgatggataattcccggaatgctcctttggctggttttggttatggcttaccaatttctcgtctctatgccaagtactttcaaggagatctgaatctctactctatgtcaggatatggaacagatgccgtcatctacttaaaggctttatcttcagagtctgtagaaaaactcccagtctttaacaaatcagccttcaaacattatcaaatgagcatcgaggccgatgactggtgtatcccaa

SEQ ID NO: 16 - DNA, 35 bp, Canis familiaris
ggtggtagagaatgctccaaacatcgatctgctag

SEQ ID NO: 17 - DNA, 535 bp, Canis familiaris
  misc_feature (41)..(41): n is a, c, g, or t
atcccctcgcgctggcagagctagtccctttacctgatctncttngcncnggntccccccatgtcnctcagtccatcagnnncccctgccaagtccnccatctgnaacctcacgaatccacctctgcntntcatcctcaccnctataaccccggttcaagcnatacctcctncctcatctggaccagacagagnctccnggcctcctgcctgacctcngggcgcttgccctgaccagtcagtcgcaagcagcagcacccaagcaggccacctgaaacacaaatctagtcactcccctttgaaaagtaacaaaggaagncctcacgaagatggagtagggaggccagagaggggagctgcaccattccacgtcagctacagaccctgatggcacagaagcctcaaccccacgacggggaaagatgcgcatcacatcccctgcaggaaatccgccagccctgcactcagccggtgagcaccggtcatcaccctgcgcgcctgcctttctgtgatagacttctattccaaacaaccct

SEQ ID NO: 18 - DNA, 291 bp, Canis familiaris
aaatcctgcaggtcagcagtgagctgagccacgtgcacgtccagtccatgctgcgccggtggtttgtggagacggagggggccgtcaaggcctacctgtgggacaacaaccagatggtggtacagtggctggaacaacactggcagacggggaacagcctgcgttccaccatccatgagaacatcaggtgcttgaagcgagactctgtcctcaaggccatcagacgcctggttcaagacaatcctgaagtggccgtggactgcatagtgtacatgagccagcacatcagcc

SEQ ID NO: 19 - DNA, 162 bp, Canis familiaris
tgtgtgaacactgctggggtccgaagaactgataaggacactgaaatatcctgcactgaacgagtgaggacctactggatcatcattgaattaaaacacaaaacaagagaaacaccttatgatacacaaagtttgcaaaatgcacttaaggagacactcaaa

SEQ ID NO: 20 - DNA, 334 bp, Canis familiaris
gatagagagttttgggcttttgaaacaaaactcagtatttgtttctcgtgccttttattctcagtgttttaggagggagaaagcaaaaggctcagtgtgcaccccgacacatgcaatgtgcaaacagatgactgcatttgattggctgttttgagaggggggggagtcttacgattgaatatgaagaaatattacatattttaactctaactcaggttgacagaattggaaaatggagtggtgccttttatgttgataaatacttaaagatgaacagttatcaaggaaaatgctgctgtttccttttaacttattaacccagttgtccattaat

SEQ ID NO: 21 - DNA, 488 bp, Canis familiaris
  misc_feature (27)..(27): n is a, c, g, or t
taccctacctatacttccagaatgatncctcccctaagaagnctatggtctcaatttcagctaagaagnctggtnncattgtttccatcctgggtgttaacatncnctttaatcatcctgttcagacttttncccttcttagaagagcnaaccnctccncaaaacatttgaattctgagggtncccttggtcttcctctcttgtgactaatttttatctaagaagagtggattgaagcttttgcagttttacatctttcccagagtaatggcttttccttttcacactttcctgatttaggcgacaagttaaaggggactcctttgcaagagttgaatggcctgggaaaaggcccagttaggaagtttcacactaactggttcccagcaaaagcaacatacccactgggtatgtctctattggaggccgtttcatgcctgtctggtnccaataaggcaaaggaagagcttagctaagcccaatgccga

SEQ ID NO: 22 - DNA, 578 bp, Canis familiaris
taatggaatcactcacgctggaagggaagtggagaaagtttttaatggacttagccacatggggagccaggccggcaaggaactggacaaaggcgtccaggggctcaacagcggcttggacaaggtagcccatgagatcaacagtggcatcggacaagcaggaaaggaagctgagaagtttgtccatggggtcaacaacgctgctggacaggctggaaaggaggcagacaaagtggtccaaggggtccacgatggggtcaaccaggccgggaaggaggcagagaaatttggccaaggggtccaccacgctgctggccaggctggaaaggaggcggagaaacttggccaaggtgtccaccatgctgctggccaggccgggaaggaggtggacaggttgccgcagaatgttcataatggggtcaaccaagccggcaaggaggccaaccagctgctgaatggcgctcatccaggcgtttccaccggccagcaccagcacggagggggcgcaaccaccacgttaacatctggagcttcggtcaacaggcctttcatcagcttcccaactctgtggcaggtga

SEQ ID NO: 23 - DNA, 39 bp, Canis familiaris
aggaaaaagcccgatcatatcaggtaccaaatggcttcc

SEQ ID NO: 24 - DNA, 329 bp, Canis familiaris
gccattcaaatgtgacatatgtggtagggcttcactcagatttcacatcttcaggcccatcagagagtccacacaggagaaaagccatacaaatgtgagacatgtggtaagggcttttgtcagagttcaaatcttcaggaacatcagagagtccatactggagagaaaccctacaaatgtgatgtgtgtgggaagggcttcagttggagctcacatcttcaagcccatcagagagtccatacaggggagaagctcaacaaatgtgaagaatgtgggaaaggcttcatcaggaactcctatcttcatgttcatcaaggatccacacggga

SEQ ID NO: 25 - DNA, 330 bp, Canis familiaris
gccattcaaatgtgacatatgtggtaagggcttcactcagatttcacatcttcaggcccatcagagagtccacacaggagaaaagccatacaaatgtgagacatgtggtaagggcttttgtcagagttcaaatcttcaggaacatcagagagtccatactggagagaaaccctacaaatgtgatgtgtgtgggaagggcttcagttggagctcacatcttcaagcccatcagagagtccatacaggggagaagctcaacaaatgtgaagaatgtgggaaaggcttcatcaggaactcctatcttcatgttcatcaaggatccacacggga

SEQ ID NO: 26 - DNA, 101 bp, Canis familiaris
aaggagaacagaccaactgtgtaagagctaatcagtgacactatgcagcattgtttgatgtcttacttgttgtttaacctttgtctctttatgttaactgg

SEQ ID NO: 27 - DNA, 151 bp, Canis familiaris
  misc_feature (30)..(31): n is a, c, g, or t
ctctcttaaaagaagggtgtgaggggaaanntccccccccaaattncctcaagaattaaccccgaaaatgtttggacccagaaagcagctctcaaaggtcaggctgttcngagccttggctaggagagtccttgaggcgactcattaaatt

SEQ ID NO: 28 - DNA, 500 bp, Canis familiaris
  misc_feature (118)..(118): n is a, c, g, or t
ttacttgctttcttgatttcaccaaaaccagatttaatttaaaggaccacgttaatttttccaagggaaggagacaattcattgtacataatgtatacacacacacaaaaaaaatatncctgtagaaatattattccagcatagcaggaaacaaaaagaaacaaaagtattgggcagtcggnaggtgagtgtgtgcgtnccgtaacccgttgtgaccccctcagcgtgcnnagtcttctagnttaacccacaaagtacattttctgtcttaatgatactgtaggttcncccttttttttttaatttccctgcaaataacgagaccnacagaagtgactctagctatctaacggttctgttcttttatacgcagcaaacacaccgtacatttccgaagaggctncagcctgaaggcattttccagtgacgttagtgcacaaacgctttaaattagactgggactgccagaatnaaatgtaaatgaggaatttctcgtaccc

SEQ ID NO: 29 - DNA, 290 bp, Canis familiaris
gtgagaattgtctgtttaacaaacagattggaataaagcctactcgatcagttaaactactttaatacacattcatttttaagaaatatttgttttaacataaacaaattgtatcagtgttcgtgaataaaatacaaaaatgattgttaatgattggtgctcttaaggtgagcttaaaaattatctaagacctctatccaaatttgtcctgtagtaatagctgtattaatagattgttggtgtttaaagatctgaaatgtgagtagaatgtattcagctgtttaacatgt

SEQ ID NO: 30 - DNA, 401 bp, Canis familiaris
ccgcccttgtactgggtggttggcagggagctcacgccctgggggagggggatgcattcatgggggtgacccctactacttgtcttagcccccagcccagccctggccttcatgtttttgtaagataaaccgtttttaacacacaccgccgtgctgtaaataagccagatgctgctgtaaatataggaaggaagaggccgagatgggagtgggggctcggagggaggggcggcccccaccagcccgggcaagcctctcccgcctccttgcacctgctctctcttcccaccctctttccacatgtacataaccttactctacaaagaagacaaatgacagattctgacatttatatttgtgtattttcctggatttatagtatgtgacttttctgattaata

SEQ ID NO: 31 - DNA, 217 bp, Canis familiaris
  misc_feature (51)..(51): n is a, c, g, or t
agcccaatgcttctcatgagacgcaccttctccacactcatgactcttctncagtagactgttccttttcttaatttaatcgtattatgtgttgtgttattgtatttgaacttatttctattatttgttttgccaaaggacacttacntgtcccccatggggatagtctaccatcattgtttctctgctattgtagatatatgaataatgcaactga

SEQ ID NO: 32 - DNA, 406 bp, Canis familiaris
  misc_feature (168)..(168): n is a, c, g, or t
tggaggcctcccgacaggacgtgaaggacaagatttgcgccatggaggcaggaagagcccggcgtggtgtaaatggagagcttgaccatccctgggatggcgccagctccaggagccgagggaggaggagaaaggaagaggctctcctcagcttaatctcccccttgnagactggaatcttccacagactccctttctcagcccagggctgtcctcaaagcatgcggtcaaggttggaggaagctaccagaagggaagagggtgcctttctgaggccttcctcagtgaccccagagacctcttgcctggaactcactggtggccctggacagagantggaggctccctcatcccctaccagccccctctttggaacgtctctgctatgttttctgaccctgttagt

SEQ ID NO: 33 - DNA, 499 bp, Canis familiaris
ctgctgctatacactcaccaataagaagatctcaattcagaggctggccagctataaaagagtcaccagcagcaagtgtcccaaagaagctgtgatcttcaagaccgtcctaaacaaggagatctgtgctgaccccaaacagaagtgggtccaggattccatggcacacctagacaagaaaagccaaacccaaactgcaaagccatgaatactcactctgaaatggaagaatctgaaactaatttattttcttctaaccttccctaaatgccctctgatattattttattataatttcaaagagtacaaactttgtttattgatatgcacacaatgccttaagtaatgttaatcttatttaaattattgatgttttaaatttattttccccaaatactagtgtattttttaaaatatggagacttgaaataaactgctttcctctgggggccccagttctacccctgggatggtgtgagggtccttgcaaggatcatta

SEQ ID NO: 34 - DNA, 510 bp, Canis familiaris
gccaactgaagttctggttacccataggatgtgcagcttttgttgtagtctatatttttggatgcatatttctttgttggcttacaaaaaagaaatatcgatccagtgtgcatgaccctaacagtgaatacatgttcatggcagcagtgaacacagccaaaaaacctggactcacaggtgtgactcataatttggaactctgtggcacccaggcatgaaccacgttggccagttccctccgacctgaagtacaagattcccacgtatcctggaccacagagtcagacttgatttgagtacatatatcttctgttggtgttttgttcaatctggaccagtgactatatcagtcaaaagggattttagcaggctgctttggtactgttgggttatcaaaacatcttcttgcaactagttatagaaagcccagcttgtgtctgtttaccaaatagaactcactgggatggaattcctatcttcacatctgcttctagcaatgcgccagacagt

SEQ ID NO: 35 - DNA, 563 bp, Canis familiaris
aggctaagcggtgccaatccttcaggggccttccaggtgctgacggcggcctaccagcggtcctcccgggctgctcagcaggtgtccgacggctcccgccggctcttccagctcagggacagccggagagaggcagagaggctggagggtcagctgggaggaggagctggagtcgggggtccccagctcgtggccctgaggctggagatggcttccttgcctgatctgacgccaactatcaacaagctctgtgggggctcccggcagacggcctgtaccccaggagcgtgccctggagagctgtgtccccgagacaacggcacggcctgtggctctcactgcagaggtgccctccccagggccgatggggccttccggatggccgggcaggtggccgagcagctgcagcgtttcaacgcccagctccagcagaccagggagatgatcagggccactgagaaagccgccttgaaggtccagtcagacgcccagcgcctggagacccaggtgagcaccagccgctcgcagatggaggaggatgtcaggcgcatgcggttcctcat

SEQ ID NO: 36 - DNA, 312 bp, Canis familiaris
gtcttcagtatattcggtgctcttcgaactggtgcttatatggtgtacattctgatgaccaaaggcctgaagcagtcagtttgtgaccagagtttttacaatggacctgtcagcaaattctgggcttatgcatttgtgctaagcaaagcacccgaactaggagatacaatattcattattctgaggaagcaaaagctgatcttcctgcactggtaccaccacatcactgtgctcctgtactcctggtactcctacaaggacatggtagccgggggtggttggttcatgaccatgaactacggcgtgcacgcc

SEQ ID NO: 37 - DNA, 528 bp, Canis familiaris
  misc_feature (170)..(170): n is a, c, g, or t
aaggctcataggattttcttgtatcttctttcgcaacctcctggaaggcataactatttcaaaataaaggttaaaaacaaacaaaatagaaaaacattaccatcaattctatccccgttcgcaaacaccttctagatatgactatccaataagcatgattggtgcgtcantacntagatgttatattcatggtggatgccttaaagaaattcacacaaatttcacttttgacagatttgatgggtaagccatcatccaattaaaaaacccacctaatctaaaaaaaataaaaataaaaataaaacataaataaataaaaatttcaaaaaaaccacctaatatctcacttaggtaacgtaggtaaaactctactatttgaagatgccaatttgaagattttaatatgtgtatttatattctgagtatttaagnattttggatcatcaagctgttgactaaggcaatgcattttaatttttaaaaagatactatcgtaagatgttatgtcacatcattatcttctgcaat

SEQ ID NO: 38 - DNA, 169 bp, Canis familiaris
  misc_feature (26)..(26): n is a, c, g, or t
agctggtaaagactgatggacattanaatcaaagaagataatcacagctgatggagccacaactttactgtgagaacttnngatgtgtcgcttaaaggtctagggatggnattccantcagctgattgaacttgttgacctgtacaggcttgattatatctttggaggt

SEQ ID NO: 39 - DNA, 208 bp, Canis familiaris
atcccaattacctgctgcttcagcatggtcaaaaggaagatccccatgcagaagctggagagctacatgaggatcaccaacagccagtgtccccaggaagctgtgatcttcaagaccaaagcatccagggagatctgtgctgaccccaagcagaagtgggtccaggattacatgaatcacctggaccaaaagtcccaagcccagaagc

SEQ ID NO: 40 - DNA, 36 bp, Canis familiaris
caaggcaaatgggtagccaagggtcagtatctgtct

SEQ ID NO: 41 - DNA, 520 bp, Canis familiaris
  misc_feature (145)..(145): n is a, c, g, or t
ggaagtgccaattaggctacccttccatttagatttttttctaagcaatatatatcaggatcttcagaaagtccatactccttggtcaaacaaatccattttcaataatctatactatggaaataaatgagaaaagaagaaaaanctttatgtaaatacaaataatacaatattatttactattctagaaaattagaaataccccaaagtctaattttaggggaaggattaaatcaagagtagtacattagttgtaatattatgtagccattacaaattacgttacgcagtcattacaaattatatcctagncattaagaatactgttaaatgaaaaagcagattacaaatttatgtttagtggtataaacatanccatataaaacaaacatgaaatcttcanagtttaggagaaaaactaaatctagaaggatttacagaaaaatgtcanctgtcattggtctggatcgtaggtgacctttcctgcttttctctatttcccaagttttctttatcatgt

SEQ ID NO: 42 - DNA, 200 bp, Canis familiaris
  misc_feature (44)..(44): n is a, c, g, or t
ctctgagcatgtccccatccttgataccagtatcaagcttggcnggcggggggggggagggggccattctcatcacccaactgactcagaaacaaagttaccttccttctcagtcttctggaacaaaaatagaactaatgccatcctgaagggatgcaggagagagaaagggtcataaacatgacatagttctcctcccc

SEQ ID NO: 43 - DNA, 453 bp, Canis familiaris
  misc_feature (37)..(37): n is a, c, g, or t
gaaatacaattttgcccctcaggaaagagagattgangccacagagctctgccaggaaccaaggnacgtgagattccttttgctggatacaaagaaatgaaaaagccaatggtgaaatttcaagtctcaaaaaccatcctttnttgaccaatcccaggcaacaaacgtttgtttccctgagtgttcttacgaacatctgggatttannnnnnntntnnnntnggantnannnnnaannntnnncttattgaatgccaaccgcaacgatgtgaaaacctatggccaaatacttgaaaacacctttccacactgcacagtggtccaatggcttatgtgaggtaaaacactagagaaatttctagtccatcatggctaaacttacagacttcaaccatgtgtcacgtcacttattagacaagtcaccagtgatctcatgcctgtaaattaagcctc

SEQ ID NO: 44 - DNA, 363 bp, Canis familiaris
  misc_feature (61)..(61): n is a, c, g, or t
aaggaagcttccttcgagtacttgcagaacgagggtgagcgtctgctcctggaagccatgnatgagctggaagtggcgttcancaacactaccgtgcgtacagactgcaaccacatcttcctgaacttcgtgcccaccgtcatcatggaccctcttaagattgaagagtccgtgcgttccatggttatgcgctatggcagccggctgtggaaactccgcgtgctgcaggctgaggtcaagatcaacatccgccagaccaccactggcagggctgttcccatccgcctgttcatcaccaatgagtcgggctactacctggacatcagcctctacaaggaagtcactgaccccagatctggaaat

SEQ ID NO: 45 - DNA, 422 bp, Canis familiaris
  misc_feature (332)..(332): n is a, c, g, or t
gttagaaattatgctcccatctccgttctgaccattctctcgtattgttaaaatgcaatcaagcctggaaatatttataccagtggagcccttgagttgagtgcgacgttcataaacagcagaagcattttgcaatttccgatctgttgaaatgtacacattccaatctggcctgcttttttaaagcctaacttcatgtgaataatcaaatagaaaaatctaaacttgtataatatgtaatgaccaactgcctttccctggcccagataaataatttcaaaggcgcccgtgttaggtccataaacggggaaagtctatggtttgggagaaanggatttgtttatccgccaataaggtccatccgggggcangctcctaaggcttgctttattgccttcctcccagcggaatgggctttgggt

SEQ ID NO: 46 - DNA, 372 bp, Canis familiaris
agagcttcagtcagagatcataccttcaaagccatcagagtgtccacactggagagagaccatatatatgtgaggtatgtgggaaaggcttcagtcagagggcatatcttcaaggtcatcagagagtccacactagagtgaaaccatataaatgtgagatgtgtgggaagggctttagtcaaagttcacgccttgaagcacatcggagggtccacacaggagggaaaccatacaaatgtgaagtatgcacaaagggcttcagtgagagttcacgccttcaagcacatcagagggtccacacaggaggccctataaatgtgaacaatgtggtaagggtttcagcgggttttcaagtcttcaggctcatcacag

SEQ ID NO: 47 - DNA, 519 bp, Canis familiaris
attgatagctctttctcgattccgtgggtggtggtgcatggccgttcttagttggtggagcgatttgtctggttaattccgataacgaacgagactctggcatgctaactagttacgcgacccccgagcggtcggcgtcccccaacttcttagagggacaagtggcgttcagccacccgagattgagcaataacaggtctgtgatgcccttagatgtccggggctgcacgcgcgctacactgactggctcagcgtgtgcctaccctacgccggcaggcgcgggtaacccgttgaaccccattcgtgatggggatcggggattgcaattattccccatgaacgaggaattcccagtaagtgcgggtcataagcttgcgttgattaagtccctgccctttgtacacaccgcccgtcgctactaccgattggatggtttagtgaggccctcggatcggccccgccggggtcggcccacggccctggcggagtgctgagaagacggtcgaacttgactatcta

SEQ ID NO: 48 - DNA, 446 bp, Canis familiaris
  misc_feature (38)..(39): n is a, c, g, or t
gacacaacacagacaaattaccatggagtctgtgctcnnctgggttttccttgtcgctattttacaagctggccctcaatgagctggtgacactgacgtgcttggtgaggggcttcaaaccaaaagatgtgctcgtacgatggctgcaagggacccaggagctaccccaagagaagtacttgacctgggagcccctgaaggagcctgaccagaccaacatgtttgccgtgaccagcatgctgagggtgacagccgaagactggaagcagggggagaagttctcctgcatggtgggccacgaggctctgcccatgtccttcacccagaagaccatcgaccgcctggcgggtaaacccacccacgtcaacgtgtctgtggtcatggcagaggtggacggcatctgctactaaaccgcccaatcttccctccctaaataaactccatgc

SEQ ID NO: 49 - DNA, 275 bp, Canis familiaris
aacaggctgctcaggtgcctctgtcctctcctctggcaggtggcaacactgtagtagatggctacggcaaggcccagaacagcacagaagtggctgtagaggagtataccctgatgagcatagacaccatcatcaacgggaaggaaggtgtgtttcctgggctgatcccaattctgaactcttaccttgaaaacatggaagtggacgtggacaccagatgtagtattctgaactacctaaagctaattaagaagagagcatctggcgaactaatg

SEQ ID NO: 50 - DNA, 411 bp, Canis familiaris
aaataacttgtgctccttggtaaagaggtgagaaagaactacagcttttaaatcaagctctggcacagtgccaaagccttgacccattcatccttcacctatttatttctttgttaaattttgtgacactaaaacaaagcaaaaaaaattgtgacactttactgggtgtctcgggaggtcaaagtatgaaatattctaaatatgaaatattctaaatattttttagaaaaccaatgcacttacccagtaaattatctccgagtatttcccaaatggttgctggaagagtaaattgagtataaaactgttgaaaatacatgatgatggctcacagaatgtcttcatatctgctgaacaaactaaaggcacactgctttgggatttaatgtccagtgtttttcgattccttac

SEQ ID NO: 51 - DNA, 279 bp, Canis familiaris
  misc_feature (34)..(34): n is a, c, g, or t
ttcatcgcctggatccacctcaaggcgctcagantgttcgtggagtcggtgctcaggtatggactcccggtgaatttccaggcggtgctcctccagcctcataagaagtcatccaccaaacgtttaagggaggtgctgaactctgtcttcagacatctggaccaagtagcagctgcaagtatactggatgcatctgtggagatcccgggcctgcagctcaacaaccaggactattttccttatgtctacttccacatcgacctcagtcttctcgactag

SEQ ID NO: 52 - DNA, 580 bp, Canis familiaris
cacatctgcttgtactaccgggagatcctggtgaaagagctgaccacatcgagcccagaaggctgtcggatctcccacgggcacacttacgatgccagtaacctggaccaggtcctgttcccctacccagaggacaatggccagaggaaaaacatcgagaaacttctgagccacttggagaggggtgtggtcctctggatggcccctgatgggctttatgccaaaagactgtgccagagcaggatctactgggatgggcccctggcactgtgcagtgacaggcccaacaaactggagagggaccagacctgcaagctcttcgacacacagcagtttttagcagagctgcaagcttttgctcaccatggccgtcccctgccaagattccaggtaactctgtgcttcggggaagagtttccagatcctcagaggcaaaggaagctcatcactgctcacgtcgaacctctactcgccagacagctgtactattttgctcagcaaaacagtggacacttcctgcggggctacgacctgcccgagcacatcggcagccccgaggactaccacaggtctattcgcc

SEQ ID NO: 53 - DNA, 467 bp, Canis familiaris
ctattttatgcaaagcccaggcctacaggcatattcttttcaatactaaaataaacatgcatggaaaactctgcatattttgtttgctttgtgaagcatacctgagatggtggcagcttcaagattcaaaccagaacactgatcctaatgacttcatcagatatgccaaggaatgggatttctatagaatgtttgcaattgcttctctagaacaaactgccttttttattggcatttttactttcctgtggatagaacgaccaatgacagcgaaaaaaaaacccaacttcactttgctgctgaaagcattattattatccagctatggaaagctcttgctcattccagctgtcatttgggagcatgactacacgcccctgtgcctccgactcattaaagtatttgtccttacatcaaattttcaggcaattagagtgaccctgaatatcagccggaagctctccttt

SEQ ID NO: 54 - DNA, 152 bp, Canis familiaris
taccttgtcaccaatacctcacattcctcgaagcccttacaagttttctagttcacctttgcggattcctggagggaacatctacatatcacccctgaagaatccatataaaatttcagaaggtgtgccaacgccaacaaaaatgactccaa

SEQ ID NO: 55 - DNA, 366 bp, Canis familiaris
gtgccttggctgtggctgtagcccagcccacagatgtagtaaaggtccggttccaggctcaggcccgggctggaagtggccggagataccaaagcactgttgatgcctacaagaccatcgcccgagaggaagggttccgaggactctggaaagggacctctcccaatgttgctcgtaatgccattgtcaactgtgctgagctggtgacctacgatctcatcaaggacgccctcctgaaggccaacctcatgacagatgacctcccttgccacttcacttctgccttcggggcaggcttctgcaccaccgtcatcgcctcccccgtcgatgtcgtcaagacgagatatatgaactctgccctaggcc

SEQ ID NO: 56 - DNA, 167 bp, Canis familiaris
ggtcccggcagcaggagccagcacagtccggccagatgacataataccaggtgtagaagacagcgtggtgaccccaggcacagaagacagcgtggtgacgccaggcgcagaagataacgtggtgactgacggtgccactgaagagccttatgagtctggcttaaccc

SEQ ID NO: 57 - DNA, 553 bp, Canis familiaris
gatgccaatgaatctgaccatttcgagaatatccagtccacaaattggcagacgatgagatttaagccccctcctccaaactctgatattggatggagagtagaattccgacccatggaggttcagttaacagactttgagaattctgcatatgtggtgtttgtggtgctgcttaccagagtgatcctttcatacaaactggattttctcattccactatcaaaggttgatgaaaatatgaaagttgcacagaaacgagatgctgtcttgcggggaatgttttatttcaggaaagatatttgcaaaggtattacattctcttggatttcgagggtcacttcaggttacaacaggctgctcagcctctgccatgtgggcacaccaaggtcctactcatttctcttcaggattgtgtttatgctggtcactaaattgtcaacttgcttaaaaaagacacttctgttaacttgtcttaattttttcccccaaggcgaactaatgacagttgccaggtggatgagagagtttattgcaaaccatcctgactacaagc

SEQ ID NO: 58 - DNA, 159 bp, Canis familiaris
gctttcagggaagcagttcaagtgcaaagtcaacaacaaagccctcccatcccccattgaggagatcatctccaagaccccagggcaggcccatcagcctaatgtgtatgtcctgccgccatcgcgggatgagatgagcaagaatacggtcaccctgac

SEQ ID NO: 59 - DNA, 440 bp, Canis familiaris
tattccaccgaactgagtgtcaccatcgccgtgggggcctccctgcttttcctcaacattttggcatttgcggctctgtactacaagaaggacaagagacgccacgagacgcacaggcgccccagcccccagagaaacacgaccaacgatattgctcacatccagaacgaggagatcatgtcgctgcagatgaagcagctggagcacgaccatgagtgtgaatccctgcaggctcacgacacgctgaggctcacctgtcctccagactacacccttacgctgcgccggtctccggatgacatcccgctcatgacaccaaacaccatcaccatgattccaaacacactgacggggatgcagcctttgcacacgttcaacactttcagcggaggacaaaacagtacaaatctcccccacggacactcgaccactagggtata

SEQ ID NO: 60 - DNA, 259 bp, Canis familiaris
cattctgtcgctcccggttttggtggtggttctgtcgatggttttggaaggtccagcccccgcccaggcggccggagaaatctccagcacttttgagcgcatcccggataagctgaaggagtttggtaacaccctggaagacaaggcccgggcagccattgagagcatcaagaagagcgacattcctgcaaagacccgaaactggttttctgaggctttcaagaaagtgaaggagcatctcaaaactgccttctcctga

SEQ ID NO: 61 - DNA, 117 bp, Canis familiaris
tgcacaagagaaatggatccaatctgtgcaaccaatggccaaacttattccaatacatgcgttttctgcagtgaacaattagaaggttctaacaaatttaaattgtctcattttgga

SEQ ID NO: 62 - DNA, 517 bp, Canis familiaris
ggacggagactgtgcgttcctgtaccactgagtcatgcaactttgtgctcgccatggtggaccccacccagacggtggagcagagactcaagttgttccagattgcatccgagaagcatcaacatctgtaccgcctcgccatgactggctctgggatcgaccgccacctcttctgtctttatgtggtttccaaatacctggccgtcgattcccccttcctgaaggaagtcttatcagagccttggagattatcgaccagccaaactcctcagcagcaggtggagctgtttgatttggagaggaatccggaatatgtgtccagcggagggggctttgggccggttgctgacgatggttatggtgtatcgtacatccttgtgggagagaacctcatcaatttccatatatcttctaagttctccagccctgagaccgactcgcatcgcttcgggaagtacctgaaacaggccatgagtgacatcatcagtttatttgggttcagctccaactccaaaaa

SEQ ID NO: 63 - DNA, 570 bp, Canis familiaris
ctgggcctcagtcagtgctcttcctcctagacaccccctatatccacccgactcccccccccgcccagcttgagctcatcggacacagtatctttgatttcatccatccctgcgaccaagaggagctccaggatgcgctgactccgcggcagagcctatccaaaaagaagccggaggtccccaccgagcgctgcttctccctgcgcatgaagagtactctcaccaaccatgggcgcaccctcaacctcaaggcagccacctggaaggtgctgcactgctctggacacatgaggtacaagccccctgcacaggcttccccagcggggagccccaacttggagcctcctttgcaatgcctggtgctcatcttcctctacgaccacctttgtgtcactcttaccctccaaacctatttctacgatggtgctactctgggtctcccacacaaaaaggcctccctctctcagctcaattcaccccctttttggaaggcacttctcactctgttttgtcatgggatggcctatccagattggtgctatgggggaagagggtctgggccctctctcc

SEQ ID NO: 64 - DNA, 383 bp, Canis familiaris
ttatgatcgctgctgtcagcctgggcttctttggttccatttttgccctgtttggaatgaagtgtaccaaagtcggaggctcagataaagccaaagctaaaattgcttgtttggctgggattgtattcatactgtcagggttatgttcaatgactggctgttccctgtatgcaaacaaaatcacaacggagttctttgatcctctctatgttgagcagaagtatgaattaggagctgctttgtttattggatgggcaggagcttcactctgcataattggtggtgtcatattctgcttttcaatatctgacaacaacaaaccccccaggatgggatatgcatacaatggtgccacatccgttctgtcttctcggacaaagtat

SEQ ID NO: 65 - DNA, 330 bp, Canis familiaris
aaccgagagaggttgcccctgattatctttgccaactggagagggttctctggtggcatgaaagacatgtatgaccagatgctgaagtttggagcctacattgtagacagtctcagacagtacaaacagcccgtcctgatctacatcccaccctatgcagagctccgggggggctcctgggtggtcgtggactcctccatcaaccccttgtgcatagaaacgtatgcagacaaggagagcagggcaaatattctggagccagagggcacagtggagattaagtaccgaaagaaagatctgataaagactatgagaaggatcgacccagtt

SEQ ID NO: 66 - DNA, 371 bp, Canis familiaris
  misc_feature (321)..(322): n is a, c, g, or t
aagagaaaccatgttgacccgcctaccgcaagccttgggggtttgctttaaaaaaagaattctgggcatttctgcagggctgctggcctcccactcactcttgtctcaataaaaggctcaggagtgtccccttccaaacagaaatagcttcctctccattgctgggaagtttgtgctagtgtctcttgaaacaacatgttttgctgcatcactgaatcaaagtgaacccaaagactccagtttccttttgctttctggtgcctggtgtgcgggatacaggtgttcttttaggcttgttttgtatcagatcatggaatctgnnttttttttttttttttacncnnanaccaactcttgagggaaccatcttc

SEQ ID NO: 67 - DNA, 420 bp, Canis familiaris
  misc_feature (232)..(232): n is a, c, g, or t
agattttgctgtcagcctcggacccaagttgcagtggtatctgaagttaaagtcctggtgggccacaaactatgtgagcgactggtgggaagagtacatctacctgcggggacgagggccactgatggtgaacagcaactactatgccatggacttgctgtacgtcttacccacttcgattcaggcagcgagagcgggcaacgccatccatgccattctgctctatagacgnannctggaccgngangagatcaagccgattcttcttttgggatccacagttcctctgtgctctgctcagtgggagcgaatgtttaatacctcgcggatcccaggagaggagacggacaccatccagcacatcagagatagcaagcacatcgtcgtgttccacaaagggcggtacttcaaggtctggct

SEQ ID NO: 68 - DNA, 174 bp, Canis familiaris
gaccactcccggtggaacgaagcagaagtgactattgatgtggtgagatcgaaccaccagctaagtccagctcatttcagtttgtgtcaactttcaatatctaattcagggtcccttgagataatcctaacaatttgggctgtctgttaggttttacatctgattgaactttta

SEQ ID NO: 69 - DNA, 233 bp, Canis familiaris
ccagcaaccagcagataagaaaggaaaaaagtgtcttcgtaccacaaagtcactcaaagccatccacctgcagttcaagaactgcactagcctgcacacttataagcccaggttctgtggggtctgtagtgatggccgatgctgcactccccacaacaccaaaaccatccaggtggagttccagtgttccccaggtcagatcatcaagaaaccagtgatggtcattgggacct

SEQ ID NO: 70 - DNA, 109 bp, Canis familiaris
agttcactgtcaccccagacggtatcgacctgcggctgagccatgaagctcttaagcaaatctatctgtctggactacattcctggaagaagaagttcatcagattcaa

SEQ ID NO: 71 - DNA, 208 bp, Canis familiaris
  misc_feature (26)..(26): n is a, c, g, or t
gaggcatcgtgggtgacccgcatttncggcccctgctggctttggcgcgtgtgacagagganggnncggcnccggcggggggcgaggcctgcggggtcgggcttccttgcagggacgcccccntcctgcggtcggaaacaggaacctggtggtggcggggacaggcttctgactctgcttgtacaaatcaatctgcttattgaagaaa

SEQ ID NO: 72 - DNA, 432 bp, Canis familiaris
  misc_feature (44)..(44): n is a, c, g, or t
gattcaagtgttacaaggtctagcttgaactactttgtggtaanttatgtgacagctctctattctatcaangcgtaangtaatgaggaangagatctttcttttttaatgaaaacattaatgtgttaataccctgtatggataatgtttgacatttagtggttaccaaaatatattagtatgttgttgaacatacatatgccttagaaatgcacgtttctgtcttctgtgggatgatgatctaattcgatatagcatatgctttttggcagtgagtttgttgccagatttcaaatagggagttaagtgatgccttcacaatccaaagatttttagttaaaaatcatgtagtctaatatgccacatttatttttttatatttttcaatgcatttaattatctcttataattctttgtactaactcagcatgt

SEQ ID NO: 73 - DNA, 504 bp, Canis familiaris
  misc_feature (32)..(32): n is a, c, g, or t
ttcggattcacatcagcctcagtcatctctgngtatccgcggtccctgtgggtttcctgtaaaaagacgtgtcctgggagctacctatcaccgtcacacttcatacggataaccggttttgtccccacgngnccccgncnccgccgccgttggggtgggcggtgggatgtgctgtgctcccgtcacagtttgtgggcctggaatggtggtgatcgagatagacccttccgccacctcattacaaagaaacaagacacgtgtggtgtcttgttagacccacaggcacagaaaagaaaaggngaactacttatccccttgntccaccgggacccacccggatgccatgcccatgccggagggtccaggccccgagggacgggcgtactcaactttgctgtgaactcaacggattagtggagttgtgcctctgaggatttgtccacggaaactacttcaaatgatcgattttgtncngtctcgtgtacttttccttgtgacgagcaa

SEQ ID NO: 74 - DNA, 392 bp, Canis familiaris
actgggagctcgtacccgacggccaccatcagctacctcccacacgcgccacccttcgctggcggccacccggcgcaactgggcctgggccgaggagcctccaccttcaaggaggaaccgcagaccgtgcctgaggcgcgcagccgcgacgccacgccaccggtgtcccccatcaatatggaagaccaggagcgcatcaaagtggagcgcaagaggctgcggaaccggctggcggccaccaagtgccggaagcggaagctggagcgcatcgcgcgcctggaggacaaggtgaagacactcaaggccgagaacgcggggctgtcgagcactgctgggctcctccgggagcaggtggcccagctcaaacagaaggtcatgacccacgtcagcaa

SEQ ID NO: 75 - DNA, 184 bp, Canis familiaris
cctactggtcctcaggtaataaatcccaggacaacaagatcccctccctttaaaactacctacttcgttttctcttcttcagcctgtgggacatctcacgtttgtggattccttgcatctatgctctgatttcatgtatctaacaaatccttacttagtgcttagaagcgctggacactatttt

SEQ ID NO: 76 - DNA, 145 bp, Canis familiaris
ggatctgtaggcctttctgtccagcgctctgtttttggagaaagaaatttcaatatacacagctccatttcacatgagagccctgcagtgaaacttatgaagcagtcaaagactatggatgtactatctagaaagttagcgacaa

SEQ ID NO: 77 - DNA, 490 bp, Canis familiaris
cttatggctcggagataacccttggagatgtgactacaatatccactacctctactactggctaaagcaccactacaacgtccactataatggcctggaatgtaaaacgcctgaagaatacaaagggtggtttctgggaaaatatatccgcacttactttgaagaatgccccaaagacaaattaccggcataccctgaaacatttgaccaggatacggaagatgacgagtgggaaaaaatacacagagatcatacaacaaaaaagcaacgtgtaagaatcactatagtgggataacttagaaattgttctgagtgcaactagttttgtatttgctatactggtgtcagaaagccgtacgtttacattgtgattaactgtgttgcctatttatgcagggttttctggctaaaggaaggtggaaggttcccttattagtattattattgtgaaaattctagcactcaagggaaaactttcatcctgcttgcc

SEQ ID NO: 78 - DNA, 207 bp, Canis familiaris
tggatttatgagtgacgagtgatgagggagcagacctcagtgctttgcccgttccattccctttccctcccatgagcaaggggccaggctcttgcaaatccagtgatttttgagaacttccttataacctggaggggacctcttggagctgggagtgcttcccgtacagtattatgggctgccatcagattaaaatgtttcatccca

SEQ ID NO: 79 - DNA, 41 bp, Canis familiaris
tggtgatagcctgaaagctttcctcactagaaaccaaatgg

SEQ ID NO: 80 - DNA, 197 bp, Canis familiaris
ttgtctgcttttgtgaagctgggcagagattatgtaccggctttatctgtgcttggctatgactctacctccaggtttctcagaccccatagaatgtgtaagagaaaagtaccaacagggaaatcagcaaaaagctttcctatgaaggtgtagccagccaccgcagaatttgaaatgtctgaggtctcatctgatga

SEQ ID NO: 81 - DNA, 367 bp, Canis familiaris
  misc_feature (47)..(47): n is a, c, g, or t
gtaagtaatttattgcctgatggagatttatgggtgaaatccgaggngttttcaccgtcnggtgaagtgattnntatatcatnccttgtctatgacagttgtattcttggactgnaaattctagctcgcttccatagccttgattcctgggtatgttagacactncatcaccggcatatttaaatcatctcacaaaggactcgattgtaggacaaggacttggctgtgctcgcacgtgtncacagaaagaggtgaaaatgacatatttttatggagaataggatctctgactctattagcaatgnaaatatgttcctgctggacagtgatcncaattgttgtaactctgtagtattttttcatatga

SEQ ID NO: 82 - DNA, 434 bp, Canis familiaris
gcaactgacccatctactcaagaagacattatggatatccatgtgtgtaattactcatgtgcaactttcagaagcattttttgataaatgtattctaatgtgtttcttttccaggatattaaagatttgaccttctctttgtcttcaaatccattatttttatgtccatttaaattaactctccagtcacatttactatatcttttgtttgccaggcactgatgctttatagtaagcacttatatttttttctattaaaattttgctaaaaaacagaaagggtgcatacatgtaaaaataagccatttcaaaaagctggtaagaaaacttgtaaatattacctgaaattatccacaagagaaaaacttgacaattagtgaccacaactcttgtatcagctcacaaattttagaccaacatgaaggctgcggaat

SEQ ID NO: 83 - DNA, 334 bp, Canis familiaris
  misc_feature (146)..(146): n is a, c, g, or t
tttcaccaatagttaaggccagagccaaatagccatgcagagcgagacgcctccatccagcactgagtcatcttccttttttgtcctccccctctcccactgctgcacctgggatcactggatgggagaatagggcgcccctcatnactgagggcagaagttgctagaagtcagagcaggatgcctgggtttccccctgcctccaccagcccctaccatttccccatgatatggtagcttctccttgggctttccttgcccctgtctcctgggatcagagagtaggatactagacatgactcatgtcttgggtacataccacatagcaaataaa

SEQ ID NO: 84 - DNA, 305 bp, Canis familiaris
  misc_feature (39)..(39): n is a, c, g, or t
tggcccctcctgtggaattggatattcttcagtttcatnctgctgctgaggaggaagggagcaagccgcagacactgtaacggtcctccggatgtttgtgcgtgtgtatgcaaaccttcncactgtgtgtgttgtgttcaatgttgtgtcaatctatgaactgactcaaaccaacaattttgaagatacagaagacaagaataatggcaaaagacacccatccacctctttttgtcagagtgcttgctatgaccttcatagctaggacaaataccataacatattcaggagcaaggtgtccttga

SEQ ID NO: 85 - DNA, 73 bp, Canis familiaris
tggatttatgagtgacgagtgatgagggagcagacctcttggagctgggagtgcttcccgtacagtattatgg

SEQ ID NO: 86 - DNA, 299 bp, Canis familiaris
  misc_feature (88)..(88): n is a, c, g, or t
cttgggacatgtggagagcctactctgacatgagagaagccaactacaaaaattcagacaaatacttccatgcccgggggaactatgncgctgcacagaggggccctgggggcgcctgggctgctaaagtgatcagcgacgccagagagaattctcagagaatcacagaccttttaaggtttggagacagcggccacggagcggaggactcgaaggctgaccaggctgccaacgaatggggccggagtggcaaagaccccaaccacttccgacctgctggcctgcctagcaaatactga

SEQ ID NO: 87 - DNA, 351 bp, Canis familiaris
gggacatttcctggacttgagttctgggtggtctgctcctaccaggtgtatgggcttggggaagttccttagaatttctgtgcctcatttgcttcctgtgaaaagatacccagtataattgcctacttcatacggtttgtgtaaggcctaaaaaccctttaggatatgtaaactgtcccaaataatgcatagtagttgcacagtaaaacttcaaaacactaatatcacgattatttaaaaagggcttatgtcattaaaaaaacagtttgcactttccaaattgatttagtgaaaacttgtttttattgaaaatgacaacaaaaaatgtggtatatttacccctatcaaata

SEQ ID NO: 88 - DNA, 525 bp, Canis familiaris
  misc_feature (40)..(40): n is a, c, g, or t
tatgagctggactgcagtcttgcctaggcgattttgtctnccgtttatgtttnnaaaagnccaaggtgctgatgtcaaagtgtaacagatattggtgttnccttgtgtcctctnccctgccaaatctcctaagaggttggacttccatgcctgcagctttcctagctcctccacccctatggccccaggcccacagagtgggaactccttttttctcatgtcaagacattttttcttactcctgccattcttctggtgctactccatgcaggggtcagtgcagcagaggaggatctagagaaggtatcagcaaagaagaaggctgaggaggagcaggaaacattggagctgcctgttcttgttaattgataacctaccgattactacacagtttggggtcggggggcttctgtgtaaacccatccacctcacttaaactaaaaaggtatgctgtcatggtcccttctggaagttgctggtgccatttctgaactgttacaacctgtatttccaaacctggttcat

SEQ ID NO: 89 - DNA, 387 bp, Canis familiaris
  misc_feature (35)..(35): n is a, c, g, or t
agaggagctcctgtcatccattgtacttgctcccntgagccactgtccagaagactaaggcnaagttacacgcgtgtgncccacgtctgcctacaacattggcccttaggtgatttggatacacaaggcctctcttctccatggtcatttgcaaacctcccaggccctctcatcagatgaagaagcagaggggnaaaaactctgcaganggtggccgagttccagatacacagcccatgtgggcatcaccgctgcctcccacctcctctgtgtcctcagaaacgctgtgtgacgctcccttaacaacaacggacttccctgtgaatgtctcttatgctagtttggggcctctgccctttgatttgtacagtggcaactcccaacagg

SEQ ID NO: 90 - DNA, 489 bp, Canis familiaris
  misc_feature (142)..(142): n is a, c, g, or t
gtctaaagtgaccagttccgggctggagagaattgaagatcatatcaaggacagaaggaatgctccagttatggctccctgggtgaacaacctggcccttttgagtgactccacaggcccaagagaagagcaaatgactcanaatggattaacatggaagttatccaggctcagggttaaaggcacataagcaagaagagacagtagaaagaaggagatactgtggtactgtcattaaaaaaaaaaaaaaaaaaaaagtggacatgtcagaaaatccctcagtactgagaaggccaggtaactccagttacacagaaactgtgaataaaggttttgaaaccgattaaaacatgctataagctttaaggtcaactgatatttatgctacatattattatatagagattgcattgaactgctgagatctctaactgggatagctatttggaaaatatgtgtttgcaaccaccgttgctattattgatgctt

SEQ ID NO: 91 - DNA, 252 bp, Canis familiaris
tcctggagagtgagcttgatgagaaagagaatctcctagaatctgttcagcgactgaaggatgaagccagagatttgcggcaggagttggctgtgcagcagaagcagaagccccggacccccatgcctagctcggtggaagccgaaaggacagacacagctgtgcaggccactggctctgtaccatccactcccatagctcatcggggacccagcactagcttaaacacaccagggacgttcagacgcggtc

SEQ ID NO: 92 - DNA, 140 bp, Canis familiaris
tttcgttatccgcacctgatgccttaaaaatcatcacagaaaatgaccacgttcttttgttttggaaaagtctggctctgaaggagaagcattttaatgaaagcaggggctacgagatacacatgtttgatagtgccatg

SEQ ID NO: 93 - DNA, 305 bp, Canis familiaris
  misc_feature (175)..(175): n is a, c, g, or t
cttactatgggaccagtcttggaagggccttctgttggaaatttgactagtagtttactgctggatagaaatgttactccaagagaaaatgtaatgattcctacctcaaggccgcagacttcagttagcaacattgaaactcacttgttcaagatgcagcagatgttggaagacngtataatcagagaactaaaagaagttgctcatgattnagaatcaggattctgtagatcttctcctttaggacctactgacgaatcaaatgtaaatcaagatcatagtactgcaagcatcacgagaatatg

SEQ ID NO: 94 - DNA, 299 bp, Canis familiaris
  misc_feature (140)..(140): n is a, c, g, or t
aaaggtttttcagacaatttcacagaaaggtgcattgacttctctctgttgagagcatttcagacagaggacttggaactgtgaatatacttcccaaaggggagggagaagggaggaagctcccatgttgtttaaaggcnataatcggagcagcttttggctgctcaactgtgaactatggccatctataatttttttttttttttgttaatttttgaagacacttgtggctggaaaagtgcattccttgttaataaactttttatttattacagccaaaagaccagtatttattatca

SEQ ID NO: 95 - DNA, 466 bp, Canis familiaris
atgggcaaacttcctatgcgtccagcaataaactcaccaactacgtgtggtattttgaaggaatctgtttctcaggtgtcatgatgcttatagtagcagcagttcttcttgtactggagaatgatagcaactataaatgttgccagagtgaaaactgcagcaaaaaatacatgacactgctgtcgatgatcttttccgcccttggaattgctttctctggatactgtttggtcatatctgctctgggcctcgttcagggtccatactgccgcaccctcgatggctgggagtatgcctttgaaggcacttcaggacgtttccttacagactccagcagatggactgagtgcctggaacctgcacatgtagtggagtggaacatcattttattttccattctcatagctctcagtgggcttcaagtaatcgtttgcctcatcagagtagtcgctcagttatccaagat

SEQ ID NO: 96 - DNA, 481 bp, Canis familiaris
  misc_feature (313)..(313): n is a, c, g, or t
atgaaccactttactacaatccctcttgctgtgttgaatatgcctgctcttgaatggctcgacatgggaagcaacaaacttgaacaacttcccgatactatagaaagaatgcaaagcctacatacattatggctgcaacggaatgaaataacatgcttgccagaaacaatcagcaatatgaaaaatctgggtactcttgttctcagcaacaataaactgcaagatattccagtgtgcatggaagagatgaacaatctgaggtttgtcaacttcagagacaatccactgaaattggaagtaacgcttccccccngtgaaagcctggatgaagaagaggaacgggaattatttggtcttcagtttatgcacgcatatatacaggagtcacggatgaaagcagataaccaagtcaactgttccactactttaccaatctccataaatactgatggataatttaactcaagatgccctgctgaag

SEQ ID NO: 97 - DNA, 289 bp, Canis familiaris
  misc_feature (31)..(31): n is a, c, g, or t
cactgtgtgtctgggtataatctgcagattnctccatctagaccagcagtctgctcttgattactctctgctatatatataacggtgctgtgaggggggcatttttcaatatatacaacttttgtactgaatttttttgtaataaacaatcaaggctacaaaaattttttttaatagaaaagagaaattttgtaagaaggcaatattaacctaatcatcatgtaagcactcttggatgaaggattccacaaaactttgttttatggttatttcttctcttagattctta

SEQ ID NO: 98 - DNA, 449 bp, Canis familiaris
atattttgctgctgaatttcgggctacttctattctgcagaaaattcatcgagatacctactcaagaagatagttgtaaagatattgtatctcctttaatatactccttaaaatgtatgttggtttagcgttgttttgtggataagaaaaatgcttgaccttgaaatattttctacttaaaaattgtggatgaaaaccctatctcccacaaagttcccatctccttgtctaagggttttttaaagtgttctgtggctgatttactaacagtaactgccatttcgtgtctgtggtaacagagtgatttgtaaaacagtggatgttctcattgtgttctcttcgtggactgtttttcctgtgggtcatactcattccttctgaagaagttgtgctaacaccagcaacagtataatggccctgtagccctgaactcttctttaacctaactg

SEQ ID NO: 99 - DNA, 448 bp, Canis familiaris
atgagctgacccaagcatgctttgcctctgcaggtccgagagcaatacaagaaatggttcaaaggtgtcaggaaaaccaaagctgagtctgagcagtacactctctccagtgggaccatgtccaatgcctccaaacacagtgtggagaattataattaacagctaaacctgatgaacttgtctcttagaaaatgaagaaaatactggctgccatcctttggacaaagaatataaaaaatgcttttttcatgtttgttacaaataactcttcttggcaccactgtgggagaaagtatgcaggaaatatttgacattcagttgctagaaacttggattttctggtgcatacaacactctcatcttactcccagtataatcaagaactattaccgaatatctcattagaagcctcttctacaaaaattcttcctcgggttgatgatatata

SEQ ID NO: 100 - DNA, 373 bp, Canis familiaris
ggatccctgaactactcagttctgaacacaaaggtgctatttaccaaacactgtccccactgattcctgccagctctgcctccttgtcaactctagactcccgagccatcttcccagaccctgcttcaacatttcttgctttagaggtaactttaggccgacagacccaagagctccttttctggcctgtggatggaaacgaagctgtgaatctgcacattgtggtgtggcatctagtctgcaaacaggtccccgcctttctaaaagcagcctcacagtctcatgcttcatctctcttttttgatgttcactttaaaaacaaaacacccagagctggactgttgagcaggcctgtctctcctattaagtaaaa

SEQ ID NO: 101 - DNA, 390 bp, Canis familiaris
tcagcaactttctgaagtcgaaagtaaatttaatgccctagaaattgagctccagtatacaagagatgctctcagagaaaagacgttggctttagaacatttacaaagaaacccaaaacaaacacagggtcagaagaaggaaattgagcagaaatctcaaaacgaacaaggtaaagtgaatgaatacatgggaaagcatgaatccttagaagagaaattatctcaactacaaagtgaaaatatgttgctccgagagcaactagatgatgcctacagcagagtggaatgtaaagaaaagactataattgatctcaaagaccagattcaggttattgtaagaatggttcgagttgcaggtgaaaaacgagctcttcaactgcgagaaagaaa

SEQ ID NO: 102 - DNA, 490 bp, Canis familiaris
aggccgtgcgtgtttttggaaatctctcccaggaccgtgacatctgcaatttcattgtgcagaaaaatgtacacaagttcatgattgcgctgctggatgctaagcatcaggatatttgcttttctgcctgtggcgttctcctaaatctcactgtggataaagacaagcgtgtcatcctaaaagaaggaggtggcattaaaaagttggtggattgtttacgacattttggtcctaccgattggcagctggcctgcttggtctgcaagactttatggaacttcagtgaaaatatcaccaatgcttcatcatgttttggagatgaagacaccaacaccctattagtcctgctgccatcatttttagatgaagaactagcactggatggcagttttgaccaagacctaaaaaactatcacaaactccactgggaaacagaattcaaacctgtggcacagcagcttctaaaccgaattcagagtcatcacacctt

SEQ ID NO: 103 - DNA, 492 bp, Canis familiaris
  misc_feature (104)..(104): n is a, c, g, or t
ccggactcttagcagctgctcaagagagcaagccccggccttcagtgtgctgcagctggggtagagaagctacctgcagacaggctgagtgccagccctgtccncccaatgagcctatagccaagcctctcaacagtcagggagaaaacccctgtcagtgtacccacagtggacataggaagtcattgcagccaattaggccaaaagcagctcagccttaacaggaggaaacattcagtccacgcaggggccactttagatacctagttctggggaccaggggggattgcactactgggcatcacaagacctctgttagacaaggccactacttccaggaccaggagatgtagctgacctacttagtgcatagaaacaaatgcagagagttagacaaaatgagacaaaggaatatgtctcaaatttaaaaacaagacaaagtcacgggatatgagctaaatgaaacagaggtggcgttcaaagttactgtca

SEQ ID NO: 104 - DNA, 565 bp, Canis familiaris
gcaccggagtctgcagttgtcgctggtcatgcgcagcgcccgggagctctacctctcggccaaggtggaagcccacgagcccgaggtgcccttgccgcccgtccgctcccccgatcctcgcctgcaccagtcgcgggaagcggaagctggagccgaggcagcgccccccgacggtgagccgacctccccggagcccatggacacgcgggaggcgccgcgagcggaggagaccccagcccgctgtgccccgcgccccacaaaagtcagccgaaagcggcgcaacagcagcctgagtgacggcggggacgtcggactggtcccaagcaagaaagcccgtctggaggaggaggaggaggaaggagcctcctcggaggtccctgatcgcctgcagccacctcccgcgcaagcggagggcgccttccccaacctggcgcgcgtcctacagaggcgcttctctggcctcctgaactgcagccccgccgcccccccgacggcgccgccggcgtgcgaggcgaagccggcctgccgccccgcggacagcatgctgaacgtgctcgtgcgggcc

SEQ ID NO: 105 - DNA, 502 bp, Canis familiaris
acattggagtgcctgagccatgcatcttcttggtctccaactttgacctggacgactttgatttcccaagactggaggaaaccctgctgaaggagctccctgttcataagcgccacatctttgcactcctgttgcccaatttatcttacacttccattgaaatgaagagagctttcttcaaggaaaagatctggctggatgccctgaaatcatcagctttgagcttcatccccttcatggcctgctttaatggctttgattttccccagcaggaaaagtgcttgaacctttaccagagccattttggtttggatgagaagtcggtcaaagggattgcagagaagctggacatgtctgtggaggagatcaagagtttcaccaagtccttggatttctggttacttgtgaaggatgacagcatagcagaaaaagccatgaagtgtgttgaatgctattgctcagtaaatggaggcctgccatccactatcttccagttctttaa

SEQ ID NO: 106 - DNA, 491 bp, Canis familiaris
  misc_feature (67)..(67): n is a, c, g, or t
accccaaggccatcatttggatctattccctgttcttcccactcatccttgtctttctcccccatgntttctttttattcctgcttgtccccacagccccatcccagggccagccctttctttattcctcttcactattactccttctccccagcctctgccccagaactgccactggcagccaggggtgcctcaggaccggagcctgcttggcccatcttgcagagtttaagctaatggaggacaaaaagaacgataaagaagaggcttcctaaaccagcctaggcttggggaggaacccccaggnccaagatctgaacagatccagtcaagctcagccaccattagaggaggacttctccccactgctcattgtatttgcttttttagttcttggggaaaggaggtgggggtggggtggggtatgtagaaaagaggcagtcatcttttctagaagagtataaatggtccatgctgtatagtatttgtca

SEQ ID NO: 107 - DNA, 549 bp, Canis familiaris
aatgacttctgcaaaacgtcctaagataaaacaagtcaccacaagcattcatcaccatcctaggcaagaggtacttaagacctaagcccccagatgtcagccaacaaaagaccatcagcacatggacattaacctgataagtagatacacaaccaaagccctgattgtcccaacttagcacacccccattgtttaagatagttctgctaaagagctcataaaatcccctaaacttagaaaccccacgggcaaccccctcaggtcctcttcccctttgagagctttgtgctattgctcaataaattttgctttgttggccatcactctttggtctacttcttcattcttctaagcggagtgacccagaccacaggcactagagtaatagaaatcctgcaacagttaagtcttctttaacatatgaaaatccttttggaaacttcctttgtccttacccctcccaacttaaaagtatacaaccaatcaccactcctcacattcccagtacagttctttctgcccattggtcttgtccccatgctttaataa

SEQ ID NO: 108 - DNA, 161 bp, Canis familiaris
atggaggtctccacggctgccctggctgtcctgcttctcatcgctgcgctctgttcccagacctgctcttccactttgggttcagatcctcctactgcctgctgcttctcttacaccctgcggaagattcctcgcaactttgtggcagattactttgagac

SEQ ID NO: 109 - DNA, 186 bp, Canis familiaris
gagagtaaagtaagtcttcccaggaagagtgataacccaagtgagtgttacaggaagcagagggagggcataccaggcaaggcagctgcgaagccaaggcatggaagaggtgtcagtgaaaaaaaaggtccagaatgattactaacctatgactcccaacagtatgacagaaaatggccttccagc

SEQ ID NO: 110 - DNA, 198 bp, Canis familiaris
  misc_feature (94)..(97): n is a, c, g, or t
aggcaaatgcagtgcagaatcatttcactgtgggcctcggtttgtttattcagaactgccagtttccctatttaaatgttggttggggtttttnnnnctcctactcatccccgaatgagggagactcttgtttcttttatgtgtctctcttaaaagaagggtgtgaggggaaaataatatttcagattcctcaagaat

SEQ ID NO: 111 - DNA, 90 bp, Canis familiaris
attcttggagactaagtaccgtggactcaatcggttggccctcctgtggattggtgggttttacagggtgaagaaggttcactaaagcgc

SEQ ID NO: 112 - DNA, 510 bp, Canis familiaris
  misc_feature (394)..(394): n is a, c, g, or t
aatgacttcctacccttgtagtggactcatttttttgtgggggtttgttttgtttgttttaaaaatattcctaacacccgcctagtaggagaaccttctaaatgagcaacccaaataatggttcttgatagaggttcagtattttctccattttgttatttaggagattttagttattgattgttgttttgttttaagccatagctttaaaagaattcagaggagaatgttcataactaacttggaatttgtaacctcagctctgggagaggatttttcctgaacgactattatctaatggaagtatgttgttgctttagggtcatggcatgcttgttcttctgttaccatatttccatagtattacgtacaagtgcccttaggggcccatacntttgggatggtatgagtaaatgtgcagtgtagcccacacttgagagtatgaatgtatacgcactgtcactttgctctgggtggaagttgtttattggtaacttgtgtcccctatgc

SEQ ID NO: 113 - DNA, 98 bp, Canis familiaris
aggaaggattagacccagactctgtactctccactcttcacctccttccttttttctatgcctggtgggagctggacagtttcatctcttgcatgtaa

SEQ ID NO: 114 - DNA, 412 bp, Canis familiaris
  misc_feature (365)..(365): n is a, c, g, or t
tgaatggaccctatgggactcctcttttccactgtgagatctacggggaagccaatcagatgatctacagcatgttgaaaaagatggacaggtggtggtacgaaaacaattcctctatggcctgcaggactggatggaaccagaccaggctgaggcatacagttcttaactttggacaatctgagagtgaaccagaatgcagtttttcttgacgtacctgttttaaaaggtttttcagacaatttcacagaaaggtgcattgacttctctctgttgagagcatttcagacagaggacttggaactgtgaatatacttcccaaaggggagggagaagggaggaagctcccatgttgtttaaaggcnataatcggagcagcttttggctgctcaactgtgaactatggccatct

SEQ ID NO: 115 - DNA, 317 bp, Canis familiaris
  misc_feature (26)..(26): n is a, c, g, or t
cctttagagcagtaacttcagggctnaangggcggncggttccnctccattcagtagctccaaccctgctgttnccagagcgtttgcttctcgtcagaagcagaagcagaagcagangcagaagcaggagagcgcggcggctcccgagagctgtgcgtggtgctggctgcctgcgggaaggtgggcanccgaatcaccagcgaccgctcggtttgcgaggatttcctctgggctttngcggtagcaancagagcagtgcgtctccgnagttaacaccccacccggaaagcattttctatgcactctactgtagaatt

SEQ ID NO: 116 - DNA, 454 bp, Canis familiaris
gttttgaataggtacagctgtgacttgggtctgactttccttctttctgtttggtttgggccaactgattttcaatttcgtggtaaggttgccgtaacgtgcaaatggcttcatttttcgatgtggcccaaaatgtggtgggtcacaatccttcgttgagataaagctggctgttattcgacgtttctcagctccagcctgagacttcagagcctaagtcttataataattcacctgtcattttatgcccccgttgggaacttacaacagtagcagcatgtactgatttccgggtagagtacttccatttatcaaaagcacattaaccaccatagcatgattcttcgaataaagggcaaaacagattttataattgacctgagtactttaagcttttgtttaaaacattttttacttaattttgcaaattaaaccattgtagcttacctgtaat

SEQ ID NO: 117 - DNA, 371 bp, Canis familiaris
aaatcttgatcatgcttctgtctctcgttacatacagcactagatttgatggctgcctgtattatgccacaagatgagtgtccatgcttccctatgatatttgaactggttctatgcttggatttgcctaagcagaatcatgatttacatttctcaagattgagttagtaccagttggtgttggaaatctcatactaactgattcctatactatgtcaccaccaagcccaatcagaccatgaccaatcagcttgaaatatatacttccatgttctacaaacccacacctcgatttgtcaagccatttcttgtttttgtaggtatctcctccttttcctgtttatatatggaatctctctagtctgtgaaac

SEQ ID NO: 118 - DNA, 335 bp, Canis familiaris
  misc_feature (100)..(100): n is a, c, g, or t
gaagcactcaggcagtgctcaggactctcagatgtctgtcatggctcctaatgagcaagccagctgccttgtgggactctgagtgatgcaagatttgttnacactcccacntttttgacttcaagagccccggatatctccatctgctaatggtgtctgaaagtgactctgttcctattagcataatgtgagcaggtggggaaattggcccacctctactcatcatgtcccttcccccaactgacctgtgttctttccctaatctgctttcctcttccagcagagatgggactgggtcattaccatccttgtctttacttcatatttggctggga

SEQ ID NO: 119 - DNA, 321 bp, Canis familiaris
tctccagactctgcggatcaatgtgtgttactggccagagaaggttcctggaagctatggcgtgtcccaggcctttccctgcttcctggtcctggacctcagcctgactccccaccacatctatgggctgccctccggagagtatcctgggttgatgaaggtctgctatcaccacggcaacaatgcagatcctgaggagcgggactgcccggaagccttctcagacatccaagacgtccagatcctgagccgcttcatcagagatcacttacccgacctagagcccaagcctgccattctggagcactgcctgtacacgaa

SEQ ID NO: 120 - DNA, 501 bp, Canis familiaris
  misc_feature (191)..(191): n is a, c, g, or t
gatgtattgggaccttctaccactccacctgtgcattctagcagtaacacaattagaagtggaatttcttctaactctaatgtggttgatggtagactgacttacccaagttttgggattggttatcctatctcctctgcatatgcattccagaataactttcctcatcctgtatctgccaaaaataccanccacccaatttcaggaccaaaggttcattttaacttgcagtttacaaaaccaaatacgtcactaggagatgttcagtcaggaacaaccattagtatgccttgctcaactgataaggaaaatggtgcaaatttagggctggaatccaaatacctgaagaaaagggaagatagcattcctttacgtgggctcagccaaaacctgttcaataatccagaccatcaaaaagatggcactttaggagccttacagacctcttcaaaacccgcagtgctcccttctccatcttcagcatacttccctgggcagtta

SEQ ID NO: 121 - DNA, 560 bp, Canis familiaris
atgacccaagttctcctgtggctgggccttgccttgctggggtccttgcaggtccagacccaggactccaccccaagtctgatcccagccccgcctccgctcaaggtgccactgcagcctgacttccagcatgaccagttccaggggaaatggtacgtcatcggcatagcagggaatatattaaagaaagaaggacatggccagcttaagatgtataccaccacctatgagctgaaagatgaccagagctacaatgtcacctccaccctactcaggaatgagcgctgtgactactggaacagagattttgtcccaagtttccagcctggccagttcagcctgggcgacattcagctttaccctggagtacagagctacctggtgcaagtggtggccaccaactacaaccagtatgccttggtgtacttccggaaagtctacaaaagccaggagtacttcaagatcaccctctatgggaggacgaaggagctgcccctggaactgaagaaggaattcatcaggtttgccaaatccataggcctcacggaagaccacatcat

SEQ ID NO: 122 - DNA, 544 bp, Canis familiaris
cagaactctaggtcgtcattgccatcagacttacggactatcagtctgcccagtgctgggcagtccgctgcctaccaggcctccagggtatctgcggtttccaattcacagcactacccacaccgtgggagtgggggtgtgcaccagtaccgactccagccgctgcaagggtcaggagtcaagactcagacaggactttcctagggcttctggatttgggcaaacagaactgaatgagcccatagctgcttccttccagctgcctctggaacctaggccgagcatattgctggggaagagggtacaaggtgccagaggactgggtctggtcgacaagaaacaagacttgtggtcgcgactggctctggccttggagaaagatgtaaatcttgtctgaagcagagactataaagaagtttctccctgctgttaagggtacattgttgacaagcaaatggtgttccggttagtaacggttctaagtgcaatgagtcgtgttgaagcctctgtctcccgtacttgcctgtagcctgtagtcacttgt

SEQ ID NO: 123 - DNA, 373 bp, Canis familiaris
  misc_feature (62)..(62): n is a, c, g, or t
catggagccagagttgacacagagttgacagacgggtttagaggggaggggtgaaggtgccncaaactggtttgggtagtggtggggaggaggccggggaggctgcccagaccgcccgcaccccttcctgcccccaccctgcagcctcagggctctgcctcagcagcaggacccccaggccttttgtgactgggcgacatctggatcctgccacccctgccaaggtcactgggagtcctggggacctgctgtgggggggtggggggatggtctttctcgggccttcactcccaggaagtcctgggcctctttgctgcctcctctgtagttggttttgtaacttatgtagatctgtttgtcatggtaattacca

SEQ ID NO: 124 - DNA, 209 bp, Canis familiaris
gaataggaacacgttttctggctagcagaaaaagaaaaccgttgtttatctttcttgctttatttgagagtgtacagtaaaagggatttttttcagattatttttatattattttagctttaattgtgttgtcattcatgaaacagagctgctccgcctctctgtcagagatgacaagggctttctcagcatcttgtttatgtgtggaa

SEQ ID NO: 125 - DNA, 531 bp, Canis familiaris
cagaaaatgaccacgttcttttgttttggaaaagtctggctctgaaggagaagcattttaatgaaagcaggggctacgagatacacatgtttgatagtgccatgaatatctcagcataccttgggaatactactgacaacttctttaaaatttccaacctgaaattgggtcataattatactttcactgtgcaagcaagatgcctttttggcagccagatttgtggggagcctgctgtcctgctgtatgatgagctggggtctggtgggggcgcctcggcgtttcaggccgccaggtctactgacgtcgctgccgtggtggtacccatcctgttcctgattctgctgagcctgggggtcgggttcgccatcctgtacacaaagcatcggaggctgcagagcagcttcaccgccttcgccaacagccactacagctcccggctgggctcggctatcttctcctcgggggatgacctgggagaggatgatgaagatgctcctatgataactggattttcagacgacgtcccca

SEQ ID NO: 126 - DNA, 457 bp, Canis familiaris
  misc_feature (327)..(327): n is a, c, g, or t
atctacaacctgattcaccttgatcccagaactcgggaaggtttcaccttgctgcatctagctgtcaactccaacaccccggttgatgatttccacaccaacgatgtctgcagctttccgaacgcacttgtcacaaagctgctgctggactgtggtgctgaggtgaatgctgtagacaatgaggggaacagtgcccttcacattattgtgcagtacaacaggcccatcagtgattttttgaccttgcactctatcatcattagcttggttgaagctggcgctcacactgacatgacaaataaacagaataagactccgctagacaanagtaccactggggtatctgagatactacttaaaactcagatgaagatgagtctcaagtgcctggctgcccgagcagttcgggctaatgacattaactaccaagaccagatccccagaactcttgaagagt

SEQ ID NO: 127 - DNA, 202 bp, Canis familiaris
  misc_feature (60)..(60): n is a, c, g, or t
gagaaccgggcagggtcccgcgcaagcccttgctgcggagggaaacagcaaagaggctgnaaagtcaggaaggatggcaaagtccaagccaagacccccgccccgtcccnatccatcgtgttctcgtggaaacttaaccaaaccgcgccgctgtgaccgggactgctgttctccgtgttgtgatctatcctctcaacagcaa

SEQ ID NO: 128 - DNA, 438 bp, Canis familiaris
taaaacccaatatcattccctcttattctgaattaatctattacttccgtgcaccctcaatgaaagaacttccagttttgaccaaaaaggcagaagactgcttcagagctgcagctttggctactggttgtacagtaagaccttttaagaaaattaaaggtggagcacgcgattattacaatgttcttcccaataagagcctatggaaagcttatgttgaaaatggaaaaaaactgggaatagaatttatttcagaagatgcaatgttgaatggcttttcaggatctactgattttggaaatgttacttttgtggttcctgggattcatccatatttttacattggatctgatgccttgaaccatacagaacaatatactgaagctgcaggatcacaagaagctcagttctacactttacgtactgccaaagctctgg

SEQ ID NO: 129 - DNA, 407 bp, Canis familiaris
  misc_feature (26)..(26): n is a, c, g, or t
cccggagagctgttcatccgagggtnctgcgtcatgctgggctactggggtgagcccgagaagnccaaggaagcaattggnccagacaagtggtatcggacaggagacatcgccgcgatagacaaacagggcttctgcaagatcgtgggccgctccaaggatatgatcatccggggcggggaaaacatctaccccgcagagcttgaggacttctttcacaaacacccgcaggtgctggaagtgcaggtggtgggagtgaaggacgctcggatgggggaggaaatctgtgcctgcattcgcctgaagaagggggagaagactacggaggaagagatcaaggctttctgcaaagggaagatctcccacttcaagattcctcgatacatcgtgtttgtcacagactaccc

SEQ ID NO: 130 - DNA, 273 bp, Canis familiaris
caggcctgcactagatactcgatgtgtgtgcaacccaggctttgggtaaagcccccgtatcatcggatgtgccagcttgagttggtttctgctctgctgttgttgtctttgctctgtggccgcagaaggtctcagggtcaccttctagagcgcctgtgagcttttctctctctcagacactgggaggatgcatgagacatgtctgcccattactttggattattacagatggtgctatctgaacaccaagtgctgttgccaatagagaccgac

SEQ ID NO: 131 - DNA, 219 bp, Canis familiaris
  misc_feature (134)..(173): n is a, c, g, or t
cttctagatcaaagacctaaggcagctgttctggaaactttccggtgcttggtattcctaatgttgtacagaattggtcatctgaagcctttgattccttccttgtcatttgcttttgaggtaacctgggtggnnnnnnnnnnnnnnnnnnnnnnnnnnnnnnnnnnnnnnnncnagggncctgnaatcaagttttgcatcaatgtttctgcctccctc

SEQ ID NO: 132 - DNA, 310 bp, Canis familiaris
actcctctgtggtgtggccttttgtttcctgggagtaggccttttgaacgcacaagtgactcaaaccccgagacaactcatcaaaaaagtgggagcgaaagttttgttgaaatgttcacagaatatggaccatgaaagaatgttctggtatcgacaagacccaggtctggggttgcggctgctctactggtcctataatattgacagtgttgagacaggagacatcccttatgggtacagtgtctcgaggaagaagaaggatgccttccccttgattctggagtctgctcgcatcaaccagacatctgtg

SEQ ID NO: 133 - DNA, 527 bp, Canis familiaris
gctatggtttcattacctaccgttatacctgtgatgcttttgctgctcttgaaaatggatacactttgcgcaggtcgaatgaaactgacttcgagctgtacttttgtggacgcaagcaatttttcaagtctaactatgcagacctagattcaaactcagatgactttgaccctgcttccaccaagagcaagtatgactctctggatttcgatagtttactgaaagaagctcagagaagcttgcgcaggtaacatgttccctggctgaagatgacagagggacggtgaatacctcacgggacagcgcgtccttccctaactgactattgcaagtcatacttaggaatttctcctactttacactctctgtataaaaacaaaacaaaacaacaacaatacaacaagaacaacaacaatggtttacatgaacacagctgctgaagaggcaagagacaggatggatatccagtaagcacatgtgtattcatgggtgtcagctttctttccctggagtctcttggtgatgga

SEQ ID NO: 134 - DNA, 94 bp, Canis familiaris
aattgatctaaaaccagtctgcaaacactgttatgaaaaaatgccagaagaatttaagaggcgacttgccaaacgggagagagaagcaaaggat

SEQ ID NO: 135 - DNA, 503 bp, Canis familiaris
  misc_feature (431)..(431): n is a, c, g, or t
ctggcatgtccagtcaattctgaattatttcctttcagcccataagaggccttgggaaattgctttaagaagagcggatcttctgattatgctagatattataagtggtgatggttctgttgctgtgaacacagcagtctgtccctgtcactgtccacccaggagtggacttgctaattcctagtggcatgtacctcccctctgagcttcaccctccctagccaccctgggcggtaggataggagaccatcctcttcctcctgaccttagcagctcatcgagacattgcagagccagaagcacccctggccgcgcctgggagacaaaccctttgatgatgaaataaaccagagatttcaaagcctggggtctcaactctgcttgaagggtaactgtctttgaaaaccactttgtgaccctccctgctcccnatggacaaaagcacattaattctgctgttacgggtactttcctcacgcgagctttcatgttcagcatgcaaa

SEQ ID NO: 136 - DNA, 547 bp, Canis familiaris
gggatgcagaaggtcctctattccacagcaatggaatccatccagggtccagggaaatctggagatgggatcatcacagagaacccggacacaatgggaggcattccagctaaaagccataagggagagaaattacttttatttatgggcattattgacattctgcagtcctataggttaatgaagaagctagaacattcctggaaagcccttgtctatgatggggacactgtttcagttcatcgaccaagcttttatgctgacagatttctaaagtttatgaattccagagttttcaagaaaattcaagcactgggatcccgacacaggccagacctggtgcctagcaccccatcactgtttgaagctgcttccttggcaaccaccatttcgtcttcttccctatatgtcgatgaacattgtccacatgacaggactgcgctctattcaaacagtaaagggttaccttccagttcaacatttaccttggaagaggggaccatctacttgacttctgagccaaacgctctggaaatgcaggatgata

SEQ ID NO: 137 - DNA, 426 bp, Canis familiaris
ttggcatctactgcatccagttcatctctatggtctttggtgggcagaagccggagaagatttcagccgtgggaaggcgctatgaaacaggtgtggacgacaccgtcactgtgctcctccagtacccaggagggatccatggcagctttacctgcagcatctctgctcagctctctaatatggcctttgtgagcggtaccaagggcataggccagatccttgacccttgctggtgcccaacggagctggtgttgaagggagagcataaggagttcccactgcccccggccccaagcaaggagttcaattttacaaatggagcaggcatggcttatgaggccaagcatgtccgggagtgcctgcgaaagggcctgaaggaaagtcctgtgattcccctggcagaaagtgaactcctggctgacatcc

SEQ ID NO: 138 - DNA, 269 bp, Canis familiaris
  misc_feature (29)..(29): n is a, c, g, or t
aagagatctttgaggactattattttgtnagttaacatcttctctttgtagtagtgaaacctgtantctgtatatgagcagcttcaaattgactttaccaatcttaaaatttgaccacagtgtcctattttgcagatctgatataaaaaccagaanttggcatagttaaaattatttatgaggtgtgtgccttanataggcttccacagtagagtttgaatgactccngnctcatccatttgngattgagagctgcctttctatttact

SEQ ID NO: 139 - DNA, 537 bp, Canis familiaris
  misc_feature (27)..(28): n is a, c, g, or t
ggtaattctgcttcttctatgtttctnntntnnnttnagtnannnanattngtnntgggtcagagcattcagatatagtcagcttctctatcacactacatcttcctccttgtcagcctagctcngctttccctagaactttccnctgnctctacatcntgctgnacacagagatgcctaaaggcagctctagggtagtgcttttgtatggtttagtcaagctctgaaatcttgggcaaaaaggtgaggagagggcaaggagaggaaaggattcctctctccagaaggtcactccaatgttacttttgattcctagagggtaaatatgactcctttttctatcccaagccaaccaagagtgctcttggaggaaaagccagctcttccttgcctgttgtcaagtctcagctgatttgcagaatatgtcttttaaaaatgtttaagcctatttatttgagagtccttgtttttgctactaattatatagttcatcatatattaccattcacaccaaccatcgtggtcataacatctttg

SEQ ID NO: 140 - DNA, 577 bp, Canis familiaris
  misc_feature (55)..(55): n is a, c, g, or t
ggacccgtcagaatggcgaggctctgaaaacccacaccaacatctctgagagccntcccaacaacaccttcagtgccatgggggaagccactgtctgcgtggaggaatgggagtcaggcgagcagttcacctgcacagtgacccacacagatctgccctcaccgctgaagaagaccatctccaggcccaaggatgtcaacaagcacatgccttctgtctacgtcctgcccccgagccgggagcagctgagcctgcgggaatcggcctcactcacctgcctggtgaaaggcttctcacccccagatgtgttcgtgcagtggctgcagaagggccagcccgtgccccctgacagctacgtgaccagcgccccgatgcccgagccccaagcccccggcctctactttgtccacagcatcctgaccgtgagtgaggaggactggaatgccggggagacctacacctgtgttgtaggccatgaggccctgccccatgtggtgaccgagaggagcgtggacaagtccaccggtaaacccaccttgtacaacgtgtccctggtcttatctgacacagccagcac

SEQ ID NO: 141 - DNA, 570 bp, Canis familiaris
atcatacacgggtcatctccatggagaagggtggcaacatgaagagagtgtttgaaagattctgtcgaggcctcaaagaggtggagcggctgatccaggagcgtggctgggagttcatgtggaatgagcgtttgggatacatcttgacctgtccgtctaacctgggcactggacttcgggcaggagtgcacatcaaactgcccctgctaagcaaagtaaaggaccgcttcccaaagatcctggagaacttaaggctccaaaaacgtggtactggaggagtggacacagctgccacaggcagcgtctttgacatctctaatttggaccgactgggcaagtcagaggtggagctggtgcagctggtcatcgatggagtaaactatttgattgattgtgaacggcgtctggagagaggccaggatatccgcgtccctccacctctcgtccacaataagcattaactccccattccccgaagatgactcaagatccccaggacttctatccattctaatgtggcccattctacttgccctggatgcccctaactcccttctgtcctagtaaaga

SEQ ID NO: 142 - DNA, 285 bp, Canis familiaris
  misc_feature (88)..(88): n is a, c, g, or t
aatgagggccaagtgggctcttctcatctctgccatctttccctttgcagcaggatgccttcttcaatatttactgctatttttggcnctcccccaggagctcctacaacaaaagggagcctgagaatcttgccagaagccagccagaccccccagctggcaggtggggctcccgtggctgctcaggaagcaccttgcccagaggtcccttcaccttcccttctcaggcccagacagaggccctcatttcctctcctctgaagacttccctacctgaccagtggg

SEQ ID NO: 143 - DNA, 371 bp, Canis familiaris
gaggccatcttggactactagaagagaagcactcgtgtaactgctgaagacagctgctccctgccccaggagtaatgggggatgaagctggaagaccatggaagacacaccagtggcactaatactgaccaactggagatccaagctgggtcaggaagagttactgttacgctttggcatgtgctgagaactttcaaaaaaacaaaacaaaacactcttctttggtttccatcacaacagcatctgttaactgacaaatatggaacatgatggaggagaggcccaaatccccccaaattcccaaagcaaaaagcgagagtgtaattgtcagaaacatgatagtactgtcaaaagtacacaacccactatgt

SEQ ID NO: 144 - DNA, 497 bp, Canis familiaris
  misc_feature (188)..(188): n is a, c, g, or t
gagaagcactgctccaagatttcccagacgccacagttgtggtgacacttgaatgggtctggatgaggccgtgtggtccacttcctcgtcctctgctcatcttagaatttcggtttccactggtcagtttttagagagaaacaaatttgtccctgtggattagaatgagaggtgggaggctttagaanccacctgagtacaaagtgatctaaatagctggtctgcagcagtctttcttggagtgcttgatgtggaacccagcttacctgagaggtaaggtgaggtggaggagaccggtgtggtcacaagtgttggcgagacgcttcatttggctctgttgtgtcccagatgtgttttggccacggctctatatgaaacacctgttaacccagtatgacagcactttataacatactagttgataatgccagtctctgattttctagccaacaatccatcatggacaaaaaagcaaagacccatttccaaactcagat

SEQ ID NO: 145 - DNA, 258 bp, Canis familiaris
gggattatgacaccagcactggcaagttcacctgcaaagtcccgggtctctactactttgtgtaccacacgtcgctgacatccaacctgtgcgtgcatctgtaccgaagtggtaccagggtgaccaccttctgtgaccacatgtccaacagcaagcaggtcagctcgggcggtgtgctgctgcggctgcagatgggtgagcaggtgtggctggcggtcaatgactacaacggcatggtgggcaccgaaggctccgaca

SEQ ID NO: 146 - DNA, 393 bp, Canis familiaris
  misc_feature (115)..(115): n is a, c, g, or t
gattctttattatttgtggcatgaccaactgcgtccgaagaagagaagacataaagtacaaagcaagctcctcatttgtgttgagcaaaatgaggcaaagatttggatacacttnctgaaaaagaaagcgatggttatggcattggaaacccccatcactatgacacaccaccccccacccccccatataagagaccactaaaccacatatatatgcacacagacacaggcagaaacagacacagggacactgccttagggagtgaggtgcgcccccccccgcaagtgtagactccagcaagtagaccccttcaggtaagcaattgaactgagggaagagcatgaatgcaagactcgcacataatnaagaccttgtttcattcttttgacatg

SEQ ID NO: 147 - DNA, 136 bp, Canis familiaris
tgctgtgtttccctatgaggtgaaaaaagatggtgcagcaaaacagcccagcgatcccagagttccagcagcccacggaagaagttaaagttgctgccatagaattgaatgatgaaaccaactctcgattatccga

SEQ ID NO: 148 - DNA, 487 bp, Canis familiaris
  misc_feature (395)..(395): n is a, c, g, or t
gagctgatggacaccacacggccggacatcgaggtcaaggactggttcgcagccaggccggactgcgggtccaagtaccagctgtgcgttcagctcctgtcgtcagcacacgcacccctggggaccttccagccagacccggcaaccatccagcagaagagcgatgccaagtggagggaggtgtgtgggccagggagagggcaggtggcatatcgcccatggccgagactgcagtcacaggcctgggttcacatcccagctctgctgcttctcacccgggaaaagacgctggtaaagccacgtccgagacggttgaccgttcctggtcacgaacgtcggctctgactactctctagaccggggggcggcaaacgttttcttaacgggccggaagnaagtattttaggccttgccggccgagctgtctctgtcaccgtgactcctcttcgacgctgctgttgtgcagcgaacgcagttttatggacct

SEQ ID NO: 149 - DNA, 319 bp, Canis familiaris
gagcggaaacgcgaagatgcaattacacaacagaacacgatgcagaatgaagctgtcagtttattagatccaggcagttcctacttgctacaagaatcatctagaacagtttcaggcagatataaaagcacaactgcctctgaagaagatgtctcaagtagatatccccgaacagatagaagtgggttcagccgatataacagggatgcaaatacttcaggtaatttggtctcaagtagcatgttggaaaagaaaattgaagatcttgaaaaggaagtagtaagagaaaggcaagaaaacttacgacttgtgagactaa

SEQ ID NO: 150 - DNA, 495 bp, Canis familiaris
tggaaaccctggcaacatgggcttccctgggcccagcggcctcatggggctccctggcataccaggacggagaggccccaagggcaaccccggaaacatcagggaccagccgcgaccggccttctcagccataaggcgcaacccacccacgggtggcaacgtggtcatcttcgacacggtcatcaccaaccaggagggcccgtaccagaaccactcgggccggttcatctgcgccgtgcccggctactactacttcactttccaggtggtgtccaagtgggacatctgcctgtccatcgtgtcctccgggagggcccagatccggcgctccctgggcttctgcgacaccaacagcaaggggatcttccaggtggtgtccgggggcatggctctccagctacagcagggagaccaggtctggattgagaaagaccccatcaagggccgcatttaccagggtcctgaggccgatagcatcttcagtggcttcctcat

SEQ ID NO: 151 - DNA, 185 bp, Canis familiaris
  misc_feature (87)..(87): n is a, c, g, or t
gatttcttctcatattcttcactttttttcaaattttaaattgttaggtgcttaggtggctcagtcgcttaagtgtcccactcttgnttttggctcaggttcatgatctcagggnnatgagatcaagccttgcattgagctctgcactgagtatggaacctgcttcagagtttctttctttctcc

SEQ ID NO: 152 - DNA, 475 bp, Canis familiaris
aaagatactcttggtctcccagttgctgggcaaacctcagaatttgttaaccaagtgttagagaagaccgcagaaggcaatcccactggaggccttgtaggactaaggataccaacatcaaaagtgtaatcagcctcattggaccactggtcagaaatgtctgtgtttgtcacgttattcattgtaaattttcattctgttttgcatgtcagtttagcattatgtaaacatttacaattaggttacattgttttaagaactaagtagcataagtgaagcatgatccaaaatacttgattattgcattttcagagcataaaccatagttaaaactgctactggcatcagaattgaaactcacatttaagtaaatgtttagatacagattgcaaaaatctgttaaagtaaaacatttgggaggagcgaagatattaataaatgccaaattgtggcaggtttatgctctgaaccacac

SEQ ID NO: 153 - DNA, 276 bp, Canis familiaris
  misc_feature (87)..(87): n is a, c, g, or t
tgaaaaccccatggacaatcccaaatatgttgtcaatgacgagaattggttgggccccagttttgggctttttgattattgaagagntttttaatattgccctaggagtttttgctttagcgggtctaactgatttgttggatggatttattgctcgaaactgggccaatcaaaaatcagctttgggaagtgctcttgatccacttgctgataaaatacttatcagtatcttatatgttagcttgacctatgcagatcttattccagttccactta

SEQ ID NO: 154 - DNA, 464 bp, Canis familiaris
  misc_feature (48)..(48): n is a, c, g, or t
tgagccctgtgattctgaggcgtatccacacattctgcaatgaatccncagcgctggaaaaccaaaactagactcttgtgtttcccaagcccttcagttaacattgctcaatgcagctaagtggagagaatgcaaatgatttatatttcactggctaggttgaagaaactcaagggagatgtgttctgcttcaagacagggggaaaaaaaatcccaaaaactgtaatgcaaaggctttgtagtatttttaaacctttgattcagtccatatgtgacatctgaacaccgccaaattctgtctgtgtttcaaaacacacacacacactcgtaacaggagagaaaagttgggttttaggagccgtggtggtgcagggaggaccggctgggatccctcttgaaattatttagtatagaaggagcctccttctatgccattccgtgtggcccatatgagcagttgctta

SEQ ID NO: 155 - DNA, 234 bp, Canis familiaris
tacaagggtcctgaagtcagttgttgcatcaaatatttcatatttggcttcaatgtcatattttggttttctgtgttcctgggaattattttcttcctggagctcactgctggggttctggcatttgttttcaaagactggatcaaagaccagctgtatttctttataaacaacaacatcagagcgtacagagatgacattgatttgcagaacctcatagacttcacacaggaa

SEQ ID NO: 156 - DNA, 529 bp, Canis familiaris
  misc_feature (310)..(310): n is a, c, g, or t
acaaggccaccctggtgtgcctcatcagcgacttctaccccagcggcgtgacagtggcctggaaggcagacggcagccccatcacccagggtgtggagaccaccaagccctccaagcagagcaacaacaagtacgcggccagcagctacctgagcctgacgcctgacaagtggaaatctcacagcagcttcagctgcctggtcacgcacgaggggagcaccgtggagaagaaggtggcccgcagagtgctcttaggtcagcccaaggcctccccctcggtcacactcttcccgccctcctctgaggagcncggcgccaacaaggccaccctggtgtgcctcatcagcgacttctaccccagcgntgtgacggtggcctggaaggcagncggcagcccngtcacccagggcgtggagaccaccaagccctccaagcagagcaacaacaagtacgcggccagcagctacctgagcctgacgcctgacaagtggaaatctcacagcagcttcagctgcctggtcacgcacga

SEQ ID NO: 157 - DNA, 130 bp, Canis familiaris
ctgcttggcttgctgatgccttttagggcttttggagacgtaaagttcaaatggagcattgaccttcaaaagagagtgatagaatcgggcccagaccagttgaacgacaacgaatacaccaagttcatcc

SEQ ID NO: 158 - DNA, 478 bp, Canis familiaris
gcagagtcttcttatgctacatcagagcttccatggagcatggaacccaccctgcaaagcatgagtctatacaagttgtgggatgatgatgcttgattcatcagattggtgtttggaagaacactgaatgcgttggaagattttgatcacaaatatgatttgcaaacttaacgataaggaaattcagaactgtcaggcccatgtggctaataggcaattagaatctttttatgtctgttttaatcatgtttttgaaataggtttaagaagttcttgtgctatgtcccaactcacaggacaggctgtagaacctcaggttcaaagaggatgcccacaaacctctagaagcttaaattctaaatccgtgtgcctttttcagaaagcctttccttcaaaccaatgaatgtcataatataacctcaaatccatactcaaatttgcagcgggacagtggggtgagattcagacctctacccac

SEQ ID NO: 159 - DNA, 539 bp, Canis familiaris
tagacgcctacgagaacacagtgagcttgtctgcgactgggttttataggacacccaaccttggctacagctttgagactaactcggggaatcccttccactacttctcctatggggtggcttgctctgaagtggagatcgattgcttaaccggggatcacaaaaacctccgtaccgacattgtcatggatgttggttccagtctgaaccctgccattgatattggacaggtggaaggagcatttgtgcagggccttggcctcttcaccctggaggagctgcactactccccagaggggagcctgcagacccgtggccccagcacctataagatccctgcgtttggcaacatccccactgagttcagagtgtccctgctccgcgactgccccaacaagaaggccatctatgcgtccaaggccgtcggggagccgcccctcttcctggcagcctctgtcttctttgccatcaaggacgccgtccgtgcagctcgtgctaggaactcagattgtaaaacgaagcttttccggctagacagc

SEQ ID NO: 160 - DNA, 382 bp, Canis familiaris
  misc_feature (53)..(53): n is a, c, g, or t
acctccagacatccttgaagtacaatattcttccagaaagggaagagtccccnattcgctttggaggtacagactctgccccaaacttgtgacagacccaaagcccacaccagcttccatatcttgctgaatgtcagttacaccgggagtcgtcctgaatccaatatggtgattactgacgtgaagatggtatctggcttcattcccctgaaaccaacagtgaaaatgcttgagagatctagccatgtgagccgaacagaagtcagcaacaaccatgtgttgatttatctggataaggtgacaaatcagacactcagcttgtccttcatggttttgcaagatgtcccagtaagagatctgaagccagccatagtgaaagtct

SEQ ID NO: 161 - DNA, 517 bp, Canis familiaris
  misc_feature (315)..(315): n is a, c, g, or t
tcgctcctcaacgtctttaacgggaatgagtgtggagctgagggctcctggcaggtgggcgtccagcaggacgtgacacacaccaatggctgcgtggcgctggggatcaaactacctcacacggaatacgagatcttcaaaatggagcaggatgcccgtggccgctatctgctgttcaacggccagaggcccagcgatgggtctagtccagacaggcctgagaagagagccacctcctaccagatgcccttagtgcagtgtgcgtcctctgtgcccagagctgaggacttctccgagaactaccggggtcaccantatgggagctgggcgcctggaaggagcgcttgtcccctgctccttgctgtactccccggccttttgactctactgcactggaatgtcctcagatagaagtttttaagaagctctatttttccagaccaggattccttataatttacagatttaccaaaagaaaggacactatttcttgtgatgcacttgaatgccccagaac

SEQ ID NO: 162 - DNA, 598 bp, Canis familiaris
gagaagaatccatcaccacggcttgtgaagacacatctaccaattgctcttcttcctaagtctttctgggagaacaactgcaagatgatttatttggctcgaaatgccaaggatgttgcagtctcatattatcattttgacttaatgaataatttggaacctgcccctggtccctgggaggagtatttggagagattcatgactggaaatgtggcctatggttcctggtttaatcatgttaagagctggtggaagaaaaaggaagaacatccaatacttttcttgtactatgaagatatgaaagagaatccaaagcgggaagtccagaagattgctagatttctagagaagaatttgaatgatgaagtcttggataagataattcatcacacctcatttgaaatgatgaaggacaatcctctggtgaattatacacatctaccaagtacagtgatggatcatagcaaatcatcttttatgcgtaaagggattgcaggtgactggaagaattacttcacagtggcccaaaatgagaaatttgatgtcatttacaagaaggaaatgtctggaaccacccttcagtttcgcacagagattt

SEQ ID NO: 163 - DNA, 211 bp, Canis familiaris
  misc_feature (97)..(97): n is a, c, g, or t
ctggtggtgggtgcccatcgtctctccgctcctgggctccattgcgggcgtattcgtgtaccagcttatgatcggctgccatctggagcagcctccnccctccactgagcaggagaatgtgaagctggcccacgtgaagcacaaggagcagatctgagtgggcaggggccacctccccgccccctcccttgagcatccacagactgtgtga

SEQ ID NO: 164 - DNA, 465 bp, Canis familiaris
gtcaatgtaggctccagacacttcgccatatattccctctgaattccatgcttcatctccaggtttgaattctagtcctacccctttgctgctaaattatttggcaatgaaataattccttttatttgcatgttttttctaagcaaataacaatcatacagggggtaataaggatagccacagagtttcgcggttgttgatttgtgacctgggtaattcagggcagcctgcgtccatcccacagcatgaggagtatcagtgaagagctttgtccatacttgctcagcctttttagcggctcccttctagcatggtgcattagaatgtattctctgagaccggcaaccctgggagactcacaatgcacagaatttgagctgttaaatccaaaacattttaaattcaaaatcccaaaattttgaattccctaacctacctgtatattattcccatcaactagctcct

SEQ ID NO: 165 - DNA, 446 bp, Canis familiaris
gaacttttggagtgcctatttcccacggattttcattctgttcctcctcactacacagagcttttaatgttccattccaaagaacgaactgattcccattcacctgtatgtcttcagcttcattataagcattctgagaataaaggaccccagggcaatcaagcaagacttagttctgttcctcagaaggcagaattacaaattaagttgaatcctgtgttttgtgagctggatatcagtattgtggacaggttaaattccttgctccaaccacaaaagcttaccacagtagagatgatggcatctcacatgtatacttcatacaataaacatattagtctgcacaaggctttcactgaagtatttctggatgattcacataatcctgcaaattgtcggatatccgtacaagttgccacaccagctttaaacctttctgttcgctt

SEQ ID NO: 166 - DNA, 468 bp, Canis familiaris
  misc_feature (39)..(39): n is a, c, g, or t
ctggcatttcaagtgttggcaatttgggatgcttcccgnggatgatttggggatctttgatgagtctctgaatgattttgactgntttcagattttctttgctttctgtgnttctctattggcttttgatgttctaaaagcagtgaatcctctttacaagaaaaaaacacacagaagggtacaggataccaatccctctagtccctccctgcagcactgactactgtggacatctgcgtggcccctctatggtgctcagggggaggatgcacccccctttgtccccacccacacccccttgtggtaggctttaggggaatgggctcatggctggcagactgctctgtaaccctgttctcaaatgttttatttagttcctactttttcactactaaaatatcagcgcagaaaagacatattggcaaaaagacactttaaaaaatttagcaaaatctctcaatccatccc

SEQ ID NO: 167 - DNA, 410 bp, Canis familiaris
  misc_feature (115)..(115): n is a, c, g, or t
gcatctgcccctctgagggtgtcttgtttaccagtcccggctggcatccaagcgcggtgcctggtgcctactgattgtgcaataaatatttcgcaagcgcgtggatggatggaanggtgcccgggntgtgtgtgggaggctcttctcgagaaccnttaaagggattcaggagtccntgcntaggccgctgttggccncnaggnctctggcctagggggctcggaagactgagggcatncctgtccacagggaaccggactncaacacctgcctcggggtgggtggctgggctcagctgtgcncccctccgtgggaacagggacaaccccccccacctgcaactcgtggcccccctgacatattgtggggacccggaggganggcttcaagcccgagaggacagaggcagt

SEQ ID NO: 168 - DNA, 522 bp, Canis familiaris
  misc_feature (49)..(49): n is a, c, g, or t
actggttgggcccaaaactctggaggcaggtcccacagcccccgttctncccctctctctgccctctgccctttctctagggccgccagcatctcttgtaacttaggtcttggagtgggggggctcttgctcttgaggcactggtcaccccaaagtgagcagctgcaatgtgtggggaggtcctgtcttggccacccctctcttgtcctggggggactgggccctggcacgcatccctccccggtcacttccacacactgcctgaggtgaggcaggggccaggccctagccctctccaagtctctcagtcctcctcccacctccctggccctggcattgggggtcatcccagaatggaccccgccccctcctaatatttgctggaaagtccagcggaggagaggtggcaggtcccccgcatggctcccagtctctctggactgggcagcccgcccccacctcagaacccttcccccagtgcggccacactctgctccctgtcctgtccccttcttttgtatt

SEQ ID NO: 169 - DNA, 285 bp, Canis familiaris
gcagcttcttggtcctggcagtgttccttatcctggggactttggcggcacaggcagctgtcacaggagttcttcctaaaggatcaggcacggacagaggtcgtgttccggtcaaaggacaagatccggttagaggtcgagatccggtcaaagccaaaggtcccgtgtccactaagcctggctcctgcccccctattctgatccagtgtgccatgctgaaccccccgaaccgctgtctgagcgacaccgagtgcccaggggccaggaagtgctgcaagggcccct

SEQ ID NO: 170 - DNA, 390 bp, Canis familiaris
ttgctgctgtttctcttaggttccccattcctagtgactcatgggtcaagatctggagagggagtagataataatgaccagaaaaccatggagatcttcctctctgctaccgtggagtatgccttacatgtattcaacttgcagagcaatgacacgaaggcctacaggttggtgcgcatcctggattccaggatagagcaggagaagaagatgatggcattttccatggagctgcagcttaaaagaacaagatgtgggaaatttgatgaagatattgacaactgtccctttcaatcaagttcaggacagaacaatatcatcacctgcttcttcaccatcagcactgatccctggaatacaatatttgaactctggaacaagacctgcttg

SEQ ID NO: 171 - DNA, 73 bp, Canis familiaris
tgaacggtcagttgagaacctgagaaggatcttacctcccgggaatcgaggggacccgaacaggtggcacaga

SEQ ID NO: 172 - DNA, 521 bp, Canis familiaris
  misc_feature (45)..(45): n is a, c, g, or t
tatgactgacataagccacggttatgaagatcttggccttttacntaaggacaaaatagctgaactgaatactaaactatccaaattgcaaaaggcccaggaagaatcaagctcaatgatgcagtggctacagaagatgaacaagactgcggcaaagtggcaccaggcacctacacctacagatactgaagctgtaaagactcaagttgagcagaataagtcatttgaggcagaactgaagcaaaatgtaaacaaagtgcaggagttgaaagacaaattgacagaactgttggaggagaacccagacactcctgaggcccccaaatggaaacagatgttgacagaaattgattctaagtggcaagaactcaatcaattaacagttgatagacaacaaaaactggaagaatcctctaataacctaacccaattccagactgtagaagctcagttaaaacagtggcttgtggaaaaagaactgatggtcagtgttcttgggcccttgtcaattgacccaaata

SEQ ID NO: 173 - DNA, 295 bp, Canis familiaris
  misc_feature (252)..(252): n is a, c, g, or t
ggacacagccttgtatgttatccaaggggaagacagggaaagccttcagatggcagtaggcccgttcctccacatcctggagagcaacctgctgaaagtcgtggatcctgccactcccccagacaagaccagaaagctgtacctctatgctgctcatgatgtgaccctcatgcctctcttaatcaccctggggatttttgaccacaaatggcccccatttgctgttgatctgaccatggaactctatcagcnccaggaatctaaggagtggtttgtgcagctctattaccgtggg

SEQ ID NO: 174 - DNA, 547 bp, Canis familiaris
cagacgcagaccttcatggctcggggcgctcgcaagcagaaacgagcagtggtggccagtctcgccggcgagcctgtgggtcctgggcggcgggcgctgtggcagggccgcgcgctgcggatcccccctgtgggcccttccatcctgcactgccgggtgctacatgtggactactccctcaaggtctgcgtggacatccctggcacatccaagttgctcctggagctgccactggtcatcggcaccatccccctgcacccttttgggagccgctcatccagcgtgggcagccacgccagcttcctgctggactgggggctgggagccccgccagagcggcctgaggcccctcccgagtactcagaggtggtggcagacggggaggtggcagccgtggggcagagctcctttccaccactgcaggacactgacttgagcttcgaaggccccttcttcgcctatatccaggaattccgctaccgcccgccacccctgtactccgaggaggatccaaacccaccctcagagaccatgaggccacgctgca

SEQ ID NO: 175 - DNA, 326 bp, Canis familiaris
cagagaattcttgatggtggaggacagaatgacatcttggaaatcaaggagctgactaaggtgggacaatttttagtagtttgcttatacagcttacgcaatctttttcagtgctttggacttctaggagttaatggggctgggaaatcatcaactttcaagatgttaactggagataccactgttaccagaggagatgctttccttaacaaaaacagtatcttatcagatatccatgaagtgcatcagaacatgggctattgccctcagtttgatgccatcacggagttactgaccggaagagagcacgtggagttctttgccct

SEQ ID NO: 176 - DNA, 577 bp, Canis familiaris
ggcagacccatactggcacggacatggccgtcttctgtctgctgtgtgggaagcgtttccaggcgcagagcgcgctccagcagcacatggaggtccacgcgggcgtgcgcagctacatctgcagcgagtgcaaccggactttccccagccacaccgccctcaaacgccacctgcgctcacacacaggcgaccacccgtatgagtgtgagttctgtggcagctgcttccgggatgagagcacactcaagagtcataaacgcatacacacgggcgagaaaccctacgagtgcaatggttgtggcaagaagttcagcctcaagcaccagctggagacacactatagggtgcacacaggcgagaagcccttcgagtgcaagctgtgccaccagcgctcccgggactactcggccatgatcaagcacctgagaacgcacaacggcgcctcgccctaccagtgcaccatctgcacggagtactgccccagcctctcctccatgcagaagcacatgaaaggccacaagcccgaggagatcccccccgactggaggatagagaagacttacctctacctgtgcta

SEQ ID NO: 177 - DNA, 341 bp, Canis familiaris
tccggcaagtcatgtatgctccctaccccaccacacacatagatgtggacgtcaatactgtcaagcagatgccaccatgtcacgaacatatttataatcagcgtagatacatgagatccgagctcacagcattctggagagccacttcagaagaagacatggctcaggatactatcatctgcacagacgaaagcttcactccagatttgaatatttttcaagatgtcttacacagagacactctagtaaaagccttcctggatcaggtctttcatttgaaacctggtttatctctcaggagtactttccttgcacagtttctactcgtccttcacagaaaa

SEQ ID NO: 178 - DNA, 565 bp, Canis familiaris
gagcccactttgagcctgcatcagagactgaagggctggagaaggacacggacgaggtagaagaggaccgtcccccagcagacgggcctcgaagtggagagcgtgtggcatatggaaggagcagccccacgtccgcgggtgcccccggggcccagccccagcagctccagcaccagcacctgagcctccagctgggcgagctcagacaggagaccaacagacttttggaacacctagttcagaaagaaagagagtaccagaatcttctacggcaaaccctggagcggaaaactcaggaattgtatcatcttcagttacaattcaaatctaatgatactacagagaattcagcatcccctcctgggtctcaggagcagaaaactgataaagagcttgtggactggttacaactgcaaggagcagattcgagtacaatagaaaagattgttgaagaaggttataccctttctgatattcttaatgacatcactagggaagatctaagacaccttagactacgtggtggttgcctctgccggctctggacagcagtctgccagtacag

SEQ ID NO: 179 - DNA, 164 bp, Canis familiaris
gtatgtaatttgctagaatgggctatctagcgctttgcttcattttattactgaaaatttatttccatcaccttttgcctcagtacatgtgatatatgtaaaaattaaagtgtgcctgtaaagattttgctttctttaaagtatgcatttcttaattgagtaaa

SEQ ID NO: 180 - DNA, 491 bp, Canis familiaris
  misc_feature (375)..(375): n is a, c, g, or t
gtatgaaagcccagcagaagcctcatcccatgagacaactcttacatcaccccaagagagagtccaagaggaagggctcgctcaagagtgagaagattgtccgccagctcctgggcgagctctacgtggacaaggagtatctggagaagctcctattggatgaagtgaccttcctgatgctaagtcaagagcccttgacaacattggcagggtttttgccagagttgggaaattccagcaagccattgacacgtgggaggagaagatccctctggcaaaaaccaccctggagaagacctggttgttccatgagatcggccgctgctacttggagctggaccaggcctggcaggcccagcattatggtgagaagtntcagcagtgtgctgaggaagaaggggacatcgagtggcagctgaacgccagcgttctggtggcacaggcacaagtcaagctgagagacttcgagtccgcggtgagcaactttgaga

SEQ ID NO: 181 - DNA, 429 bp, Canis familiaris
  misc_feature (99)..(99): n is a, c, g, or t
aattgcccaagaagccaagggtcttgctcatgaagctacaaaactggcaacaggtcctcagggttcattaaaggaaggtgccaaaggctctcttcaganaagctttgggattcttaatgaagccaagaagttagcanatgctgtgaaagaaaacgatgactatctgaatggcttaacaaccagattagaaaacgcagatgctagaaatggggatctcctgagagctttgaatgacactttgggaaagttgtcagccattccaaatgacacagccgctaagctgcaggccgtcaaggaaaaagcaagacaagccaatgacacagcaaaagacgtattggcacagattaaagatctgcaccagaacctggatggcctgaagaaaaactacaatcaactagcagacagcgtcgccaaaacaaatgcggtggt

SEQ ID NO: 182 - DNA, 407 bp, Canis familiaris
  misc_feature (33)..(33): n is a, c, g, or t
tccttatctcactgctgagccagaggtcacttnccnccggttaaggccacaggataaatttctagtgttggctactgatgggttgtgggagnccatgcataggcaggatgtggttaggattgtgggtgagtacctganccggcatgcatcnccaacagccaatagctgttggtggctacaaggtgactctaggacagatgcatggccttttaacggaaaggagagccaagatgtcctcagtattcgaggaccagaatgcagcaacccatctcattcgtcacgcagtgggcaacaacgagttcgggactgttgatcacgagcgcctctccaaaatgcttagtcttcctgaggagcttgcccggatgtacagagacgacattnccatcatcgtggttcagttcaattct

SEQ ID NO: 183 - DNA, 467 bp, Canis familiaris
atgtggctgcactaatgcacctgtcagtgggttcatccatatttacacaaccatcacccagtaaccctgtggtcagggcgggaatgctgcatttgggccgcacatttcaaaattgcccggtgggaaaccgatcccacatatgacgtcatcagatgatcatcccgtgtgatgagggaaaatacatacctggcacgtggacaaatgatgacaccttccaagggaaactccctttacaattttaggttgagattgccactttaaatgtatcttatttacaggcctcgatctgtatttgtatatcctggcacagtgatgaaaatcactgtaacaccccattgtgtcgtgctggctgccaagctagaaaacactgtaataaaggagttggatgagagatttctctgaaaatcaggcgtgtgaattttaaagtttttggtaagttactaagttgctgactctctagtgtacga

SEQ ID NO: 184 - DNA, 211 bp, Canis familiaris
  misc_feature (44)..(44): n is a, c, g, or t
acaaggccaccctggtgtgcctcatcagcgacttctaccccagnggcgtgacggtggcctggaaggcagncggcagcccngtcacccagggcgtggagaccaccaagccctccaagcagagcaacaacaagtacgcggccagcagctacctgagcctgacgcctgacaagtggaaatctcacagcagcttcagctgcctggtcacgcacga

SEQ ID NO: 185 - DNA, 351 bp, Canis familiaris
tggcgaccctgatgatgacaactctctttttccaacaagagagccaattaaccccttcttcccctttgatctgttcccaacatgtccatttggatgccagtgctactcaagagttgtacactgctctgatctaggtttgtcctccgtcccaagcaacattccatttgatactcaaatgattgatcttcaaaacaataaaattaaggaaatcaaagaaaatgattttaaaggactcacttcactttacgctttgattctgaacaacaataagctaacaaagattcacccaaaagcctttctaaccacaaagaagttgaggaggctctatctgtcccacaatcaactaagcga

SEQ ID NO: 186 - DNA, 462 bp, Canis familiaris
  misc_feature (275)..(275): n is a, c, g, or t
attcctaaatggtaagtctgacttactttatgacaaaaaatttcaacagaattttgcaaaactattgctacatcaggatcaagagaaacaagcatctactgcagtttccttttgtatacaaatgattttgtataaatcatatgcttgaacattcttttctcagtaacaaaaaaaataacacattcagtttttaacactttgttatcaagtgcatttaaaaaagaactgtactgtaattggaatgcttgacttagcaaaatttgtgctcttttatntgctgttatgttagcaaaccaagattgacaagaatggttacgagaagagtacattacactcttcttattctttctttaataactcgagtagtactgtaatacttctttctgttaatgttgaagtatggtttcatataatcttattcacatnntcctatcttagagctttactgtctttatgttaaac

SEQ ID NO: 187 - DNA, 484 bp, Canis familiaris
ccagtgtctggtgtcgatctcgaaccgcgctgcggtgccggagcacgggggcgccccggacgccgagcgactgcgactccctgagcgcgaggtggccaaggagcacggcgacccgggggacgcctggaaggattactgcacgctggtcaccatcgccaagagcctgttggacctgaacaagtaccgacccatccagaccccctcggtgtgcagcgacagtctggagagtccggatgaggatatgggatccgacagcgacgtgaccaccgaatctgggtcgagtccttctcacagccccgaggagagacaggatcctggcggcgcgcccagcccgctctccctcctccaccccggagtggctgcgaaggggaaacacgcctccgaaaagaggcacaagtgcccctacagtggctgtgggaaagtctatggaaaatcctcccatctcaaagcccattacagagtgcatacaggttagcggcggctc

SEQ ID NO: 188 - DNA, 439 bp, Canis familiaris
gtgaatgagaaaaatcccttctttggttacgccgggagtaagaagcacacagaaaagaaactactttgtgatgtttttaagaagggagatgtttactttaacacaggagacttaatggtacaagatgaggagaatttcctttatttttgggaccgtattggagacactttcaggtggaaaggagaaaatgttgccaccacagaggttgctgatgttattgggatgttggatttcatacaagaaacaaacgtctacggggtggcagtgtcagattatgaaggaaaagcaggaatggcttctattattttaaaacaaaataagtctttagacttggagaaggtttacgaacaagttgtaacatttctaccagcttatgcctgcccacgatttttaagaattcaggagaagatggaaacaacagggacattcaaactacaga

SEQ ID NO: 189 - DNA, 525 bp, Canis familiaris
  misc_feature (50)..(50): n is a, c, g, or t
aggcttagcctagagcttgccaggccccaacctctcccgtctgctccccnctnaaggggtgnaaaggagcagagaagcttgcatttttttccttaaaggcccagaatagtaaccancggtaggcttgcatttccagaggacaaantcaaaggtgatttgtaggtacttcctaaagccatgtaatcatgtaaacacggtaaaaaacattcttagctcatcggccctacaaatcaggccctggctatattaggcctgttggctgtagttagctgatcccaatccctgccatataccctccaaagggccctgtcaccttgggtggctttggtgggatttttgagaacaagctgctgcaggataggagtttggggcaagactacttcctcttttaccttttatttagatctagaggatgtgcacacctgagtccaaatccacctgtcctgaagtggagataaattacatgggttggagaaccacagactgcctaactgctttctgaggcatttcccagatccaaggatt

SEQ ID NO: 190 - DNA, 550 bp, Canis familiaris
tccagcattggaggtcggcttgcaattagtggagggggctatactccctcaaaatatgcagtagaaggcttcaatgacagcttaaggcgggacatgaaagcttttggtgtgcacgtttcatgtatcgaaccaggattattcaagacaggattatcagatccaataaaagccactgagaaaaaacttgccatttggaagcacctgtctcctgatatcaaacaacagtatggagaaagttacattgaaaaaagtctagacaaactgaaagacactacttcctttgtgaacgtggacctctccctggtggtggagtgcatggaccacgctctcacaagtctcttccctaagacccgttatgctgctggaagagatgccaagaccttctggattcctctgtctcacatgccagcagttttgcaagactttctattgttgaaacagaaagtagagctggctaatcccaaagccgtatgacacagctgacaacaaatgcctgcccccgggctatgagattggctgctttcaaagacacatctcctttttggtctca

SEQ ID NO: 191 - DNA, 533 bp, Canis familiaris
  misc_feature (446)..(446): n is a, c, g, or t
tcccactgcagattttcaagtaagagcaactgggagaatcactagggaactcattagaaactggaacacagtattactctgtgttatcttctgtagtcaatacagacagatttttttttttctctgcagccccagaagaaattaggtgaaagcaggtaaccacacttgtctgtttatataaggtaattccttttatctggacagaatatttagttcaatcttataaaaatgattagaatattaaaattgctgttatgatacattgtaggtacctaacctaccatcggtgtggtccatactgttaatttcatggaatgaatttttataaattgaccaaaaaatgtattaacttagaacctgatttctttggaatgagttggcttattttttctattttaaaatctttgaattaaaatattttatttttaaaaaataactcaagagancgtcggtgtttctcaagctttactattctttccccatgaaatgtttttaatgaaaacggtaaattttcctttcatgtgttggcacag

SEQ ID NO: 192 - DNA, 132 bp, Canis familiaris
cttcacccagaagaccatcgaccgcctggcgggtaaacccacccacgtcaacgtgtctgtggtcatggcagaggtggacggcatctgctactaaaccgcccaatcttccctccctaaataaactccatgctt

SEQ ID NO: 193 - DNA, 168 bp, Canis familiaris
gggagagcctattcctgatgtaaatagagcgacgtgtacaaacctacagtttgtggggggttttttggtgctttttgttttgggtccaactctgacccgtttgctactacattcctagttattttccctatgaaataatacgtgaattataataaaagctgtcgactt

SEQ ID NO: 194 - DNA, 551 bp, Canis familiaris
  misc_feature (166)..(166): n is a, c, g, or t
tgccaagggtgaatcgggagactataaggccacccagaagatcgccttctccgccatgaggaccatcaacatccccctgcggcgggaccagaccatccgcttcgaccacatggtcaccaacgagaaccgcaactacgaaccccgcagcggcaagttcacctgcaangtgcccggcatttactacttcgcctaccacgccagctccagagggaacctgtgcgtgaacgtcatgcggggccgggagcgcatgcagaaggtggtcaccttctgcgactacgtccagaacaccttccaggtcaccacgggcagtgtggtcctcaagctgagccagggggagaacgtctacctgcaggccacagacaagaactccctgctgggcatggaaggtgccaatagcatcttctccggcttcctgctcttcccngatgcagaggcctgagctgtggggctgaatcccttcctcccccgcccaacccccagcaatgctcactttacccccaacaccacccacacccggccaaagcacacagtagggctccacagatgttgct

SEQ ID NO: 195 - DNA, 533 bp, Canis familiaris
gtcggagttcaggtcttatttcacatcatagagttcacactggggagaagccctacacttgtattgaatgtgggaaagcctttagccgtagttcaaatcttactcaacatcaaagaatgcacaaaggaaaaaaagtttacaaatgtaaggagtgtgggaaaacatgtgtttctaatacaaagattatagaccatcagagaattcacacaggggagaagccttacgagtgtgatgagtgtggaaaaactttcattttaaggaagacccttagtgaacatcagagacttcaccgtagagagaaaccttacaaatgtaatgaatgtgggaaagcttttacttctaatcgaaaccttattgatcatcagagagttcacactggagagaaaccctataaatgtaacgaatgtggaaaaaccttcaggcagacttctcaagttattttacatttgagaacccatactaaggagaaaccctataaatgtagtgagtgtgggaaagcctatcgttacagctcgcagcttattcaacaccag

SEQ ID NO: 196 - DNA, 502 bp, Canis familiaris
gaatgggactcctgggctgaaaacagactcaaggtggcctctagggaaggactcactccacactctccaccgcaagaagtagggagcatcccagtaaccaaacctgtattgccaaagaaaccaaatgctggccttatacgaagtgttaatcatgaaattctgggaggagggcccatggctgagagccctgacggtgggaagaaagtcccaactcctgctccccggcctttgttgccgaagaaatcagtttccttggaaaaccccaaccctacaactttgctgaaaccagtcaccgttcctccccgactctcagtggcatcacaggccaaagcattcaggtcactgggagaagggcccccaggcaactccccagctccagctgtgcaaagcaagcctccaggggacattgacctcatcagttttgatgatgatgttttacccaccccagctgggaacctggttgaagaatctgttggttcagagatggttctggatccctttc

SEQ ID NO: 197 - DNA, 542 bp, Canis familiaris
aaggttcagtcttacgatccagaaaccaattcttggctacttcgtgcagctatcccaattgccaagaggtgtataacagccgtatccttaaacaacttgatatatgttgctggtgggctgaccaaggcaatatactgttatgatccagttgaagattactggatgcacgtacagaatacattcagccgacaggaaaactgtggtatgtctgtatgtaatggtaaaatatatatcctgggtggaagacgggaaaatggagaagccacagacactattctctgttatgatccggcaacaagtatcatcacaggggtagctgcaatgcccaggccagtgtcctatcacggctgtgtgactatacatagatacaatgaaaaatgctttaagctctaatgccaggatacctcacccaagaagccacactgatccaagatgagaggttttaagagctccagaatgggaacttgacatatctcctttgtgccatgtacacaaaaaagtaaagataataattgtggtgccttttcccctgaaattcaatc

SEQ ID NO: 198 - DNA, 447 bp, Canis familiaris
atacttggcaggttaaatggatgaaaaatgaggaatgtgcaatgaaaaacaaatacaaagcattccagttgacaaaatggaaaacttagtaagttttacggtttgttctccaaagactttaagtttcactgtaagttctgaaatatcacaaacatatattgtgtgaagatttcaggttgctgatgctacaaactcaaactctgtatgcagagtttaccagagaaacactggtgaaatcccagtatttgtaaaactttaagttttggcaaatattgtaaatatcacaattagaaagtcttgatgaatgcagaagagaaaatccatttcataaatatttcatttgattaataaaaggcttgcatgttctaaataatctgagaaaaatatctttaaaaaaaaaggctacagtttaacagtaatctgcatgtatctttagctgccttaaaa

SEQ ID NO: 199 - DNA, 35 bp, Canis familiaris
acagaattatattcacctatatgccaacacctaca

SEQ ID NO: 200 - DNA, 478 bp, Canis familiaris
  misc_feature (61)..(61): n is a, c, g, or t
acccagtgtttaccttaggcattatttttgaaaagcccccatttgtatacatttgtcctancatagcaattattatagtttttcattgtcattttatctgtnccaagtatttatttttaattattaaataaagtctgttaggactatggctcaataatttaaaattatttcaagttaatactgtttaaagcaaaacaaanccaatgaataaacaaacagaaagcagaatcagactacagagaataaactgttggttgccagaggaaagggatgggcagatggataacataggaaggggagggggagataacaggcttccagttatgaaatgaataagtcanangaataaaaggcataaggaagagtcaatgatactgtaatagtgtcatatggtgacagcgtggctagccatacttgtggcgacagagcataatgtatagagaaattgaatcactacgttgtacacctgaactaatgt

SEQ ID NO: 201 - DNA, 474 bp, Canis familiaris
taagcgcagatctatgtcccagaagtattcttggtcttctgaaggctggttaccttggagtcctgagagccagagatcccactggcagcaaagttcttatttacagaattgcacaatgggacccaaaagttttcacagcttatgatgtttttcgtgtaagtctaatcacatccgagcttattgtacaggaggtagaaacgcaacggaatggtatcaaggctgtctttgatctagaaggctggcagttttctcatgcttttcaaattaccccatctgtagccaagaagattgctgctgttcttacagattccttcccattaaaagttcgtggtatccatttgataaatgagccgataattttccatgctgtcttttccatgattaaaccattcctgactgaaaaaattaaggaacggattcacatgcatgggaacaattacaaacaaagcttacttcagcatttcccagacattc

SEQ ID NO: 202 - DNA, 436 bp, Canis familiaris
  misc_feature (210)..(210): n is a, c, g, or t
aatggtgtttttgccagatccttttttaacatcatgtgcatctcttgggatccagcaaaaatgttaagccacaatgcccttgtgccttttaatataccacagtgccagttaaactagcatttctgttgcttgggagttattttcaagagtgattcagcaaatcttatgataaaggacaggccaaagagtggacaagcacagacgaagctncagagcactgagcagaggcttgacgaaaaaggaaaagtctcgcacgctgaactgtaatgatgtggacagtacagggtaaccagaggtggccagggcctcaccactatggaagagtgtctactgaggctgcagatgggcccaggagtggcaccatgctgttgggatggccatccccactaggcttctccttgaaacacaactgcagctatattctgcatcactggaa

SEQ ID NO: 203 - DNA, 409 bp, Canis familiaris
  misc_feature (55)..(55): n is a, c, g, or t
tgcttgctctttatctgggtttcagccattcccaggttgggtgtagattgagccntgggcccccttctctgtgtcatgggatgccccctttcatggaagaaaggaagagccctctttcttggtcctggtggattcacgagcacttcccacagtgcctatattcctgtcatggtggtgaggtggacctgacctgggcctctccctgctcctccctagattgggtcatagtcttgggccccctccatattctccccagacttgcctgggactcaacggacagagtccatcagtgcactggtcttgagccccagccctgcactggccccagcgaaggctccagcatccctcccttttcttatctagttcaataaccttgggaagtagtagtctttaggcttgagtcctccaa

SEQ ID NO: 204 - DNA, 303 bp, Canis familiaris
tgcaagcctccctggcatgtgggaacgcaccctgaccattggcagcgctgggaagagttttagcgccactggctggaaggtgggctgggtcctgggcccagatagcctcgtgaagcacctgcgcaccgtgcaccagaactctatctatcactgccccacgcagggccaggctgcagtagcccagagcttccaacatgagcagctgcactttggccaacccagcagctactttgtgcagttcccacaagccatgcagcgctgtcgggaccacatgatacgaagcctgcagtctgtaggcctgaa

SEQ ID NO: 205 - DNA, 201 bp, Canis familiaris
tccaggagcctctccgaagaggagatccccttccctcccacttccatccttttcctcctggcctgcatctttctcagcaagtttctagcagccagcgccctctgggctgcggcctggcgtgggcagaaactggggacaccccaggccagtgagctggattgcagctgtgacccaggttatcagctccagaccctaacaggt

SEQ ID NO: 206 - DNA, 134 bp, Canis familiaris
gtctccagagccgagagacatgtgagagaagaggacctcacctgggaaggggctctgctggaaccaccccagcctgcacttacccctcggccaccaaggctcctgggtctgctttggcaaaaggccgcccttcc

SEQ ID NO: 207 - DNA, 48 bp, Canis familiaris
cccctaaatgagttgattccactagtatatattcaggacccaaaagga

SEQ ID NO: 208 - DNA, 288 bp, Canis familiaris
  misc_feature (222)..(223): n is a, c, g, or t
gtcaatgggtaagcagtctgaatttatactaatcaagacaaacctctgaaaggttacactaagtacagaacttttaaaccttgctttgtatgaattgtactatttgaacataagctgcacttttattttctaatgcagaggatgaataagttaaatacgtgctttaagggtagaagcagacattctgtttggaaccacgttataatctgctttttttttttnnanannanncnnntnncnnnannaannnnnggcagagatgggagggcaaaatacatccgacagatg

SEQ ID NO: 209 - DNA, 440 bp, Canis familiaris
  misc_feature (153)..(153): n is a, c, g, or t
gaaattgaagtgtccgctgagttttcagtgtttcaggactcaaggaggcatcgcgtgtttatctgtgctgggagaaggggcctgccgcagagcaaggtggcatcatcatcactatcttttttctccccccaaagttctcccgtggcctccatnccttgtgccacccggggagtgagaagggtccttctccnagttggtggcccaactgggactgcgcagccgggacgcccagctctgattcaggactcgccctccgaaagcgaatcacagtggctgctttcaacgccagggaaatgcactttagacgtacttgggtagcatcctactttaagtgaagggacgacgacgagagagggcaggaaaccttcttaggtagaagcattttaagtaaggcaagagattgcttgtaaacgttgaaccacaaaactgtactgcctgtt

SEQ ID NO: 210 - DNA, 297 bp, Canis familiaris
aggacctcaagatcgtccccttggggcgcaaagaagtcaaggccaaggacctggtgcggcagttgcagctagaggctgaggagcataggaagcagaagaaaagacagaacgtctcaggcctccacaggtcgggcctcctctctggctgctgcactcaccccacagcgggaggcctgcctaccagcctccgtttacccacaggctgccataacttgaaaacatttccctatcagtgtttgaaacatctcccagactgtattccctgtgctgtcaggaaaatcaacaagtacactctag

SEQ ID NO: 211 - DNA, 363 bp, Canis familiaris
aatttgtttctaggactgtctgtagtttttgatgatatttgcatgattagggcacagccattcaaatttatccctagaaaatgtgatagttaacaagggttctattttatatgttgatgttttttagtacaacagacacacccacacctccttaattgaacatcaacattgattttttgaatatatatactacctctttaattcagaagatatgagaatccagtggtcgaattaatctccttaaatatctaaattcacacttggttatgaaaatgatgatctgtttctcattaagattaataccaatgatattccattactctggtttacctcactcattatacttaggcacatttcacagaa

SEQ ID NO: 212 - DNA, 304 bp, Canis familiaris
attgaagagatttccgggtacctgaaaatccggagatcctatgctcttgtgtctctttcctttttccggaagttgcggctgattcgaggggagaccttggagatcgggaactactctttctatgccttggacaaccagaacctaaggcagctatgggactggagcaaacacaacctcaccatcactcaggggaaactcttcttccactataatcccaaactctgcttgtctgagatccacaagatggaggaagtctcaggaaccaaggggcgccaggagaggaacgacattgccctgaagacca

SEQ ID NO: 213 - DNA, 584 bp, Canis familiaris
gaatcagcgccgactgggagatgctgcaaagaaagcgatcagcaagctccaggtcagaaccatcaagaagggtgacaaggaaacagagcccgattttgataactgtgcagtttgcattgaagggtacaagcccaatgatgtcgtccggattttgccctgccggcatcttttccacaaatcctgtgttgacccctggcttctagaccatcgtacctgtcccatgtgcaagatgaacattcttaaagccctagggatcccgcccaatgccgactgcatggacgacctgcccaccgacttcgaggggtccctgggaggcccgcccaccaaccagatcacgggcgccagcgacacgactgtaaacgaaagttcagtcactctggaccctgccgtgcggaccgtgggagccttgcaggtggttccggacgcggagcccaccccgcaggacggagaggtcgtctttaccgccaatggcgagccggagccggctgtcagcagcgactctgacatctcgctgatcatggcaatggaagtcgggctgtcggacgtggagctctccaccgacccagactgcgacgaggcgaaat

SEQ ID NO: 214 - DNA, 392 bp, Canis familiaris
aggaggaatcttactgtgcggaatccagggacatgtgagcccaagcccccgagtatggtacgtgccatcctcaacttcacctgctgctgtaatctcgaggtaggacaagttatgatgcccacagttcacttctgtccacttcattaagtaagaagggatgcttcgagtcattgacttgggacctagggcctgcaaaattacagaaactgctgaacatgtagtgtcttgtcactgtttctgctgtaaacaccaaacatcaggtaggacaccccctttcaatagcaaatgtgtctttcttctcatgggcttggaagatgagtgccagatttttgtcatttaaaaatgacagtttgttaaattaaaaaccaatcactctcatactctggtttctc

SEQ ID NO: 215 - DNA, 410 bp, Canis familiaris
ggcagccttcagctctgtagttaaatgggcgtgctcacccctacctggtaatttaagattctggagagtggctctgccctgtgcccaaggaacggtgctgattgtacccctcccaactgccctcacccccccccagagccacccccaaagagatgccttgatattctcaatgcagccctgccttgggctgccttggtgctgtcacacacttcaggctcttaccttgccacgattttctgtggctcacagcaccctcggagccaacagacggaaactgtctcaagagggcactggtggcctgacagcccggcgtggcatggggcagtgggacaggggcgcggatgtggccgccccacacacctggcttctcactgctggctgccttaggacctctcctacattagcagttt

SEQ ID NO: 216 - DNA, 230 bp, Canis familiaris
  misc_feature (48)..(48): n is a, c, g, or t
tacagaaggttggtgttacccataaagtggtgtgtgcaacggaggctncggtgcaactagggcagtttttggtgtgggttgttttctgcagtagggagggcaagagtggttaaggtgtttgtgtagcagaataatgataagttctaagtactgtgattattgataaagtatttagaatgtattgggtacatcgaatagtcaataagtaaaggagtctttcatgtcagatt

SEQ ID NO: 217 - DNA, 398 bp, Canis familiaris
  misc_feature (30)..(30): n is a, c, g, or t
gaccaactgcctgcggtaggttctaagggnaaactacgaaaaagagaggggccgggccgttcctgctgatgaccgtgaanggccactgagggcctgtcagcctctgtggggagtcggggcagataccctgttactcccttcttgaacaacacggtgtgctcgagggctgacatcccagcttggacaggcctacaagggccaccgcttcctgcgctgagtctcgctggtgaagggagnacagccctgtgcctctctgagcttcagctccccggagggcagtaggaagcttcggtggtcacttttccagggtccttggtgcactactcggcattgagatgcttgggaacgtctctcttccctcggcatgctctgggtttcctgaagctcacttcctaatt

SEQ ID NO: 218 - DNA, 439 bp, Canis familiaris
  misc_feature (27)..(27): n is a, c, g, or t
gaagaaggctcagctgtggcgccaacncagcctaatnatttctagagaagaaaataagatctgctcatnccaaattctaancaaagtgactgtactaagtcatcactaatcactctggtaaaacagggaaggtagaaggtattctgatctttggcaaaanctgcaattacagtcctcaacaattacaagacgttggggtttttgtttttctgttggtttcgttcatttaatcaattctgggaacgcaggcgtaccgaaccctgctccgtgctaagcagtcnactccacgggcttctcagggtttcggccctaaatgctgtgcttggatcaatacacattgaggaaaacaaaagattgaaggtctcacctctgtatttctcattaccttgaacgtagcgcagccgactactttctgttgctagattcctgctacctagga

SEQ ID NO: 219 - DNA, 486 bp, Canis familiaris
ggtgacatatgggtacttcctagaagtccctaaaagaaaagcagcaagtattctttatcttatttctctatcttgctgccagaattgtggatacaacaggaggcttggcaactgtccaggaggataaaggaatgccagttagaaactaaaaagatcttgacttcttcattatttgattcccttatcctatatttgacccacagtatatttgggtttctttatatgaattcaaaactaacctattctggccttaagttttttgtcatgatgctgtttaacactgtttgaaagaactagattgcagcaaatcttacctttaaaagttttaaaggataatgtgtaattcgcattagaattgattttccattgttaattagttatactcatgttcttgccttgatctttcattagatattttgtatctgcttggaaaataccatcttctttataactgtgtaataggtacttgctaaaactctgtaatct

SEQ ID NO: 220 - DNA, 559 bp, Canis familiaris
atgaaacagttccagggcatgcccccttgcacatacacaatgccaagtcagtttcttccacagcaggccacttactttcccccgtcaccaccaagctcagagccgggaagtccagatagacaagcagagatgctccagaatttgaccccacctccatcctatgctgctacaattgcttccaaactggcaattcacaatccaaatttacctgccaccctgccagttaattcacaaagcatccagcctgtcagatacaatagaaggagtaaccccgatttggagaaacgacgcatccactactgcgattaccctggctgcacaaaagtttatacaaagtcttctcatttaaaagctcacctgaggactcatactggtgagaagccgtataagtgcacctgggagggctgtgactggaggtttgcgcgctcggacgagctgacccgccactaccgaaagcacaccggggccaagccgttccagtgtggcgtgtgcaaccgcagcttctcccgctccgaccacctggccctgcacatgaagaggcatcagaactgagccccgc

SEQ ID NO: 221 - DNA, 256 bp, Canis familiaris
  misc_feature (49)..(49): n is a, c, g, or t
gaaatgtaacatgtattccttgagaagcatacccatggaggcaaactgnagctacccnccatcaaatgaatataagaaaannnnnaagaattattattttacccctgagtcctatgnattttctgtggtcatttaaggncagttanatctgtcntttttttttaaatgaaagatatgattaactgtaaacttgaaatcaaggtaagctcaggatagtacttagggatgacttacttttcctgtggtattgactaca

SEQ ID NO: 222 - DNA, 499 bp, Canis familiaris
gcacttatgtttgaattggccagagggatggagagtgactttttttatgaagacatggaatctttgactcagatgcttagggctttggctacagatggaaacaagcaccgtgccaaagtggacaagagaaagcagcggtcagtgttcagagacatcctgagggcagtggaggaacgggattttccaacagaaactgttaaatttggtcctgaacgaatgtatattgattgctgggtcaaaaaacacacctatgacacctttaaggaagttcttggatcagggatgcagtatcatttgcagtccaatgaattccttcgtaatgtatttgagcttggaccccccgtgatgcttgatgctgcaacacttaaaacaatgaagatctctcgttttgaaaggcacctgtataactctgcagccttcaaagctcgaacaaaagctcgaagcaaatgtcgagataagagagcagatgttggagaattcttctagattttcagcactt

SEQ ID NO: 223 - DNA, 575 bp, Canis familiaris
tcgtgtcaataagtttcagagagttgatcctgatgttctgaaagcctgtgagaacagctgcatcttatacagtgatctaggcttaccaaaggaactcactctgtgggtggatccatgtgaggtgtgctgtcggtatggagagaaaaacaatgcattcattgttgccagctttgaaaatgaggatgagaacaaggatgaaatctccaagaaagttaccagggctcttgataaggttacctctgattatcattcaggatcctcttcttcagatgaagaaacaagtaaagaagtagaagtgaaacccagttcagtgactgcgaccccaagccctgtgtaccagatttcagaattaatattcccgcctcttccaatgtggcaccctttgcccagaaaaaagccaggaatgtaccgagggaatggtcatcagaatcactaccctcctcctattccatttggttatccaaatcagggaagaaagaataaaccatatcgcccaattccagtaacatgggtacctcctcctggaatgcattgtgaccggaatcactggattaatcctcacatgttagcacctc

SEQ ID NO: 224 - DNA, 501 bp, Canis familiaris
  misc_feature (56)..(56): n is a, c, g, or t
gtagcttctgtgtaaagccgtggctgggctgaaaagtggcggggcttgcatttccnttcgggtcttgatgatagatggtatgaaaatcatcgaagtatgcttttgaccatcatctccccgtacgagtttatttttaccatccatttcagagtaggtaaggcctccgttgaaaagatgacatgaccaagagggggaagcatttccttctggttctaattccctaagttgttttccttatgntgcatttaatacatggagattgagaatacagagggatatttgagtcattcagatttcataaagcagttccttggattatagctgcattaacttggaaagaacccagttaggctggaagacagaaactccaacgggcagaaaatcaagtaaccnaggggggaaaataaaatccacaggagtattgaatttaccttacttaaatgtatttgttaaatttattttactaaacaaaatgaactgctttttgtctctgaaatgata

SEQ ID NO: 225 - DNA, 505 bp, Canis familiaris
aatgcccttcttgggactacttacatccttaagaaaaaaataattacatagtggcttgacaaactactaatatacagagttaaaatgaatgtagttttttatctccaaatatatatatatattttataaatatgtttatacataatattccatttctaagaatttaatttattggctggagtgagagattttggaaggtagccatttcaacatttggactattttcttcataggaatatttttcaagatgtcttacacagagacactctagtaaaagccttcctggatcaggtaaatgttaagcctaagattgtcaaaatgaatgttatggtatgttgtctttttaatgtatactacattgcctgtgctatatatgtaatatcatagaattaagtcgttatttttttttaagtcattatctttttgtaatgtttcaggtctttctaaaatgtctacatggtatttcttttgattgttaatgggcgttagcctgtatattaacaat

SEQ ID NO: 226 - DNA, 359 bp, Canis familiaris
tctttagcttccttgtagaccccaagtcttctcagggtctgtggagtccagaccccatgctttgtatatcacgctggctgagtgaaccaaatgaaattccaaaacatctaagcatctgagagctcattcaattcttactgagaaggcaaggtactctgtatgacgtgggacatcatttttagttgggcatgtaaaaaaaaataagctaaactagaacacattcatgaagtcatttagaagtctaaaataataccttcaatattttttcctcatatgtagtttttcatggggaaatagtttgttataaatgcccagtatattcctgtaagtcaatgacatttctttcagtttagggatta

SEQ ID NO: 227 - DNA, 36 bp, Canis familiaris
gggccttgctacacacagacaggtgggaaggagcct

SEQ ID NO: 228 - DNA, 308 bp, Canis familiaris
atgttcgccatgaagtacctgtcgctgctcaagttcctcatggacctcggctgcgacggcgagccctgcttctcctgccgcttcggctacgagccccccccccccaaggcattgcctttggtccggggagccccagcaccaatgcgtgatctcttccagttctgcgagatcctgtccaccccggaagtgagccgctgggcagggcccatcatcgacatcctcctggactatgtgggcaacgtgcagctgtgctcgcggctgaaggaacacatcgagagcttcgaggactgggccgtcatcaaggagaa

SEQ ID NO: 229 - DNA, 94 bp, Canis familiaris
acatctcagacttcaagaagaagatgcctgacttgcctggtgacaaggatgagccttacgacagacgcttcgtcaagtggatgatcaagaacaa

SEQ ID NO: 230 - DNA, 505 bp, Canis familiaris
  misc_feature (38)..(38): n is a, c, g, or t
accctggtgtgcctcatcagcgacttctaccccagcgntgtgacggtggcctggaaggcagncggcagcccngtcacccagggcgtggagaccaccaagccctccaagcagagcaacaacaagtacgcggccagcagctacctgagcctgacgcctgacaagtggaaatctcacagcagcttcagctgcctggtcacacacgaggggagcactgtggagaagaaggtggcccccgcacagcccaaggcctccccctcggtcacactcttcccgccctcctctgaggagcncggcgccaacaaggccaccctggtgtgcctcatcagcgacttctaccccagcgntgtgacggtggcctggaaggcagncggcagcccngtcacccagggcgtggagaccaccaagccctccaagcagagcaacaacaagtacgcggccagcagctacctgagcctgacgcctgacaagtggaaatctcacagcagcttcagctgcctggtcacgc

SEQ ID NO: 231 - DNA, 449 bp, Canis familiaris
  misc_feature (166)..(166): n is a, c, g, or t
agaagggctgcctcattctgagctacctgaatgagacggtgactgtaagtgctaccttggagtccgtcagggagaataggagcctcttcactgacctggtgggggaaaaggacttattcagctgtatctccttcactgtgccaagatctccatccaatgaagaggncatgttcctcactatccaagtgaaaggaccaacacaagaattcaagagacggaccacagtggtggttaaaaaccaagagagtctggtctttgtccagacagacaaacccatctacaaaccagaccagacagtgaagtttcgtgttgtctcattggatgaaaattttcaccccctaaatgagttgattccactagtatatattcaggacccaaaaggaaatcgcatcacacaatggcagaaactcagattagagaaatggtctcaagcaattgagctttcccct

SEQ ID NO: 232 - DNA, 329 bp, Canis familiaris
  misc_feature (36)..(36): n is a, c, g, or t
cgcccgtctcaatgccaagatggcactccggggganggcactgcgcacccagctggacctgcgccggttccgaatctactcgaaccagtctgcattggagtcactggcactgatcccactgcaggcccctttgaagaccatgctgcagattggagtgatgcccatgctcaatgagaggatgtggcggggagtgcagatcccactacctgagggtatcaactttgtgcgtgaggtggtgacgaaccatgcgggcttcctcaccattggggccgacctccactttgccaaagggcttcgagaggtgattgagaagaacaggcctgcctcca

SEQ ID NO: 233 - DNA, 372 bp, Canis familiaris
gggagctggaagactatttttactattctcagctccgtagtcaaggtattgatacaatggagaccagaaaggtgtcggaacacatttgcctgtcagagcttccttttgtcatgagagcaattggcttttacccatcggaagaaaagattgaagatatgtttaatgaaatcagatttagtgagtatgtggatactgggaagctaattgacaaaatcaatttaccggatttcttcaaagtttaccttaatcacagaccaccttttggtaacaccatgagcggcatccagcatagctttgacattcttggttttaccaattcgaaaggaaagaaggtcatacgaagagaagatttcctgaaactgcttctaacga

SEQ ID NO: 234 - DNA, 183 bp, Canis familiaris
ccatcggagccaaaagtgagagtgtaatctctgagaatactccatgcagagcaaagatgcctaacgaagcacactcggaattgcttggtgagggcgcctcggaccccagagaaaatcccggccaaaagagaaatggactgtgcacagataaacattccctgctcagtaagaggctcaagacat

SEQ ID NO: 235 - DNA, 324 bp, Canis familiaris
gatctgcttttaggctccaatgccagcctcacatgcacactgagtggcctgaaagaccccaagggtgccaccttcacctggaacccctccaaagggaaggaacccatccagaagaatcctgagcgtgactcctgtggctgctacagtgtgtccagtgtcctaccaggctgtgctgatccatggaaccatggggacaccttctcctgcacagccacccaccctgaatccaagagcccgatcactgtcagcatcaccaaaaccacagagcacatcccgccccaggtccacctgctgccgccgccgtcggaagagctggccctcaat

SEQ ID NO: 236 - DNA, 47 bp, Canis familiaris
ccgagttcccttcccgaagacgcatccaagaacctcgatgtactctg

SEQ ID NO: 237 - DNA, 595 bp, Canis familiaris
  misc_feature (494)..(494): n is a, c, g, or t
taaacctgttgcagaagctccttcagctttcgcgttgggctcaaaaactaagttaagtgactcttctggaagtcagattggaacaggatttaaaagtaacttttctgaaaaagcttttaaatttggtactacagaacaaggatttaaatttgggcatgtggatcaagaaagtacaccttcatttacatttcagggttcttctagtacagattctaagtcaacaaaagaaggatttagtttttctgtccctatgtctgctgatggatttaaatttggcattcaggagcctggaaatcaagagaagaaaagtgaaaagccccttgaaaatgacactggtgttcaggctcaggatatcagtggtcagaagaatgatagtggtgtgatttttggtcaaactggtagcacttttacctttgcagatcttgcaaaatcaacttcaggagaaggatttcagtttggcaaaaaagaccctaatttcaaaggattttcaggtncgggagaaaaattattctcatcacaaagtggtaaaatggctgataaagctgacacttctgctgaccttgagaaagatgatgatgcctatcggactgaggac

SEQ ID NO: 238 - DNA, 184 bp, Canis familiaris
gaagagcgattagttcttattgagaaaggtgttgactcaacagccacatctgatgaatctcacaaattagaccatatcaatatgaaccttaataaacttgtaactaatgatacatttcaaccagagatagtggaaagatcaaagacgcaggatatagtgcttggaacagactttttaagcatta

SEQ ID NO: 239 - DNA, 137 bp, Canis familiaris
ggctcaagacatgagagattagcattttaaagcaggatgagtgtccagggttatagagaatcaatggaagtgtgtcttctgttagtagctggatcctaccctccaatagtaaaatttgcattccatttagatgtgta

SEQ ID NO: 240 - DNA, 566 bp, Canis familiaris
aggaactctggaatgcaggctgccagatggtggccatgaacgtgcagaccgcggggctggagatggacatctgtgacgggctcttccgccagaacggtggctgcggctatgtgctgaagccagacttcctgcgcgatgcccagagttccttccaccctgagaggcccgtcagccctttcagagcccagaccctcctcatccaggtgatcagtgggcagcaactccccaaggaggacatgtctaaagagcggtccattgtggatccactggtgagagtagagatctttggcgtccgcccggacacaacccggcaagagaccagctacgtggagaacaacggttttaatccatactgggggcagacactatgcttccgggtcctggtgcctgaactggccctgctgcgtttcgtggtcaaggattacaactggaaatcccgaaatgattttatcgggcagtacacactaccttggacctgcatgcagcaaggctaccgccacatacacctgctctccaaggatggcaccagcctccacccagcttccatcttcgtgcatatcaacatc

SEQ ID NO: 241 - DNA, 490 bp, Canis familiaris
ttgtgtttaattttgctgtggtctagaattgtggtgtttctgtagtggccataaaggcctgaaatgatttattatggatgtcagactttatcatgagaagtcctaggtaatgacttttcttgatggtgggactctgactcatgcatttatcaattacacaaaattatttgcaatagattatttgacttttcagtttttactgaaattttaaacattttgcatgtaaatacttgtatttaccaaagatttaaagcagttgattaattaatccaaacactgtgaactgtctttgaaatactagagaaaaagaaatgttaatatcacagtaacatcaactatactgtataaatagagttctataatacagaaatagtctacattggtatttgtgaaatctatggaagaactttaggattctagcagatggatactgaatgttcaggcccacttaagttattgatgtataaattgtgtttttgtctctatgtac

SEQ ID NO: 242 - DNA, 342 bp, Canis familiaris
caaggcattgcctttggtccggggagccccagcaccaatgcgtgatctcttccagttctgcgagatcctgtccaccccggaagtgagccgctgggcagggcccatcatcgacatcctcctggactatgtgggcaacgtgcagctgtgctcgcggctgaaggaacacatcgagagcttcgaggactgggccgtcatcaaggagaaggcagaacttcccagacctctggctcacctgtgccggctgcaagtgagaaaggctgtcgggaagcaccgcataaaactcctagacctattgccgctcccgggcaggctgattaggtacctgaagtacgagaacactca

SEQ ID NO: 243 - DNA, 383 bp, Canis familiaris
  misc_feature (225)..(225): n is a, c, g, or t
agcagcctcctgacgtcatgtgaagaccaagcaccccccattaaaggataggtttgggagccatggagcaggagctccagagcctgacatgcagtcatttgctaaagagcagagaaatttggggcttttctgtccccatgccctgggggctagacctttctaagtgaaatatgaagggtggactttataggcgagaattgggtgcattcagagcgggaaggtacncctcggtcgtggcctaaaggcggtcgccccactgccctgacctgtctgctcttggggttccttactggtcctgtgcatcatggtgggccactgaaaccgaagcagaggtggccgnctgggggcccccgccgacgacagttcagctgagggacaccaga

SEQ ID NO: 244 - DNA, 222 bp, Canis familiaris
gcctggtgctcatctgtgaagccatcccccacccgggcagcttggagcccccactaggccggggggccttcctcagccgccacagcctggacatgaagttcacttactgtgatgagaggatcgcagaggtcgctggctacagccctgatgacctgattggctgctctgcctatgagtacattcacgctctggactcggacgccgttggccagagcatccaca

SEQ ID NO: 245 - DNA, 105 bp, Canis familiaris
cacctgttcctgatgtatatagagcaagcaacatgtacaaacctgcagttttaaagcatttttttcatatcactctgacttgttttctacattcccgtttgtata

SEQ ID NO: 246 - DNA, 114 bp, Canis familiaris
gagaacatccgttcctgatgtaaatagagcaacatgcacaaacctgcagttttttttactttttcattctaccctgatccagttgctacattcctgtttctatgaaatctttga

SEQ ID NO: 247 - DNA, 566 bp, Canis familiaris
acttggcctctgtgcagactgccaggcggaaggggacatcccgggctgtgccatgatgctagctccttgcagcgacagccggcagcaacagcacctgaagcacaccagcaggaagcagatccactatggcagcccacagcacctgtgctttgatgtcaggcaagagcaggtgattcttcagaactgcaccaaggaaggccctgctatccatcaacagcactgggacttccaagagaatggaatgattgtccacattctttctggaaaatgcatggaagctgtggtgcaggaaaacagtaaagatttgtacttgcaccagtgtgatggaaaagccagccagctgtggcgatttgacaatgtcaacactgtggatgaacggtgactgtcaaagggaaaacagaagtttagtcatcgcagttcagccccaagagaactgaaagagcatgtttatttcatgaagcagacccttttgtgtttgtgcttctggtgacagtagaaaagaaagctccgtgaatgaatatagaaagtctttgcttcttacaccttattgcattgactgctggctg

SEQ ID NO: 248 - DNA, 528 bp, Canis familiaris
gcttcatcagtgcagaatccatctgcaatccctcctggtcccaaatgaatcagagtagatgatgaagaaaagggagattttttcagaattattttcaggacacctaagatactaaacgttcttcttgggttctgccacattttccctcatttgagtttactttccactgtactttttcattccattgtggtattttatatagctcatcattctgtagaactgtgcagtttgtacatagaacactgcacacagaaatgttttttcactgcaaatcctatcctctttgatacttgatttttttaaaaaataccgttcagaatgctttaataagctcagcttgagcattaccatttccaggacacttttcatgaattgctgagaagcccccattttttactgctgctatctgcagctggatgatactttaaaatgtataaaaactatttaaattgatattccccaaacgtggacgaagtgagaccctccaaaagctggagtcttatgggattcttcacatgtttatcttag

SEQ ID NO: 249 - DNA, 193 bp, Canis familiaris
agaccaacatgtttgccgtgaccagcatgctgagggtgacagccgaagactggaagcagggggagaagttctcctgcatggtgggccacgaggctctgcccatgtccttcacccagaagaccatcgaccgcctggcgggtaaacccacccacgtcaacgtgtctgtggtcatggcagaggtggacggcatctg

SEQ ID NO: 250 - DNA, 240 bp, Canis familiaris
gaaagtttgcaatcaacaggatcacgagaccattcaaatctctccattccttcaagagctgctcttcctgctgacacagttagtgtgggggattttttgccattgaaagctgaactggatacaacttacactttcttaaaggagacatttttaaatcctctgacgtcatctcactcctcttcagtgacaatgtctgctaatgccaaacgaccaacgcagattggataatgactttgtgaa

SEQ ID NO: 251 - DNA, 508 bp, Canis familiaris
  misc_feature (333)..(333): n is a, c, g, or t
taggacggagtaggcccctcagagagaatacttgagacttcactcagtggcccccagaggtgctccatggagaaggcaagccatggggatgaaaccccacatggtctagtggaaccatggcagccacccatgtacattccccatggtcggtcccagccccttcattccagttgcctcccactgtagaggttgtaaatcccaattcccactgttccagcttcctttgcagcttggggtgacaagtgaccaggtatggccagtgcaatttaagaggcagtcttacaggggcttctgggaaagtttatgcttttctgataaaagaggcagacatgngtaacacctctctgcccctccattcttcctgccttgattgaggatgtgatgcctggagccccaacanccaccttgctacnatgagaaaaaggcnaagagaatctcacagacactgacctaccctaccatcatttgaccaagctgatgccggcagccatactccttcagaattctt

SEQ ID NO: 252 - DNA, 208 bp, Canis familiaris
aatgacaggatgtacgggaacagacgagggaatgtgcctcctaaggactatggtggtgatgtcacagagcagtcaaacatacgcatttcatctgctggaaatagaagtgctcgagacaatgaaccatccaaatctactaaccgagagatctgtagtccttttgcgggaatgctctttggaggtgaagatcgagaacttattcagagaa

SEQ ID NO: 253 - DNA, 570 bp, Canis familiaris
gattgaaatcaatgcctcctggtctgccttcttcactgatgtacctatctttagaaaataattcaatttcttctataccagaaaattacttcaacgaacttcccaaacttcaggctgtacgaatgtcacacaacaaactacaagatatcccatataatatttttaatctttccaacctaatagagctcaatgttggacacaacaaactgaagcaagcattttatattccaagaaatttacaacacctatacctacagaacaatgaaatcgaaagtatcaatgttacagtgatgtgtccgtctgttgacccactgcattaccaccatttaacatacattcgtttggaccaaaataagctaaaagagccaataacctcatacatttccctctgcttcccttatatacacactatttattatggtgagcaaagaagcagtaatggtcaaacaatacaactgaagacccaagttttcagatttcaggatgatgctgatagtgaagaacatgaggatcaccaagaaggtccagaacaagaagaaacagaagaaaacactgaccatcactactatg

SEQ ID NO: 254 - DNA, 443 bp, Canis familiaris
  misc_feature (49)..(49): n is a, c, g, or t
tattcttctgaattctaatacagttaaaaagccaaatacttatcttcantattaagtaaaattaaatctcatcaatcttgactttcacagatgaagcacagaccaggcaataaaccacactactacatctaccaagactgatcaaatggtaggctgaaatttggaattcattaatagaaaaataatttaaaaaaattaaaaccttactgttttaactaaaatttcagtattagtaaaattacttcttttaaatgaaacaaattatagccgaaattttttcttttaaataagaaaacaaaaacaaatgaagtcaaacacacagganaaaagtggggacttcccgtcaatattatttctctaactcaaaatagtaaagcaaaagtaatcagaaattttcctctatagtaataaggagaaagaatgactttcaccccaagttctaa

SEQ ID NO: 255 - DNA, 187 bp, Canis familiaris
caagatatagccaaataaccccagctagcagtgaacaaaactgacagattggtgtgctaggaagggtgggctgggacacaattccatgttttttgcactaaaaaccttctctgtaaatagggataagagaaactcttactatgcagataacgtttttgaatggtgaacaggctattttgtacatcaa

SEQ ID NO: 256 - DNA, 477 bp, Canis familiaris
ggtcatctgggctctgtgcttcctgctgcttctcctggaagccggttctgccaagaatctctggaaacgggcattgcaccccaggctggccgagaagccccgcgccgaggaggcggcgggtccccggcaaccccgcgccgaccgctgccccccgccgccgcgttcgctgccccccggcgcctgccaggctgcgcgatgccaggcggactccgagtgcccgcggcaccggcgctgctgctacaacggctgtgcctacgcctgcctggaggccgtgccgccccctccagttttagactggctagtgcagccgaaacctcgatggctcggtggcaacggctggcttctggatggccctgaggaggtgttgcaagccgaggcctgcagcaccacggaggacggggcggagcctctcctctgcccctcaggttatgagtgccacatcctgagccctggtgatgtggccgagggcatccccaa

SEQ ID NO: 257 - DNA, 57 bp, Canis familiaris
ccgctgtgacagtggtaatgtacttcaggagatgttcaatctctaccaaaaatgact

SEQ ID NO: 258 - DNA, 107 bp, Canis familiaris
cagaatgtaaggaaaatgcctcggaaatgtatacgtgtagttattaactcttgagtaaagattttgtatctccagccaattagatatgatggtagaagatccaaaaa

SEQ ID NO: 259 - DNA, 443 bp, Canis familiaris
  misc_feature (48)..(48): n is a, c, g, or t
ttcgtggcagttttgtcctactgcttccccagcagagcccccttaggnccaaggcctcatccaaactgtcttggcctctttgtagtttcctatgtccgacacaaggaacacacctactacaagtggtggaaagancttagcatgcctaacctgatgttagggaagggaagaatccgggtagagggacctagtggatctcaggtactggatgggtgttgagggcccacttgtatggcgtcactgcagagagggcaggacccccgtcaggagctataaggaggacagttttagcacagggtgggaatagccaacgggtagagagctgctcaacatgagagcagctgtccactgtgaggngctggcggttaaaagaagttggattatggaaagtgttcctccgtgggatgatacataagaccagatcttcgatgcttaactttggg

SEQ ID NO: 260 - DNA, 298 bp, Canis familiaris
  misc_feature (176)..(176): n is a, c, g, or t
agctttggctttgcctccagccaggccctgcttcccctccctctctcctttccctttccttgcactccctttcccaccacctccctttcctctccctacattctcaccccctcttccctacctcatctccccactgcctctcaatcccagtagagttgatctcagttaacactgtngggaggcctggcaccagccaggacccaagacttcagtcccttgaaaagttaagcccagctctttgntctgcccccaancccagccnagccagcttgtcacccataaaacaggttcaaccttg

SEQ ID NO: 261 - DNA, 470 bp, Canis familiaris
  misc_feature (438)..(438): n is a, c, g, or t
agtcctgctcttggttttgggcggcgcggggctccgggtcccggcagcaggagccagcacagtccggccagatgacataataccaggtgtagaagacagcgtggtgaccccaggcacagaagacagcgtggtgacgccaggcgcagaagataacgtggtgactgacggtgccactgaagagccttatgagtctggcttaacccctctggtgacaaaaaatacagagagtgtaacagacttacacttagaggatgggccgactcaagaaagcacagtccatgccaaagaagagagccagagcaccacaaccttgaatgtagtgactagtcactccagagagaaagtaggtgaagacaccgagacaacggttgagaaagatggcctggcaacagtgaccctggttgggatcatagttggagtcttactagccattggatnaattggtgggatcatcattgtggttgctcgaa

SEQ ID NO: 262 - DNA, 298 bp, Canis familiaris
  misc_feature (72)..(72): n is a, c, g, or t
gcctgggcccagtcaagcaaggcaagccggggtgggtggtgtgccctcttccctttcaagggcctttgtggncatcctccnggccacatttggtttgggtgtcaaaccttctaaagctggtttttggttttttttggttttantttggtcctgttttgctttttttaaagacaccgggcttgctgtcttaataaaatgggaatttgtncccttgggggcaacttaaaagttctcttctgcctgctaaatggancgattgattgtcgtgtctctgtgaccccacctcaccgtgtaaaga

SEQ ID NO: 263 - DNA, 365 bp, Canis familiaris
  misc_feature (27)..(27): n is a, c, g, or t
gctctggcctgagaaatagaacctgtnaggctcctaacattactttctgcctcagaggaaagacgtaggacatcataataatnctgtaattttaagaaagcaggtggattcaaagncttgatgtaaaaaaataaaagaaatgcccatcttggtgacacgtaccncncgccctacatggggtcatcattggaaaggtcgtcaaagtagaggncccacctgccatcaggtgggcaccatccattcagctcagtacatttcttcccgtaggtcagttgacttcttttacccaaacacaaagattttacgctnagnagatgctctgtcacggttgtgcttgtgcaggtctgttatccttgcaactcgta

SEQ ID NO: 264 - DNA, 88 bp, Canis familiaris
tcttacagacaccttgttccccttagccagagagccctacagaattcagatttttagagaaattgctcacaaaagttagttacagttg

SEQ ID NO: 265 - DNA, 234 bp, Canis familiaris
gagcataacgaaactgtgaaacttcccaaagccttgtcagtggttaaaagtatttaacactctcctgttcatgacagatgttctgtttttataacctaccaaaaggaaactagaggcttctgggtgaagaagatttttttgtgaagtgggttctgcaagcagcctgcaagccaagggtagtgtccattgctgggaatggttaaacatgaaaggctgatagctggtataacatag

SEQ ID NO: 266 - DNA, 328 bp, Canis familiaris
gagaaaccttcagttcactaccgtatccttaaatttcagtactactgaaacagaaggcttaattgtatggataggaaaagctcaaaatgaagaaaatgattttctggcaattggtctccataatcagtccttgaaaatagcagtcaacttaggagaaagcatttctgtgcatatgacctatagcaacggcacattccattgtaataaatggcaccatgtaagagtaattcaaaatcagactcttattaaggcctacctagatgacaatctaattctctctgaggatattgaccctcacaaaaaatttgttgctctgaactatgatggc

SEQ ID NO: 267 - DNA, 307 bp, Canis familiaris
gaataacccactctatggtgacataggtttggaggaagctatggaagaaagaaaaaagaatccctcatggaccgctgaggaatatgacaggcattccctgcatacaaacctttctgggcatctgaaggaaaaccctaatgacctacggttttggttgggagacatgtacacacccggttttgacaccttattgaaaaaggaagaggaacaagaaaagcattcaaaatattgtcgtgtaggtctgattttgctccttgttgcctgcatcttggttttcatagtgacagttagcacatttttcacctga

SEQ ID NO: 268 - DNA, 314 bp, Canis familiaris
  misc_feature (42)..(42): n is a, c, g, or t
ggtttcagagcccaagacagacagaaatcgagggagaaaggnaatattccagaatcaggctgggtaggaaaatctggggggaagccaaactttacctcagctttccncnccagggctctggtcctcccatacacacgtagccaaggccgtggttctccatccccttctgagcactcataaagcctcaaattcagggattctgactaccaagaattcgtgtaaaaggacattttactgagctgagcacatcagtttttactcattgatttattgttaaaactgtttcatttctgttgcaggttttcattatgctt

SEQ ID NO: 269 - DNA, 374 bp, Canis familiaris
  misc_feature (67)..(67): n is a, c, g, or t
gaattgtcagaatcacactccaaattctttatttttgtagttacttttttccatttagtaacacatntagcatatgcatatatttatatatacatatatgcacaaatatatatattaataactgtgcctgaactttgaagaccttgggagaaaaaattaaagaataaatacatttggggtaataactatacctactatttccttcttgatatcctttcattttctgcaaactggcttaatttggaatttaccttagtctaatataaatttggacagaacagatttccagatgtatgtcagtaatcctagtgagtactatttatgcttgaatgtcttgagaaatatttatgctccagaggtccttttctcctata

SEQ ID NO: 270 - DNA, 523 bp, Canis familiaris
  misc_feature (32)..(32): n is a, c, g, or t
gcctcgtgccgtggatttactaatgaagtttncccattcccncgattgcaaatagctctnaatcttattaaacnaaccttcccattgtaaccagaacacagaggctaagtatctgttgcatgtacttataattcacttaagataataaatgcataaagatggtgactcttccctatggttctgcatggccagactccgtttcttacctctactgaatattttagcaagttacacaatttacagagtgattaacagaggtctgtttgtaaaggtattattattcctttccttaattattccgtagtgtgcagataacagagcagccaccttctcatccaagtggcctacggagatcatgtctcttctggtcatttcaagtctatatttatttatgtatgtaatnnaaaaaaagtgatttgtgtgtgcgtctgtcatgttatttagagagaagtaatcttgtaaaaaaacattttgtaaaaacaaaaaagtattgtaaatagcctgatattctgtgactcattat

SEQ ID NO: 271 - DNA, 439 bp, Canis familiaris
ggatcaggagatcagaagtcttcgagagaaacttaacaaactcagacaacagaatgcttgtttggtctcacagaatcattctttaatgactaaaatggaatctgtccattttgaattaacacagtcaagagcaaaagtttctatgcttgaatctgttcaacaacaggcagccagtgtgccaatcttagaagaacagattataaatttggaagcagaagtttcagcacaagataaagttttgagagaggcagaagataaattagagcagagccagaaaatggtgattgaaaaggaacaaagtttgcaaaagtctcaagatgaatgtataaagttaaaggtggacttacttgaacaaagtaaacaaggaaagagagctgaacgacaaaggaatgaagcactatataatgccgaagagctgagtaaagctttccaacattat

SEQ ID NO: 272 - DNA, 207 bp, Canis familiaris
ttttccatctacctcacaacttaatttacatcagagaattcatactgatgagaaatactatgaatgtaaggaatgtgggaaagcctttacccgtccctcacaccttcttcgacatcagagaattcatactggtgagaaaccccataaatgtaatgaatgtgggaaagcttttcgttatgacactcaacttagtcttcatcagataat

SEQ ID NO: 273 - DNA, 424 bp, Canis familiaris
  misc_feature (147)..(147): n is a, c, g, or t
agattggacccaccagggcatcgcgaagctgtaccacaaagtgagccaaaacggctataagttcctgtactgctccgcgcgggccatcgggatggcagacatgacgaggggctacctgcactgggtgaacgagcggggtacggtgcngccccaggggcccctgctgctcagccccagcagtctcttctcggccttgcacagagaagtgattgagaagaagccagagaagtttaaagtccagtgtttgacagacatcaaaaacctgtttttcccaaatacagaacccttttatgctgcttttggaaaccgaccggctgatgtgtattcatataagcaagtaggagtgtctttgaacagaatatttactgtcaatcccaaaggagaacttgtccaggaacacgcaaagaccaacatttcctcgtat

SEQ ID NO: 274 - DNA, 291 bp, Canis familiaris
ggaactcttcagatactgcactctccctgagattctgaaatatgtggagtgcttcactggacccaatattatggccatgcatacaatgctgataaacaaaccaccagattccggcaagaagacatcccgccatcccttgcaccaggatctgcactacttccccttcaggcccagcaataaaattgtctgtgcttggacagccatggagcacatcgacaggaacaatggctgtctgtgtgtgttcccaggcacacacaaaggctatctgaagccacacaattatccccaatg

SEQ ID NO: 275 - DNA, 64 bp, Canis familiaris
aaagaactatcaggacatggttgtggagggttgtgggtgtcgttagcacagcaaaatgaaatat

SEQ ID NO: 276 - DNA, 383 bp, Canis familiaris
ggattagcaggtctttgcaccaagacgagcacagctggtcacaaatcaggccactgctagtaacgtttggccacgatgggaagggacaccccctccacaaaagagaaaagcgtcaagcaaaacacaaacagcgcaaacgccttaagtccagctgtaagagacaccctttgtacgtggacttcagtgacgtggggtggaatgactggatcgtagctcccccggggtaccacgccttttattgccatggggaatgcccctttcccctggcagatcacctgaactccactaaccacgccattgttcagacgttggtcaactcggttaattctaagatccccaaggcgtgctgtgtaccaacagaactcagtgctatctccatgctg

SEQ ID NO: 277 - DNA, 523 bp, Canis familiaris
  misc_feature (246)..(246): n is a, c, g, or t
catgcttcctgacaattttatccaatatataaaccagggagctatggtgcactctcatttatacacgtgctgtaagctgtgtcgtgcaaacgtagggggcctccctgaaggtgaactcgtgacttcacccgacacaggcgcgtttccagtccagaagcagtgtgggctcctacggcaggcgtgggtcctggctgactctgacttccaatacaatagccatcactagcacggtgttatttttttttngtttgtttaaccaacgtagttgtattagtagttctataaagagaactgcttttaacattagggactgggaacagtccattgagataaaaaggaaagtgttttnctcacgggaaaacatgtcaggnaaaaataaaaaacactttctacctctgtttcaaatttttgaaacgcttattttaaaccaaattttaatttctgggtccaaaataagtttcaaggacatctgttcttccatacaaaataggtaaggctgtctatttcttacggagctcatgga

SEQ ID NO: 278 - DNA, 537 bp, Canis familiaris
  misc_feature (54)..(54): n is a, c, g, or t
atctgggattcggagctgcacattgcaggcctcagacttctcaacagcctctcnctgcccgactatgtacatccacagctgcgacgggtgatgcctgccttgatggagatcctgcagtcagactatatcctggcacaggtncaagctatacgattgctgagctacttggcacagaaaaancgnccttctctatgatattctcaactgccaggtgcattccaacttcctgaacctgtttcagnccacacagccagggagtctgctgttcgaggtactggtgtttgctgagcggctaagtgagggccggaattcaccccnctaccgtgctgtgaagtggcattacaatgaacaatctctgcatgaagctctttttggggatgagtcacgactggcagaccggctgcttgccctggtcatccaccctgaggaggaggttcagatccaggcctgcaaggtcatagtcagcctgcagtgctcccaggatgtgggagtccggccctcctcctgccggcccagtcattcctacttcaatagcgg

SEQ ID NO: 279 - DNA, 534 bp, Canis familiaris
aagactctggattttactcctgcggaatctacaaatcctctgagatccttacttacagaaatatccatcttgtggtgtctccagagtctatcatggctgtgtgggtggagagagggatggcttctgggcagaaatttggctcccactttcctcacctggctggttgtcccttattccaaagcccccaaagcaactggcatctcatcgttcccagctccacggtggctgtcctgctgctgctggtgctcaccttcctcatgatcctgtacttgaggaaagcccgaagaagctccgggaaaggtgaggacaaatcccaccacatctacaacaacatctcagtccagaaggaacagaccaccggctccagtcaacatatgggctctgaagaggacactggggccatctgctatgcctcacttacccacctgaaccactttgtgcctgaggactccatctatgtcaacatccaactcaacctgaagcccatacctgacccccttctgactgtggaatatgccgacatcgctggagcca

SEQ ID NO: 280 - DNA, 535 bp, Canis familiaris
tagccattgtccatccttttacttaccgagtgctgcccaagcgttactatgccttcctaacgtgtggatttgtgtgggcaacagttttcttatacatgctgccattcttcatcctgaagcaagagtattatcttgtccagcaagacatcaaaacctgccatgatgtccacaacacatgcgagtcctcgtctcccttccaactctactacttcatctccttggcattctttggattcttaattccctttgtggtcattatctactgctatacagccatcattcagacacttaatgcctatgatgacagatggttgtgctatgttaaggcgagtctccttgttcttgtgatttttaccatttgctttgctccaagcaatattatacttattattcaccatgccaactactactacaacaacactgatggcttatattttatctatctcatagctttgtgccttgggagcctaaatagttgcctagatccactcctttattttctaatgtcaaaaatcatagatcactccaccgttta

SEQ ID NO: 281 - DNA, 340 bp, Canis familiaris
  misc_feature (44)..(44): n is a, c, g, or t
gaagagaagatttccctgaacctgcttctaacgaaaggtgagcncatgccagaagaggagatgggtgactgctttgctaccctgtttggactgaatcctgaggggtggaagtccgagcctgcagcctcctctgtcaaaggttcagaaatctgctttgaagaagancttccagacgaaatcnctgcagaaatattcacaactgacattcttggcctaaccatttcncaagatttcagacaagatccctacggtcagtgaagttgtaaagaatgtttgaagcacaaaggactttgtgtgtgggtttttttttcccccaagaagcactgctttccctgcattt

SEQ ID NO: 282 - DNA, 228 bp, Canis familiaris
tctcgcctctgttgttagccgaagactcgcctcttagccgcccgccgcacagacgcacgagtaaaaagtgcagctccatcggctgatcctcgctacgctccgagtttaggcggcaccgggcgtcccacgatgccgaagaataagaagcggaacgctccccaccgcggtggcagtggtggcagcggctcaggggcagctgcagcgacggcggcgacagcaggtggccaa

SEQ ID NO: 283 - DNA, 211 bp, Canis familiaris
gtggctatcaacttagtgttcatcagagatttcacactggtaaaaacccttattgtacggaatttgggaagacctttgttaatggctcagaccttgttcaacacaagagaactcatagtaatgataaaccttatgaatataaaggttgcgaggaagcctttatatggacaacttactcaaatgagacagttgatactggtgatactttatg

SEQ ID NO: 284 - DNA, 223 bp, Canis familiaris
atgccacccagctaggctgttcatcatactgtacactcacagagaccatccccaaagctctgtgcatccagctagccagcccaagccctagacccttatgatatcagagcagtagggactttaggatctaatctaacatcttgacttttctgacctggaaagtgatatttaaatgtgactgaggtgtcttgggagtcccccatgcttgttcctctgcagttat

SEQ ID NO: 285 - DNA, 420 bp, Canis familiaris
  misc_feature (165)..(165): n is a, c, g, or t
ccccattcatctgtggttcacagttaaggaggttgtggggggaggagcccactctgccccagggcaggcgtggcttccagtcaaagctggatcagtcgctgtcggagccacatcaatcctgtgctattgaccactgaccttagtgttcacatgtgaattcaaacnttgacgtgtagcacttaaacatatttgggcttgtttctcaggtttaaatatttcaaatgtaattaattgttttaaccagtgcagttatcaatgcaattttatatttcctaaaagaaggaagagtgggtttttattgtacatgttgaaaagaagagggaagagtattatgtatttaatttaatttggaataagccatagtcttaaagagctgtggtttgaatttgttatcttgggctgtccactgcatgtagatac

SEQ ID NO: 286 - DNA, 455 bp, Canis familiaris
atggccagcgagtatgatgccgtaaacttgggccaaggcttccccgacttcccacccccagacttcgcacttcaagcctttcagctggctctcagcagtgacttcatgctcaaccagtacaccaaggcgtttggttacccacctctgacaaaaatcctggcaagtttctttgggaagctgctgggacaggagatagacccactcaagaacgtgttggtgaccgtgggtgcctacggggccctgttcacagccttccaggccctggtggatgaaggagacgaggtcatcatcatagagccattttttgactgttatgaacccatgacgttgatggcagggggtcaccctgtgtttgtgaccctgaagccgagccccacccaggatggggaactggattccgccagcaactggcagctggaccccacagagctggccagcaagtttacttctcgtac

SEQ ID NO: 287 - DNA, 243 bp, Canis familiaris
gctgagactgcttttaaaaggaaccttagtcttttaatggacatagaagcagcagaaaagtctctacagacccggattcactcaattccatcaccagaggtggtttctcttgagactctttactgggcatcagtagaagaatatattcccaaatgggaacagtttcttttaggaagagcaccatatcctatcggtgttgaaaatgaaaacgaagcagaaaataccattcaaaatgaggctcag

SEQ ID NO: 288 - DNA, 396 bp, Canis familiaris
  misc_feature (276)..(276): n is a, c, g, or t
aagtcaaaggtccttgtcaggccctgtcctgtcatcatcaaatgtcatctcagcgggccacatcagccttctgttttaagccagatcgcctctgttagatgctcacagggacatgtatttctcgttcaaagctctttaccacagtggcagggttgcatttgttggcatgattgtttgacaccagtcttcctctctagactgtgagctccatgaaggcaagggcatgtctatttttttccatatcaaatgctcctggcccagcacaggacctgctcncactgaattagtgaactcagattcgtaaggcagctactgcatgagtggtaacaggtgaattatttcacctctttcattttcttacttacaaaagggattatatccgttctatgtccctgt

SEQ ID NO: 289 - DNA, 565 bp, Canis familiaris
  misc_feature (502)..(502): n is a, c, g, or t
agttctaccgcgatgatgaccgagatgagaagaccgtccagagcttgcggatttcggccgtcatcctgcaccccaactacgaccccatcctgctggacacggaccttgccatcctgaagctgctggacaaggctcgcgtcagcacccgcgtccagcccatctgcctggcggccccgcgggacctgagcacctccttccaggaggcccgcatcaccgtggccggctggaatgttctggcagatgcgagaagccccggcttcaaaaacgacacgctgcgctcaggggtggtcagggtggtggactcgctgctgtgcgaggagcagcacgaagagcacggcatccccgtgagcgtcacggacaacatgttctgtgccagccgggaccctgctgccccttcggatatctgcatggcccagactggaggcatcgccgcgatggcctttccgggacgggcgtcccccgagccacgctggcacctggtggggctggtcagctggagctntgacaaagcatgcagccacagcctgtacacggccttcagcaaggtcctgccttttaaagactg

SEQ ID NO: 290 - DNA, 522 bp, Canis familiaris
  misc_feature (214)..(214): n is a, c, g, or t
aagcttcctcagtaactcatttgcccattgagaacctgaagccaaacacaaggtattattttaaggttcaagccaaaaatccacatggctacggacccatcagcccttcggtctcatttgttacagaatcagacaatcctctgcttgttgtgaggccaccaggtggtgagcccatctggatcccatttgctttcaaacatgaccctagctacancgactgccacggccggcagtatgtgaagcggacgtggtatagaaaatttgtgggagttgttctttgtaattcactgcggtataagatctacctcagtgacaacctgaaagatacattctacagcattggggacagctggggaagaggggaagaccactgccagtttgtggattcacaccttgatggaagaacagggcctcagtcctacgtagaagctctccctaccattcaaggctacttccgccagtatcgccaggagccggtcagctttggcaacatcgggtttggaactccctactactatgtgg

SEQ ID NO: 291 - DNA, 354 bp, Canis familiaris
  misc_feature (185)..(185): n is a, c, g, or t
tccactttctgctaagtcttggaaacagttggataaaacatgatctgacccccagaggtcatgctcagctccacagagcatgaccccagagatacttagagacaagacacgtggctcccaatgtcagcatgacccgcctggctgggtgactttgtgggagccccttgccagcaccggatccctgntttattatcctgaagatccagcacgtctgacctggcgatcccacaggcctcgggaagaggtcaccgtctttgggagctgtatgaagccccattctaagacagagttcagcatcacatttcttccagatttgactctggaaggcacgtacctatcccaaatgttctcaaa

SEQ ID NO: 292 - DNA, 531 bp, Canis familiaris
agagctctggacttacatatcctgaggataaacttgtatatggcatacaggagccatcagctggcactagcactctggcagttcaaggatttgcaggtgcaacaggaactttgggacaactagattcttcagatgaggaggatcaggatgggagtcaaggtctgggcaagagaaagagggtcaaactaagcagtagcaccaaagatcaatctataatggatgttttgaaacataaaagttttctagaagaactgttgttttggactataaagtatgaattccctcagaagatggtaactttcttactcaacatgcttccagatcaagagtataaggttgcttttacaaaaacttttgttcagcattatgctttcattatgaaaacactgaagaaaagtcatgaatcagatacaatgtctaacagaattgtgcatattagtgttcagttgttcagcaatgaagagttagccagacaggtaacagaagaatgtcaactgctggatattatggtcactgtgctattatacatga

SEQ ID NO: 293 - DNA, 515 bp, Canis familiaris
agatgttctgatgccagtgccacagcctgacaactccctgatggattttgttgtgacctgccaagggatgatgccaacagaggtctgtaccgtggtttctgaccctacctgccagatcacgtatgacacagtctgcgaccctgtaggtgaggacatgggtcatgagtgtttgctgaccgtgagaagagccttcagtgggtctgggacgtactgtatgaacctcactctgggagataatacaagtctggccctcacaagcacccttgtctctatccctgcaagagacccagcttccccttcaagaatggcaaatggtgtcctggttgccgttggttgcttagccatatttgtcactgtgatcaccctttgggtatacaaaaaacacaaggaatacaacctgatagaaaatagtactgggatcgtggtcaaaggcaaaggcctgaatgtcttcctcaaccgtgcaaaggccatgttctaccctggaatccaagaaaaagatccactgctcaagcacc

SEQ ID NO: 294 - DNA, 52 bp, Canis familiaris
aagacacacgctaatgcatgcccggagtcaataaagacatggctcccacaga

SEQ ID NO: 295 - DNA, 154 bp, Canis familiaris
  misc_feature (107)..(107): n is a, c, g, or t
ggcaccagtcaagatgtcactgagagggatattgtagggattggacataaattatatggaattccgaaagacaccagcctgaaggaggtgtctagatatcgagcccngctggtgaaaggaagaaggatcaacctttgaagtagctgcctcctgg

(19) United States
(12) Patent Application Publication
(10) Pub. No.: US 2009/0217398 A1
(43) Pub. Date: Aug. 27, 2009

(54) METHODS TO IDENTIFY FAT AND LEAN ANIMALS USING CLASS PREDICTORS

(75) Inventors: Samer Al Murrani, Topeka, KS (US); Kim Gene Friesen, Carthage, IN (US); Ryan Michael Yamka, Topeka, KS (US); William D. Schoenherr, Hoyt, KS (US); Sukhaswami Malladi, Lawrence, KS (US); Xiangming Gao, Topeka, KS (US)

Correspondence Address: COLGATE-PALMOLIVE COMPANY, 909 RIVER ROAD, PISCATAWAY, NJ, 08855, US

(73) Assignee: Hill's Pet Nutrition, Inc., Topeka, KS (US)

(21) Appl. No.: 12/281,408

(22) PCT Filed: Mar. 2, 2007
(86) PCT No.: PCT/US07/05438

§ 371 Date: Nov. 13, 2008

Related U.S. Application Data
(60) Provisional application No. 60/778,567, filed on Mar. 2, 2006.
(60) Provisional application No. 60/824,318, filed on Sep. 1, 2006.

(51) Int. Cl. A61K 31/7105; C12Q 1/68; C12M 1/00; C40B 30/04; G01N 33/53; A01K 67/027

(52) U.S. Cl. 800/13; 435/6; 435/287.2; 506/9; 435/7.2; 800/21; 800/25; 514/44 R

(57) ABSTRACT

A combination comprising two or more polynucleotides that are differentially expressed in fat animals compared to lean animals or two or more proteins produced by the expression of such polynucleotides is disclosed. The combination and probes based upon the combination are used for formulating a prognosis that an animal is likely to become fat, developing a diagnosis that an animal is fat, screening substances to determine if they are useful for modulating the amount of adipose tissue on an animal, and detecting the differential expression of one or more genes differentially expressed in fat animals compared to lean animals in a sample. Methods for using class predictor gene profiles to identify fat and lean animals are also disclosed.

This application claims benefit of U.S. Provisional No. 60/778,567 filed Mar. 2, 2006 and U.S. Provisional application No. 60/824,318 filed Sep. 1, 2006, PCT/US07/05438, filed Mar. 2, 2007, which are both hereby incorporated by reference for all purposes.

A Sequence Listing is submitted on duplicate compact discs labeled CFR (computer readable form), Copy 1 and Copy 2. The contents of the CFR, Copy 1, and Copy 2 compact disks are the same. The Sequence Listing information on the CFR, Copy 1, and Copy 2 compact disks are identical. The Sequence Listing is in a file named “8123.txt.” The file was created on Feb. 24, 2006 at 3:13 PM and contains 188 KB of data. The file was created using an IBM PC compatible computer running the Windows 2002 operating system. The Sequence Listing in 8123.txt is incorporated herein by reference in its entirety.

### BACKGROUND OF THE INVENTION

1. Field of the Invention

The present invention relates generally to genes differentially expressed in animals and particularly to genes differentially expressed in fat animals compared to lean animals.

2. Description of the Related Art

It is generally accepted in the scientific community that genes play a role in animal development and that the regulation of gene expression plays a key role in the development of some diseases or conditions that affect an animal's health and well being. Similarly, the differential expression of genes is one factor in the development of such diseases and conditions and the evaluation of gene expression patterns has become recognized as crucial to understanding the development and control of such diseases and conditions at the molecular level. To advance the understanding of genes and their relationship to disease, a number of methods have been developed for studying differential gene expression, e.g., DNA microarrays, expressed tag sequencing (EST), serial analysis of gene expression (SAGE), subtractive hybridization, subtractive cloning and differential display (DD) for mRNA, RNA-arbitrarily primed PCR (RAP-PCR), Representational Difference Analysis (RDA), two-dimensional gel electrophoresis, mass spectrometry, and protein microarray based antibody-binding for protein.

Gene expression in fat animals compared to lean animals has not been thoroughly investigated. Therefore, a need exists to identify genes and proteins encoded by genes that are differentially expressed in fat animals compared to lean animals. Such genes, proteins, and their fragments would be useful for formulating a prognosis that an animal is likely to become fat, developing a diagnosis that an animal is fat, screening substances to determine if they are likely to be useful for modulating the amount of adipose tissue on an animal, and using such substances to modulate the amount of adipose tissue on an animal.

Fat animals can be defined as those animals having an excess of body adipose tissue. Generally, animals such as humans, canines, and felines weighing more than 15% of their ideal body weight are considered fat. The most common cause of an animal being fat is an over consumption of food that results in an excess intake of calories. However, there are other factors that can increase an animal's chances for being fat, e.g., lifestyle, health, eating habits, breed, spaying, and neutering. Also, the incidence of animals becoming fat generally increases with age due to a general decrease in metabolic rate and in physical activity. Surveys estimate that 25% of dogs in the United States that visit veterinary clinics are fat to the point of being obese. Studies have shown that fat animals are significantly more at risk for diseases such as arthritis, heart disease, respiratory disease, diabetes, bladder cancer, hypothyroidism, and pancreatitis.

Modulating the amount of adipose tissue on an animal, including preventing an animal from becoming fat or treating a fat animal to reduce the amount of adipose tissue on the animal or treating a lean animal to increase the amount of adipose tissue in the animal, is difficult. Increasing the amount of adipose tissue on an animal usually involved increasing the amount of food consumed. The most effective and easiest way to prevent an animal from becoming fat or to reduce the amount of fat on an animal is with dietary restriction and exercise. However, it is often difficult to ensure compliance with diet and exercise programs. Other methods involve the use of drugs such as phentermine, fenfluramine, sibutramine, orlistat, and phenylpropanolamine. Unfortunately, side effects occur with these drugs. For example, the administration of fenfluramine and phentermine for the treatment of human obesity can result in cardiac valve damage in humans. Sibutramine can increase blood pressure and orlistat may have unpleasant gastrointestinal side effects.

Given the problems with current methods for dealing with adipose tissue on an animal, there is a continuing need for new methods and compositions useful for formulating a prognosis that an animal is likely to become fat, developing a diagnosis that an animal is fat, screening substances to determine if they are likely to be useful for modulating the amount of adipose tissue on an animal, and using such substances to modulate the amount of adipose tissue on an animal.

### SUMMARY OF THE INVENTION

It is, therefore, an object of the present invention to provide one or more genes or gene segments that are differentially expressed in fat animals compared to lean animals.

It is another object of the present invention to provide combinations of two or more polynucleotides or polypeptides that are differentially expressed in fat animals compared to lean animals.

It is another object of the present invention to provide compositions of two or more polynucleotide or polypeptide probes suitable for detecting the expression of genes differentially expressed in fat animals compared to lean animals and devices such as substrate arrays containing the probes.

It is a further object of the present invention to provide methods and compositions for detecting the differential expression of one or more genes differentially expressed in fat animals compared to lean animals in a sample.

It is another object of the present invention to provide a method for measuring the effect of a test substance on the expression profile of one or more genes differentially expressed in fat animals compared to lean animals as a method for screening a test substance to determine if it is likely to be useful for modulating the amount of adipose tissue on an animal.

It is another object of the invention to provide methods for formulating a prognosis that an animal is likely to become fat or developing a diagnosis that an animal is fat.

It is another object of the invention to provide methods and compositions for modulating the expression of one or more genes differentially expressed in fat animals compared to lean animals or for modulating the amount of adipose tissue on an animal.

One or more of these other objects are achieved using novel combinations of 295 polynucleotide probes representing 254 genes and gene segments that are differentially expressed in fat animals compared to lean animals. The polynucleotides are used to produce compositions, probes, devices based on the probes, and methods for determining the status of polynucleotides differentially expressed in fat animals compared to lean animals useful for achieving the above-identified objects, e.g., prognosing and diagnosing conditions relating to animal adipose tissue and for screening substances to determine if they are likely to be useful for modulating the amount of adipose tissue on an animal. Such substances, once identified, may be used to modulate the amount of adipose tissue on an animal. Various kits comprising combinations of probes, devices utilizing the probes, and substances are also provided.

It is also an object of this invention to provide methods for using “class predictor” gene profiles to differentiate between fat and lean animals. Class predictor technology can be used to facilitate the clinical diagnosis of an animal's body type, e.g., class prediction can be used in a blood-based test to make a positive determination as to whether an animal is fat or lean or has the propensity to become fat or lean. This and other objects disclosed herein may be achieved using novel combinations of 65 polynucleotide probes identified herein that can act as class predictors for fat and lean animals using blood samples taken from fat and lean animals. These class predictor genes can be used e.g., to develop blood-based test kits to predict if an animal is fat or has the propensity to become fat or they can be used to predict if a lean animal can maintain its leanness. Class predictors can also be used to define the body condition score of an animal and as such may have various useful applications in veterinary clinics.

It is also a further object of this invention to provide methods for using class predictor gene profiles to accurately identify fat animals and follow their progression at the biochemical level and indicate whether their gene expression profiles are consistent with being fat or lean.

It is also an object of this invention to provide methods to modulate the amount of adipose tissue in an animal in vivo by administration of one or more active ingredients that are shown in vitro to modulate the expression of genes involved in fat metabolism.

Further objects of the invention include use of the polynucleotides, probes, active ingredients and class predictor data disclosed herein in the manufacture of compositions, devices and kits as described herein, e.g., for modulating the expression of one or more genes differentially expressed in fat animals compared to lean animals or for modulating the amount of adipose tissue on an animal, for detecting the expression of genes differentially expressed in fat animals compared to lean animals and for predicting or diagnosing the body condition score of an animal, including the identification of fat animals from lean animals, and in methods for detecting the expression of genes differentially expressed in fat animals compared to lean animals, for modulating the expression of one or more genes differentially expressed in fat animals compared to lean animals, for measuring the effect of a test substance on the expression profile of one or more genes differentially expressed in fat animals compared to lean animals, for screening a test substance to determine if it is likely to be useful for modulating the amount of adipose tissue on an animal, for formulating a prognosis that an animal is likely to become fat or developing a diagnosis that an animal is fat or for modulating the amount of adipose tissue on an animal.

Other and further objects, features, and advantages of the present invention will be readily apparent to those skilled in the art.

### DETAILED DESCRIPTION OF THE INVENTION

### Definitions

The term “animal” means a human or other animal, including avian, bovine, canine, equine, feline, hicrine, murine, ovine, and porcine animals, that has adipose tissue. When the term is used in the context of comparing fat to lean animals, the animals that are compared are animals of the same species and possibly of the same race or breed. In preferred embodiments, the animal is a canine or feline, most preferably a canine.

The term “antibody” means any immunoglobulin that binds to a specific antigen, including IgG, IgM, IgA, IgD, and IgE antibodies. The term includes polyclonal, monoclonal, monovalent, humanized, heteroconjugate, antibody compositions with polyepitopic specificity, chimeric, bispecific antibodies, diabodies, single-chain antibodies, and antibody fragments such as Fab, Fab′, F(ab′)₂, and Fv, or other antigen-binding fragments.

The term “array” means an ordered arrangement of at least two probes on a substrate. At least one of the probes is a control or standard and at least one of the probes is a diagnostic probe. The arrangement of from about two to about 40,000 probes on a substrate assures that the size and signal intensity of each labeled complex formed between a probe and a sample polynucleotide or polypeptide is individually distinguishable.

The term “body condition score” (BCS) means a method for body composition analysis based upon an animal's body size and shape. Several methods are known to skilled artisans, e.g., methods disclosed in U.S. Pat. No. 6,691,639 and in the reference entitled “Small Animal Clinical Nutrition”, 4^{th} Edition, in Chapter 13 (ISBN 0-945837-05-4).

The term “Body Mass Index” (BMI) means an animal's weight (in kilograms) divided by its height (in meters) squared.

The term “Class Predictor” as used herein refers to a genomic, proteomic or metabolomic profile that is generated using supervised learning methods employing algorithms such as, but not limited to, Weighted Voting, Class Neighbors, K-Nearest Neighbors and Support Vector Machines from a group of pre-defined samples (“the training set”) to establish a prediction rule that then can be applied to classify new samples (“the test set”).

The term “DEXA” means body composition analysis dual-energy X-ray absorptiometry.

The term “differential expression” or “differentially expressed” means increased or unregulated gene expression or means decreased or down-regulated gene expression as detected by the absence, presence, or at least two-fold change in the amount of transcribed messenger RNA or translated protein in a sample.

The term “fat” as applied to an animal means any animal that is determined to have an excess amount of body adipose tissue or an animal that is prone to developing an excess amount of body adipose tissue using techniques and methods known to health care providers and other skilled artisans. An animal is prone to becoming fat if the animal has an inclination or a higher likelihood of developing excess adipose tissue when compared to an average animal in the general population. Generally, without limiting the definition, an animal is considered fat if (1) the animal has a BMI of 25 or more (a number considered to include “overweight” and “obese” in some methods of characterizing animal conditions), (2) the animal's weight is 15% or more than its “ideal” body weight as defined by health care professionals or related skilled artisans, (3) an animal's percent body fat is 27% or more as determined by DEXA, or (4) an animal has a body condition score of more than 3 as determined by skilled artisans using the method disclosed in “Small Animal Clinical Nutrition”, 4^{th} Edition, in Chapter 13 (ISBN 0-945837-05-4) or its equivalent using other BCS methods.

The term “fat-associated genes” means all or a subset of the genes identified by SEQ ID NOs:1-295, particularly the 254 genes identified herein as differentially expressed in fat animals compared to lean animals.

The term “fold” when used as a measure of differential gene expression means an amount of gene expression in an animal that is a multiple or a fraction of gene expression compared to the amount of gene expression in a comparison animal, e.g., a fat animals compared to a lean animal. For example, a gene that is expressed three times as much in the animal as in the comparison animal has a 3 fold differential gene expression and a gene that is expressed one-third as much in the animal as in the comparison animal also has a 3 fold differential gene expression.

The term “fragment” means (1) an oligonucleotide or polynucleotide sequence that is a portion of a complete sequence and that has the same or similar activity for a particular use as the complete polynucleotide sequence or (2) a peptide or polypeptide sequence that is a portion of a complete sequence and that has the same or similar activity for a particular use as the complete polypeptide sequence. Such fragments can comprise any number of nucleotides or amino acids deemed suitable for a particular use. Generally, oligonucleotide or polynucleotide fragments contain at least about 10, 50, 100, or 1000 nucleotides and polypeptide fragments contain at least about 4, 10, 20, or 50 consecutive amino acids from the complete sequence. The term encompasses polynucleotides and polypeptides variants of the fragments.

The term “gene” or “genes” means a complete or partial segment of DNA involved in producing a polypeptide, including regions preceding and following the coding region (leader and trailer) and intervening sequences (introns) between individual coding segments (exons). The term encompasses any DNA sequence that hybridizes to the complement of gene coding sequences.

The term “genes differentially expressed in fat animals” means genes from which the amount of mRNA expressed or the amount of gene product translated from the mRNA is detectably different, either more or less, in tissue from fat animals as compared to lean animals.

The term “homolog” means (1) a polynucleotide, including polynucleotides from the same or different animal species, having greater than 30%, 50%, 70%, or 90% sequence similarity to a polynucleotide identified by SEQ ID NOs:1-295 and having the same or substantially the same properties and performing the same or substantially the same function as the complete polynucleotide, or having the capability of specifically hybridizing to a polynucleotide identified by SEQ ID NOs:1-295 under stringent conditions or (2) a polypeptide, including polypeptides from the same or different animal species, having greater than 30%, 50%, 70%, or 90% sequence similarity to a polypeptide identified by the expression of polynucleotides identified by SEQ ID NOs:1-295 and having the same or substantially the same properties and performing the same or substantially the same function as the complete polypeptide, or having the capability of specifically binding to a polypeptide identified by the expression of polynucleotides identified by SEQ ID NOs:1-295. Sequence similarity of two polypeptide sequences or of two polynucleotide sequences is determined using methods known to skilled artisans, e.g., the algorithm of Karlin and Altschul (Proc. Natl. Acad. Sci. USA 87:2264-2268 (1990)). Such an algorithm is incorporated into the NBLAST and XBLAST programs of Altschul et al. (J. Mol. Biol. 215:403-410 (1990)). To obtain gapped alignments for comparison purposes, Gapped Blast can be utilized as described in Altschul et al. (Nucl. Acids Res. 25: 3389-3402 (1997)). When utilizing BLAST and Gapped BLAST programs, the default parameters of the respective programs (e.g., XBLAST and NBLAST) are used. See http://ww.ncbi.nlm.nih.gov.

The term “hybridization complex” means a complex that is formed between sample polynucleotides when the purines of one polynucleotide hydrogen bond with the pyrimidines of the complementary polynucleotide, e.g., 5′-A-G-T-C-3′ base pairs with 3′-T-C-A-G-5′. The degree of complementarily and the use of nucleotide analogs affect the efficiency and stringency of hybridization reactions.

The term “in conjunction” means that a drug, food, or other substance is administered to an animal (1) together in a composition, particularly food composition, or (2) separately at the same or different frequency using the same or different administration routes at about the same time or periodically. “Periodically” means that the substance is administered on a dosage schedule acceptable for a specific substance. “About the same time” generally means that the substance (food or drug) is administered at the same time or within about 72 hours of each other. “In conjunction” specifically includes administration schemes wherein substances such as drugs are administered for a prescribed period and compositions of the present invention are administered indefinitely.

The term “lean” as applied to an animal means any animal that is determined not to be fat using techniques and methods known to health care providers and other skilled artisans. Generally, without limiting the definition, an animal is considered lean if (1) the animal has a BMI of less than 25 or (2) the animal's weight is less than 15% more than its “ideal” body weight as defined by health care professionals or related skilled artisans, (3) an animal's percent body fat is less than 27% as determined by DEXA, or (4) an animal has a body condition score of 3 or less as determined by skilled artisans using the method disclosed in “Small Animal Clinical Nutrition”, 4^{th} Edition, in Chapter 13 (ISBN 0-945837-05-4) or it equivalent using other BCS methods.

The term “modulating the amount of adipose tissue on an animal” means causing the animal to lose adipose tissue, causing the animal to gain adipose tissue, or causing the animal to maintain the amount of adipose tissue on the animal if the animal is prone to gaining or losing adipose tissue. Thus, modulating the amount of adipose tissue on an animal encompasses preventing a lean animal from becoming fat and treating a fat animal to reduce the amount of adipose tissue on the animal, as well as treating a lean animal to add adipose tissue in appropriate circumstances, e.g., when treating a lean animal that is determined by skilled artisans to be so underweight that the addition of adipose tissue is desirable. Conventional methods may be used to assess the amount of adipose tissue on an animal, as well as to determine the animal's lean muscle mass and/or bone mineral content, information which may be of relevance in such an assessment.

The term “polynucleotide” or “oligonucleotide” means a polymer of nucleotides. The term encompasses DNA and RNA (including cDNA and mRNA) molecules, either single or double stranded and, if single stranded, its complementary sequence in either linear or circular form. The term also encompasses fragments, variants, homologs, and alleles, as appropriate for the sequence, that have the same or substantially the same properties and perform the same or substantially the same function as the original sequence. The sequences may be fully complementary (no mismatches) when aligned or may have up to about a 30% sequence mismatch. Preferably, for polynucleotides, the chain contains from about 50 to 10,000 nucleotides, more preferably from about 150 to 3,500 nucleotides. Preferably, for oligonucleotides, the chain contains from about 2 to 100 nucleotides, more preferably from about 6 to 30 nucleotides. The exact size of a polynucleotide or oligonucleotide will depend on various factors and on the particular application and use of the polynucleotide or oligonucleotide. The term includes nucleotide polymers that are synthesized and that are isolated and purified from natural sources. The term “polynucleotide” is inclusive of “oligonucleotide.”

The term “polypeptide,” “peptide,” or “protein” means a polymer of amino acids. The term encompasses naturally occurring and non-naturally occurring (synthetic) polymers and polymers in which artificial chemical mimetics are substituted for one or more amino acids. The term also encompasses fragments, variants, and homologs that have the same or substantially the same properties and perform the same or substantially the same function as the original sequence. The term encompass polymers of any length, preferably polymers containing from about 2 to 1000 amino acids, more preferably from about 5 to 500 amino acids. The term includes amino acid polymers that are synthesized and that are isolated and purified from natural sources.

The term “probe” means (1) an oligonucleotide or polynucleotide, either RNA or DNA, whether occurring naturally as in a purified restriction enzyme digest or produced synthetically, that is capable of annealing with or specifically hybridizing to a polynucleotide with sequences complementary to the probe or (2) a peptide or polypeptide capable of specifically binding a particular protein or protein fragment to the substantial exclusion of other proteins or protein fragments. An oligonucleotide or polynucleotide probe may be either single or double stranded. The exact length of the probe will depend upon many factors, including temperature, source, and use. For example, for diagnostic applications, depending on the complexity of the target sequence, an oligonucleotide probe typically contains about 10 to 100, 15 to 50, or 15 to 25 nucleotides. In certain diagnostic applications, a polynucleotide probe contains about 100-1000, 300-600, nucleotides, preferably about 300 nucleotides. The probes herein are selected to be “substantially” complementary to different strands of a particular target sequence. This means that the probes must be sufficiently complementary to specifically hybridize or anneal with their respective target sequences under a set of predetermined conditions. Therefore, the probe sequence need not reflect the exact complementary sequence of the target. For example, a noncomplementary nucleotide fragment may be attached to the 5′ or 3′ end of the probe, with the remainder of the probe sequence being complementary to the target sequence. Alternatively, noncomplementary bases or longer sequences can be interspersed into the probe provided that the probe sequence has sufficient complementarity with the sequence of the target polynucleotide to specifically anneal to the target polynucleotide. A peptide or polypeptide probe may be any molecule to which the protein or peptide specifically binds, including DNA (for DNA binding proteins), antibodies, cell membrane receptors, peptides, cofactors, lectins, sugars, polysaccharides, cells, cell membranes, organelles and organellar membranes.

The term “sample” means any animal tissue or fluid containing, e.g., polynucleotides, polypeptides, antibodies, metabolites, and the like, including cells and other tissue containing DNA and RNA. Examples include adipose, blood, cartilage, connective, epithelial, lymphoid, muscle, nervous, sputum, and the like. A sample may be solid or liquid and may be DNA, RNA, cDNA, bodily fluids such as blood or urine, cells, cell preparations or soluble fractions or media aliquots thereof, chromosomes, organelles, and the like.

The term “single package” means that the components of a kit are physically associated in or with one or more containers and considered a unit for manufacture distribution, sale, or use. Containers include, but are not limited to, bags, boxes, bottles, shrink wrap packages, stapled or otherwise affixed components, or combinations thereof. A single package may be containers of individual food compositions physically associated such that they are considered a unit for manufacture, distribution, sale, or use.

The term “useful variations” means (1) for a polynucleotide, the complements of the polynucleotide; the homologs of the polynucleotide and its complements; the variants of the polynucleotide, its complements, and its homologs; and the fragments of the polynucleotide, its complements, its homologs, and its variants and (2) for a polypeptide, the homologs of the polypeptide; the variants of the polypeptide and its homologs; and the fragments of the polynucleotide, its homologs, and its variants.

The term “virtual package” means that the components of a kit are associated by directions on one or more physical or virtual kit components instructing the user how to obtain the other components, e.g., in a bag containing one component and directions instructing the user to go to a website, contact a recorded message, view a visual message, or contact a caregiver or instructor to obtain instructions on how to use the kit.

The term “standard” means (1) a control sample that contains tissue from a lean animal if a fat animal is being tested or tissue from a fat animal if a lean animal is being tested or (2) a control sample that contains tissue from a lean or fat test animal that has not been exposed to a test substance being examined in the corresponding lean or fat animal to determine if the test substance causes differential gene expression, as appropriate for the context of its use.

The term “stringent conditions” means (1) hybridization in 50% (vol/vol) formamide with 0.1% bovine serum albumin, 0.1% Ficoll., 0.1% polyvinylpyrrolidone, 50 mM sodium phosphate buffer at pH 6.5 with 750 mM NaCl, 75 mM sodium citrate at 42° C., (2) hybridization in 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C.; with washes at 42° C. in 0.2×SSC and 0.1% SDS or washes with 0.015 M NaCl, 0.0015 M sodium citrate, 0.1% Na₂SO₄ at 50° C. or similar procedures employing similar low ionic strength and high temperature washing agents and similar denaturing agents.

The term “substance” means an element, compound, molecule, or a mixture thereof or any other material that could potentially be useful for diagnosing, prognosing, or modulating the amount of adipose tissue on animals, including any drug, chemical entity, or biologic entity.

The term “siRNA” means a polynucleotide that forms a double stranded RNA that reduces or inhibits expression of a gene when the siRNA is expressed in the same cell as the gene. The term encompasses double stranded RNA formed by complementary strands. The siRNA complementary portions that hybridize to form the double stranded molecule typically have substantial or complete identity. Typically, siRNA contains at least about 15-50 nucleotides and the double stranded siRNA contains about 15-50 base pairs, preferably about 20-30 nucleotides and base pairs.

The term “specifically bind” means a special and precise interaction between two molecules which is dependent upon their structure, particularly their molecular side groups. For example, the intercalation of a regulatory protein into the major groove of a DNA molecule, the hydrogen bonding along the backbone between two single stranded nucleic acids, or the binding between an epitope of a protein and an agonist, antagonist, or antibody.

The term “specifically hybridize” means an association between two single stranded polynucleotides of sufficiently complementary sequence to permit such hybridization under predetermined conditions generally used in the art (sometimes termed “substantially complementary”). For example, the term may refer to hybridization of a polynucleotide probe with a substantially complementary sequence contained within a single stranded DNA or RNA molecule according to an aspect of the invention, to the substantial exclusion of hybridization of the polynucleotide probe with single stranded polynucleotides of non-complementary sequence.

The term “variant” means (1) a polynucleotide sequence containing any substitution, variation, modification, replacement, deletion, or addition of one or more nucleotides from or to a polynucleotide sequence and that has the same or substantially the same properties and performs the same or substantially the same function as the original sequence and (2) a polypeptide sequence containing any substitution, variation, modification, replacement, deletion, or addition of one or more amino acids from or to a polypeptide sequence and that has the same or substantially the same properties and performs the same or substantially the same function as the original sequence. The term therefore includes single nucleotide polymorphisms (SNPs) and allelic variants and includes conservative and non-conservative amino acid substitutions in polypeptides. The term also encompasses chemical derivatization of a polynucleotide or polypeptide and substitution of nucleotides or amino acids with nucleotides or amino acids that do not occur naturally, as appropriate.

The invention is not limited to the particular methodology, protocols, and reagents described herein because they may vary. Further, the terminology used herein is for the purpose of describing particular embodiments only and is not intended to limit the scope of the present invention. As used herein and in the appended claims, the singular forms “a,” “an,” and “the” include plural reference unless the context clearly dictates otherwise, e.g., reference to “a variant” includes a plurality of variants. Further, defined terms include variations of the terms used in the proper grammatical context, e.g., the term “specifically binds” includes “specific binding” and other forms of the term. Similarly, the words “comprise”, “comprises”, and “comprising” are to be interpreted inclusively rather than exclusively.

Unless defined otherwise, all technical and scientific terms and any acronyms used herein have the same meanings as commonly understood by one of ordinary skill in the art in the field of the invention. Although any compositions, methods, articles of manufacture, or other means or materials similar or equivalent to those described herein can be used in the practice of the present invention, the preferred compositions, methods, articles of manufacture, or other means or materials are described herein.

All patents, patent applications, publications, and other references cited or referred to herein are incorporated herein by reference to the extent allowed by law. The discussion of those references is intended merely to summarize the assertions made therein. No admission is made that any such patents, patent applications, publications or references, or any portion thereof, is relevant prior art for the present invention and the right to challenge the accuracy and pertinence of such patents, patent applications, publications, and other references is specifically reserved.

In one aspect, the present invention provides one or more genes or gene segments (“genes” as defined herein) that are differentially expressed in fat animals compared to lean animals. The invention is based upon the discovery of 295 polynucleotides representing 254 genes that are differentially expressed in fat animals compared to lean animals. The genes were identified by comparing the expression of genes in adipose tissue from animals diagnosed as fat with genes in adipose tissue from animals diagnosed as lean using Affymetrix GeneChip® technology. The polynucleotides are shown in the Sequence Listing and referenced in Table 1 as SEQ ID NOs:1-295. Table 1 also shows the Affymetrix Probe Identification Number (herein “APIN”) in Column 2, fold expression (fat/lean) in Column 3, Accession Number of Highest BLAST Hit in Column 4, and Accession Number of Highest BLAST Hit for a Human Sequence in Column 5 (column descriptions are also relevant for Tables 2 and 3). A description of the putative or actual gene function can be obtained from the BLAST database using methods known to skilled artisans. Generally, the putative or actual gene function is determined by (1) identifying the APIN for each gene that had 2 fold or greater gene expression in fat animals compared to lean animals, (2) determining the nucleotide sequence of each such gene by inputting the APIN into the publicly available Affymetrix database that correlates AIPN numbers with sequences, and (3) inputting the nucleotide sequence into the BLAST database provided by the National Institutes of Health and determining the putative or actual gene function from the resulting sequence matches to homologous sequences in the database. Table 4 shows the gene description obtained for the highest blast hit accession number for the corresponding SEQ ID NO and Table 5 shows the gene description for the highest blast hit for a human sequence accession number for the corresponding SEQ ID NO.

The polynucleotides are divided into groups based upon several criteria. First, the polynucleotides are divided into three groups based upon a an analysis of expression that determines the amount of or fold differential gene expression between fat and lean animals. Group 1 corresponds to the polynucleotides identified by SEQ ID NOs:1-295. These polynucleotides are differentially expressed in fat animals compared to lean animals by at least 2 fold. Group 2 corresponds to the polynucleotides identified by SEQ ID NOs:1-70. These polynucleotides are differentially expressed in fat animals compared to lean animals by at least 2.5 fold. Group 3 corresponds to the polynucleotides identified by SEQ ID NOs:1-25. These polynucleotides are differentially expressed in fat animals compared to lean animals by at least 3 fold. Second, the polynucleotides are divided into a group based upon their function. Group 4 corresponds to the polynucleotides identified in Table 2. These polynucleotides are associated with lipid and glucose metabolism pathways in animals. Third, the polynucleotides are divided into a group based upon their relevance. Group 5 corresponds to the polynucleotides identified in Table 3. These polynucleotides were identified as particularly relevant to fat animals compared to lean animals because they were identified by more than one probe when the differential expression analysis was conducted.

The polynucleotides and genes are identified by measuring differences in gene expression from adipose tissue from canines diagnosed as fat with gene expression in adipose tissue from canines diagnosed as lean. Changes in gene expression can be determined by any method known to skilled artisans. Generally, changes in gene expression are determined by measuring transcription (determining the amount of mRNA produced by a gene) or measuring translation (determining the amount of protein produced by a gene). The amount of RNA or protein produced by a gene can be determined using any method known to skilled artisans for quantifying polynucleotides and proteins. Generally, RNA expression is determined using polymerase chain reaction (PCR) (including, without limitation, reverse transcription-PCR (RT-PCR) and quantitative real-time PCR (qPCR)), RNase protection, Northern blotting, and other hybridization methods. The RNA measured is typically in the form of mRNA or reverse transcribed mRNA. Protein or polypeptide expression is determined using various colormetric and spectroscopic assays and methods such as the lowry assay, the biuret assay, fluorescence assays, turbidimetric methods, the bicinchoninic assay, protein chip technology, infrared absorbance, ninhydrin, the bradford assay, and ultraviolet absorbance. In a preferred method, changes in gene expression are determined using Affymetrix Canine-1 and Canine-2 gene chips available for purchase from Affymetrix, Inc. and the instructions for using such chips to determine gene expression.

Generally, differential gene expression in fat animals compared to lean animals is determined by measuring the expression of at least one gene. Preferably, the expression of two or more differentially expressed genes is measured to provide a gene expression pattern or gene expression profile. More preferably, the expression of a plurality of differentially expressed genes is measured to provide additional information for a more significant gene expression pattern or profile.

The polynucleotides, genes, proteins encoded by the polynucleotides and genes, and the complements, homologs, variants, or fragments based upon the sequences are useful in a variety of prognostic and diagnostic assays relating to the amount of adipose tissue on an animal and are useful for screening test substances to determine if the substances are useful for modulating the amount of adipose tissue on an animal. Other uses will be apparent from the description of the invention contained herein.

In another aspect, the invention provides a combination comprising two or more polynucleotides that are differentially expressed in fat animals compared to lean animals or two or more proteins produced by the expression of two or more polynucleotides that are differentially expressed in fat animals compared to lean animals. In one embodiment, the combination comprises two or more polynucleotides or proteins expressed from polynucleotides selected from SEQ ID NOs:1-295. In another, the combination comprises two or more polynucleotides or proteins expressed from polynucleotides selected from SEQ ID NOs:1-70. In another, the combination comprises two or more polynucleotides or proteins expressed from polynucleotides selected from SEQ ID NOs:1-25. In another, the combination comprises two or more polynucleotides or proteins expressed from polynucleotides selected from the SEQ ID NOs identified in Table 2. In a further, the combination comprises two or more polynucleotides or proteins expressed from polynucleotides selected from the SEQ ID NOs identified in Table 3. In another, the combination comprises useful variations of such polynucleotides. Preferably, the combination comprises a plurality of polynucleotides or proteins expressed from polynucleotides, generally about 10, 20, 50, 100, 200, or more polynucleotides or proteins, as appropriate for a particular Group and use. When the combination comprises one or more fragments, the fragments can be of any size that retains the properties and function of the original polynucleotide or protein, preferably from about 30%, 60%, or 90% of the original. The polynucleotides and proteins can be from any animal, preferably canines and felines, most preferable canines.

In another aspect, the invention provides a composition comprising two or more oligonucleotide or polynucleotide probes suitable for detecting the expression of genes differentially expressed in fat animals compared to lean animals. In one embodiment, the probes comprise polynucleotides selected from SEQ ID NOs:1-295. In another, the probes comprise polynucleotides selected from SEQ ID NOs:1-70. In a further, the probes comprise polynucleotides selected from SEQ ID NOs:1-25. In another, the probes comprise polynucleotides selected from the SEQ ID NOs identified in Table 2. In another, the probes comprise polynucleotides selected from the SEQ ID NOs identified in Table 3. In another, the probes comprise useful variations of such polynucleotides. The probes contain a sufficient number of nucleotides to specifically hybridize substantially exclusively with appropriate complementary polynucleotides. Preferably, the probes comprise at least about 10, 15, 20, 25, or 30 nucleotides. In some embodiments, the probes contain more nucleotides and comprise at least about 30, 50, 70, 90 or 100 nucleotides, or more. The probes may comprise full length functional genes of the present invention. Preferably, the composition comprises a plurality of polynucleotide probes suitable for detecting genes differentially expressed in fat animals compared to lean animals, generally about 10, 50, 200, 500, 1000, or 2000, or more probes. The polynucleotide probes are made or obtained using methods known to skilled artisans, e.g., in vitro synthesis from nucleotides, isolation and purification from natural sources, or enzymatic cleavage of the genes of the present invention.

In another aspect, the invention provides a device suitable for detecting the expression of a plurality of genes differentially expressed in fat animals compared to lean animals. The device comprises a substrate having a plurality of the oligonucleotide or polynucleotide probes of the present invention affixed to the substrate at known locations. The device is essentially an immobilized version of the oligonucleotide or polynucleotide probes described herein. The device is useful for rapid and specific detection of genes and polynucleotides and their expression patterns and profiles. Typically, such probes are linked to a substrate or similar solid support and a sample containing one or more polynucleotides (e.g., a gene, a PCR product, a ligase chain reaction (LCR) product, a DNA sequence that has been synthesized using amplification techniques, or a mixture thereof) is exposed to the probes such that the sample polynucleotide(s) can hybridize to the probes. Either the probes, the sample polynucleotide(s), or both, are labeled, typically with a fluorophore or other tag such as streptavidin, and detected using methods known to skilled artisans. If the sample polynucleotide(s) is labeled, hybridization may be detected by detecting bound fluorescence. If the probes are labeled, hybridization is typically detected by label quenching. If both the probe and the sample polynucleotide(s) are labeled, hybridization is typically detected by monitoring a color shift resulting from proximity of the two bound labels. A variety of labeling strategies and labels are known to skilled artisans, particularly for fluorescent labels. Preferably, the probes are immobilized on substrates suitable for forming an array (known by several names including DNA microarray, gene chip, biochip, DNA chip, and gene array) comparable to those known in the art.

In another aspect, the invention provides a composition comprising two or more peptide or polypeptide probes suitable for detecting the expression of genes differentially expressed in fat animals compared to lean animals. In one embodiment, the probes comprise peptides or polypeptides that specifically bind to proteins produced by the expression of one or more polynucleotides comprising sequences selected from SEQ ID NOs:1-295. In another, the probes comprise peptides or polypeptides that specifically bind to proteins produced by expression of one or more polynucleotides comprising sequences selected from SEQ ID NOs:1-70. In another the probes comprise peptides or polypeptides that specifically bind to proteins produced by expression of one or more polynucleotides selected from SEQ ID NOs:1-25. In a further the probes comprise peptides or polypeptides that specifically bind to proteins produced by expression of one or more polynucleotides selected from the SEQ ID NOs identified in Table 2. In another, the probes comprise peptides or polypeptides that specifically bind to proteins produced by expression of one or more polynucleotides selected from the SEQ ID NOs identified in Table 3. In another, the probes comprise peptides or polypeptides that specifically bind to proteins produced by expression of one or more useful variations of such polypeptides. The probes contain a sufficient number of amino acids to specifically bind to the appropriate polypeptides. Preferably, the probes comprise at least about 4, 10, 20, 40, or 80 amino acids. In some embodiments, the probes contain more amino acids and comprise at least about 100 or more amino acids. The probes may comprise full length functional proteins derived from the expression of full length functional genes identified by the present invention. Preferably, the invention provides a plurality of polypeptide probes suitable for detecting genes differentially expressed in fat animals compared to lean animals, more preferably a collection of about 10, 50, 100, 500, or 1000 or more of such probes. In one embodiment, the probes are antibodies, preferably monoclonal antibodies.

The polypeptide probes may be made according to conventional methods, e.g., using the nucleotide sequence data provided for polynucleotides of the present invention and methods known in the art. Such methods include, but are not limited to, isolating polypeptide directly from cells, isolating or synthesizing DNA or RNA encoding the polypeptides and using the DNA or RNA to produce recombinant products, synthesizing the polypeptides chemically from individual amino acids, and producing polypeptide fragments by chemical cleavage of existing polypeptides.

In another aspect, the invention provides a device suitable for detecting the expression of a plurality of genes differentially expressed in fat animals compared to lean animals. The device comprises a substrate having a plurality of the peptide or polypeptide probes of the present invention affixed to the substrate at known locations. The device is essentially an immobilized version of the peptide or polypeptide probes described herein. The device is useful for the rapid and specific detection of proteins and their expression patterns. Typically, such probes are linked to a substrate and a sample containing one or more proteins is exposed to the probes such that the sample proteins can hybridize to the probes. Either the probes, the sample proteins, or both, are labeled and detected, typically with a fluorophore or other agent known to skilled artisans. Generally, the same methods and instrumentation used for reading polynucleotide microarrays is applicable to protein arrays. Preferably, the probes are immobilized on a substrate suitable for forming an array.

Methods for determining the amount or concentration of protein in a sample are known to skilled artisans. Such methods include radioimmunoassays, competitive-binding assays, Western blot analysis, and ELISA assays. For methods that use antibodies, polyclonal and monoclonal antibodies are suitable. Such antibodies may be immunologically specific for a protein, protein epitope, or protein fragment.

Some embodiments of the invention utilize antibodies for the detection and quantification of proteins produced by expression of the polynucleotides of the present invention. Although proteins may be detected by immunoprecipitation, affinity separation, Western blot analysis, protein arrays, and the like, a preferred method utilizes ELISA technology wherein the antibody is immobilized on a solid support and a target protein or peptide is exposed to the immobilized antibody. Either the probe, or the target, or both, can be labeled using known methods.

In some embodiments, expression patterns or profiles of a plurality of genes differentially expressed in fat animals compared to lean animals are observed utilizing an array of probes for detecting polynucleotides or polypeptides. In one embodiment, arrays of oligonucleotide or polynucleotide probes may be utilized, whereas another embodiment may utilize arrays of antibodies or other proteins that specifically bind to the differentially expressed gene products of the present invention. Such arrays may be commercially available or they may be custom made using methods known to skilled artisans, e.g., in-situ synthesis on a solid support or attachment of pre-synthesized probes to a solid support via micro-printing techniques. In various embodiments, arrays of polynucleotides or polypeptides probes are custom made to specifically detect transcripts or proteins produced by the differentially expressed genes of the present invention.

In one embodiment, arrays of polynucleotide or polypeptide probes are custom made to specifically detect transcripts or proteins produced by two or more polynucleotides or genes identified in Table 2. These probes are designed to detect genes associated with lipid and glucose metabolism pathways in animals. In another embodiment, arrays of polynucleotide or polypeptide probes are custom made to specifically detect transcripts or proteins produced by two or more polynucleotides or genes identified in Table 3. These probes are designed to detect genes that are particularly relevant to fat animals compared to lean animals.

In a further aspect, the invention provides a method for detecting the differential expression of one or more genes differentially expressed in fat animals compared to lean animals in a sample. The method comprises (a) hybridizing a combination comprising a plurality of polynucleotide probes that are differentially expressed in fat animals compared to lean animals with polynucleotides in the sample to form one or more hybridization complexes; (b) optionally, hybridizing a combination comprising a plurality of polynucleotide probes that are differentially expressed in fat animals compared to lean animals with polynucleotides in a standard to form one or more hybridization complexes; (c) detecting the hybridization complexes from the sample and, optionally, the standard from step (b); and (d) comparing the hybridization complexes from the sample with the hybridization complexes from a standard, wherein a difference in the amount of hybridization complexes between the standard and sample indicate differential expression of genes differentially expressed in fat animals compared to lean animals in the sample. In various embodiments, the plurality of polynucleotide probes are selected from SEQ ID NOs:1-295 with difference of 2 fold or more, SEQ ID NOs:1-70 with difference of 2.5 fold or more, SEQ ID NOs:1-25 with difference of 3 fold or more, polynucleotides identified in Table 2 with difference of 2 fold or more, polynucleotides identified in Table 3 with difference of 2 fold or more, and useful variations of such polynucleotides with the appropriate fold for the Group. These polynucleotides are used to prepare probes that hybridize with sample polynucleotides to form hybridization complexes that are detected and compared with those of the standard. In some embodiments, the sample polynucleotides are amplified prior to hybridization. In some embodiments, the probes are bound to a substrate, preferably in an array.

Step (b) and part of step (c) are optional and are used if a relatively contemporaneous comparison of two or more test systems is to be conducted. However, in a preferred embodiment, the standard used for comparison is based upon data previously obtained using the method.

These probes are exposed to a sample to form hybridization complexes that are detected and compared with those of a standard. The differences between the hybridization complexes from the sample and standard indicate differential expression of polynucleotides and therefore genes differentially expressed in fat animals compared to lean animals in the sample. In a preferred embodiment, probes are made to specifically detect polynucleotides or fragments thereof produced by one or more of the genes or gene fragments identified by the present invention. Methods for detecting hybridization complexes are known to skilled artisans.

In one embodiment, the method further comprises exposing the animal or sample to a test substance before hybridization. Then, the comparison is indicative of whether the test substance altered the expression of genes differentially expressed in fat animals compared to lean animals, particularly fat-associated genes, in the sample.

In another aspect, the invention provides a method for detecting the differential expression of genes differentially expressed in fat animals compared to lean animals in a sample. The method comprises (a) reacting a combination comprising a plurality of polypeptide probes with proteins in the sample under conditions that allow specific binding between the probes and the proteins to occur, wherein the proteins bound by the probes are differentially expressed in a fat animal compared to a lean animal; (b) optionally, reacting a combination comprising a plurality of polypeptide probes with proteins in a standard under conditions that allow specific binding between the probes and the proteins to occur, wherein the proteins bound by the probes are differentially expressed in a fat animal compared to a lean animal; (c) detecting specific binding in the sample and, optionally, the standard from step (b); and (d) comparing the specific binding in the sample with that of a standard, wherein differences between the specific binding in the standard and the sample indicate differential expression of genes differentially expressed in fat animals compared to lean animals in the sample.

In various embodiments, the plurality of polypeptide probes are probes that specifically bind to proteins produced by expression of one or more polynucleotides selected from SEQ ID NOs:1-295 with difference of 2 fold or more, SEQ ID NOs:1-70 with difference of 2.5 fold or more, SEQ ID NOs:1-25 with difference of 3 fold or more, polynucleotides identified in Table 2 with difference of 2 fold or more, polynucleotides identified in Table 3 with difference of 2 fold or more, and useful variations of such polynucleotides with the appropriate fold for the Group. These polynucleotides are used to prepare probes that specifically bind to proteins that are detected and compared with those of the standard. In some embodiments, the probes are bound to a substrate, preferably in an array. In one embodiment the probes are antibodies.

Step (b) and part of step (c) are optional and are used if a relatively contemporaneous comparison of two or more test systems is to be conducted. However, in a preferred embodiment, the standard used for comparison is based upon data previously obtained using the method.

These probes are exposed to a sample to form specific binding that is detected and compared with those of a standard. The differences between the specific binding from the sample and standard indicate differential expression of proteins and therefore genes differentially expressed in fat animals compared to lean animals, particularly fat-associated genes, in the sample. In a preferred embodiment, probes are made to specifically detect proteins or fragments thereof produced by one or more of the genes or gene fragments identified by the present invention.

In one embodiment, the method further comprises exposing the animal or sample to a test substance before reacting the polypeptides with the proteins. Then, the comparison is indicative of whether the test substance altered the expression of genes differentially expressed in fat animals compared to lean animals, particularly fat-associated genes, in the sample.

In another aspect, the method for detecting the expression of genes differentially expressed in fat animals compared to lean animals in a sample is used to monitor an animal's progress when attempting to modulate the amount of adipose tissue on the animal in response to an adipose tissue modulation program. The method is performed at intervals, preferably set intervals, during the modulation program and the animal's progress monitored by comparing the results of the method at two or more points during the modulation program. A change in expression of one or more of the genes differentially expressed in fat animals compared to lean animals, particularly fat-associated genes, or in the pattern of gene expression, or the tack of any change, resulting from the comparison indicates the effectiveness of the modulation program. For example, an adipose tissue modulation program designed to reduce the amount of adipose tissue on an animal could be monitored and shown to be effective if the amount of gene expression for genes differentially expressed in fat animals compared to lean animals, particularly fat-associated genes, declines over time in response to the stimulus in the program. Similarly, a program to increase adipose tissue in a lean or overly lean animal should increase the expression profile for such genes. The modulation program can be any plan to modulate the amount of adipose tissue on the animal such as a diet, exercise, drug, or other similar program.

In a further aspect, the invention provides a method for measuring the effect of a test substance on the expression profile of one or more genes differentially expressed in fat animals compared to lean animals and a method for screening a test substance to determine if it is likely to be useful for modulating the amount of adipose tissue on an animal. The methods comprise (a) determining a first expression profile by measuring the transcription or translation products of two or more polynucleotides selected from SEQ ID NOs:1-295 or useful variations thereof in a test system in the absence of the test substance; (b) determining a second expression profile by measuring the transcription or translation products of two or more polynucleotides selected from SEQ ID NOs:1-295 or useful variations thereof in a test system in the presence of the test substance; and (c) comparing the first expression profile to the second expression profile.

A change in the second expression profile compared to the first expression profile of 2 fold or more indicates that the test substance effects the expression of genes differentially expressed in fat animals compared to lean animals and that the test substance is likely to be useful for modulating the amount of adipose tissue on an animal. In a preferred embodiment, the genes differentially expressed in fat animals compared to lean animals are fat-associated genes and the change is a 2 fold or more change in expression of at least two genes between the first expression profile to the second expression profile. The invention also provides the substances identified using the method.

In one embodiment, the polynucleotides are selected from SEQ ID NOs:1-70 or useful variations thereof and the change is 2.5 fold or higher. In another, the polynucleotides are selected from SEQ ID NOs:1-25 or useful variations thereof and the change is 3 fold or higher. In a further, the polynucleotides are identified in Table 2 or Table 3, or useful variations thereof, and the change is 2 fold or higher.

In one embodiment, the test system is an in vitro test system such as a tissue culture, cell extract, or cell line. In another, the test system is an in vivo test system, i.e., an animal such as a canine. In other embodiments, the test system is an ex vivo tissue system or an in silico system.

Test substances can be any substance that may have an effect on polynucleotides or genes differentially expressed in fat animals compared to lean animals, particularly fat-associated genes. Test substances include, but are not limited to, amino acids; proteins, peptides, polypeptides, nucleic acids, oligonucleotides, polynucleotides, small molecules, macromolecules, vitamins, minerals, simple sugars; complex sugars; polysaccharides; carbohydrates; medium-chain triglycerides (MCTs), triacylglycerides (TAGs); n-3 (omega-3) fatty acids including DHA, EPA, ALA; n-6 (omega-6) fatty acids including LA, γ-linolenic acid (GLA) and ARA; SA, conjugated linoleic acid (CLA); choline sources such as lecithin; fat-soluble vitamins including vitamin A and precursors thereof such as carotenoids (e.g., β-carotene), vitamin D sources such as vitamin D₂ (ergocalciferol) and vitamin D₃ (cholecalciferol), vitamin E sources such as tocopherols (e.g., α-tocopherol) and tocotrienols, and vitamin K sources such as vitamin K₁ (phylloquinone) and vitamin K₂ (menadione); water-soluble vitamins including B vitamins such as riboflavin, niacin (including nicotinamide and nicotinic acid), pyridoxine, pantothenic acid, folic acid, biotin and cobalamin; and vitamin C (ascorbic acid); antioxidants, including some of the vitamins listed above, especially vitamins E and C; also bioflavonoids such as catechin, quercetin and theaflavin; quinones such as ubiquinone; carotenoids such as lycopene and lycoxanthin; resveratrol; and α-lipoic acid, L-carnitine; D-limonene; glucosamine; S-adenosylmethionine; and chitosan. In a preferred embodiment test substances are nutrients that may be added to food or consumed as a supplement. Examples include, but are not limited to, fatty acids such as omega-3 fatty acids (e.g., DHA and EPA) and omega-6 fatty acids (erg., ARA), carnitine, methionine, vitamin C, vitamin E, and vitamin D.

In a preferred embodiment, the substances useful for affecting the expression of genes differentially expressed in fat animals compared to lean animals, particularly fat-associated genes, may be identified using methods discloses in co-pending U.S. Provisional Patent Application No. 60/657,980, filed Mar. 2, 2005, and any subsequent US or foreign patent application that claims priority thereto.

In a further aspect, the invention provides a method for formulating a prognosis that an animal is likely to become fat or developing a diagnosis that an animal is fat. The method comprises determining if one or more polynucleotides selected from SEQ ID NOs:1-295 or useful variations thereof or one or more polypeptides that specifically bind to proteins produced by expression of one or more polynucleotides selected from SEQ ID NOs:1-295 or useful variations thereof are differentially expressed in the animal compared to one or more lean animals. The animal is determined to be likely to become fat or determined to be fat if the comparison indicates that the polynucleotides are differentially expressed in the animal compared to the lean animals by a fold of 2 or more.

In various embodiments, the prognosis or diagnosis is based upon the polynucleotides selected from SEQ ID NOs:1-70, SEQ ID NOs:1-25, the sequences identified in Table 2, the sequences identified in Table 3, or useful variations of such polypeptides.

The expression profile for lean animals used in the comparison can be obtained from one or more lean animals contemporaneously with the expression profile for the animal being tested of from a database of lean animal expression profiles. Preferably, a database of expression profiles for lean animals accumulated over time is available for use as a reference.

Determining if the polynucleotides or polypeptides are differentially expressed can be accomplished by detecting the polynucleotides or polypeptides using methods known to skilled artisans some of which are described herein.

In another aspect, the invention provides a method for manipulating the genome or the expression of the genome of an animal, particularly a non-human animal. The method comprises disrupting the expression of one or more genes differentially expressed in fat animals compared to lean animals, preferably using oligonucleotides or polynucleotides constructed using polynucleotides selected from SEQ ID NOs:1-295 or useful variations thereof.

Methods of manipulating the genome are known to those of skilled in the art. Such methods include the production of transgenic and knockout animals and the disruption of transcription or translation. In one embodiment, one or more polynucleotides selected from SEQ ID NOs:1-295 or useful variations thereof are used to prepare a construct useful to disrupt or “knock out” the corresponding endogenous gene in an animal. This method produces an animal having a null mutation for that gene locus. In other embodiments, the animals exhibit a reduction or complete elimination of the expression of one or more genes differentially expressed in fat animals compared to lean animals, particularly fat-associated genes. The invention also provides an animal produced using the method. In various embodiments, the genome is manipulated using the one or more polynucleotides selected from SEQ ID NOs:1-70, SEQ ID NOs:1-25, the sequences identified in Table 2, the sequences identified in Table 3, or useful variations of such sequences. The transgenic animals are preferably mammals, e.g., rodents such as mice and rats, but may be other mammal such as felines and canines.

Methods of manipulating the expression of genome are known to those of skilled in the art. Such methods include the use of antisense or siRNA molecules and using such molecules to disrupt the translation or transcription of the genome. In one embodiment, one or more polynucleotides selected from SEQ ID NOs:1-295 or useful variations thereof are used to prepare antisense and similar DNA binding molecules that are useful for disrupting transcription or to prepare short (small) interfering RNAs (siRNA) useful for functionally disrupting translation. Briefly, gene expression is inhibited by antisense molecules through binding to DNA and preventing transcription and a siRNA through RNA interference (RNAi) or post-transcriptional gene silencing (PTGS). siRNA molecules target homologous mRNA molecules for destruction by cleaving the mRNA molecule within the region spanned by the siRNA molecule, Accordingly, siRNAs capable of targeting and cleaving a mRNA transcribed from a fat-associated gene is used to decrease or eliminate expression of one or more of such genes. In other embodiments, antisense molecules capable of binding to DNA and siRNAs capable of targeting and cleaving mRNA transcribed from one or more polynucleotides or genes selected from Group 2, Group 3, Group 4, or Group 5 polynucleotides or genes may be used to decrease or eliminate expression of one or more of these genes. In preferred embodiments, siRNAs are constructed from the transcripts of polynucleotides selected from SEQ ID NOs:1-295 or useful variations thereof.

In another aspect, the invention provides a composition suitable for manipulating the genome of an animal. The composition comprises one or more substances that interfere with the expression of one or more genes differentially expressed in fat animals compared to lean animals, particularly fat-associated genes. Preferably, substances comprise oligonucleotides or polynucleotides that bind to one or more of the genes or their transcription products and interferes with their replication, transcription, or translation, most preferably oligonucleotides or polynucleotides constructed using polynucleotides selected from SEQ ID NOs:1-295 or useful variations thereof. In various embodiments, the substances comprise antisense molecules or siRNAs.

In another aspect, the invention provides a method for modulating the expression of one or more genes differentially expressed in fat animals compared to lean animals, particularly fat-associated genes, or modulating the amount of adipose tissue on an animal comprising administering to the animal a gene expression or tissue modulating amount of a composition comprising one or more of DHA, EPA, EPA and DHA, ALA, LA, ARA, and SA. In preferred embodiments the composition comprises, in milligrams per kilogram of body weight per day (mg/kg/day), DHA in amounts of from about 1 to about 30, preferably from about 3 to about 15; EPA in amounts of from about 1 to about 30, preferably from about 3 to about 15; EPA/DHA Combo (1.5:1 ratio) in amounts of from about 412 to about 30/45, preferably from about 9/6 to about 18/12; ALA in amounts of from about 10 to about 100, preferably from about 30 to about 60; LA in amounts of from about 30 to about 600, preferably from about 60 to about 300; ARA in amounts of from about 5 to about 50, preferably from about 15 to about 30; SA in amounts of from about 3 to about 60, preferably from about 6 to about 30; and CLA (as a control) in amounts of from about 6 to about 120, preferably from about 12 to about 60. The composition can be administered to the animal in any manner or form suitable for the composition. Preferably, the composition is administered to the animal orally in the form of a food composition or a supplement. The food composition may be of any form, e.g., a nutritionally balanced food composition known in the art such as dry foods, semi-moist foods, and wet foods for animals, particularly companion animals such as feline and canine animals. Supplements include dosage forms such as tablets, capsules, and similar forms. In a further aspect, the composition is administered in combination with one or more drugs or other substances that modulate the amount of adipose tissue on an animal. The drugs or substances include, but are not limited to, substances that suppress appetite, increase metabolism, or interfere with the absorption of specific nutrients, particularly from food. Examples include, but are not limited to, orlistat (blocks fat breakdown and absorption), anorexigenics such as dexedrine (suppresses appetite), anorectics such as fenfluramine and phentermine, and sibutramine, and phenylpropanolamine.

In another aspect, the invention provides a composition suitable for modulating the expression of one or more genes differentially expressed in fat animals compared to lean animals, particularly fat-associated genes, or modulating the amount of adipose tissue on an animal. The composition comprises a gene expression or tissue modulating amount of one or more of DHA, EPA, EPA and DHA, ALA, LA, ARA, and SA. In various embodiments, the composition comprises, in mg/kg/day, DHA in amounts sufficient to administer to an animal from about 1 to about 30; EPA in amounts sufficient to administer to an animal from about 1 to about 30; EPA/DHA Combo (1.5:1 ratio) in amounts sufficient to administer to an animal from about 4/2 to about 30/45; ALA in amounts sufficient to administer to an animal from about 10 to about 100; LA in amounts sufficient to administer to an animal from about 30 to about 600; ARA in amounts sufficient to administer to an animal from about 5 to about 50; SA in amounts sufficient to administer to an animal from about 3 to about 60; and CLA (as a control) in amounts sufficient to administer to an animal from about 6 to about 120. Such substances are useful for modulating the amount of adipose tissue on an animal, Preferably, the substances affect the expression of a plurality of such genes. In one embodiment, the composition further comprises one or more drugs or other substances that modulate the amount of adipose tissue on an animal.

In another aspect, the invention provides a method for selecting an animal for inclusion in one or more groups or subgroups. The method comprises determining the expression profile of the animal for (a) polynucleotides selected from SEQ ID NOs:1-295 or useful variations thereof or (b) polypeptides each of which specifically binds to proteins produced by expression of one or more polynucleotides selected from SEQ ID NOs:1-295 or useful variations thereof and assigning the animal to a group based upon the expression profile. The groups can be any useful groups, preferably those involved in a research experiment, trial, clinical trial, or other similar category. For example, the groups can be groups involved in a research experiment or clinical trial that requires a one or more control groups and one or more treatment groups. In one embodiment, the control group comprises lean animals and the treatment group comprises fat animals, or vice versa in another. The expression profile for a plurality of animals can be determined and the animals assigned to the control group or treatment group based upon the results of the profile, i.e., animals with a differential expression of 2 fold or more compared to a standard are assigned to the fat group and animals with a differential expression of 2 fold or less compared to a standard are assigned to the lean group. The method is particularly useful for assigning animals to a clinical trial when testing potential drugs or other substances for their ability to reduce the amount of adipose tissue on the animal.

In another aspect, the invention provides a computer system suitable for manipulating data relating to one or more genes differentially expressed in fat animals compared to lean animals, particularly fat-associated genes. The system comprises a database containing information identifying the expression level of one or more polynucleotides selected from SEQ ID NOs:1-295 or useful variations thereof and/or polypeptides that specifically bind to proteins produced by the expression of one or more polynucleotides selected from SEQ ID NOs:1-295 or useful variations thereof in lean animals and/or fat animals and a user interface to interact with the database, particularly to input, manipulate, and review the information for different animals or categories or animals, e.g., lean or fat animals. In one embodiment, the database further contains information identifying the activity level of one or more polypeptides encoded by one or more polynucleotides selected from SEQ ID NOs:1-295 or useful variations thereof. In another, the database further comprises sequence information for one or more of the polynucleotides selected from SEQ ID NOs:1-295 or useful variations thereof. In other embodiments, the database contains additional information describing the putative description of the genes in one or more animal species. The computer system is any electronic device capable of containing and manipulating the data and interacting with a user., e.g., a typical computer or an analytical instrument designed to facilitate using the present invention and outputting the results relating to the status of an animal.

In another aspect, the invention provides a method for using a computer system or the present invention to present information identifying the expression profile of one or more genes differentially expressed in fat animals compared to lean animals, particularly fat-associated genes. The method comprises comparing the expression level of two or more polynucleotides or proteins expressed from polynucleotides selected from SEQ ID NOs:1-295 form a sample to the expression profile of the polynucleotides or proteins in the computer system.

In a further aspect, the present invention provides kits suitable for determining the differential expression of one or more genes differentially expressed in fat animals compared to lean animals, particularly fat-associated genes, in a test system. The kits comprise in separate containers in a single package or in separate containers in a virtual package, as appropriate for the use and kit component, two or more probes suitable for detecting the expression of genes differentially expressed in fat animals compared to lean animals, the probes comprising (a) polynucleotides selected from SEQ ID NOs:1-295 or useful variations thereof or (b) polypeptides that specifically bind to proteins produced by the expression of one or more polynucleotides selected from SEQ ID NOs:1-295 or useful variations thereof and at least one of (1) instructions for how to use the probes of the present invention; (2) reagents and equipment necessary to use the probes, (3) a composition suitable for modulating the expression of one or more genes differentially expressed in fat animals compared to lean animals; (4) a composition suitable for disrupting the expression of one or more genes differentially expressed in fat animals compared to lean animals; (5) a food composition suitable for modulating the amount of adipose tissue on an animal; and (6) one or more drugs or other substances that that modulate the amount of adipose tissue on an animal. In one preferred embodiment, the probes are bound to a substrate, preferably in an array.

When the kit comprises a virtual package, the kit is limited to instructions in a virtual environment in combination with one or more physical kit components. In one embodiment, the kit contains probes and/or other physical components and the instructions for using the probes and other components are available via the internet. The kit may contain additional items such as a device for mixing samples, probes, and reagents and device for using the kit, e.g., test tubes or mixing utensils.

In another aspect, the present invention provides a means for communicating information about or instructions for one or more of (1) using the polynucleotides of the present invention for detecting the expression of genes differentially expressed in fat animals compared to lean animals in a sample, (2) using the polynucleotides of the present invention for measuring the effect of a test substance on the expression of one or more genes differentially expressed in fat animals compared to lean animals, (3) using the polynucleotides of the present invention for screening a test substance to determine if it is likely to be useful for modulating the amount of adipose tissue on an animal, (4) using the polynucleotides of the present invention for formulating a prognosis that an animal is likely to become fat or developing a diagnosis that an animal is fat, (5) using the polynucleotides of the present invention for manipulating the genome of a non-human animal or the expression of the genome of an animal, (6) using the polynucleotides of the present invention for modulating the expression of one or more genes differentially expressed in fat animals compared to lean animals, particularly fat-associated genes, or modulating the amount of adipose tissue on an animal, (7) using the polynucleotides of the present invention for selecting an animal for inclusion in one or more groups (8) using the polynucleotides of the present invention for using computer system to manipulate data relating to genes differentially expressed in fat animals compared to lean animals, particularly fat-associated genes, (9) administering substances of the present invention to an animal, alone or in combination with the other elements of the present invention, (10) using the substances of the present invention for modulating the amount of adipose tissue on an animal, (11) using the computer system of the present invention, (12) using the kits of the present invention, and (13) instructions for using the methods and compositions of the present invention with one or more drugs or other substances that that modulate the amount of adipose tissue on an animal. The means comprises a document, digital storage media, optical storage media, audio presentation, or visual display containing the information or instructions. In certain embodiments, the communication means is a displayed web site, visual display, kiosk, brochure, product label, package insert, advertisement, handout, public announcement, audiotape, videotape, DVD, CD-ROM, computer readable chip, computer readable card, computer readable disk, computer memory, or combination thereof containing such information or instructions. Useful information includes one or more of (1) methods for promoting the health and wellness of animals and (2) contact information for the animal's caregivers to use if they have a question about the invention and its use. Useful instructions include techniques for using the probes, instructions for performing a gene expression assay, and administration amounts and frequency for the substances. The communication means is useful for instructing on the benefits of using the present invention.

Disclosed herein are typical preferred embodiments of the invention and, although specific terms are employed, they are used in a generic and descriptive sense only and not for purposes of limitation as many modifications and variation of the invention are possible in light of the teachings contained herein. The invention can be further illustrated by the following examples, although it will be understood that these examples are included merely for purposes of illustration and are not intended to limit the scope of the invention unless otherwise specifically indicated.

### EXAMPLES

### Materials and Methods

Isolation of Ribonucleic Acid (RNA) from Tissue

Tissue samples that have been collected, frozen in liquid nitrogen, and thawed are homogenized and processed using a TRIzol® RNA extraction method to produce good quality RNA which is then subjected to further genomic analysis.

Materials: ice, liquid nitrogen, frozen canine or feline tissue, TRIzol® lysis reagent, chloroform minimum 99%, isopropyl alcohol, 70% ethanol (prepared with ethanol, absolute and deionized, RNase-free water), RNase Zap®, deionized water, RNA Storage Solution®, from Ambion.

Equipment: Ultra-Turrax T25 Power Homogenizer, Beckman Coulter Allegra 25R Centrifuge, Eppendorf Centrifuge, forceps, scalpel, hard cutting surface, i.e. cutting board, 1.5 mL DNase and RNase free/sterile microcentrifuge tubes, 50 mL DNase and RNase free/sterile disposable polypropylene tubes, P1000, P200, P20, P10 and P2 Rainin Pipetman pipettes, filter pipette tips for P1000, P200, P20, P10 and P2 pipettes, DNase and RNase free/sterile, and lint free wipes.

Preparations: Prepare 50 mL polypropylene tubes with 4 mL TRIzol® (one tube for each tissue selected for RNA isolation).

Tissue Homogenization: Fill a container capable of holding liquid nitrogen with 3-4 scoops of liquid nitrogen. Place a piece of frozen tissue immediately into the aforementioned container (the tissue should be about the size of a pea) and place the tissue into the appropriate labeled 50 mL polypropylene tube (that already contains 4 mL TRIzol®). Immediately begin homogenization using the Ultra-Turrax T25 Power Homogenizer. Homogenize on the highest setting (6) for 10-15 seconds. Cool the sample on ice for another 10-15 seconds and then repeat. Continue until the tissue is fully homogenized and the solution is cloudy. Upon complete homogenization, cap the 50 mL tube and return to the ice. Incubate the homogenized tissues at room temperature for 5 minutes before proceeding with the isolation procedure.

RNA Isolation: The procedures given in the Invitrogen instructions provided with the TRIzol® reagent are generally followed. Separate the homogenized sample into four 1 mL aliquots in four 1.5 mL microcentrifuge tubes. Add 200 uL of chloroform to each 1 mL aliquot. Cap the tubes, vortex for 15 seconds and then shake up and down. The result should be a pink milky liquid. Incubate the tubes at room temperature for 2-3 minutes. Centrifuge the tubes for 15 minutes at 14,000 rpm and 4° C. Transfer the aqueous phase (top layer) to a sterile 1.5 mL microcentrifuge tube. The typical volume of the aqueous phase which should be transferred to the new tube is about 500 uL. Be sure not to transfer any of the intermediate or lower phase. Precipitate the RNA from solution by adding 500 uL of Isopropyl Alcohol to each microcentrifuge tube containing the aqueous layer. Shake the tubes up and down for at least 20 seconds. Incubate the samples at room temperature for 10 minutes. Centrifuge the samples for 10 minutes, 14,000 rpm at 4° C. Remove the supernatant carefully by aspirating off the liquid being sure not to lose the pellet. Add 1 mL of 70% ethanol to wash the pellet. Dislodge the pellet by flicking the tube (or tapping the tube on the bench top) and shake to mix. Centrifuge for 5 minutes, 8,200 rpm at 4° C. Remove the supernatant carefully by aspirating off the liquid being sure not to lose the pellet. Using a lint free wipe carefully soak up excess ethanol to make sure the pellet is dry. Resuspend each pellet into 30 uL of RNA Storage Solution. Mix gently by pipetting until the RNA goes back into solution and then store at −80° C. It may be necessary to vortex the sample for a few seconds at a low speed to facilitate the resuspension of the RNA. If this is necessary spin down the samples, using the microcentrifuge, prior to freezing.

RNA Cleaning: The procedures given in the RNeasy® Mini Handbook are followed.

RNA Isolation from Cells Cultured in OptiCell Chambers Using the RNeasy Mini Kit.

Cells cultured from mammalian cell lines are used to isolate good quality RNA which is then used for future downstream genomic analysis. All work related to the culturing of the cells is to be done under strict aseptic conditions.

Reagents: 10×PBS, deionized H₂O, absolute ethanol, RNA Storage Solution, β-Mercaptoethanol, RNase Zap®, Buffer RLT, and Buffer RW1 and Buffer RPE (provided in the RNeasy Mini Kit)

Equipment/Materials: RNeasy Mini Kit, QIAshredder spin columns, OptiCell knife, 20 mL sterile syringe, OptiCell tips, Cell scraper, P1000 Pipetman pipette, Rainin, P200 Pipetman pipette, Rainin, 100-100 uL filtered pipette tips, 1-200 uL filtered pipette tips, sterile transfer pipettes, 55 mL sterile solution basin, 1.5 mL sterile microcentrifuge tubes, and Eppendorf Microcentrifuge.

Solutions: Buffer RLT (stock provided in RNeasy Mini Kit); —Add 100 uL of β-Mercaptoethanol per 10 mL of Buffer RLT prior to beginning protocol. 70% Ethanol: Make 50 mL of 70% ethanol by adding 35 mL absolute ethanol to 15 mL deionized, RNase-free water. 1×PBS: RNase-free water. Filter the solution using a 0.22 um filter.

Procedure: Removing Cells from the OptiCell Chamber (proceed one OptiCell at a time). Check the cells under a microscope to ensure that the cells are alive before isolating RNA. Remove and discard the cell culture medium. Using the OptiCell knife cut away the top membrane exposing the cells on the lower membrane. Wash the membrane to which the cells are attached three times with 1×PBS. Pipette 600 uL of the Buffer RLT solution (containing β-Mercaptoethanol) onto the center of the membrane to which the cells are attached. Using the cell scraper, gently spread the Buffer RLT over the entire surface of the membrane, and then collect the liquid in one corner. Pipette off the entire volume of Buffer RLT and place into a QIAshredder spin column.

RNA Isolation: Centrifuge the QIAshredder spin columns at 14,000 rpm for 2 minutes. Discard the spin column but keep the collection tube and its contents. Add 600 uL of 70% ethanol to the collection tube and mix well by pipetting (the total volume now 1.2 mL). Transfer 600 uL of the cell lysate to an RNeasy mini column and centrifuge for 15 seconds at 14,000 rpm. Discard the flow through but keep the collection tube and the spin column. Transfer the remaining volume of cell lysate (˜600 uL) to the spin column and repeat the centrifugation. Discard the flow through but keep the collection tube and the spin column. Add 700 uL Buffer RW1 to the spin column. Centrifuge for 15 seconds at 14,000 rpm to wash the column. Discard the flow through and the collection tube. Transfer the spin column to a new 2 mL collection tube and add 500 uL Buffer RPE to the column. Centrifuge for 15 seconds at 14,000 rpm. Discard the flow through, keep the collection tube/column. Add another 500 uL Buffer RPE to the column. Centrifuge for 2 minutes at 14,000 rpm. Transfer the spin column to a 1.5 mL collection tube. Add 30 uL of RNA Storage Solution directly to the silica gel membrane and centrifuge for 1 minute at 14,000 rpm to elute the RNA. Store the final RNA at −70° C.

### RNA 6000 Nano Assay

Using the Agilent 2100 Bioanalyzer and the RNA 6000 Nano Assay, analyze RNA isolated from cultured mammalian cells, lymphocytes or tissues for quality.

Reagents: RNA 6000 Nano gel matrix, RNA 6000 Nano dye concentrate, RNA 6000 Nano Marker, (all of the above reagents are contained in the RNA 6000 Nano Assay kit, Agilent), RNA 6000 ladder, RNase Zap, and RNase-free water, from Ambion.

Equipment/Other Materials: Agilent Chip Priming Station, Agilent, RNA 6000 chip, Agilent, electrode cleaners, P2, P10, P200, and P1000 Rainin Pipetman pipettes, sterile, DNase/RNase free filtered pipette tips, 1.5 mL microcentrifuge tubes, sterile, vortex, IKA vortex mixer, microcentrifuge, and heating block.

Procedure: The procedure is given in the Reagent Kit Guide, RNA 6000 Nano Assay, Edition November 2003, by Agilent Technologies. The procedures are followed as given in the Guide, with the following modifications: Preparing the Gel, pg. 17-rather than separating the filtered gel into aliquots of 65 uL each, keep the stock filtered gel in the original microcentrifuge tube and aliquot the 65 uL as needed. Loading the RNA 6000 Nano Marker, pg. 22—add 1 uL of RNase-free water (instead of RNA 6000 Nano Marker) to each sample well that will not contain sample. Not only will this conserve the amount of Marker used but also serves as a negative control to see that none of the reagents are contaminated, including the RNase-free water. Loading the Ladder and Samples, pg. 23—heat denature the samples and RNA 6000 Ladder for an additional 30 seconds (total of 2.5 minutes) at 71° C. Starting the Chip Run, pg. 26-choose the “Eukaryote Total RNA Nano” option from the assay menu.

### Affymetrix Genechip Expression Analysis

Gene expression is analyzed using Affymetrix Canine 1 and Canine 2 GeneChip® Arrays are available commercially from Affymetrix, Inc., Santa Clara, Calif. 95051. Total RNA is reverse transcribed into cDNA. The cDNA is used to generate cRNA which is fragmented and used as probes for GeneChip hybridization. The gene chip is washed and the hybridization signal is measured with an Affymetrix laser scanner. The hybridization data is then validated and normalized for further analysis.

Materials: Affymetrix provides most of the reagents and kit. Other reagents listed in the Affymetrix Manual but not supplied in the kit may be obtained separately (refer to GeneChip Expression Analysis Technical Manual (701021 Rev.4) for details), RNase Zap® and deionized water.

Equipment: Eppendorf microcentrifuge, 1.5 mL DNase and RNase free/sterile microcentrifuge tubes, 50 mL DNase and RNase free/sterile disposable polypropylene tubes, P1000, P200, P205 P10 and P2 Rainin Pipetman pipettes, Filter pipette tips for P1000, P200, P20, P10 and P2 pipettes, DNase and RNase free/sterile, and Peltier Thermal Cycler PTC-200.

Procedure: follow all procedures exactly as described in GeneChip Expression Analysis Technical Manual (Affymetrix Copyright 1999-2003). Use 5 microgram of total RNA for the first strand cDNA synthesis. Use either Peltier Thermal Cycler PTC-200 or heat block for temperature control on reactions and probe denaturing. The quality control is performed using RNA NanoDrop chips with BioAnalyer 2100. Use 100 Format (Midi Array) for the canine genechip.

### Example 1

### Determining Differential Gene Expression Between Adipose Tissue Samples from Fat and Lean Animals

Adipose tissue samples are obtained from 16 (3 lean and 13 fat) canine animals diagnosed as either “fat” or “lean” using conventional methods. The “fatness” or “leanness” of an animal is determined based on measurements by DEXA using conventional methods or based on a 5 point body condition scoring system. For example, an animal is considered lean if it has a body condition score of 2 or 2.5 and/or a DEXA total body fat percentage of 27% or less. An animal is considered to be fat if it has a body condition score of 4 or higher and a total body fat percentage of 30% or higher. All tissue samples are snap frozen in liquid nitrogen immediately after removal from the animal.

The tissues are analyzed using Affymetrix “Canine-2” canine gene chip according to conventional methods in order to determine which genes, if any, are differentially expressed in fat animals compared to lean animals. Data from the fat and lean samples are compared and analyzed using the GeneSpring and R-Bioconductor software. For any given gene to be assigned a “present” call, it had to exhibit a 2-fold change in expression level to be considered for further scrutiny. Furthermore, genes that are present in only one condition and are either “absent” or “marginal” in the other group are also selected for further scrutiny. Results are provided in the tables below:

TABLE 1
Genes Differentially Expressed at least 2 fold
in Adipose Tissue in Fat Animals compared to Lean Animals
Column

| 1 | 2 | 3 | 4 | 5 |
|---|---|---|---|---|
| 1 | Cfa.6562.1.A1_at | 6.48 | XM_516142 | BC065271 |
| 2 | CfaAffx.26065.1.S1_at | 3.94 | XM_547914 | AF111167 |
| 3 | CfaAffx.2782.1.S1_s_at | 3.78 | XM_538649 | AJ243425 |
| 4 | CfaAffx.2790.1.S1_s_at | 3.66 | XM_538649 | BC073983 |
| 5 | Cfa.18367.1.S1_at | 3.19 | NM_001032284 | AC013418 |
| 6 | Cfa.9039.1.A1_at | 3.07 | XM_547914 | BX647104 |
| 7 | CfaAffx.7975.1.S1_at | 3.06 | NM_182490 | NM_182490 |
| 8 | CfaAffx.24964.1.S1_at | 3.05 | XM_543892 | NM_032803 |
| 9 | Cfa.3011.1.A1_a_at | 0.33 | XM_782177 | AC005227 |
| 10 | CfaAffx.14652.1.S1_at | 0.33 | XM_848392 | BT020098 |
| 11 | Cfa.15689.1.A1_at | 0.33 | XM_844220 | AC020550 |
| 12 | CfaAffx.2909.1.S1_at | 0.33 | XM_538880 | NM_004117 |
| 13 | CfaAffx.4844.1.S1_s_at | 0.33 | XM_538481 | BT019766 |
| 14 | Cfa.12840.1.A1_at | 0.32 | BC034770 | AL157823 |
| 15 | CfaAffx.4097.1.S1_s_at | 0.32 | XM_539427 | BC040239 |
| 16 | CfaAffx.20841.1.S1_at | 0.31 | XM_537163 | AC107394 |
| 17 | Cfa.15420.1.A1_at | 0.3 | NM_077876 | AC061958 |
| 18 | CfaAffx.17336.1.S1_s_at | 0.28 | AJ575592 | NM_001093 |
| 19 | CfaAffx.4844.1.S1_at | 0.26 | XM_538481 | BT019766 |
| 20 | Cfa.8932.1.A1_at | 0.25 | AB089806 | AC006431 |
| 21 | Cfa.15612.1.A1_at | 0.24 | U09019 | AC073838 |
| 22 | CfaAffx.11400.1.S1_at | 0.21 | XM_850381 | NM_198538 |
| 23 | CfaAffx.20763.1.S1_at | 0.16 | XM_719217 | AC090018 |
| 24 | CfaAffx.732.1.S1_x_at | 0.14 | NM_181756 | AK095351 |
| 25 | CfaAffx.732.1.S1_at | 0.12 | NM_181756 | NM_181756 |
| 26 | Cfa.2343.1.S1_at | 2.97 | XM_532944 | CR617129 |
| 27 | Cfa.13082.1.A1_s_at | 2.83 | D38312 | AC072022 |
| 28 | Cfa.9807.1.A1_at | 2.77 | NM_005458 | AL591502 |
| 29 | CfaAffx.4729.1.S1_at | 2.76 | XM_532014 | NM_003692 |
| 30 | Cfa.1213.1.S1_s_at | 2.73 | X97226 | BC016147 |
| 31 | Cfa.15795.1.A1_s_at | 2.66 | XM_582039 | X53683 |
| 32 | Cfa.14576.1.A1_at | 2.65 | Z73942 | AK097232 |
| 33 | Cfa.3851.1.S1_s_at | 2.64 | NM_001003297 | M28226 |
| 34 | CfaAffx.19953.1.S1_s_at | 2.63 | AY342349 | AB023135 |
| 35 | CfaAffx.18514.1.S1_at | 2.63 | XM_547393 | BC058922 |
| 36 | CfaAffx.17954.1.S1_at | 2.6 | XM_545023 | NM_024090 |
| 37 | Cfa.3093.1.A1_at | 2.59 | AJ011893 | AC018680 |
| 38 | Cfa.19016.1.S1_at | 2.58 | XM_843279 | BC061637 |
| 39 | CfaAffx.28084.1.S1_s_at | 2.58 | NM_001005255 | NM_005623 |
| 40 | CfaAffx.19953.1.S1_at | 2.53 | AC150702 | AL109797 |
| 41 | Cfa.1980.1.S1_at | 2.52 | BC014339 | NM_138786 |
| 42 | Cfa.13370.1.A1_at | 0.4 | NM_001021464 | AL356954 |
| 43 | Cfa.15388.1.S1_at | 0.4 | XM_532002 | AF131836 |
| 44 | Cfa.7478.1.A1_s_at | 0.4 | BC028417 | NM_001093 |
| 45 | Cfa.3749.1.S1_at | 0.4 | NM_001003220 | NM_001006624 |
| 46 | CfaAffx.7949.1.S1_s_at | 0.39 | AK023099 | NM_013380 |
| 47 | CfaAffx.52.1.S1_at | 0.39 | AF159295 | X03205 |
| 48 | Cfa.4556.3.A1_x_at | 0.39 | L36871 | BC073765 |
| 49 | CfaAffx.4308.1.S1_at | 0.39 | XM_861344 | NM_001498 |
| 50 | Cfa.16772.1.A1_at | 0.38 | AF488410 | AB060808 |
| 51 | Cfa.15343.1.A1_s_at | 0.38 | XM_851829 | NM_144583 |
| 52 | CfaAffx.14437.1.S1_at | 0.38 | XM_865312 | BC015752 |
| 53 | CfaAffx.18491.1.S1_s_at | 0.38 | XM_546093 | NM_022786 |
| 54 | CfaAffx.7597.1.S1_at | 0.38 | XM_534118 | AL136960 |
| 55 | CfaAffx.9291.1.S1_s_at | 0.38 | AB020887 | CR626508 |
| 56 | CfaAffx.25065.1.S1_at | 0.38 | NM_001003220 | NM_006474 |
| 57 | CfaAffx.4309.1.S1_s_at | 0.38 | XM_861358 | NM_001498 |
| 58 | Cfa.3478.1.S1_at | 0.37 | AF354266 | NA |
| 59 | CfaAffx.17532.1.S1_s_at | 0.37 | XM_843264 | AY358562 |
| 60 | Cfa.8843.1.A1_s_at | 0.37 | XM_847490 | AY889090 |
| 61 | CfaAffx.28117.1.S1_at | 0.37 | XM_892932 | AC013265 |
| 62 | CfaAffx.16813.1.S1_at | 0.37 | XM_533208 | NM_001876 |
| 63 | CfaAffx.7431.1.S1_at | 0.37 | XM_533636 | BC080551 |
| 64 | CfaAffx.9128.1.S1_s_at | 0.36 | XM_534163 | NM_182848 |
| 65 | CfaAffx.17376.1.S1_s_at | 0.36 | AJ575592 | NM_001093 |
| 66 | Cfa.15138.1.A1_at | 0.36 | NM_001093 | AC007637 |
| 67 | Cfa.101.1.S1_s_at | 0.35 | XM_533208 | BC000185 |
| 68 | Cfa.12375.1.A1_at | 0.35 | XM_538880 | BC042605 |
| 69 | CfaAffx.2191.1.S1_at | 0.34 | XM_532317 | AY082381 |
| 70 | CfaAffx.22979.1.S1_s_at | 0.34 | XM_533208 | AJ420748 |
| 71 | Cfa.15036.1.A1_at | 2.49 | AB169961 | AC005331 |
| 72 | Cfa.2753.1.A1_at | 2.48 | NM_052832 | NM_052832 |
| 73 | Cfa.12493.1.A1_at | 2.45 | XM_860169 | AK168808 |
| 74 | CfaAffx.26260.1.S1_at | 2.45 | XM_542043 | NM_002229 |
| 75 | Cfa.14626.3.S1_at | 2.41 | XM_857812 | AC090341 |
| 76 | Cfa.19427.1.S1_s_at | 2.38 | XM_537080 | AB003698 |
| 77 | CfaAffx.7333.1.S1_at | 2.38 | NM_001031692 | NM_001031692 |
| 78 | Cfa.15094.1.S1_a_at | 2.37 | XM_533973 | AL136962 |
| 79 | Cfa.20568.1.S1_at | 2.36 | NM_003105 | NM_003105 |
| 80 | Cfa.98.1.S1_at | 2.31 | AF479316 | S64152 |
| 81 | Cfa.16947.1.A1_at | 2.31 | XM_543596 | AL512286 |
| 82 | Cfa.5178.2.A1_at | 2.26 | XM_863647 | AC104391 |
| 83 | Cfa.719.1.S1_at | 2.24 | XM_863084 | AB169815 |
| 84 | Cfa.13618.1.A1_at | 2.22 | U10047 | AC002546 |
| 85 | Cfa.15094.2.S1_a_at | 2.22 | XM_847625 | AC107464 |
| 86 | CfaAffx.23392.1.S1_x_at | 2.2 | M59174 | CR542241 |
| 87 | Cfa.1803.1.S1_at | 2.17 | BK001590 | BK001591 |
| 88 | Cfa.9482.1.A1_at | 2.17 | XM_601210 | AY164533 |
| 89 | Cfa.7527.1.A1_at | 2.17 | NM_001018072 | NM_001018072 |
| 90 | Cfa.18826.1.S1_at | 2.17 | XM_853197 | BC015510 |
| 91 | CfaAffx.28599.1.S1_s_at | 2.17 | XM_584816 | BC001421 |
| 92 | CfaAffx.18323.1.S1_at | 2.17 | XM_536545 | NM_003105 |
| 93 | Cfa.19768.1.S1_at | 2.16 | XM_847004 | AC117525 |
| 94 | CfaAffx.11365.1.S1_at | 2.16 | XM_535242 | AF059617 |
| 95 | CfaAffx.13216.1.S1_s_at | 2.15 | XM_534302 | AL833134 |
| 96 | CfaAffx.30642.1.S1_s_at | 2.15 | XM_547262 | NM_144620 |
| 97 | Cfa.19447.1.S1_at | 2.13 | NM_005573 | NM_005573 |
| 98 | Cfa.9467.1.A1_at | 2.13 | XM_540392 | NM_173054 |
| 99 | Cfa.17456.1.S1_at | 2.13 | XM_542013 | AK123265 |
| 100 | Cfa.12527.1.A1_at | 2.13 | XM_586687 | AL513326 |
| 101 | CfaAffx.22739.1.S1_at | 2.12 | XM_548713 | AL162272 |
| 102 | CfaAffx.6614.1.S1_at | 2.11 | AB168572 | AB168572 |
| 103 | Cfa.5981.1.A1_at | 2.11 | XM_603519 | AC100793 |
| 104 | CfaAffx.822.1.S1_s_at | 2.11 | XM_542033 | M62831 |
| 105 | CfaAffx.1705.1.S1_at | 2.1 | XM_538592 | AL008720 |
| 106 | Cfa.5059.1.A1_at | 2.1 | NM_014656 | CR618094 |
| 107 | Cfa.5178.1.S1_at | 2.09 | XM_863647 | AL050322 |
| 108 | CfaAffx.27806.1.S1_x_at | 2.09 | XM_537720 | AY766458 |
| 109 | CfaAffx.9557.1.S1_x_at | 2.09 | BC106930 | BC106930 |
| 110 | Cfa.12326.1.A1_at | 2.08 | XM_723947 | AC072022 |
| 111 | Cfa.13636.1.A1_at | 2.08 | XM_857774 | U49732 |
| 112 | Cfa.825.1.S2_at | 2.08 | AY357941 | AL606517 |
| 113 | CfaAffx.13129.1.S1_at | 2.07 | XM_882294 | AC074032 |
| 114 | CfaAffx.11365.1.S1_s_at | 2.07 | XM_854900 | NM_006622 |
| 115 | Cfa.8466.1.A1_at | 2.06 | XM_756136 | AL591004 |
| 116 | Cfa.1200.1.S1_s_at | 2.06 | AJ560716 | AK093922 |
| 117 | Cfa.15627.1.A1_at | 2.06 | AC097712 | AC097712 |
| 118 | Cfa.14528.1.A1_at | 2.05 | Z25418 | AJ420250 |
| 119 | CfaAffx.28832.1.S1_at | 2.05 | XM_548297 | AF134593 |
| 120 | CfaAffx.30647.1.S1_at | 2.04 | XM_547263 | BX641109 |
| 121 | CfaAffx.30748.1.S1_at | 2.04 | XM_857229 | BT019397 |
| 122 | CfaAffx.9190.1.S1_at | 2.03 | XM_857374 | XM_371614 |
| 123 | Cfa.6729.1.A1_at | 2.03 | BC000671 | BC000671 |
| 124 | Cfa.3358.1.S1_at | 2.03 | NM_024090 | AK027031 |
| 125 | CfaAffx.18323.1.S1_s_at | 2.03 | XM_536545 | U60975 |
| 126 | Cfa.10880.1.A1_s_at | 2.01 | XM_535526 | BC010122 |
| 127 | Cfa.9325.1.A1_x_at | 2.01 | XM_848389 | U50912 |
| 128 | CfaAffx.5584.1.S1_at | 0.5 | XM_539037 | BC045583 |
| 129 | Cfa.11382.1.A1_s_at | 0.5 | XM_537673 | BC012053 |
| 130 | Cfa.13871.1.A1_at | 0.5 | AP001099 | AP001099 |
| 131 | Cfa.1794.1.S1_at | 0.5 | XM_532481 | BC108676 |
| 132 | CfaAffx.22344.1.S1_s_at | 0.5 | AF082505 | AJ841720 |
| 133 | CfaAffx.25283.1.S1_at | 0.5 | XM_846648 | AF186379 |
| 134 | CfaAffx.3950.1.S1_at | 0.5 | XM_531769 | XM_290985 |
| 135 | Cfa.11227.1.A1_at | 0.5 | XM_610609 | AC130450 |
| 136 | CfaAffx.3808.1.S1_s_at | 0.5 | XM_617831 | NM_003558 |
| 137 | Cfa.3648.1.S1_s_at | 0.5 | NM_001003160 | NM_014475 |
| 138 | Cfa.12746.1.S1_at | 0.5 | XM_538481 | AC093840 |
| 139 | Cfa.204.1.S1_s_at | 0.49 | U91844 | U01120 |
| 140 | CfaAffx.28227.1.S1_at | 0.49 | AF306861 | BX161420 |
| 141 | CfaAffx.20380.1.S1_at | 0.49 | XM_854986 | NM_020990 |
| 142 | Cfa.15430.1.A1_at | 0.49 | XM_516700 | AK127468 |
| 143 | Cfa.20429.1.S1_at | 0.49 | BC006523 | Z98752 |
| 144 | Cfa.15663.1.A1_at | 0.49 | BC077424 | AC027237 |
| 145 | Cfa.10921.1.S1_s_at | 0.49 | XM_544508 | AF087892 |
| 146 | Cfa.18524.1.S1_at | 0.49 | XM_546093 | NM_022786 |
| 147 | CfaAffx.28491.1.S1_at | 0.49 | NM_001003186 | AL596025 |
| 148 | Cfa.20844.1.S1_at | 0.49 | XM_582329 | CR749368 |
| 149 | CfaAffx.9612.1.S1_at | 0.49 | XM_849157 | CR536549 |
| 150 | CfaAffx.22561.1.S1_s_at | 0.49 | XM_535367 | BC030153 |
| 151 | Cfa.1465.1.S1_at | 0.49 | AF165917 | NA |
| 152 | CfaAffx.25677.1.S1_s_at | 0.49 | XM_847754 | AK122675 |
| 153 | CfaAffx.9880.1.S1_at | 0.49 | XM_844822 | NM_019095 |
| 154 | Cfa.4978.1.A1_at | 0.49 | AF281074 | AF281074 |
| 155 | CfaAffx.16101.1.S1_s_at | 0.49 | XM_845625 | AC108159 |
| 156 | CfaAffx.21065.1.S1_s_at | 0.49 | XM_844257 | NA |
| 157 | CfaAffx.14411.1.S1_at | 0.49 | XM_535129 | BC064978 |
| 158 | Cfa.12167.1.A1_at | 0.49 | XM_857472 | CR614711 |
| 159 | CfaAffx.9452.1.S1_s_at | 0.49 | XM_857591 | U06117 |
| 160 | Cfa.6037.1.S1_s_at | 0.49 | XM_534893 | CR749334 |
| 161 | CfaAffx.28621.1.S1_at | 0.48 | XM_537333 | CR592932 |
| 162 | CfaAffx.5225.1.S1_s_at | 0.48 | XM_532395 | NM_014465 |
| 163 | Cfa.21549.1.S1_s_at | 0.48 | XM_849503 | AL356218 |
| 164 | Cfa.2610.1.A1_at | 0.48 | AY136628 | AL033519 |
| 165 | CfaAffx.27146.1.S1_at | 0.48 | XM_537549 | AK055200 |
| 166 | Cfa.5002.1.A1_at | 0.48 | CR749631 | NM_019885 |
| 167 | Cfa.7057.1.A1_at | 0.48 | NM_017688 | AC096766 |
| 168 | Cfa.11035.1.A1_at | 0.48 | XM_240178 | AC136767 |
| 169 | CfaAffx.15155.1.S1_s_at | 0.48 | XM_534433 | AL049767 |
| 170 | CfaAffx.7365.1.S1_at | 0.48 | XM_542677 | BC029656 |
| 171 | CfaAffx.18625.1.S1_at | 0.48 | AP008207 | NA |
| 172 | Cfa.349.1.A1_s_at | 0.48 | XM_861720 | NM_015548 |
| 173 | Cfa.14387.1.A1_s_at | 0.48 | XM_533030 | CR620074 |
| 174 | CfaAffx.23219.1.S1_s_at | 0.48 | XM_860432 | BC022516 |
| 175 | CfaAffx.5036.1.S1_s_at | 0.48 | XM_538773 | AK027864 |
| 176 | CfaAffx.20922.1.S1_at | 0.48 | XM_856200 | BC026902 |
| 177 | CfaAffx.3482.1.S1_s_at | 0.48 | XM_538691 | BC068445 |
| 178 | CfaAffx.20220.1.S1_at | 0.48 | XM_548885 | NM_001001671 |
| 179 | Cfa.18842.1.S1_at | 0.48 | XM_862359 | AF268387 |
| 180 | CfaAffx.24173.1.S1_s_at | 0.48 | BC025390 | BC025390 |
| 181 | Cfa.19533.1.S1_s_at | 0.48 | AB208922 | AB208922 |
| 182 | Cfa.11839.1.A1_s_at | 0.48 | XM_535129 | BC064978 |
| 183 | Cfa.12915.1.A1_at | 0.48 | NM_145693 | AC012456 |
| 184 | Cfa.4465.2.S1_s_at | 0.48 | XM_845215 | NA |
| 185 | Cfa.4590.1.S1_s_at | 0.48 | XM_848228 | NM_017680 |
| 186 | Cfa.533.1.S1_at | 0.48 | NM_001034309 | AY358329 |
| 187 | CfaAffx.3714.1.S1_at | 0.48 | XM_541288 | AL162390 |
| 188 | CfaAffx.2004.1.S1_s_at | 0.47 | XM_531894 | NM_001017372 |
| 189 | Cfa.9853.1.A1_at | 0.47 | XM_656697 | AC105753 |
| 190 | CfaAffx.18687.1.S1_at | 0.47 | XM_545513 | NM_199204 |
| 191 | Cfa.3524.1.S2_at | 0.47 | AF336151 | AY506357 |
| 192 | Cfa.4556.3.A1_a_at | 0.47 | L36871 | BC073765 |
| 193 | Cfa.3542.1.S1_at | 0.47 | AB028042 | AC140113 |
| 194 | Cfa.16857.1.S1_at | 0.47 | XM_544507 | BC008983 |
| 195 | CfaAffx.21305.1.S1_at | 0.47 | XM_548543 | AC099669 |
| 196 | CfaAffx.12845.1.S1_at | 0.47 | XM_539763 | NM_001009555 |
| 197 | CfaAffx.18456.1.S1_s_at | 0.47 | XM_535819 | BX648812 |
| 198 | Cfa.16364.1.A1_at | 0.47 | AB220502 | AB220502 |
| 199 | CfaAffx.12483.1.S1_at | 0.47 | NM_175920 | AL080312 |
| 200 | Cfa.10092.1.A1_at | 0.47 | NM_000480 | AC021914 |
| 201 | Cfa.3491.1.S1_s_at | 0.47 | XM_535088 | BC041784 |
| 202 | Cfa.11292.1.A1_at | 0.47 | XM_532002 | AK056752 |
| 203 | Cfa.12131.1.A1_at | 0.47 | XM_548431 | Y17448 |
| 204 | CfaAffx.30657.1.S1_s_at | 0.47 | XM_548431 | NM_004059 |
| 205 | CfaAffx.3284.1.S1_at | 0.47 | XM_846138 | NM_018965 |
| 206 | CfaAffx.3283.1.S1_at | 0.47 | XM_874820 | AY204749 |
| 207 | Cfa.18689.1.S1_at | 0.47 | XM_534893 | CR749334 |
| 208 | Cfa.16744.1.S1_at | 0.47 | XM_873620 | AL162390 |
| 209 | Cfa.12462.1.A1_at | 0.46 | DQ113909 | AC098799 |
| 210 | CfaAffx.29802.1.S1_at | 0.46 | XM_546742 | NM_020710 |
| 211 | Cfa.16431.1.A1_at | 0.46 | CR860237 | AF495544 |
| 212 | CfaAffx.27879.1.S1_s_at | 0.46 | XM_542108 | BC047591 |
| 213 | CfaAffx.6394.1.S1_at | 0.46 | XM_844361 | NM_020724 |
| 214 | Cfa.12296.1.A1_at | 0.46 | BC046475 | AC010243 |
| 215 | CfaAffx.14851.1.S1_s_at | 0.46 | XM_535300 | BC002576 |
| 216 | Cfa.14057.1.A1_at | 0.46 | XM_536086 | NM_003413 |
| 217 | Cfa.5948.1.A1_at | 0.46 | NM_080165 | AC010887 |
| 218 | Cfa.12143.1.A1_at | 0.46 | XM_850039 | AP002414 |
| 219 | Cfa.9627.1.A1_at | 0.46 | AF526382 | AF526382 |
| 220 | CfaAffx.8473.1.S1_s_at | 0.46 | XM_852658 | BC042131 |
| 221 | Cfa.14620.1.A1_at | 0.46 | XM_532317 | CR623165 |
| 222 | CfaAffx.5908.1.S1_at | 0.46 | XM_860659 | NM_001550 |
| 223 | CfaAffx.13161.1.S1_s_at | 0.46 | XM_544819 | CR620760 |
| 224 | Cfa.11008.1.A1_at | 0.46 | BC102635 | BC014225 |
| 225 | Cfa.3324.1.S1_at | 0.46 | XM_538691 | AL451123 |
| 226 | Cfa.10039.1.A1_at | 0.46 | XM_844773 | AF374726 |
| 227 | Cfa.17677.1.S1_at | 0.46 | XM_838131 | AL031676 |
| 228 | CfaAffx.26949.1.S1_s_at | 0.46 | XM_547958 | AL132642 |
| 229 | CfaAffx.30657.1.S1_at | 0.46 | XM_548431 | NM_004059 |
| 230 | CfaAffx.21066.1.S1_s_at | 0.46 | XM_844290 | NA |
| 231 | Cfa.18689.1.S1_s_at | 0.46 | XM_534893 | BC040071 |
| 232 | CfaAffx.15436.1.S1_at | 0.45 | XM_543027 | BC019898 |
| 233 | CfaAffx.12835.1.S1_at | 0.45 | XM_854906 | NM_144668 |
| 234 | Cfa.19549.1.S1_s_at | 0.45 | BC022526 | AJ298293 |
| 235 | Cfa.4555.1.S1_s_at | 0.45 | L36871 | AY647979 |
| 236 | Cfa.18258.2.S1_a_at | 0.45 | NM_001009867 | NA |
| 237 | CfaAffx.3919.1.S1_s_at | 0.45 | XM_515679 | AC010095 |
| 238 | Cfa.19518.2.A1_s_at | 0.45 | XM_535260 | NM_018695 |
| 239 | Cfa.19549.1.S1_at | 0.45 | AJ298293 | AJ298293 |
| 240 | CfaAffx.22832.1.S1_s_at | 0.45 | XM_536069 | NM_032726 |
| 241 | Cfa.2282.1.S1_at | 0.45 | XM_539427 | AK096428 |
| 242 | CfaAffx.26949.1.S1_at | 0.45 | XM_547958 | AL137735 |
| 243 | Cfa.10854.1.S1_at | 0.45 | XM_532878 | AL513550 |
| 244 | CfaAffx.7437.1.S1_s_at | 0.45 | XM_533636 | AB054067 |
| 245 | Cfa.3664.1.S1_s_at | 0.45 | NM_001003173 | AC004485 |
| 246 | Cfa.15462.1.A1_at | 0.45 | NM_001003173 | AL161729 |
| 247 | CfaAffx.9797.1.S1_s_at | 0.44 | XM_534252 | AL831925 |
| 248 | CfaAffx.25159.1.S1_at | 0.44 | NM_001002838 | NM_020922 |
| 249 | Cfa.4556.3.A1_s_at | 0.44 | L36871 | AY647979 |
| 250 | Cfa.16500.1.S1_at | 0.44 | XM_531940 | AB070011 |
| 251 | Cfa.18258.3.S1_at | 0.44 | XM_844019 | AB169867 |
| 252 | CfaAffx.11992.1.S1_s_at | 0.44 | XM_535094 | NM_024790 |
| 253 | CfaAffx.4328.1.S1_at | 0.44 | XM_848235 | NM_005014 |
| 254 | Cfa.19653.1.A1_at | 0.44 | BC081135 | AC112777 |
| 255 | CfaAffx.27578.1.S1_at | 0.44 | XM_547618 | NM_022751 |
| 256 | CfaAffx.30551.1.S1_at | 0.44 | XM_546802 | AF302109 |
| 257 | Cfa.13268.1.A1_at | 0.44 | AB126596 | AC026778 |
| 258 | Cfa.18183.1.S1_at | 0.44 | XM_857210 | AC008387 |
| 259 | Cfa.6019.1.A1_at | 0.44 | XM_724777 | AC147004 |
| 260 | Cfa.93.1.S1_at | 0.44 | CR860955 | AL137918 |
| 261 | CfaAffx.25065.1.S1_s_at | 0.44 | NM_001003220 | AF030428 |
| 262 | Cfa.13033.1.A1_at | 0.43 | CR858688 | NA |
| 263 | Cfa.5688.1.A1_at | 0.43 | NM_015336 | AL158196 |
| 264 | Cfa.15343.1.A1_a_at | 0.43 | XM_843630 | NM_144583 |
| 265 | CfaAffx.25467.1.S1_at | 0.43 | XM_537659 | NM_006310 |
| 266 | CfaAffx.4613.1.S1_at | 0.43 | XM_538986 | XM_498111 |
| 267 | CfaAffx.2014.1.S1_at | 0.43 | XM_214555 | AC008591 |
| 268 | Cfa.9659.1.A1_at | 0.43 | AB051389 | AC108046 |
| 269 | Cfa.1465.2.A1_at | 0.43 | NM_213992 | AC106768 |
| 270 | Cfa.11205.1.A1_at | 0.43 | NM_000216 | S60088 |
| 271 | CfaAffx.30851.1.S1_s_at | 0.43 | XM_537071 | BC056667 |
| 272 | CfaAffx.10452.1.S1_s_at | 0.43 | XM_590483 | AC009194 |
| 273 | CfaAffx.6374.1.S1_s_at | 0.43 | XM_851910 | AB168681 |
| 274 | CfaAffx.8051.1.S1_at | 0.43 | XM_844206 | BC102460 |
| 275 | CfaAffx.9808.1.S1_at | 0.43 | XM_534351 | AL035668 |
| 276 | Cfa.7153.1.A1_s_at | 0.43 | XM_534351 | AL035668 |
| 277 | Cfa.15136.1.S1_at | 0.43 | NM_001206 | AL162390 |
| 278 | Cfa.5277.1.A1_s_at | 0.42 | XM_532120 | NM_145028 |
| 279 | CfaAffx.3288.1.S1_at | 0.42 | XM_846150 | AL138898 |
| 280 | CfaAffx.14664.1.S1_s_at | 0.42 | XM_844773 | AF374726 |
| 281 | Cfa.13412.1.A1_at | 0.42 | XM_854906 | BC028421 |
| 282 | Cfa.20984.1.S1_at | 0.42 | XM_539525 | AB169259 |
| 283 | CfaAffx.8004.1.S1_s_at | 0.42 | XM_850120 | XM_371174 |
| 284 | Cfa.5715.1.S1_at | 0.42 | XM_532133 | AL133404 |
| 285 | Cfa.1286.1.A1_at | 0.42 | XM_583309 | CR599853 |
| 286 | Cfa.17433.1.S1_s_at | 0.42 | XM_548431 | NM_004059 |
| 287 | CfaAffx.11552.1.S1_s_at | 0.41 | XM_533773 | BC017772 |
| 288 | Cfa.13150.1.A1_at | 0.41 | NM_177737 | AC044787 |
| 289 | CfaAffx.11212.1.S1_s_at | 0.41 | XM_540544 | AL832391 |
| 290 | CfaAffx.1928.1.S1_at | 0.41 | XM_541178 | NM_032532 |
| 291 | Cfa.11092.1.A1_at | 0.41 | NM_021197 | AC009123 |
| 292 | CfaAffx.19304.1.S1_at | 0.41 | XM_846272 | AC079151 |
| 293 | CfaAffx.5035.1.S1_s_at | 0.41 | XM_858105 | CR625459 |
| 294 | Cfa.3648.1.S1_at | 0.41 | XM_517428 | AC006320 |
| 295 | Cfa.11104.1.S1_at | 0.41 | XM_535184 | AC083886 |

TABLE 2
Genes associated with lipid and glucose metabolism differentially
expressed in adipose tissue from fat compared to lean animals (group 4)
Column

| 1 | 2 | 3 | 4 | 5 |
|---|---|---|---|---|
| 55 | CfaAffx.9291.1.S1_s_at | 0.38 | AB020887 | CR626508 |
| 60 | Cfa.8843.1.A1_s_at | 0.37 | XM_847490 | AY889090 |
| 81 | Cfa.16947.1.A1_at | 2.31 | XM_543596 | AL512286 |
| 112 | Cfa.825.1.S2_at | 2.08 | AY357941 | AL606517 |
| 133 | CfaAffx.25283.1.S1_at | 0.5 | XM_846648 | AF186379 |
| 204 | Cfa.204.1.S1_s_at | 0.49 | U91844 | U01120 |
| 188 | CfaAffx.2004.1.S1_s_at | 0.47 | XM_531894 | NM_001017372 |
| 212 | CfaAffx.27879.1.S1_s_at | 0.46 | XM_542108 | BC047591 |
| 216 | Cfa.14057.1.A1_at | 0.46 | XM_536086 | NM_003413 |
| 232 | CfaAffx.15436.1.S1_at | 0.45 | XM_543027 | BC019898 |
| 240 | CfaAffx.22832.1.S1_s_at | 0.45 | XM_536069 | NM_032726 |
| 274 | CfaAffx.8051.1.S1_at | 0.43 | XM_844206 | BC102460 |
| 295 | Cfa.11104.1.S1_at | 0.41 | XM_535184 | AC083886 |
| 11 | Cfa.15689.1.A1_at | 0.33 | XM_844220 | AC020550 |
| 241 | Cfa.2282.1.S1_at | 0.45 | XM_539427 | AK096428 |
| 15 | CfaAffx.4097.1.S1_s_at | 0.32 | XM_539427 | BC040239 |
| 44 | Cfa.7478.1.A1_s_at | 0.4 | BC028417 | NM_001093 |
| 18 | CfaAffx.17336.1.S1_s_at | 0.28 | AJ575592 | NM_001093 |
| 65 | CfaAffx.17376.1.S1_s_at | 0.36 | AJ575592 | NM_001093 |
| 66 | Cfa.15138.1.A1_at | 0.36 | NM_001093 | AC007637 |
| 124 | Cfa.3358.1.S1_at | 2.03 | NM_024090 | AK027031 |
| 36 | CfaAffx.17954.1.S1_at | 2.6 | XM_545023 | NM_024090 |
| 285 | Cfa.1286.1.A1_at | 0.42 | XM_583309 | CR599853 |
| 67 | Cfa.101.1.S1_s_at | 0.35 | XM_533208 | BC000185 |
| 62 | CfaAffx.16813.1.S1_at | 0.37 | XM_533208 | NM_001876 |
| 70 | CfaAffx.22979.1.S1_s_at | 0.34 | XM_533208 | AJ420748 |
| 182 | Cfa.11839.1.A1_s_at | 0.48 | XM_535129 | BC064978 |
| 157 | CfaAffx.14411.1.S1_at | 0.49 | XM_535129 | BC064978 |
| 183 | Cfa.12915.1.A1_at | 0.48 | NM_145693 | AC012456 |
| 243 | Cfa.10854.1.S1_at | 0.45 | XM_532878 | AL513550 |
| 273 | CfaAffx.6374.1.S1_s_at | 0.43 | XM_851910 | AB168681 |

TABLE 3
Genes identified as particularly relevant to fat
animals compared to lean animals (group 5)
Column

| 1 | 2 | 3 | 4 | 5 |
|---|---|---|---|---|
| 6 | Cfa.9039.1.A1_at | 3.07 | XM_547914 | BX647104 |
| 2 | CfaAffx.26065.1.S1_at | 3.94 | XM_547914 | AF111167 |
| 3 | CfaAffx.2782.1.S1_s_at | 3.78 | XM_538649 | AJ243425 |
| 4 | CfaAffx.2790.1.S1_s_at | 3.66 | XM_538649 | BC073983 |
| 274 | CfaAffx.8051.1.S1_at | 0.43 | XM_844206 | BC102460 |
| 295 | Cfa.11104.1.S1_at | 0.41 | XM_535184 | AC083886 |
| 11 | Cfa.15689.1.A1_at | 0.33 | XM_844220 | AC020550 |
| 68 | Cfa.12375.1.A1_at | 0.35 | XM_538880 | BC042605 |
| 12 | CfaAffx.2909.1.S1_at | 0.33 | XM_538880 | NM_004117 |
| 241 | Cfa.2282.1.S1_at | 0.45 | XM_539427 | AK096428 |
| 15 | CfaAffx.4097.1.S1_s_at | 0.32 | XM_539427 | BC040239 |
| 44 | Cfa.7478.1.A1_s_at | 0.4 | BC028417 | NM_001093 |
| 18 | CfaAffx.17336.1.S1_s_at | 0.28 | AJ575592 | NM_001093 |
| 65 | CfaAffx.17376.1.S1_s_at | 0.36 | AJ575692 | NM_001093 |
| 66 | Cfa.15138.1.A1_at | 0.36 | NM_001093 | AC007637 |
| 13 | CfaAffx.4844.1.S1_s_at | 0.33 | XM_538481 | BT019766 |
| 19 | CfaAffx.4844.1.S1_at | 0.26 | XM_538481 | BT019766 |
| 138 | Cfa.12746.1.S1_at | 0.5 | XM_538481 | AC093840 |
| 25 | CfaAffx.732.1.S1_at | 0.12 | NM_181756 | NM_181756 |
| 24 | CfaAffx.732.1.S1_x_at | 0.14 | NM_181756 | AK095351 |
| 124 | Cfa.3358.1.S1_at | 2.03 | NM_024090 | AK027031 |
| 36 | CfaAffx.17954.1.S1_at | 2.6 | XM_545023 | NM_024090 |
| 56 | CfaAffx.25065.1.S1_at | 0.38 | NM_001003220 | NM_006474 |
| 45 | Cfa.3749.1.S1_at | 0.4 | NM_001003220 | NM_001006624 |
| 261 | CfaAffx.25065.1.S1_s_at | 0.44 | NM_001003220 | AF030428 |
| 49 | CfaAffx.4308.1.S1_at | 0.39 | XM_861344 | NM_001498 |
| 57 | CfaAffx.4309.1.S1_s_at | 0.38 | XM_861358 | NM_001498 |
| 63 | CfaAffx.7431.1.S1_at | 0.37 | XM_533636 | BC080551 |
| 244 | CfaAffx.7437.1.S1_s_at | 0.45 | XM_533636 | AB054067 |
| 285 | Cfa.1286.1.A1_at | 0.42 | XM_583309 | CR599853 |
| 67 | Cfa.101.1.S1_s_at | 0.35 | XM_533208 | BC000185 |
| 62 | CfaAffx.16813.1.S1_at | 0.37 | XM_533208 | NM_001876 |
| 70 | CfaAffx.22979.1.S1_s_at | 0.34 | XM_533208 | AJ420748 |
| 78 | Cfa.15094.1.S1_a_at | 2.37 | XM_533973 | AL136962 |
| 85 | Cfa.15094.2.S1_a_at | 2.22 | XM_847625 | AC107464 |
| 92 | CfaAffx.18323.1.S1_at | 2.17 | XM_536545 | NM_003105 |
| 79 | Cfa.20568.1.S1_at | 2.36 | NM_003105 | NM_003105 |
| 125 | CfaAffx.18323.1.S1_s_at | 2.03 | XM_536545 | U60975 |
| 94 | CfaAffx.11365.1.S1_at | 2.16 | XM_535242 | AF059617 |
| 114 | CfaAffx.11365.1.S1_s_at | 2.07 | XM_854900 | NM_006622 |
| 158 | Cfa.12167.1.A1_at | 0.49 | XM_857472 | CR614711 |
| 159 | CfaAffx.9452.1.S1_s_at | 0.49 | XM_857591 | U06117 |
| 182 | Cfa.11839.1.A1_s_at | 0.48 | XM_535129 | BC064978 |
| 157 | CfaAffx.14411.1.S1_at | 0.49 | XM_535129 | BC064978 |
| 185 | Cfa.4590.1.S1_s_at | 0.48 | XM_848228 | NM_017680 |
| 186 | Cfa.533.1.S1_at | 0.48 | NM_001034309 | AY358329 |
| 205 | CfaAffx.3284.1.S1_at | 0.47 | XM_846138 | NM_018965 |
| 206 | CfaAffx.3283.1.S1_at | 0.47 | XM_874820 | AY204749 |
| 184 | Cfa.4465.2.S1_s_at | 0.48 | XM_845215 | NA |
| 230 | CfaAffx.21066.1.S1_s_at | 0.46 | XM_844290 | NA |
| 207 | Cfa.18689.1.S1_at | 0.47 | XM_534893 | CR749334 |
| 231 | Cfa.18689.1.S1_s_at | 0.46 | XM_534893 | BC040071 |
| 160 | Cfa.6037.1.S1_s_at | 0.49 | XM_534893 | CR749334 |
| 242 | CfaAffx.26949.1.S1_at | 0.45 | XM_547958 | AL137735 |
| 228 | CfaAffx.26949.1.S1_s_at | 0.46 | XM_547958 | AL132642 |
| 245 | Cfa.3664.1.S1_s_at | 0.45 | NM_001003173 | AC004485 |
| 246 | Cfa.15462.1.A1_at | 0.45 | NM_001003173 | AL161729 |
| 183 | Cfa.12915.1.A1_at | 0.48 | NM_145693 | AC012456 |
| 243 | Cfa.10854.1.S1_at | 0.45 | XM_532878 | AL513550 |
| 273 | CfaAffx.6374.1.S1_s_at | 0.43 | XM_851910 | AB168681 |
| 275 | CfaAffx.9808.1.S1_at | 0.43 | XM_534351 | AL035668 |
| 276 | Cfa.7153.1.A1_s_at | 0.43 | XM_534351 | AL035668 |
| 187 | CfaAffx.3714.1.S1_at | 0.48 | XM_541288 | AL162390 |
| 277 | Cfa.15136.1.S1_at | 0.43 | NM_001206 | AL162390 |
| 208 | Cfa.16744.1.S1_at | 0.47 | XM_873620 | AL162390 |
| 203 | Cfa.12131.1.A1_at | 0.47 | XM_548431 | Y17448 |
| 229 | CfaAffx.30657.1.S1_at | 0.46 | XM_548431 | NM_004059 |
| 204 | CfaAffx.30657.1.S1_at | 0.47 | XM_548431 | NM_004059 |
| 286 | Cfa.17433.1.S1_s_at | 0.42 | XM_548431 | NM_004059 |

TABLE 4
Gene Description - Highest BLAST Hit Accession Number

| SEQ ID NO | Gene Description - Highest BLAST Hit Accession Number |
|---|---|
| 1 | PREDICTED: Pan troglodytes similar to hypothetical protein ARM (LOC460002), |
| | mRNA |
| 2 | PREDICTED: Canis familiaris similar to Proto-oncogene protein c-fos (Cellular |
| | oncogene fos) (G0/G1 switch regulatory protein 7), transcript variant 1 |
| | (LOC490792), mRNA |
| 3 | PREDICTED: Canis familiaris similar to Early growth response protein 1 (EGR-1) |
| | (Krox-24 protein) (ZIF268) (Nerve growth factor-induced protein A) (NGFI-A) |
| | (Transcription factor ETR103) (Zinc finger protein 225) (AT225), transcript variant |
| | 2 (LOC481528), mRNA |
| 4 | PREDICTED: Canis familiaris similar to Early growth response protein 1 (EGR-1) |
| | (Krox-24 protein) (ZIF268) (Nerve growth factor-induced protein A) (NGFI-A) |
| | (Transcription factor ETR103) (Zinc finger protein 225) (AT225), transcript variant |
| | 2 (LOC481528), mRNA |
| 5 | Homo sapiens thymopoietin (TMPO), transcript variant 3, mRNA |
| 6 | PREDICTED: Canis familiaris similar to Proto-oncogene protein c-fos (Cellular |
| | oncogene fos) (G0/G1 switch regulatory protein 7), transcript variant 1 |
| | (LOC490792), mRNA |
| 7 | Homo sapiens zinc finger protein 227 (ZNF227), mRNA |
| 8 | PREDICTED: Canis familiaris similar to solute carrier family 7 (cationic amino |
| | acid transporter, y+ system), member 3 (LOC486765), mRNA |
| 9 | PREDICTED: Strongylocentrotus purpuratus similar to CG31108-PA |
| | (LOC582217), partial mRNA |
| 10 | PREDICTED: Canis familiaris similar to serum/glucocorticoid regulated kinase 2 |
| | isoform beta (LOC610835), mRNA |
| 11 | PREDICTED: Canis familiaris similar to phytanoyl-CoA hydroxylase precursor |
| | (LOC478001), mRNA |
| 12 | PREDICTED: Canis familiaris similar to FK506-binding protein 5 (Peptidyl-prolyl |
| | cis-trans isomerase) (PPlase) (Rotamase) (51 kDa FK506-binding protein) |
| | (FKBP-51) (54 kDa progesterone receptor-associated immunophilin) (FKBP54) |
| | (P54) (FF1 antigen) (HSP90-binding immunophilin) ( . . . (LOC481759), mRNA |
| 13 | PREDICTED: Canis familiaris similar to Tumor-associated calcium signal |
| | transducer 1 precursor (Major gastrointestinal tumor-associated protein GA733-2) |
| | (Epithelial cell surface antigen) (Epithelial glycoprotein) (EGP) (Adenocarcinoma- |
| | associated antigen) (KSA) (KS 1/4 antigen) . . . (LOC481360), mRNA |
| 14 | Homo sapiens, clone IMAGE: 5171802, mRNA |
| 15 | PREDICTED: Canis familiaris similar to [Pyruvate dehydrogenase [lipoamide]] |
| | kinase isozyme 4, mitochondrial precursor (Pyruvate dehydrogenase kinase |
| | isoform 4) (LOC482310), mRNA |
| 16 | PREDICTED: Canis familiaris similar to niban protein isoform 2 (LOC480041), |
| | mRNA |
| 17 | Caenorhabditis elegans BMP receptor Associated protein family member (bra-1) |
| | (bra-1) mRNA, complete cds |
| 18 | Homo sapiens mRNA for Acetyl-CoA carboxylase 2 (ACACB gene) |
| 19 | PREDICTED: Canis familiaris similar to Tumor-associated calcium signal |
| | transducer 1 precursor (Major gastrointestinal tumor-associated protein GA733-2) |
| | (Epithelial cell surface antigen) (Epithelial glycoprotein) (EGP) (Adenocarcinoma- |
| | associated antigen) (KSA) (KS 1/4 antigen) . . . (LOC481360), mRNA |
| 20 | Mus musculus Murr1 and U2af1-rs1 genes, partial and complete cds |
| 21 | Campylobacter jejuni 81-176 (pflA) gene, complete cds, orf1 and orf2, partial cds |
| 22 | PREDICTED: Canis familiaris similar to suprabasin (LOC612650), mRNA |
| 23 | Plasmodium yoelii yoelii str. 17XNL hypothetical protein (PY04060) mRNA, partial |
| | cds |
| 24 | Homo sapiens zinc finger protein 233 (ZNF233), mRNA |
| 25 | Homo sapiens zinc finger protein 233 (ZNF233), mRNA |
| 26 | PREDICTED: Canis familiaris hypothetical LOC130733 (LOC475737), mRNA |
| 27 | Homo sapiens bcl6 gene, 5′ flanking region |
| 28 | Homo sapiens G protein-coupled receptor 51 (GPR51), mRNA |
| 29 | PREDICTED: Canis familiaris similar to transmembrane protein with EGF-like |
| | and two follistatin-like domains 1, transcript variant 1 (LOC612942), mRNA |
| 30 | C. familiaris mRNA for orphan nuclear receptor dNGFI-B protein |
| 31 | PREDICTED: Bos taurus putative MIP1-beta protein (LOC414347), mRNA |
| 32 | L. japonicus mRNA for small GTP-binding protein, RAB7C |
| 33 | Canis familiaris chemokine (C-C motif) ligand 2 (CCL2), mRNA |
| 34 | Canis familiaris inducible T-cell co-stimulator (ICOS) mRNA, complete cds |
| 35 | PREDICTED: Canis familiaris laminin beta 3 (LOC442953), mRNA |
| 36 | PREDICTED: Canis familiaris similar to ELOVL family member 6, elongation of |
| | long chain fatty acids (FEN1/Elo2, SUR4/Elo3-like, yeast) (LOC487900), mRNA |
| 37 | Nicotiana tabacum mRNA for cyclin D3.1 protein (CycD3.1) |
| 38 | PREDICTED: Canis familiaris similar to Protein C14orf119 (LOC607014), mRNA |
| 39 | Canis familiaris chemokine (C-C motif) ligand 8 (CCL8), mRNA |
| 40 | Oryza sativa (japonica cultivar-group) chromosome 11 clone B1356E08, |
| | complete sequence |
| 41 | Homo sapiens transmembrane 4 L six family member 18, mRNA (cDNA clone |
| | MGC: 23935 IMAGE: 3828466), complete cds |
| 42 | Schizosaccharomyces pombe 972h-isoleucine-tRNA ligase (SPBC8D2.06), |
| | partial mRNA |
| 43 | PREDICTED: Canis familiaris similar to tropomodulin 1, transcript variant 1 |
| | (LOC474771), mRNA |
| 44 | Homo sapiens acetyl-Coenzyme A carboxylase beta, mRNA (cDNA clone |
| | IMAGE: 4824130), complete cds |
| 45 | Canis familiaris podoplanin (PDPN), mRNA |
| 46 | Homo sapiens cDNA FLJ13037 fis, clone NT2RP3001268, highly similar to Homo |
| | sapiens zinc finger protein ZNF228 (ZNF228) mRNA |
| 47 | Homo sapiens serine/threonine protein kinase Kp78 splice variant CTAK75a |
| | mRNA, complete cds |
| 48 | Canis familiaris IgA heavy chain constant region gene, partial cds |
| 49 | PREDICTED: Canis familiaris similar to Glutamate--cysteine ligase catalytic |
| | subunit (Gamma-glutamylcysteine synthetase) (Gamma-ECS) (GCS heavy |
| | chain), transcript variant 3 (LOC609822), mRNA |
| 50 | Homo sapiens cyclin-dependent kinase inhibitor mRNA, partial cds |
| 51 | PREDICTED: Canis familiaris similar to ATPase, H+ transporting, lysosomal |
| | 42 kDa, V1 subunit C isoform 2, transcript variant 4 (LOC475667), mRNA |
| 52 | PREDICTED: Bos taurus similar to Interferon regulatory factor 4 (IRF-4) |
| | (Lymphocyte specific interferon regulatory factor) (LSIRF) (NF-EM5) (Multiple |
| | myeloma oncogene 1), transcript variant 2 (LOC506141), mRNA |
| 53 | PREDICTED: Canis familiaris similar to ARV1 homolog, transcript variant 1 |
| | (LOC488975), mRNA |
| 54 | PREDICTED: Canis familiaris retinoblastoma 1 (RB1), mRNA |
| 55 | Canis familiaris ucp2 mRNA for uncoupling protein 2, complete cds |
| 56 | Canis familiaris podoplanin (PDPN), mRNA |
| 57 | PREDICTED: Canis familiaris similar to Glutamate--cysteine ligase catalytic |
| | subunit (Gamma-glutamylcysteine synthetase) (Gamma-ECS) (GCS heavy |
| | chain), transcript variant 4 (LOC609822), mRNA |
| 58 | Canis familiaris immunoglobulin gamma heavy chain C mRNA, complete cds |
| 59 | PREDICTED: Canis familiaris similar to X-linked neuroligin 4, transcript variant 1 |
| | (LOC607406), mRNA |
| 60 | PREDICTED: Canis familiaris similar to Apolipoprotein C-I precursor (Apo-CI) |
| | (ApoC-I), transcript variant 2 (LOC476437), mRNA |
| 61 | PREDICTED: Mus musculus hypothetical protein LOC628198 (LOC628198), |
| | mRNA |
| 62 | PREDICTED: Canis familiaris carnitine palmitoyl transferase I isoform (CPT1), |
| | mRNA |
| 63 | PREDICTED: Canis familiaris similar to hypoxia-inducible factor-3 alpha isoform |
| | a (LOC476429), mRNA |
| 64 | PREDICTED: Canis familiaris similar to claudin 10 isoform b, transcript variant 1 |
| | (LOC476963), mRNA |
| 65 | Homo sapiens mRNA for Acetyl-CoA carboxylase 2 (ACACB gene) |
| 66 | Homo sapiens acetyl-Coenzyme A carboxylase beta (ACACB), mRNA |
| 67 | PREDICTED: Canis familiaris carnitine palmitoyl transferase I isoform (CPT1), |
| | mRNA |
| 68 | PREDICTED: Canis familiaris similar to FK506-binding protein 5 (Peptidyl-prolyl |
| | cis-trans isomerase) (PPlase) (Rotamase) (51 kDa FK506-binding protein) |
| | (FKBP-51) (54 kDa progesterone receptor-associated immunophilin) (FKBP54) |
| | (P54) (FF1 antigen) (HSP90-binding immunophilin) ( . . . (LOC481759), mRNA |
| 69 | PREDICTED: Canis familiaris similar to NOV protein homolog precursor (NovH) |
| | (Nephroblastoma overexpressed gene protein homolog) (LOC475083), mRNA |
| 70 | PREDICTED: Canis familiaris carnitine palmitoyl transferase I isoform (CPT1), |
| | mRNA |
| 71 | Nitella japonica chromoplast atpB gene for ATP synthase beta subunit, partial |
| | cds, strain: S090 |
| 72 | Homo sapiens solute carrier family 26, member 7 (SLC26A7), transcript variant 1, |
| | mRNA |
| 73 | PREDICTED: Canis familiaris similar to NHP2-like protein 1 (High mobility group- |
| | like nuclear protein 2 homolog 1) ([U4/U6.U5] tri-snRNP 15.5 kDa protein) |
| | (Sperm specific antigen 1) (Fertilization antigen 1) (FA-1), transcript variant 2 |
| | (LOC609886), mRNA |
| 74 | PREDICTED: Canis familiaris similar to Transcription factor jun-B (LOC484927), |
| | mRNA |
| 75 | PREDICTED: Canis familiaris similar to Collagen alpha 1(III) chain precursor, |
| | transcript variant 3 (LOC478835), mRNA |
| 76 | PREDICTED: Canis familiaris similar to Cell division cycle 7-related protein |
| | kinase (CDC7-related kinase) (HsCdc7) (huCdc7) (LOC479955), mRNA |
| 77 | Homo sapiens leucine rich repeat containing 17 (LRRC17), transcript variant 1, |
| | mRNA |
| 78 | PREDICTED: Canis familiaris similar to expressed in non-metastatic cells 1, |
| | protein (NM23A) (nucleoside diphosphate kinase) (LOC476767), mRNA |
| 79 | Homo sapiens sortilin-related receptor, L(DLR class) A repeats-containing |
| | (SORL1), mRNA |
| 80 | Canis familiaris dystrophin (DMD) mRNA, 5′ untranslated region, alternatively |
| | spliced |
| 81 | PREDICTED: Canis familiaris similar to cholesterol 25-hydroxylase |
| | (LOC486470), mRNA |
| 82 | PREDICTED: Canis familiaris hypothetical protein LOC612422 (LOC612422), |
| | mRNA |
| 83 | PREDICTED: Canis familiaris aldolase C, transcript variant 4 (LOC480622). |
| | mRNA |
| 84 | Pisum sativum ribosomal protein L34 homolog (RPL34) mRNA, complete cds |
| 85 | PREDICTED: Canis familiaris similar to expressed in non-metastatic cells 1, |
| | protein (NM23A) (nucleoside diphosphate kinase) (LOC609873), mRNA |
| 86 | Canis familiaris serum amyloid A protein (SAA) mRNA, partial cds |
| 87 | TPA: Homo sapiens chromosome 17 middle SMS-REP low-copy repeat, genomic |
| | sequence |
| 88 | PREDICTED: Bos taurus similar to heparin-binding EGF-like growth factor |
| | (LOC522921), mRNA |
| 89 | Homo sapiens BTB (POZ) domain containing 11 (BTBD11), transcript variant 3, |
| | mRNA |
| 90 | PREDICTED: Canis familiaris similar to Regulator of G-protein signaling 1 |
| | (RGS1) (Early response protein 1R20) (B-cell activation protein BL34), transcript |
| | variant 2 (LOC488585), mRNA |
| 91 | PREDICTED: Bos taurus similar to nuclear distribution gene E homolog 1 |
| | (LOC508088), mRNA |
| 92 | PREDICTED: Canis familiaris similar to sortilin-related receptor containing LDLR |
| | class A repeats preproprotein (LOC479408), mRNA |
| 93 | PREDICTED: Canis familiaris similar to ankyrin repeat domain 26 (LOC609691), |
| | mRNA |
| 94 | PREDICTED: Canis familiaris similar to polo-like kinase 2, transcript variant 1 |
| | (LOC478063), mRNA |
| 95 | PREDICTED: Canis familiaris similar to Transmembrane 4 L6 family member 1 |
| | (Tumor-associated antigen L6) (Membrane component, surface marker 1) |
| | (M3S1) (LOC477107), mRNA |
| 96 | PREDICTED: Canis familiaris similar to leucine rich repeat containing 39, |
| | transcript variant 1 (LOC490141), mRNA |
| 97 | Homo sapiens lamin B1 (LMNB1), mRNA |
| 98 | PREDICTED: Canis familiaris similar to reelin isoform b, transcript variant 1 |
| | (LOC483273), mRNA |
| 99 | PREDICTED: Canis familiaris similar to egf-like module containing, mucin-like, |
| | hormone receptor-like sequence 2 isoform d (LOC484897), mRNA |
| 100 | PREDICTED: Bos taurus similar to BTG2 protein (NGF-inducible protein TIS21) |
| | (LOC539364), mRNA |
| 101 | PREDICTED: Canis familiaris similar to ankyrin repeat domain 26 (LOC491592), |
| | mRNA |
| 102 | Macaca fascicularis testis cDNA clone: QtsA-13105, similar to human armadillo |
| | repeat containing 2 (ARMC2), mRNA, RefSeq: NM_032131.3 |
| 103 | PREDICTED: Bos taurus similar to glycerophosphodiester phosphodiesterase |
| | domain containing 4 (LOC525172), mRNA |
| 104 | PREDICTED: Canis familiaris similar to immediate early response 2 |
| | (LOC484917), mRNA |
| 105 | PREDICTED: Canis familiaris similar to interferon gamma inducible protein 47 |
| | (LOC481471), mRNA |
| 106 | Homo sapiens KIAA0040 (KIAA0040), mRNA |
| 107 | PREDICTED: Canis familiaris hypothetical protein LOC612422 (LOC612422), |
| | mRNA |
| 108 | PREDICTED: Canis familiaris similar to Small inducible cytokine A3-like 1 |
| | precursor (Tonsillar lymphocyte LD78 beta protein) (LD78-beta(1-70)) (G0/G1 |
| | switch regulatory protein 19-2) (G0S19-2 protein) (PAT 464.2) (LOC480600), |
| | mRNA |
| 109 | Homo sapiens thyroid hormone receptor, beta (erythroblastic leukemia viral (verb- |
| | a) oncogene homolog 2, avian), mRNA (cDNA clone MGC: 126110 |
| | IMAGE: 40033200), complete cds |
| 110 | Plasmodium yoelii yoelii str. 17XNL hypothetical protein (PY01308) mRNA, partial |
| | cds |
| 111 | PREDICTED: Canis familiaris similar to mitogen-activated protein kinase kinase 6 |
| | isoform 1, transcript variant 3 (LOC480454), mRNA |
| 112 | Homo sapiens glucose transporter 14 short isoform mRNA, complete cds; |
| | alternatively spliced |
| 113 | PREDICTED: Bos taurus similar to LAG1 longevity assurance homolog 5, |
| | transcript variant 2 (LOC530776), mRNA |
| 114 | PREDICTED: Canis familiaris similar to polo-like kinase 2, transcript variant 3 |
| | (LOC478063), mRNA |
| 115 | Ustilago maydis 521 hypothetical protein (UM05082.1), mRNA |
| 116 | Canis familiaris mRNA for putative secreted frizzled related protein 2 (sfrp2 gene) |
| 117 | Homo sapiens BAC clone RP11-216H12 from 4, complete sequence |
| 118 | C. familiaris MHC class Ib gene (DLA-79) gene, complete CDS |
| 119 | PREDICTED: Canis familiaris similar to Peroxisomal sarcosine oxidase (PSO) (L- |
| | pipecolate oxidase) (L-pipecolic acid oxidase) (LOC491177), mRNA |
| 120 | PREDICTED: Canis familiaris similar to spindle assembly abnormal protein 6 |
| | (LOC490142), mRNA |
| 121 | PREDICTED: Canis familiaris similar to Neutrophil gelatinase-associated lipocalin |
| | precursor (NGAL) (P25) (25 kDa alpha-2-microglobulin-related subunit of MMP-9) |
| | (Lipocalin 2) (Oncogene 24p3), transcript variant 2 (LOC491320), mRNA |
| 122 | PREDICTED: Canis familiaris similar to myeloid/lymphoid or mixed-lineage |
| | leukemia 5, transcript variant 11 (LOC476542), mRNA |
| 123 | Homo sapiens claudin 4, mRNA (cDNA clone MGC: 1778 IMAGE: 3349211), |
| | complete cds |
| 124 | Homo sapiens ELOVL family member 6, elongation of long chain fatty acids |
| | (FEN1/Elo2, SUR4/Elo3-like, yeast) (ELOVL6), mRNA |
| 125 | PREDICTED: Canis familiaris similar to sortilin-related receptor containing LDLR |
| | class A repeats preproprotein (LOC479408), mRNA |
| 126 | PREDICTED: Canis familiaris similar to fem-1 homolog b (LOC478352), mRNA |
| 127 | PREDICTED: Canis familiaris similar to FXYD domain-containing ion transport |
| | regulator 6 (LOC610831), mRNA |
| 128 | PREDICTED: Canis familiaris hypothetical LOC481916 (LOC481916), mRNA |
| 129 | PREDICTED: Canis familiaris similar to F46E10.1a (LOC480551), mRNA |
| 130 | Homo sapiens genomic DNA, chromosome 18 clone: RP11-883A18, complete |
| | sequence |
| 131 | PREDICTED: Canis familiaris similar to BE10.2 (LOC475247), mRNA |
| 132 | Canis familiaris T cell receptor beta chain hcvb3 (hcvb3) mRNA, partial cds |
| 133 | PREDICTED: Canis familiaris similar to peroxisome proliferative activated |
| | receptor, gamma, coactivator 1 alpha, transcript variant 1 (LOC479127), mRNA |
| 134 | PREDICTED: Canis familiaris similar to LIM and senescent cell antigen-like |
| | domains 1, transcript variant 1 (LOC474540), mRNA |
| 135 | PREDICTED: Bos taurus similar to heparan sulfate D-glucosaminyl 3-O- |
| | sulfotransferase 2 (LOC532099), partial mRNA |
| 136 | PREDICTED: Bos taurus similar to phosphatidylinositol-4-phosphate 5-kinase, |
| | type I, beta isoform 2, transcript variant 1 (LOC537654), mRNA |
| 137 | Canis familiaris dihydrodiol dehydrogenase (dimeric) (DHDH), mRNA |
| 138 | PREDICTED: Canis familiaris similar to Tumor-associated calcium signal |
| | transducer 1 precursor (Major gastrointestinal tumor-associated protein GA733-2) |
| | (Epithelial cell surface antigen) (Epithelial glycoprotein) (EGP) (Adenocarcinoma- |
| | associated antigen) (KSA) (KS 1/4 antigen) . . . (LOC481360), mRNA |
| 139 | Canis familiaris glucose-6-phosphatase mRNA, complete cds |
| 140 | Tursiops truncatus IgM heavy chain mRNA, complete cds |
| 141 | PREDICTED: Canis familiaris similar to creatine kinase, mitochondrial 1B |
| | precursor, transcript variant 3 (LOC478277), mRNA |
| 142 | PREDICTED: Pan troglodytes kinase related protein, telokin (LOC460640), |
| | mRNA |
| 143 | Homo sapiens serum/glucocorticoid regulated kinase 2, mRNA (cDNA clone |
| | IMAGE: 2988475), containing frame-shift errors |
| 144 | Xenopus laevis ubiquitously transcribed tetratricopeptide repeat gene, Y-linked, |
| | mRNA (cDNA clone MGC: 82191 IMAGE: 3401210), complete cds |
| 145 | PREDICTED: Canis familiaris similar to complement component 1, q |
| | subcomponent, gamma polypeptide (LOC487382), mRNA |
| 146 | PREDICTED: Canis familiaris similar to ARV1 homolog, transcript variant 1 |
| | (LOC488975), mRNA |
| 147 | Canis familiaris nitric oxide synthase 2A (inducible, hepatocytes) (NOS2A), |
| | mRNA |
| 148 | PREDICTED: Bos taurus similar to F-box protein 44 isoform 1 (LOC505957), |
| | mRNA |
| 149 | PREDICTED: Canis familiaris similar to PRKC, apoptosis, WT1, regulator |
| | (LOC611487), mRNA |
| 150 | PREDICTED: Canis familiaris similar to Complement C1q subcomponent, A |
| | chain precursor (LOC478194), mRNA |
| 151 | Canis familiaris triadin isoform 3 mRNA, complete cds |
| 152 | PREDICTED: Canis familiaris similar to CG13624-PC, isoform C, transcript |
| | variant 2 (LOC612888), mRNA |
| 153 | PREDICTED: Canis familiaris similar to CG4774-PA, isoform A, transcript variant |
| | 2 (LOC607530), mRNA |
| 154 | Homo sapiens neuropilin 2 (NRP2) gene, complete cds, alternatively spliced |
| 155 | PREDICTED: Canis familiaris similar to Tetraspanin-5 (Tspan-5) |
| | (Transmembrane 4 superfamily member 9) (Tetraspan NET-4), transcript variant |
| | 2 (LOC478486), mRNA |
| 156 | PREDICTED: Canis familiaris similar to Ig lambda chain C regions (LOC607541), |
| | mRNA |
| 157 | PREDICTED: Canis familiaris similar to pyruvate dehydrogenase phosphatase |
| | precursor (LOC477941), mRNA |
| 158 | PREDICTED: Canis familiaris similar to Xanthine dehydrogenase/oxidase, |
| | transcript variant 3 (LOC483028), mRNA |
| 159 | PREDICTED: Canis familiaris similar to Xanthine dehydrogenase/oxidase, |
| | transcript variant 8 (LOC483028), mRNA |
| 160 | PREDICTED: Canis familiaris similar to Alpha-2-macroglobulin precursor (Alpha- |
| | 2-M) (LOC477699)), mRNA |
| 161 | PREDICTED: Canis familiaris hypothetical LOC480209 (LOC480209), mRNA |
| 162 | PREDICTED: Canis familiaris sulfotransferase family, cytosolic, 1B, member 1 |
| | (SULT1B1), mRNA |
| 163 | PREDICTED: Canis familiaris similar to Aquaporin 3 (LOC611792), mRNA |
| 164 | Petunia integrifolia subsp. inflata S2 self-incompatibility ribonuclease (S2-RNase) |
| | and S2-locus F-box protein (SLF2) genes, complete cds |
| 165 | PREDICTED: Canis familiaris similar to Protein C14orf103 homolog |
| | (LOC480428), mRNA |
| 166 | Homo sapiens mRNA; cDNA DKFZp686G0638 (from clone DKFZp686G0638) |
| 167 | Homo sapiens B-box and SPRY domain containing (BSPRY), mRNA |
| 168 | PREDICTED: Rattus norvegicus apoptotic chromatin condensation inducer 1 |
| | (predicted) (Acin1_predicted), mRNA |
| 169 | PREDICTED: Canis familiaris similar to Elafin precursor (Elastase-specific |
| | inhibitor) (ESI) (Skin-derived antileukoproteinase) (SKALP) (WAP four-disulfide |
| | core domain protein 14) (Protease inhibitor WAP3) (LOC477241), mRNA |
| 170 | PREDICTED: Canis familiaris similar to cystatin 9-like precursor (LOC485559), |
| | mRNA |
| 171 | Oryza sativa (japonica cultivar-group) genomic DNA, chromosome 1, complete |
| | sequence |
| 172 | PREDICTED: Canis familiaris dystonin, transcript variant 16 (DST), mRNA |
| 173 | PREDICTED: Canis familiaris similar to acid phosphatase 6, lysophosphatidic |
| | (LOC475822), mRNA |
| 174 | PREDICTED: Canis familiaris similar to arrestin domain containing 2 isoform 2, |
| | transcript variant 3 (LOC609489), mRNA |
| 175 | PREDICTED: Canis familiaris similar to ATP-binding cassette, sub-family A |
| | member 1 (LOC481651), mRNA |
| 176 | PREDICTED: Canis familiaris similar to promyelocytic leukemia zinc finger |
| | protein, transcript variant 3 (LOC489398), mRNA |
| 177 | PREDICTED: Canis familiaris similar to Protein C9orf72 homolog, transcript |
| | variant 1 (LOC481569), mRNA |
| 178 | PREDICTED: Canis familiaris similar to mitogen-activated protein kinase kinase |
| | kinase 5 (LOC491765), mRNA |
| 179 | PREDICTED: Canis familiaris similar to zinc finger protein 403, transcript variant |
| | 4 (LOC480594), mRNA |
| 180 | Homo sapiens tetratricopeptide repeat domain 25, mRNA (cDNA clone |
| | IMAGE: 4831078), complete cds |
| 181 | Homo sapiens mRNA for laminin alpha 2 subunit precursor variant protein |
| 182 | PREDICTED: Canis familiaris similar to pyruvate dehydrogenase phosphatase |
| | precursor (LOC477941), mRNA |
| 183 | Homo sapiens lipin 1 (LPIN1), mRNA |
| 184 | PREDICTED: Canis familiaris similar to Immunoglobulin lambda-like polypeptide |
| | 1 precursor (Immunoglobulin-related 14.1 protein) (Immunoglobulin omega |
| | polypeptide) (Lambda 5) (CD179b antigen) (LOC608248), mRNA |
| 185 | PREDICTED: Canis familiaris similar to Asporin precursor (LOC610685), mRNA |
| 186 | Bos taurus similar to Asporin precursor (Periodontal ligament associated protein |
| | 1) (PLAP-1) (MGC128677), mRNA |
| 187 | PREDICTED: Canis familiaris similar to Transcription factor BTEB1 (Basic |
| | transcription element binding protein 1) (BTE-binding protein 1) (GC box binding |
| | protein 1) (Krueppel-like factor 9) (LOC484172), mRNA |
| 188 | PREDICTED: Canis familiaris similar to solute carrier family 27 (fatty acid |
| | transporter), member 6 (LOC474666), mRNA |
| 189 | Aspergillus nidulans FGSC A4 hypothetical protein (AN4185.2), mRNA |
| 190 | PREDICTED: Canis familiaris similar to NADP-dependent retinol |
| | dehydrogenase/reductase (LOC488391), mRNA |
| 191 | Sus scrofa epidermal growth factor precursor (EGF) mRNA, complete cds |
| 192 | Canis familiaris IgA heavy chain constant region gene, partial cds |
| 193 | Canis familiaris mRNA for metallothionein-II, complete cds |
| 194 | PREDICTED: Canis familiaris similar to complement component 1, q |
| | subcomponent, beta polypeptide precursor (LOC487381), mRNA |
| 195 | PREDICTED: Canis familiaris similar to zinc finger protein 660 (LOC491422), |
| | mRNA |
| 196 | PREDICTED: Canis familiaris similar to SH3 domain protein D19, transcript |
| | variant 1 (LOC482645), mRNA |
| 197 | PREDICTED: Canis familiaris similar to DRE1 protein (LOC478647), mRNA |
| 198 | Macaca fascicularis mRNA, clone QnpA-12979: similar to Homo sapiens |
| | neuroepithelial cell transforming gene 1 (NET1), mRNA NM_005863.2 |
| 199 | Homo sapiens leucyl/cystinyl aminopeptidase (LNPEP), transcript variant 2, |
| | mRNA |
| 200 | Homo sapiens adenosine monophosphate deaminase (isoform E) (AMPD3), |
| | transcript variant 1, mRNA |
| 201 | PREDICTED: Canis familiaris tocopherol (alpha) transfer protein (TTPA), mRNA |
| 202 | PREDICTED: Canis familiaris similar to tropomodulin 1, transcript variant 1 |
| | (LOC474771), mRNA |
| 203 | PREDICTED: Canis familiaris similar to Kynurenine--oxoglutarate transaminase I |
| | (Kynurenine aminotransferase I) (KATI) (Glutamine--phenylpyruvate |
| | transaminase) (Glutamine transaminase K) (GTK) (Cysteine-S-conjugate beta- |
| | lyase) (LOC491310), mRNA |
| 204 | PREDICTED: Canis familiaris similar to Kynurenine--oxoglutarate transaminase I |
| | (Kynurenine aminotransferase I) (KATI) (Glutamine--phenylpyruvate |
| | transaminase) (Glutamine transaminase K) (GTK) (Cysteine-S-conjugate beta- |
| | lyase) (LOC491310), mRNA |
| 205 | PREDICTED: Canis familiaris similar to Triggering receptor expressed on myeloid |
| | cells 2 precursor (Triggering receptor expressed on monocytes 2) (TREM-2) |
| | (LOC608965), mRNA |
| 206 | PREDICTED: Bos taurus similar to Triggering receptor expressed on myeloid |
| | cells 2 precursor (Triggering receptor expressed on monocytes 2) (TREM-2), |
| | transcript variant 2 (LOC506467), mRNA |
| 207 | PREDICTED: Canis familiaris similar to Alpha-2-macroglobulin precursor (Alpha- |
| | 2-M) (LOC477699), mRNA |
| 208 | PREDICTED: Bos taurus similar to Transcription factor BTEB1 (Basic |
| | transcription element binding protein 1) (BTE-binding protein 1) (GC box binding |
| | protein 1) (Krueppel-like factor 9), transcript variant 3 (LOC539139), mRNA |
| 209 | Hordeum vulgare subsp. vulgare cultivar Morex inducer of CBF expression 2 |
| | (ICE2) gene, partial cds |
| 210 | PREDICTED: Canis familiaris similar to Y54E10A.6 (LOC489622), mRNA |
| 211 | Pongo pygmaeus mRNA; cDNA DKFZp469L0319 (from clone DKFZp469L0319) |
| 212 | PREDICTED: Canis familiaris similar to Insulin receptor precursor (IR) (CD220 |
| | antigen) (LOC484990), mRNA |
| 213 | PREDICTED: Canis familiaris similar to ring finger protein 150 (LOC607611), |
| | mRNA |
| 214 | Homo sapiens cDNA clone MGC: 51010 IMAGE: 5270267, complete cds |
| 215 | PREDICTED: Canis familiaris matrix metalloproteinase-2 (MMP-2), mRNA |
| 216 | PREDICTED: Canis familiaris similar to acyl-CoA synthetase long-chain family |
| | member 3, transcript variant 1 (LOC478927), mRNA |
| 217 | Drosophila melanogaster CG18211-PA (betaTry) mRNA, complete cds |
| 218 | PREDICTED: Canis familiaris similar to retinoic acid receptor responder |
| | (tazarotene induced) 1 isoform 1 (LOC612298), mRNA |
| 219 | Homo sapiens protein upregulated in metastatic prostate cancer mRNA, |
| | complete cds |
| 220 | PREDICTED: Canis familiaris similar to Krueppel-like factor 5 (Intestinal-enriched |
| | krueppel-like factor) (Colon krueppel-like factor) (Transcription factor BTEB2) |
| | (Basic transcription element binding protein 2) (BTE-binding protein 2) (GC box |
| | binding protein 2) . . ., transcript variant 3 (LOC612788), mRNA |
| 221 | PREDICTED: Canis familiaris similar to NOV protein homolog precursor (NovH) |
| | (Nephroblastoma overexpressed gene protein homolog) (LOC475083), mRNA |
| 222 | PREDICTED: Canis familiaris similar to interferon-related developmental |
| | regulator 1, transcript variant 2 (LOC482408), mRNA |
| 223 | PREDICTED: Canis familiaris similar to BTG3 protein (Tob5 protein) (Abundant in |
| | neuroepithelium area protein), transcript variant 2 (LOC487695), mRNA |
| 224 | Bos taurus homeodomain only protein, mRNA (cDNA clone MGC: 127764 |
| | IMAGE: 7963031), complete cds |
| 225 | PREDICTED: Canis familiaris similar to Protein C9orf72 homolog, transcript |
| | variant 1 (LOC481569), mRNA |
| 226 | PREDICTED: Canis familiaris similar to Proteinase activated receptor 3 precursor |
| | (PAR-3) (Thrombin receptor-like 2) (Coagulation factor II receptor-like 2) |
| | (LOC607963), mRNA |
| 227 | Leishmania major strain Friedlin hypothetical protein (LMJ_1048) mRNA, partial |
| | cds |
| 228 | PREDICTED: Canis familiaris similar to ankyrin repeat and SOCS box-containing |
| | protein 2 (predicted), transcript variant 1 (LOC490836), mRNA |
| 229 | PREDICTED: Canis familiaris similar to Kynurenine--oxoglutarate transaminase I |
| | (Kynurenine aminotransferase I) (KATI) (Glutamine--phenylpyruvate |
| | transaminase) (Glutamine transaminase K) (GTK) (Cysteine-S-conjugate beta- |
| | lyase) (LOC491310), mRNA |
| 230 | PREDICTED: Canis familiaris similar to Immunoglobulin lambda-like polypeptide |
| | 1 precursor (Immunoglobulin-related 14.1 protein) (Immunoglobulin omega |
| | polypeptide) (Lambda 5) (CD179b antigen) (LOC607558), mRNA |
| 231 | PREDICTED: Canis familiaris similar to Alpha-2-macroglobulin precursor (Alpha- |
| | 2-M) (LOC477699), mRNA |
| 232 | PREDICTED: Canis familiaris similar to Phospholipid transfer protein precursor |
| | (Lipid transfer protein II) (LOC485903), mRNA |
| 233 | PREDICTED: Canis familiaris similar to WD repeat domain 66, transcript variant |
| | 2 (LOC477466), mRNA |
| 234 | Homo sapiens alanine-glyoxylate aminotransferase 2-like 1, mRNA (cDNA clone |
| | MGC: 26665 IMAGE: 4797767), complete cds |
| 235 | Canis familiaris IgA heavy chain constant region gene, partial cds |
| 236 | Felis catus CD8 antigen, beta polypeptide (CD8B), mRNA |
| 237 | PREDICTED: Pan troglodytes RAN binding protein 2 (LOC459477), mRNA |
| 238 | PREDICTED: Canis familiaris similar to ERBB2 interacting protein isoform 7 |
| | (LOC478082), mRNA |
| 239 | Homo sapiens mRNA for alanine:glyoxylate aminotransferase 2 homolog 1, |
| | splice form 1 (AGXT2L1 gene) |
| 240 | PREDICTED: Canis familiaris similar to phospholipase C, delta 4 (LOC478910), |
| | mRNA |
| 241 | PREDICTED: Canis familiaris similar to [Pyruvate dehydrogenase [lipoamide]] |
| | kinase isozyme 4, mitochondrial precursor (Pyruvate dehydrogenase kinase |
| | isoform 4) (LOC482310), mRNA |
| 242 | PREDICTED: Canis familiaris similar to ankyrin repeat and SOCS box-containing |
| | protein 2 (predicted), transcript variant 1 (LOC490836), mRNA |
| 243 | PREDICTED: Canis familiaris similar to lipin 1, transcript variant 1 (LOC475670), |
| | mRNA |
| 244 | PREDICTED: Canis familiaris similar to hypoxia-inducible factor-3 alpha isoform |
| | a (LOC476429), mRNA |
| 245 | Canis familiaris metallothionein 1X (MT1X), mRNA |
| 246 | Canis familiaris metallothionein 1X (MT1X), mRNA |
| 247 | PREDICTED: Canis familiaris similar to UDP-N-acetyl-alpha-D- |
| | galactosamine:polypeptide N-acetylgalactosaminyltransferase-like 2 |
| | (LOC477056), mRNA |
| 248 | Homo sapiens WNK lysine deficient protein kinase 3 (WNK3), transcript variant 2, |
| | mRNA |
| 249 | Canis familiaris IgA heavy chain constant region gene, partial cds |
| 250 | PREDICTED: Canis familiaris similar to Myosin-3 (Myosin heavy chain A) (MHC |
| | A) (LOC474713), mRNA |
| 251 | PREDICTED: Canis familiaris similar to downregulated in renal cell carcinoma |
| | (LOC607380), mRNA |
| 252 | PREDICTED: Canis familiaris similar to centrosome spindle pole associated |
| | protein (LOC477902), mRNA |
| 253 | PREDICTED: Canis familiaris similar to Osteomodulin precursor (Osteoadherin) |
| | (OSAD) (Keratan sulfate proteoglycan osteomodulin) (KSPG osteomodulin) |
| | (LOC610693), mRNA |
| 254 | Xenopus laevis MGC83953 protein, mRNA (cDNA clone MGC: 83953 |
| | IMAGE: 6862234), complete cds |
| 255 | PREDICTED: Canis familiaris hypothetical LOC490496 (LOC490496), mRNA |
| 256 | PREDICTED: Canis familiaris similar to WAP four-disulfide core domain 1 |
| | precursor (LOC489682), mRNA |
| 257 | Pongo pygmaeus C6 gene for complement component 6, partial cds |
| 258 | PREDICTED: Canis familiaris similar to ankyrin repeat, family A (RFXANK-like), |
| | 2, transcript variant 3 (LOC478097), mRNA |
| 259 | Plasmodium yoelii yoelii str. 17XNL hypothetical protein (PY02022) mRNA, partial |
| | cds |
| 260 | Pongo pygmaeus mRNA; cDNA DKFZp470P1633 (from clone DKFZp470P1633) |
| 261 | Canis familiaris podoplanin (PDPN), mRNA |
| 262 | Pongo pygmaeus mRNA; cDNA DKFZp468I0813 (from clone DKFZp468I0813) |
| 263 | Homo sapiens zinc finger, DHHC-type containing 17 (ZDHHC17), mRNA |
| 264 | PREDICTED: Canis familiaris similar to ATPase, H+ transporting, lysosomal |
| | 42 kDa, V1 subunit C isoform 2, transcript variant 3 (LOC475667), mRNA |
| 265 | PREDICTED: Canis familiaris similar to aminopeptidase puromycin sensitive |
| | (LOC480538), mRNA |
| 266 | PREDICTED: Canis familiaris similar to CG7245-PA (LOC481865), mRNA |
| 267 | PREDICTED: Rattus norvegicus similar to DD1 (predicted) (LOC291580), mRNA |
| 268 | Bos taurus mRNA for VSGP/F-spondin, complete cds |
| 269 | Sus scrofa estrogen sulfotransferase (STE), mRNA |
| 270 | Homo sapiens Kallmann syndrome 1 sequence (KAL1), mRNA |
| 271 | PREDICTED: Canis familiaris similar to sarcoma antigen NY-SAR-41 |
| | (LOC479946), mRNA |
| 272 | PREDICTED: Bos taurus similar to zinc finger protein 420 (LOC512882), mRNA |
| 273 | PREDICTED: Canis familiaris similar to lipin 1, transcript variant 4 (LOC475670), |
| | mRNA |
| 274 | PREDICTED: Canis familiaris similar to phytanoyl-CoA hydroxylase precursor |
| | (LOC607509), mRNA |
| 275 | PREDICTED: Canis familiaris similar to Bone morphogenetic protein 2 precursor |
| | (BMP-2) (BMP-2A), transcript variant 1 (LOC477162), mRNA |
| 276 | PREDICTED: Canis familiaris similar to Bone morphogenetic protein 2 precursor |
| | (BMP-2) (BMP-2A), transcript variant 1 (LOC477162), mRNA |
| 277 | Homo sapiens Kruppel-like factor 9 (KLF9), mRNA |
| 278 | PREDICTED: Canis familiaris hypothetical LOC474886, transcript variant 2 |
| | (LOC474886), mRNA |
| 279 | PREDICTED: Canis familiaris similar to triggering receptor expressed on myeloid |
| | cells-like 4 (LOC608975), mRNA |
| 280 | PREDICTED: Canis familiaris similar to Proteinase activated receptor 3 precursor |
| | (PAR-3) (Thrombin receptor-like 2) (Coagulation factor II receptor-like 2) |
| | (LOC607963), mRNA |
| 281 | PREDICTED: Canis farniliaris similar to WD repeat domain 66, transcript variant |
| | 2 (LOC477466), mRNA |
| 282 | PREDICTED: Canis familiaris similar to interferon-related developmental |
| | regulator 1, transcript variant 1 (LOC482408), mRNA |
| 283 | PREDICTED: Canis familiaris similar to Zinc finger protein 283 (LOC613011), |
| | mRNA |
| 284 | PREDICTED: Canis familiaris similar to triggering receptor expressed on myeloid |
| | cells-like 1 (LOC474898), mRNA |
| 285 | PREDICTED: Bos taurus similar to Carnitine O-palmitoyltransferase I, |
| | mitochondrial liver isoform (CPT I) (CPTI-L) (Carnitine palmitoyltransferase 1A) |
| | (LOC506812), partial mRNA |
| 286 | PREDICTED: Canis familiaris similar to Kynurenine--oxoglutarate transaminase I |
| | (Kynurenine aminotransferase I) (KATI) (Glutamine--phenylpyruvate |
| | transaminase) (Glutamine transaminase K) (GTK) (Cysteine-S-conjugate beta- |
| | lyase) (LOC491310), mRNA |
| 287 | PREDICTED: Canis familiaris hypothetical LOC476569 (LOC476569), mRNA |
| 288 | Mus musculus expressed sequence AI854703 (AI854703), mRNA |
| 289 | PREDICTED: Canis familiaris similar to regeneration associated muscle protease |
| | isoform b (LOC483426), mRNA |
| 290 | PREDICTED: Canis familiaris similar to fibronectin type III domain containing 1 |
| | (LOC484061), mRNA |
| 291 | Homo sapiens WAP four-disulfide core domain 1 (WFDC1), mRNA |
| 292 | PREDICTED: Canis familiaris similar to CG1530-PA (LOC609071), mRNA |
| 293 | PREDICTED: Canis familiaris similar to glycoprotein (transmembrane) nmb |
| | isoform b precursor, transcript variant 3 (LOC482355), mRNA |
| 294 | PREDICTED: Pan troglodytes similar to sprouty homolog 1, antagonist of FGF |
| | signaling; sprouty, Drosophila, homolog of, 1 (antagonist of FGF signaling); |
| | sprouty (Drosophila) homolog 1 (antagonist of FGF signaling) (LOC461476), |
| | mRNA |
| 295 | PREDICTED: Canis familiaris similar to phytanoyl-CoA hydroxylase precursor |
| | (LOC478000), mRNA |

TABLE 5
Gene Description - Highest BLAST Hit
for a Human Sequence Accession Number

| SEQ ID NO | Gene Description - Highest BLAST Hit for a Human Sequence Accession Number |
|---|---|
| 1 | Homo sapiens armadillo repeat containing 9, mRNA (cDNA clone MGC: 74894 |
| | IMAGE: 6165433), complete cds |
| 2 | Homo sapiens jun dimerization protein gene, partial cds; cfos gene, complete |
| | cds; and unknown gene |
| 3 | Homo sapiens EGR1 gene for early growth response protein 1 |
| 4 | Homo sapiens early growth response 1, mRNA (cDNA clone MGC: 88036 |
| | IMAGE: 6188360), complete cds |
| 5 | Homo sapiens 12 BAC RP11-181C3 (Roswell Park Cancer Institute Human BAC |
| | Library) complete sequence |
| 6 | Homo sapiens mRNA; cDNA DKFZp686J04124 (from clone DKFZp686J04124) |
| 7 | Homo sapiens zinc finger protein 227 (ZNF227), mRNA |
| 8 | Homo sapiens solute carrier family 7 (cationic amino acid transporter, y+ system), |
| | member 3 (SLC7A3), mRNA |
| 9 | Homo sapiens PAC clone RP5-1003N18 from 14q24.3, complete sequence |
| 10 | Homo sapiens serum/glucocorticoid regulated kinase 2 mRNA, complete cds |
| 11 | Homo sapiens BAC clone RP11-198M19 from 2, complete sequence |
| 12 | Homo sapiens FK506 binding protein 5 (FKBP5), mRNA |
| 13 | Synthetic construct Homo sapiens tumor-associated calcium signal transducer 1 |
| | mRNA, partial cds |
| 14 | Human DNA sequence from clone RP3-510O8 on chromosome 6 Contains the 5′ |
| | end of the FKBP5 gene for FK506 binding protein 5 (FKBP51), four novel genes |
| | (including FLJ25390), a UMP-CMP (uridine monophosphate - cytidine |
| | monophosphate) kinase pseudogene, the CLPS gene for pancreatic colipase, the |
| | 5′ end of a novel gene and two CpG islands, complete sequence |
| 15 | Homo sapiens pyruvate dehydrogenase kinase, isozyme 4, mRNA (cDNA clone |
| | MGC: 5281 IMAGE: 3047987), complete cds |
| 16 | Homo sapiens BAC clone RP11-617I14 from 4, complete sequence |
| 17 | Homo sapiens chromosome 3 clone RP11-6B7, complete sequence |
| 18 | Homo sapiens acetyl-Coenzyme A carboxylase beta (ACACB), mRNA |
| 19 | Synthetic construct Homo sapiens tumor-associated calcium signal transducer 1 |
| | mRNA, partial cds |
| 20 | Homo sapiens 12 BAC RP11-451H11 (Roswell Park Cancer Institute Human |
| | BAC Library) complete sequence |
| 21 | Homo sapiens BAC clone RP11-398G12 from 2, complete sequence |
| 22 | Homo sapiens suprabasin (SBSN), mRNA |
| 23 | Homo sapiens 3 BAC RP11-1D19 (Roswell Park Cancer Institute Human BAC |
| | Library) complete sequence |
| 24 | Homo sapiens cDNA FLJ38032 fis, clone CTONG2013352, moderately similar to |
| | ZINC FINGER PROTEIN 228 |
| 25 | Homo sapiens zinc finger protein 233 (ZNF233), mRNA |
| 26 | full-length cDNA clone CS0DI004YK24 of Placenta Cot 25-normalized of Homo |
| | sapiens (human) |
| 27 | Homo sapiens 3 BAC RP11-211G3 (Roswell Park Cancer Institute Human BAC |
| | Library) complete sequence |
| 28 | Human DNA sequence from clone RP11-199C17 on chromosome 9 Contains the |
| | 5′ end of the TBC1D2 gene for TBC1 domain family, member 2 (PARIS1, PARIS- |
| | 1, DKFZP761D1823, DKFZp761D1823), the 3′ end of the GPR51 gene for G |
| | protein-coupled receptor 51 (HG20, GABBR2, GPRC3B, GABABR2) and a CpG |
| | island, complete sequence |
| 29 | Homo sapiens transmembrane protein with EGF-like and two follistatin-like |
| | domains 1 (TMEFF1), mRNA |
| 30 | Homo sapiens nuclear receptor subfamily 4, group A, member 1, transcript |
| | variant 1, mRNA (cDNA clone MGC: 9485 IMAGE: 3921259), complete cds |
| 31 | Human LAG-1 mRNA |
| 32 | Homo sapiens cDNA FLJ39913 fis, clone SPLEN2018643, highly similar to |
| | PROBABLE G PROTEIN-COUPLED RECEPTOR APJ |
| 33 | Human JE gene encoding a monocyte secretory protein mRNA, complete cds |
| 34 | Homo sapiens mRNA for activation-inducible lymphocyte immunomediatory |
| | molecule AILIM, complete cds |
| 35 | Homo sapiens cDNA clone IMAGE: 5175186, containing frame-shift errors |
| 36 | Homo sapiens ELOVL family member 6, elongation of long chain fatty acids |
| | (FEN1/Elo2, SUR4/Elo3-like, yeast) (ELOVL6), mRNA |
| 37 | Homo sapiens BAC clone RP11-384E2 from 4, complete sequence |
| 38 | Homo sapiens chromosome 14 open reading frame 119, mRNA (cDNA clone |
| | MGC: 74723 IMAGE: 5532778), complete cds |
| 39 | Homo sapiens chemokine (C-C motif) ligand 8 (CCL8), mRNA |
| 40 | Human DNA sequence from clone RP5-1172N10 on chromosome Xp11.3-11.4 |
| | Contains the 3′ end of the USP9X gene for X chromosome ubiquitin specific |
| | protease 9 (fat facets-like Drosophila), a novel gene and a CpG island, complete |
| | sequence |
| 41 | Homo sapiens transmembrane 4 L six family member 18 (TM4SF18), mRNA |
| 42 | Human DNA sequence from clone RP11-520F24 on chromosome 13 Contains an |
| | HNRPA1 (heterogenous nuclear ribonucleoprotein A1) pseudogene, an ELL- |
| | related RNA polymerase II, elongation factor (ELL2) pseudogene and a ribosomal |
| | protein L37 (RPL37) pseudogene, complete sequence |
| 43 | Homo sapiens clone 25081 tropomodulin mRNA sequence |
| 44 | Homo sapiens acetyl-Coenzyme A carboxylase beta (ACACB), mRNA |
| 45 | Homo sapiens podoplanin (PDPN), transcript variant 3, mRNA |
| 46 | Homo sapiens zinc finger protein 228 (ZNF228), mRNA |
| 47 | Human 18S ribosomal RNA |
| 48 | Homo sapiens cDNA clone MGC: 88772 IMAGE: 4765168, complete cds |
| 49 | Homo sapiens glutamate-cysteine ligase, catalytic subunit (GCLC), mRNA |
| 50 | Homo sapiens gene for p16/CDKN2A, complete cds |
| 51 | Homo sapiens ATPase, H+ transporting, lysosomal 42 kDa, V1 subunit C isoform |
| | 2 (ATP6V1C2), mRNA |
| 52 | Homo sapiens interferon regulatory factor 4, mRNA (cDNA clone MGC: 23069 |
| | IMAGE: 4861223), complete cds |
| 53 | Homo sapiens ARV1 homolog (yeast) (ARV1), mRNA |
| 54 | Human DNA sequence from clone RP11-174I10 on chromosome 13 Contains the |
| | 3′ end of the RB1 gene for retinoblastoma 1 (including osteosarcoma) and the 5′ |
| | end of a novel gene, complete sequence |
| 55 | full-length cDNA clone CS0DJ010YA15 of T cells (Jurkat cell line) Cot 10- |
| | normalized of Homo sapiens (human) |
| 56 | Homo sapiens podoplanin (PDPN), transcript variant 1, mRNA |
| 57 | Homo sapiens glutamate-cysteine ligase, catalytic subunit (GCLC), mRNA |
| 58 | NA |
| 59 | Homo sapiens clone DNA44205 NLGN4 (UNQ365) mRNA, complete cds |
| 60 | Synthetic construct Homo sapiens clone FLH025847.01X apolipoprotein C-I |
| | (APOC1) mRNA, complete cds |
| 61 | Homo sapiens BAC clone RP11-197H3 from 2, complete sequence |
| 62 | Homo sapiens carnitine palmitoyltransferase 1A (liver) (CPT1A), nuclear gene |
| | encoding mitochondrial protein, transcript variant 1, mRNA |
| 63 | Homo sapiens hypoxia inducible factor 3, alpha subunit, mRNA (cDNA clone |
| | MGC: 99497 IMAGE: 6250259), complete cds |
| 64 | Homo sapiens claudin 10 (CLDN10), transcript variant 1, mRNA |
| 65 | Homo sapiens acetyl-Coenzyme A carboxylase beta (ACACB), mRNA |
| 66 | Homo sapiens 12q24 BAC RGPI11-443D10 (Roswell Park Cancer Institute |
| | Human BAC Library) complete sequence |
| 67 | Homo sapiens carnitine palmitoyltransferase 1A (liver), transcript variant 2, |
| | mRNA (cDNA clone MGC: 1772 IMAGE: 3352642), complete cds |
| 68 | Homo sapiens FK506 binding protein 5, mRNA (cDNA clone MGC: 34489 |
| | IMAGE: 4539929), complete cds |
| 69 | Homo sapiens NOVH protein mRNA, complete cds |
| 70 | Homo sapiens partial CPT1A gene for carnitine O-palmitoyltransferase 1, |
| | promoter region, CDS and slice variants a and b |
| 71 | Homo sapiens chromosome 19, cosmid R31341, complete sequence |
| 72 | Homo sapiens solute carrier family 26, member 7 (SLC26A7), transcript variant 1, |
| | mRNA |
| 73 | Mus musculus 17 days embryo stomach cDNA, RIKEN full-length enriched |
| | library, clone: I920056H18 product: NHP2-like protein 1 (High mobility group-like |
| | nuclear protein 2 homolog 1) ([U4/U6.U5] tri-snRNP 15.5 kDa protein) (OTK27) |
| | homolog [Homo sapiens], full insert sequence |
| 74 | Homo sapiens jun B proto-oncogene (JUNB), mRNA |
| 75 | Homo sapiens chromosome 8, clone RP11-734H6, complete sequence |
| 76 | Homo sapiens mRNA for Cdc7-related kinase, complete cds |
| 77 | Homo sapiens leucine rich repeat containing 17 (LRRC17), transcript variant 1, |
| | mRNA |
| 78 | Human DNA sequence from clone RP11-273F15 on chromosome 13 Contains a |
| | pseudogene similar to part of NADH dehydrogenase 3 (NADH dehydrogenase, |
| | subunit 3 (complex I)) (MTND3) and a non-metastatic cells 1, protein (NM23A) |
| | expressed in (NME1)(NM23, NM23-H1) pseudogene, complete sequence |
| 79 | Homo sapiens sortilin-related receptor, L(DLR class) A repeats-containing |
| | (SORL1), mRNA |
| 80 | dystrophin {5′ region, alternatively spliced} [human, cerebellar Purkinje neurons, |
| | mRNA Partial, 320 nt] |
| 81 | Human DNA sequence from clone RP11-45J1 on chromosome X Contains a |
| | prefoldin 4 (PFDN4) pseudogene, the 5′ end of a novel gene and a CpG island, |
| | complete sequence |
| 82 | Homo sapiens chromosome 8, clone CTD-3071K10, complete sequence |
| 83 | Macaca fascicularis brain cDNA, clone: QccE-21970, similar to human aldolase |
| | C, fructose-bisphosphate (ALDOC), mRNA, RefSeq: NM_005165.1 |
| 84 | Homo sapiens chromosome 17, clone 193h18, complete sequence |
| 85 | Homo sapiens BAC clone RP11-1191J2 from 4, complete sequence |
| 86 | Homo sapiens full open reading frame cDNA clone RZPDo834A0126D for gene |
| | SAA1, serum amyloid A1; complete cds, without stopcodon |
| 87 | TPA: Homo sapiens chromosome 17 proximal SMS-REP low-copy repeat, |
| | genomic sequence |
| 88 | Homo sapiens diphtheria toxin receptor (heparin-binding epidermal growth factor- |
| | like growth factor) (DTR) gene, complete cds |
| 89 | Homo sapiens BTB (POZ) domain containing 11 (BTBD11), transcript variant 3, |
| | mRNA |
| 90 | Homo sapiens regulator of G-protein signalling 1, mRNA (cDNA clone MGC: 9198 |
| | IMAGE: 3916789), complete cds |
| 91 | Homo sapiens nudE nuclear distribution gene E homolog 1 (A. nidulans), mRNA |
| | (cDNA clone MGC: 1075 IMAGE: 3140369), complete cds |
| 92 | Homo sapiens sortilin-related receptor, L(DLR class) A repeats-containing |
| | (SORL1), mRNA |
| 93 | Homo sapiens chromosome 5 clone RP11-1152B5, complete sequence |
| 94 | Homo sapiens serum-inducible kinase mRNA, complete cds |
| 95 | Homo sapiens mRNA; cDNA DKFZp313N1532 (from clone DKFZp313N1532) |
| 96 | Homo sapiens leucine rich repeat containing 39 (LRRC39), mRNA |
| 97 | Homo sapiens lamin B1 (LMNB1), mRNA |
| 98 | Homo sapiens reelin (RELN), transcript variant 2, mRNA |
| 99 | Homo sapiens cDNA FLJ41271 fis, clone BRAMY2036396 |
| 100 | Human DNA sequence from clone RP11-134P9 on chromosome 1 Contains the |
| | 3′ end of a novel gene, a novel gene, the BTG2 gene for BTG family, member 2 |
| | and a CpG island, complete sequence |
| 101 | Human DNA sequence from clone RP11-145E8 on chromosome 10 Contains a |
| | novel gene (KIAA1074), the 3′ end of the YME1L1 gene for YME1-like 1 (S. cerevisiae) |
| | and a CpG island, complete sequence |
| 102 | Macaca fascicularis testis cDNA clone: QtsA-13105, similar to human armadillo |
| | repeat containing 2 (ARMC2), mRNA, RefSeq: NM_032131.3 |
| 103 | Homo sapiens chromosome 17, clone CTD-3193K9, complete sequence |
| 104 | Human transcription factor ETR101 mRNA, complete cds |
| 105 | Human DNA sequence from clone CTA-343C1 on chromosome 22, complete |
| | sequence |
| 106 | full-length cDNA clone CS0DI069YJ22 of Placenta Cot 25-normalized of Homo |
| | sapiens (human) |
| 107 | Human DNA sequence from clone RP4-727I10 on chromosome 20 Contains a |
| | novel gene, ESTs, STSs and GSSs, complete sequence |
| 108 | Homo sapiens CC chemokine ligand 4L2f (CCL4L) mRNA, CCL4L-2 allele, |
| | complete cds, alternatively spliced |
| 109 | Homo sapiens thyroid hormone receptor, beta (erythroblastic leukemia viral (verb- |
| | a) oncogene homolog 2, avian), mRNA (cDNA clone MGC: 126110 |
| | IMAGE: 40033200), complete cds |
| 110 | Homo sapiens 3 BAC RP11-211G3 (Roswell Park Cancer Institute Human BAC |
| | Library) complete sequence |
| 111 | Human MAP kinase kinase MEK6 (MEK6) mRNA, complete cds |
| 112 | Human DNA sequence from clone RP11-182I10 on chromosome 1 Contains the |
| | 5′ end of the JAK1 gene for anus kinase 1 (a protein tyrosine kinase), a NADH |
| | dehydrogenase (ubiquinone) 1 alpha subcomplex 4 9 kDa (NDUFA4) |
| | pseudogene, a SIPL protein (SIPL) pseudogene, part of a novel gene, a solute |
| | carrier family 2 (facilitated glucose transporter) member 14 (SLC2A14) |
| | pseudogene and a CpG island, complete sequence |
| 113 | Homo sapiens 12 BAC RP4-605O3 (Roswell Park Cancer Institute Human BAC |
| | Library) complete sequence |
| 114 | Homo sapiens polo-like kinase 2 (Drosophila) (PLK2), mRNA |
| 115 | Human DNA sequence from clone RP11-497F24 on chromosome 6, complete |
| | sequence |
| 116 | Homo sapiens cDNA FLJ36603 fis, clone TRACH2015180, highly similar to |
| | Frizzled protein-2 |
| 117 | Homo sapiens BAC clone RP11-216H12 from 4, complete sequence |
| 118 | Homo sapiens HLA-C gene for MHC class I antigen, CW*15021 allele, exons 1-8 |
| 119 | Homo sapiens L-pipecolic acid oxidase (LPIPOX) mRNA, complete cds |
| 120 | Homo sapiens mRNA; cDNA DKFZp686C24224 (from clone DKFZp686C24224) |
| 121 | Synthetic construct Homo sapiens lipocalin 2 (oncogene 24p3) mRNA, partial cds |
| 122 | PREDICTED: Homo sapiens hypothetical protein FLJ10707 (FLJ10707), mRNA |
| 123 | Homo sapiens claudin 4, mRNA (cDNA clone MGC: 1778 IMAGE: 3349211), |
| | complete cds |
| 124 | Homo sapiens cDNA: FLJ23378 fis, clone HEP16248 |
| 125 | Homo sapiens gp250 precursor, mRNA, complete cds |
| 126 | Homo sapiens fem-1 homolog b (C. elegans), mRNA (cDNA clone MGC: 19792 |
| | IMAGE: 3840453), complete cds |
| 127 | Human XIST gene, poly purine-pyrimidine repeat region |
| 128 | Homo sapiens aminoacylase 1-like 2, mRNA (cDNA clone IMAGE: 5262663), |
| | partial cds |
| 129 | Homo sapiens hypothetical protein FLJ20920, mRNA (cDNA clone MGC: 19867 |
| | IMAGE: 4577089), complete cds |
| 130 | Homo sapiens genomic DNA, chromosome 18 clone: RP11-883A18, complete |
| | sequence |
| 131 | Homo sapiens hypothetical protein LOC392636, mRNA (cDNA clone |
| | MGC: 131748 IMAGE: 6152531), complete cds |
| 132 | Homo sapiens partial BV03S1J2.2 gene for T-cell receptor beta, variable region |
| 133 | Homo sapiens ligand effect modulator-6 (LEM6) mRNA, complete cds |
| 134 | PREDICTED: Homo sapiens similar to LIM and senescent cell antigen-like |
| | domains 1 (LOC440895), mRNA |
| 135 | Homo sapiens chromosome 16 clone CTA-237H1, complete sequence |
| 136 | Homo sapiens phosphatidylinositol-4-phosphate 5-kinase, type I, beta (PIP5K1B), |
| | transcript variant 2, mRNA |
| 137 | Homo sapiens dihydrodiol dehydrogenase (dimeric) (DHDH), mRNA |
| 138 | Homo sapiens BAC clone RP11-433O3 from 4, complete sequence |
| 139 | Human glucose-6-phosphatase mRNA, complete cds |
| 140 | human full-length cDNA clone CS0DD006YL02 of Neuroblastoma of Homo |
| | sapiens (human) |
| 141 | Homo sapiens creatine kinase, mitochondrial 1B (CKMT1B), nuclear gene |
| | encoding mitochondrial protein, mRNA |
| 142 | Homo sapiens cDNA FLJ45560 fis, clone BRTHA3003417 |
| 143 | Human DNA sequence from clone RP1-138B7 on chromosome 20q13.12 |
| | Contains the 3′ end of the L3MBTL gene for l(3)mbt-like (Drosophila), the SGK2 |
| | gene for serum/glucocorticoid regulated kinase 2, the 5′ end of the C20orf9 gene |
| | (NGD5, CGI-53), an HSPC194 pseudogene and a CpG island, complete |
| | sequence |
| 144 | Homo sapiens chromosome 15, clone RP11-253M7, complete sequence |
| 145 | Homo sapiens C1q-C mRNA, complete cds |
| 146 | Homo sapiens ARV1 homolog (yeast) (ARV1), mRNA |
| 147 | Mouse DNA sequence from clone RP23-215H18 on chromosome 11 Contains a |
| | novel gene and the 3′ end of a gene that is a possible ortholog of human dynein |
| | axonemal heavy polypeptide 9 (DNAH9), complete sequence |
| 148 | Homo sapiens mRNA; cDNA DKFZp781J0852 (from clone DKFZp781J0852) |
| 149 | Homo sapiens full open reading frame cDNA clone RZPDo834F0920D for gene |
| | PAWR, PRKC, apoptosis, WT1, regulator; complete cds, incl. stopcodon |
| 150 | Homo sapiens complement component 1, q subcomponent, alpha polypeptide, |
| | mRNA (cDNA clone MGC: 29490 IMAGE: 4850418), complete cds |
| 151 | NA |
| 152 | Homo sapiens cDNA FLJ16122 fis, clone BLADE2008995 |
| 153 | Homo sapiens chromosome 20 open reading frame 155 (C20orf155), mRNA |
| 154 | Homo sapiens neuropilin 2 (NRP2) gene, complete cds, alternatively spliced |
| 155 | Homo sapiens chromosome 4 clone RP11-603B8, complete sequence |
| 156 | NA |
| 157 | Homo sapiens protein phosphatase 2C, magnesium-dependent, catalytic subunit, |
| | mRNA (cDNA clone IMAGE: 6158636), partial cds |
| 158 | full-length cDNA clone CS0DI070YL04 of Placenta Cot 25-normalized of Homo |
| | sapiens (human) |
| 159 | Human xanthine dehydrogenase (XDH) mRNA, complete cds |
| 160 | Homo sapiens mRNA; cDNA DKFZp779B086 (from clone DKFZp779B086) |
| 161 | full-length cDNA clone CS0DI068YG02 of Placenta Cot 25-normalized of Homo |
| | sapiens (human) |
| 162 | Homo sapiens sulfotransferase family, cytosolic, 1B, member 1 (SULT1B1), |
| | mRNA |
| 163 | Human DNA sequence from clone RP11-311H10 on chromosome 9 Contains the |
| | 3′ end of the NFX1 gene for X-box binding nuclear transcription factor 1, the |
| | AQP7 and AQP3 genes for aquaporin 7 and 3, a novel gene, the gene for |
| | nucleolar RNA-associated protein alpha, beta and gamma and a CpG island, |
| | complete sequence |
| 164 | Human DNA sequence from clone RP3-340B19 on chromosome 6p21.2-21.3 |
| | Contains the TULP1 gene for tubby like protein 1, a novel gene, ribosomal protein |
| | S15A (RPS15A) and L36 (RPL36) pseudogenes, the 3′ end of the FKBP5 gene |
| | for FK506 binding protein 5 (FKBP51) and two CpG islands, complete sequence |
| 165 | Homo sapiens cDNA FLJ30638 fis, clone CTONG2002721, weakly similar to |
| | VACUOLAR PROTEIN SORTING-ASSOCIATED PROTEIN VPS13 |
| 166 | Homo sapiens cytochrome P450, family 26, subfamily B, polypeptide 1 |
| | (CYP26B1), mRNA |
| 167 | Homo sapiens BAC clone RP11-678H22 from 4, complete sequence |
| 168 | Homo sapiens chromosome 11, clone RP13-25N22, complete sequence |
| 169 | Human DNA sequence from clone RP1-172H20 on chromosome 20q12-13.12 |
| | Contains the PI3 gene for skin-derived protease inhibitor 3 (SKALP)the SEMG1 |
| | gene for semenogelin I, the SEMG2 gene for semenogelin II, complete sequence |
| 170 | Homo sapiens cystatin 9-like (mouse), mRNA (cDNA clone MGC: 34724 |
| | IMAGE: 5163974), complete cds |
| 171 | NA |
| 172 | Homo sapiens dystonin (DST), transcript variant 1eA, mRNA |
| 173 | full-length cDNA clone CS0DL009YM20 of B cells (Ramos cell line) Cot 25- |
| | normalized of Homo sapiens (human) |
| 174 | Homo sapiens arrestin domain containing 2, transcript variant 1, mRNA (cDNA |
| | clone MGC: 26574 IMAGE: 4817429), complete cds |
| 175 | Homo sapiens cDNA FLJ14958 fis, clone PLACE4000052, highly similar to Homo |
| | sapiens ATP cassette binding transporter 1 (ABC1) mRNA |
| 176 | Homo sapiens zinc finger and BTB domain containing 16, transcript variant 2, |
| | mRNA (cDNA clone MGC: 24908 IMAGE: 4944546), complete cds |
| 177 | Homo sapiens chromosome 9 open reading frame 72, mRNA (cDNA clone |
| | MGC: 86985 IMAGE: 5298741), complete cds |
| 178 | Homo sapiens mitogen-activated protein kinase kinase kinase 15 (MAP3K15), |
| | mRNA |
| 179 | Homo sapiens laryngeal carcinoma related protein 1 mRNA, complete cds |
| 180 | Homo sapiens tetratricopeptide repeat domain 25, mRNA (cDNA clone |
| | IMAGE: 4831078), complete cds |
| 181 | Homo sapiens mRNA for laminin alpha 2 subunit precursor variant protein |
| 182 | Homo sapiens protein phosphatase 2C, magnesium-dependent, catalytic subunit, |
| | mRNA (cDNA clone IMAGE: 6158636), partial cds |
| 183 | Homo sapiens BAC clone RP11-484O9 from 2, complete sequence |
| 184 | NA |
| 185 | Homo sapiens asporin (LRR class 1) (ASPN), mRNA |
| 186 | Homo sapiens clone DNA34392 ASPN (UNQ215) mRNA, complete cds |
| 187 | Human DNA sequence from clone RP11-386J22 on chromosome 9 Contains the |
| | SMC5L1 gene for SMC5 structural maintenance of chromosomes 5-like 1 (yeast) |
| | (SMC5, KIAA0594), the BTEB1 gene for basic transcription element binding |
| | protein 1 (BTEB, KLF9) and three CpG islands, complete sequence |
| 188 | Homo sapiens solute carrier family 27 (fatty acid transporter), member 6 |
| | (SLC27A6), transcript variant 2, mRNA |
| 189 | Homo sapiens chromosome 3 clone RP11-189A1, complete sequence |
| 190 | Homo sapiens dehydrogenase/reductase (SDR family) member 9 (DHRS9), |
| | transcript variant 2, mRNA |
| 191 | Homo sapiens truncated epidermal growth factor (beta-urogastrone) (EGF) gene, |
| | complete cds |
| 192 | Homo sapiens cDNA clone MGC: 86772 IMAGE: 4765168, complete cds |
| 193 | Homo sapiens PAC clone RP1-85D24 from Y, complete sequence |
| 194 | Homo sapiens complement component 1, q subcomponent, beta polypeptide, |
| | mRNA (cDNA clone MGC: 17227 IMAGE: 4212848), complete cds |
| 195 | Homo sapiens chromosome 3 clone RP11-944L7, complete sequence |
| 196 | Homo sapiens SH3 domain protein D19 (SH3D19), mRNA |
| 197 | Homo sapiens mRNA; cDNA DKFZp686E15208 (from clone DKFZp686E15208) |
| 198 | Macaca fascicularis mRNA, clone QnpA-12979: similar to Homo sapiens |
| | neuroepithelial cell transforming gene 1 (NET1), mRNA, NM_005863.2 |
| 199 | Human DNA sequence from clone RP5-1025A1 on chromosome 20p11.21-11.23 |
| | Contains the 5′ part of the ACAS2L gene for acetyl-Coenzyme A synthetase |
| | (AMP forming)-like, the VSX1 gene for visual system homeobox 1 (zebrafish) |
| | homolog (CHX10-like), variants L1 and S1 and four CpG islands, complete |
| | sequence |
| 200 | Homo sapiens chromosome 11, clone RP11-68C8, complete sequence |
| 201 | Homo sapiens tocopherol (alpha) transfer protein (ataxia (Friedreich-like) with |
| | vitamin E deficiency), mRNA (cDNA clone IMAGE: 4593015), partial cds |
| 202 | Homo sapiens cDNA FLJ32190 fis, clone PLACE6002102 |
| 203 | Homo sapiens CCBL1 gene, last two exons |
| 204 | Homo sapiens cysteine conjugate-beta lyase; cytoplasmic (glutamine |
| | transaminase K, kyneurenine aminotransferase) (CCBL1), mRNA |
| 205 | Homo sapiens triggering receptor expressed on myeloid cells 2 (TREM2), mRNA |
| 206 | Homo sapiens chromosome 21 open reading frame 24 isoform 7 (C21orf24) |
| | mRNA, complete cds |
| 207 | Homo sapiens mRNA; cDNA DKFZp779B086 (from clone DKFZp779B086) |
| 208 | Human DNA sequence from clone RP11-386J22 on chromosome 9 Contains the |
| | SMC5L1 gene for SMC5 structural maintenance of chromosomes 5-like 1 (yeast) |
| | (SMC5, KIAA0594), the BTEB1 gene for basic transcription element binding |
| | protein 1 (BTEB, KLF9) and three CpG islands, complete sequence |
| 209 | Homo sapiens BAC clone RP11-642E20 from 4, complete sequence |
| 210 | Homo sapiens leucine rich repeat containing 47 (LRRC47), mRNA |
| 211 | Homo sapiens translation initiation factor 2 (MTIF2) gene, exons 6 through 9; |
| | nuclear genes for mitochondrial products |
| 212 | Homo sapiens insulin receptor, mRNA (cDNA clone IMAGE: 4823710), partial cds |
| 213 | Homo sapiens ring finger protein 150 (RNF150), mRNA |
| 214 | Homo sapiens chromosome 5 clone CTC-361G14, complete sequence |
| 215 | Homo sapiens matrix metallopeptidase 2 (gelatinase A, 72 kDa gelatinase, 72 kDa |
| | type IV collagenase), mRNA (cDNA clone MGC: 2313 IMAGE: 3161383), |
| | complete cds |
| 216 | Homo sapiens Zic family member 3 heterotaxy 1 (odd-paired homolog, |
| | Drosophila) (ZIC3), mRNA |
| 217 | Homo sapiens BAC clone RP11-395A12 from 2, complete sequence |
| 218 | Homo sapiens genomic DNA, chromosome 18 clone: RP11-815J4, complete |
| | sequence |
| 219 | Homo sapiens protein upregulated in metastatic prostate cancer mRNA, |
| | complete cds |
| 220 | Homo sapiens Kruppel-like factor 5 (intestinal), mRNA (cDNA clone MGC: 52153 |
| | IMAGE: 5454169), complete cds |
| 221 | full-length cDNA clone CS0DK004YO15 of HeLa cells Cot 25-normalized of |
| | Homo sapiens (human) |
| 222 | Homo sapiens interferon-related developmental regulator 1 (IFRD1), transcript |
| | variant 1, mRNA |
| 223 | full-length cDNA clone CS0DK010YA19 of HeLa cells Cot 25-normalized of |
| | Homo sapiens (human) |
| 224 | Homo sapiens homeodomain-only protein, mRNA (cDNA clone MGC: 20820 |
| | IMAGE: 4335211), complete cds |
| 225 | Human DNA sequence from clone RP11-27J8 on chromosome 9 Contains the |
| | gene for interferon kappa precursor (IFNK), the 5′ UTR of a novel gene |
| | (FLJ13204), a novel gene, includes FLJ25077 and FLJ11109 (MGC23980) and 2 |
| | CpG islands, complete sequence |
| 226 | Homo sapiens coagulation factor II receptor-like 2 (F2RL2) gene, complete cds |
| 227 | Human DNA sequence from clone RP4-753D4 on chromosome 20q12 Contains |
| | part of the PTPRT gene for protein tyrosine phosphatase receptor type T and a |
| | novel gene, complete sequence |
| 228 | Human chromosome 14 DNA sequence BAC R-131H24 of library RPCI-11 from |
| | chromosome 14 of Homo sapiens (Human), complete sequence |
| 229 | Homo sapiens cysteine conjugate-beta lyase; cytoplasmic (glutamine |
| | transaminase K, kyneurenine aminotransferase) (CCBL1), mRNA |
| 230 | NA |
| 231 | Homo sapiens alpha-2-macroglobulin, mRNA (cDNA clone MGC: 47683 |
| | IMAGE: 6056126), complete cds |
| 232 | Homo sapiens phospholipid transfer protein, transcript variant 1, mRNA (cDNA |
| | clone MGC: 30183 IMAGE: 4992839), complete cds |
| 233 | Homo sapiens WD repeat domain 66 (WDR66), mRNA |
| 234 | Homo sapiens mRNA for alanine:glyoxylate aminotransferase 2 homolog 1, |
| | splice form 1 (AGXT2L1 gene) |
| 235 | Homo sapiens immunoglobulin alpha 2m(1) heavy chain constant region gene, |
| | partial cds |
| 236 | NA |
| 237 | Homo sapiens BAC clone RP11-348G16 from 2, complete sequence |
| 238 | Homo sapiens erbb2 interacting protein (ERBB2IP), transcript variant 2, mRNA |
| 239 | Homo sapiens mRNA for alanine:glyoxylate aminotransferase 2 homolog 1, |
| | splice form 1 (AGXT2L1 gene) |
| 240 | Homo sapiens phospholipase C, delta 4 (PLCD4), mRNA |
| 241 | Homo sapiens cDNA FLJ39109 fis, clone NTONG2005137, highly similar to |
| | [PYRUVATE DEHYDROGENASE(LIPOAMIDE)] KINASE ISOZYME 4, |
| | MITOCHONDRIAL PRECURSOR (EC 2.7.1.99) |
| 242 | Homo sapiens mRNA; cDNA DKFZp586M2121 (from clone DKFZp586M2121) |
| 243 | Human DNA sequence from clone RP11-98I9 on chromosome 6 Contains the |
| | gene for hexaprenyldihydroxybenzoate methyltransferase, mitochondrial |
| | precursor (COQ3), the gene for SR rich protein (FLJ14992), the USP45 gene for |
| | ubiquitin specific protease 45, the 3′ end of the gene for a novel protein similar to |
| | ubiquitin carboxyl-terminal hydrolase 16 (EC 3.1.2.15) and 2 CpG islands, |
| | complete sequence |
| 244 | Homo sapiens HIF-3A mRNA for hypoxia-inducible factor-3 alpha, complete cds |
| 245 | Homo sapiens BAC clone CTB-118E13 from 7, complete sequence |
| 246 | Human DNA sequence from clone RP11-435O5 on chromosome 9q22.1-22.33 |
| | Contains the PTCH gene for patched homolog, a novel gene, the gene for a |
| | novel protein similar to a metallothionein protein (MT1) and three CpG islands, |
| | complete sequence |
| 247 | Homo sapiens mRNA; cDNA DKFZp313I2220 (from clone DKFZp313I2220); |
| | complete cds |
| 248 | Homo sapiens WNK lysine deficient protein kinase 3 (WNK3), transcript variant 1, |
| | mRNA |
| 249 | Homo sapiens immunoglobulin alpha 2m(1) heavy chain constant region gene, |
| | partial cds |
| 250 | Macaca fascicularis testis cDNA clone: QtsA-11169, similar to human hypothetical |
| | protein C9orf93, mRNA, NM_173550.1 |
| 251 | Macaca fascicularis brain cDNA, clone: QflA-10289, similar to human TU3A |
| | protein (TU3A), mRNA, RefSeq: NM_007177.1 |
| 252 | Homo sapiens centrosome and spindle pole associated protein 1 (CSPP1), |
| | mRNA |
| 253 | Homo sapiens osteomodulin (OMD), mRNA |
| 254 | Homo sapiens 12 BAC RP11-424C20 (Roswell Park Cancer Institute Human |
| | BAC Library) complete sequence |
| 255 | Homo sapiens family with sequence similarity 59, member A (FAM59A), mRNA |
| 256 | Homo sapiens ps20 WAP-type four-disulfide core domain protein mRNA, |
| | complete cds |
| 257 | Homo sapiens chromosome 5 clone CTC-428I11, complete sequence |
| 258 | Homo sapiens chromosome 5 clone CTC-229P9, complete sequence |
| 259 | Homo sapiens chromosome 8, clone RP13-895A16, complete sequence |
| 260 | Human chromosome 14 DNA sequence BAC R-442G21 of library RPCI-11 from |
| | chromosome 14 of Homo sapiens (Human), complete sequence |
| 261 | Homo sapiens lung type-I cell membrane-associated protein hT1a-2 (hT1a-2) |
| | mRNA, complete cds |
| 262 | NA |
| 263 | Human DNA sequence from clone RP11-90M2 on chromosome 13 Contains |
| | gene FLJ10956, the gene for HSPC126 protein (DRIP36), a novel gene similar to |
| | polymerase (RNA) II (DNA directed) polypeptide K, 7.0 kDa (POLR2K), a novel |
| | gene and two CpG islands, complete sequence |
| 264 | Homo sapiens ATPase, H+ transporting, lysosomal 42 kDa, V1 subunit C isoform |
| | 2 (ATP6V1C2), mRNA |
| 265 | Homo sapiens aminopeptidase puromycin sensitive (NPEPPS), mRNA |
| 266 | PREDICTED: Homo sapiens similar to dJ22I17.2 (novel protein with EGF-like |
| | and laminin G domains) (LOC442228), mRNA |
| 267 | Homo sapiens chromosome 5 clone CTC-575N7, complete sequence |
| 268 | Homo sapiens BAC clone RP11-308K2 from 4, complete sequence |
| 269 | Homo sapiens chromosome 5 clone RP11-270H9, complete sequence |
| 270 | ADMLX = putative adhesion molecule [human, mRNA, 4121 nt, segment 2 of 2] |
| 271 | Homo sapiens coiled-coil domain containing 18, mRNA (cDNA clone |
| | IMAGE: 4686590), partial cds |
| 272 | Homo sapiens, clone RP11-44B13, complete sequence |
| 273 | Macaca fascicularis testis cDNA clone: QtsA-14119, similar to human lipin 1 |
| | (LPIN1), mRNA, RefSeq: NM_145693.1 |
| 274 | Bos taurus phytanoyl-CoA hydroxylase [human: Refsum disease], mRNA (cDNA |
| | clone MGC: 127428 IMAGE: 7949271), complete cds |
| 275 | Human DNA sequence from clone RP5-859D4 on chromosome 20p12.1-13 |
| | Contains the BMP2 gene for bone morphogenetic protein 2, a novel gene and a |
| | CpG island, complete sequence |
| 276 | Human DNA sequence from clone RP5-859D4 on chromosome 20p12.1-13 |
| | Contains the BMP2 gene for bone morphogenetic protein 2, a novel gene and a |
| | CpG island, complete sequence |
| 277 | Human DNA sequence from clone RP11-386J22 on chromosome 9 Contains the |
| | SMC5L1 gene for SMC5 structural maintenance of chromosomes 5-like 1 (yeast) |
| | (SMC5, KIAA0594), the BTEB1 gene for basic transcription element binding |
| | protein 1 (BTEB, KLF9) and three CpG islands, complete sequence |
| 278 | Homo sapiens chromosome 6 open reading frame 81 (C6orf81), mRNA |
| 279 | Human DNA sequence from clone RP11-401F24 on chromosome 10 Contains |
| | gene FLJ20909, the gene for a novel protein (MGC35403), a novel gene |
| | (LOC219731), the 3′ end of the UPF2 gene for UPF2 regulator of nonsense |
| | transcripts homolog (yeast) (FLJ38872) and four CpG islands, complete |
| | sequence |
| 280 | Homo sapiens coagulation factor II receptor-like 2 (F2RL2) gene, complete cds |
| 281 | Homo sapiens WD repeat domain 66, mRNA (cDNA clone MGC: 33630 |
| | IMAGE: 4826893), complete cds |
| 282 | Macaca fascicularis testis cDNA, clone: QtsA-18294, similar to human interferon- |
| | related developmental regulator 1 (IFRD1), mRNA, RefSeq: NM_001550.1 |
| 283 | PREDICTED: Homo sapiens zinc finger protein 283 (ZNF283), mRNA |
| 284 | Human DNA sequence from clone RP1-238O23 on chromosome 6 Contains part |
| | of the a novel gene, the gene for triggering receptor expressed on myeloid cells 2 |
| | (TREM2), a novel gene, part of a novel gene, a pseudogene similar to soluble |
| | adenylyl cyclase (SAC),, complete sequence |
| 285 | full-length cDNA clone CS0DK009YI05 of HeLa cells Cot 25-normalized of Homo |
| | sapiens (human) |
| 286 | Homo sapiens cysteine conjugate-beta lyase; cytoplasmic (glutamine |
| | transaminase K, kyneurenine aminotransferase) (CCBL1), mRNA |
| 287 | Homo sapiens chromosome 3 open reading frame 14, mRNA (cDNA clone |
| | MGC: 22227 IMAGE: 4307022), complete cds |
| 288 | Homo sapiens chromosome 15 clone CTD-2270N23 map 15q21, complete |
| | sequence |
| 289 | Homo sapiens mRNA; cDNA DKFZp667H2312 (from clone DKFZp667H2312) |
| 290 | Homo sapiens fibronectin type III domain containing 1 (FNDC1), mRNA |
| 291 | Homo sapiens chromosome 16 clone RP11-486L19, complete sequence |
| 292 | Homo sapiens BAC clone RP11-178D14 from 2, complete sequence |
| 293 | full-length cDNA clone CS0DN005YJ09 of Adult brain of Homo sapiens (human) |
| 294 | Homo sapiens PAC clone RP5-839O24 from 7, complete sequence |
| 295 | Homo sapiens 3 BAC RP11-364F11 (Roswell Park Cancer Institute Human BAC |
| | Library) complete sequence |

### Example 2

### Determining the Effect of Various Substances or Ingredients on Gene Expression in Canine Cell Lines

Affymetrix canine gene chips Canine-1 and Canine-2 are used to determine the effect of various test substances or ingredients such as MCTs; TAGs; ALA; EPA; DHA; linoleic acid; stearic acid (SA), conjugated linoleic acid (CLA), GLA; arachidonic acid; lecithin; vitamin A, vitamin D, vitamin E, vitamin K, riboflavin, niacin, pyridoxine, pantothenic acid, folic acid, biotin vitamin C, catechin, quercetin, theaflavin; ubiquinone; lycopene, lycoxanthin; resveratrol; α-lipoic acid; L-carnitine; D-limonene; glucosamine; S-adenosylmethionine; chitosan, various materials containing one or more of these compounds, and various combination thereof on gene expression in four canine cell lines and appropriate controls. Each ingredient is tested in two concentrations as illustrated for selected sample ingredients shown in Table 6. The solvent at the higher of the two concentrations is used as a control. Four canine cell lines are used: CCL34 (kidney), CRL1430 (thymus), CCL183 (bone) (obtained from The American Tissue Culture Collection) and CTAC (thyroid) (See, Measurement of NK Activity in Effector Cells Purified from Canine Peripheral Lymphocytes, Veterinary Immunology and Immunopathology, 35 (1993) 239-251). A cell line treated with an ingredient at a specific concentration is referred to as “treatment” and an untreated sample is referred to as “control.” The words “genes” and “probes” are used synonymously in this method. Gene expression is measured for the treatment cell lines and controls using the instructions provided with the Affymetrix chips.

The gene expression data is determined to be either “up” or “down”-regulated for any given treatment. The decision on whether a gene is “up” or “down” is based on the fold change, which is calculated as treatment intensity/control intensity for each individual probe. The fold change is considered down-regulated if its value is <1/1.5 (for across all 4 cell lines analysis) or <½ (for within cell lines analysis) and is up-regulated if it is >1.5 (for across all 4 cell lines analysis) or >2 (for within cell lines analysis). Also, a probe is considered significant for further scrutiny if it is called as present in only one of the conditions being compared (treatment or control) and is “absent” or “marginal” in the other and the fold change is significant according to the software used. Probes that appear to be regulated in opposite directions in the two treatments are excluded from further analysis.

The raw data is analyzed using GeneSpring version 7.0 (GS) software (Agilent Corporation) and validated using the R-Bioconductor (RB) freeware. Both software packages are used to compute probe intensities from the CEL files generated by the Affymetrix Instrument. The Present/Absent/Marginal calls per probe and P-values are computed using the R-Bioconductor and GeneSpring software separately.

Two schemes are used for data analysis. First; “across cell lines” and “within individual cell lines.” In the first scheme, genes are selected for scoring provided they are found to be significant and common across all cell-lines. The “across cell lines” yields the highest confidence data with minimum noise and may provide the best possible clues as to which genes are affected by individual ingredients. In the second scheme, only those genes that show a significant fold change in the two treatments according to both software packages within an individual cell lines are scored. A sample of the data obtained from these experiments is shown in Table 7. Table 7 shows the correlation between treatment substance (Column 1), Probe (data link) (Column 2), Direction (Column 3), Best BLAST Annotation (determined statistically) (Column 4), and Human Accession Number (Column 5). The information for all ingredients tested is stored in a database for reference.

Based upon the physiological condition of the canines (a diagnosis as fat) and a comparison of the information from the Tables1-7, i.e, noting genes that are influenced by a test substance or ingredient and are also differentially expressed in fat canines compared to lean canines, a nutritional formula useful for selecting and preparing a food composition for fat canines would be believed to contain one or more of the following ingredients in the following amounts (in vivo amounts in milligrams per kilogram of body weight per day (mg/kg/day) are based upon extrapolation from amounts used in vitro, for example: DHA—from about 1 to about 30; EPA—from about 1 to about 30; EPA/DHA Combo (1.5:1 ratio)—from about 412 to about 30/45; ALA—from about 10 to about 100; LA—from about 30 to about 600; ARA—from about 5 to about 50; and SA—from about 3 to about 60. Based upon these data, a food composition and related diet containing one or more of these ingredients can be prepared and used to regulate the genes that are differentially expressed in fat animals compared to lean animals. Such regulation will cause the modulation of the amount of adipose tissue on the animal and, therefore, in one embodiment, promote a shift to a desirable or normal (more lean) status and promote better health and wellness of the animal.

TABLE 6
Ingredients Tested in Canine Cell Lines

| Substance | Concentration 1 | Concentration 2 | Solvent |
|---|---|---|---|
| DHA | 0.005 mg/ml (5 micro | 0.025 mg/ml (25 micro | ETOH |
| | g/ml) | g/ml) | |
| EPA | 0.005 mg/ml (5 micro | 0.025 mg/ml (25 micro | ETOH |
| | g/ml) | g/ml) | |
| EPA/DHA | 0.015 mg/ml EPA & 0.010 mg/ml | 0.030 mg/ml EPA & 0.02 mg/ml | ETOH |
| Combo 1.5:1 | DHA (total is 0.025 mg/ml) | DHA (total is 0.050 mg/ml) | |
| ratio (like in | | | |
| fish oil) | | | |
| Alpha | 0.05 mg/ml (50 micro | 0.1 mg/ml (100 micro g/ml) | ETOH |
| linolenic acid | g/ml) | | |
| Linoleic acid | 0.1 mg/ml (100 micro | 0.5 mg/ml (500 micro g/ml) | ETOH |
| | g/ml) | | |
| Arachidonic | 0.025 mg/ml (25 micro | 0.05 mg/ml (50 micro g/ml) | ETOH |
| acid | g/ml) | | |
| Stearic acid | 0.01 mg/ml (10 micro | 0.05 mg/ml (50 micro g/ml) | ETOH |
| | g/ml) | | |
| Conjugated | 0.02 mg/ml (20 micro | 0.1 mg/ml (100 micro g/ml) | MEOH |
| Linoleic acid | g/ml) | | |

TABLE 7
Expression Profiling Results From Canine Cell Lines
in the Presence of Listed Ingredients
Column

| 1 | 2 | 3 | 4 | 5 |
|---|---|---|---|---|
| DHA | 1582387_at | DOWN | Canis familiaris type I | AC027016 |
| | | | iodothyronine deiodinase (dio 1) | |
| | | | mRNA, complete cds | |
| DHA | 1582824_at | UP | PREDICTED: Canis familiaris | BC000185 |
| | | | carnitine palmitoyl transferase I | |
| | | | isoform (CPT1), mRNA | |
| DHA | 1584133_at | UP | PREDICTED: Canis familiaris | BC038344 |
| | | | similar to dynein, cytoplasmic, | |
| | | | heavy polypeptide 2 (LOC479461), | |
| | | | mRNA | |
| DHA | 1584742_at | UP | Human DNA sequence from clone | AL591206 |
| | | | RP11-151J10 on chromosome 9 | |
| | | | Contains the 5′ end of a novel | |
| | | | gene (FLJ20060) (contains | |
| | | | FLJ12902, KIAA1574), the ADFP | |
| | | | gene for adipose differentiation- | |
| | | | related protein (ADRP) | |
| DHA | 1584951_at | UP | PREDICTED: Canis familiaris | CR605429 |
| | | | similar to Adipophilin (Adipose | |
| | | | differentiation-related protein) | |
| | | | (ADRP) (LOC474720), mRNA | |
| DHA | 1585355_at | UP | PREDICTED: Canis familiaris | CR597463 |
| | | | similar to Adipophilin (Adipose | |
| | | | differentiation-related protein) | |
| | | | (ADRP) (LOC474720), mRNA | |
| DHA | 1586474_at | DOWN | Mus musculus RIKEN cDNA | AC078834 |
| | | | 1500031L02 gene | |
| | | | (1500031L02Rik), mRNA | |
| DHA | 1587029_at | UP | Homo sapiens 12 BAC RP11- | AC089999 |
| | | | 545P7 (Roswell Park Cancer | |
| | | | Institute Human BAC Library) | |
| | | | complete sequence | |
| DHA | 1587141_at | UP | PREDICTED: Canis familiaris | CR456571 |
| | | | similar to SEC14-like protein 2 | |
| | | | (Alpha-tocopherol associated | |
| | | | protein) (TAP) (hTAP) | |
| | | | (Supernatant protein factor) (SPF) | |
| | | | (Squalene transfer protein) | |
| | | | (LOC477539), mRNA | |
| DHA | 1587268_at | UP | Canis familiaris urate oxidase | NA |
| | | | (UOX) mRNA, complete cds | |
| DHA | 1587328_at | UP | Homo sapiens mRNA; cDNA | AP001324 |
| | | | DKFZp686O1232 (from clone | |
| | | | DKFZp686O1232) | |
| DHA | 1587418_at | DOWN | PREDICTED: Canis familiaris | AJ417060 |
| | | | similar to RPGR-interacting protein | |
| | | | 1 isoform b (LOC475400), mRNA | |
| DHA | 1587734_at | UP | PREDICTED: Canis familiaris | BC017952 |
| | | | similar to Na/Pi cotransporter 4 | |
| | | | (LOC478741), mRNA | |
| DHA | 1588058_at | DOWN | Momo sapiens toll-interleukin 1 | BC032474 |
| | | | receptor (TIR) domain containing | |
| | | | adaptor protein, mRNA (cDNA | |
| | | | clone MGC: 40573 | |
| | | | IMAGE: 5216171), complete cds | |
| DHA | 1588088_at | UP | Homo sapiens hypoxia-inducible | BC008573 |
| | | | protein 2, mRNA (cDNA clone | |
| | | | MGC: 17005 IMAGE: 4182067), | |
| | | | complete cds | |
| DHA | 1589548_at | DOWN | Mus musculus chromosome 14 | AC115282 |
| | | | clone RP24-304G19, complete | |
| | | | sequence | |
| DHA | 1590835_at | DOWN | Homo sapiens interleukin 8 | AC055863 |
| | | | receptor, beta pseudogene, mRNA | |
| | | | (cDNA clone IMAGE: 5450999), | |
| | | | with apparent retained intron | |
| DHA | 1591083_at | UP | Homo sapiens clone DNA22780 | AC010323 |
| | | | NL2 (UNQ171) mRNA, complete | |
| | | | cds | |
| DHA | 1591971_at | UP | PREDICTED: Canis familiaris | AK055183 |
| | | | similar to complement C1s | |
| | | | (LOC486714), mRNA | |
| DHA | 1592507_at | DOWN | Homo sapiens prodynorphin | BC026334 |
| | | | (PDYN), mRNA | |
| DHA | 1593226_at | UP | Human DNA sequence from clone | AL358074 |
| | | | RP11-423C15 on chromosome 9 | |
| | | | Contains the 5′ end of the | |
| | | | MAPKAP1 gene for mitogen- | |
| | | | activated protein kinase associated | |
| | | | protein 1, a novel gene, the 5′ end | |
| | | | of the PBX3 gene f | |
| DHA | 1593388_at | DOWN | PREDICTED: Canis familiaris | BC063797 |
| | | | similar to SDA1 domain containing | |
| | | | 1 (LOC478431), mRNA | |
| DHA | 1593590_at | DOWN | Homo sapiens lymphocyte adaptor | AB208911 |
| | | | protein, mRNA (cDNA clone | |
| | | | IMAGE: 4861744), complete cds | |
| DHA | 1593831_at | DOWN | PREDICTED: Canis familiaris | BC015854 |
| | | | similar to Clathrin heavy chain 1 | |
| | | | (CLH-17) (LOC480578), mRNA | |
| DHA | 1594976_at | UP | PREDICTED: Bos taurus similar to | AL035698 |
| | | | glutamate receptor, metabotropic 1 | |
| | | | (LOC540485), mRNA | |
| DHA | 1596448_at | UP | PREDICTED: Canis familiaris | AK095036 |
| | | | similar to sperm associated antigen | |
| | | | 16 (LOC478899), mRNA | |
| DHA | 1596711_at | DOWN | Homo sapiens cDNA: FLJ21199 | AK024852 |
| | | | fis, clone COL00235 | |
| DHA | 1597677_at | UP | Homo sapiens, clone | AC012516 |
| | | | IMAGE: 5271096, mRNA | |
| DHA | 1597789_at | UP | Homo sapiens 12 BAC RP11- | AC130404 |
| | | | 337L12 (Roswell Park Cancer | |
| | | | Institute Human BAC Library) | |
| | | | complete sequence | |
| DHA | 1597832_at | DOWN | Homo sapiens hypothetical protein | NM_207311 |
| | | | LOC92558 (LOC92558), mRNA | |
| DHA | 1598607_at | DOWN | PREDICTED: Canis familiaris | AC099518 |
| | | | similar to Thioredoxin domain | |
| | | | containing protein 6 (Thioredoxin- | |
| | | | like protein 2) (Txl-2) | |
| | | | (LOC485685), mRNA | |
| DHA | 1598932_at | DOWN | PREDICTED: Canis familiaris | AL354836 |
| | | | similar to SAP90/PSD-95 | |
| | | | associated protein 2 (LOC488556), | |
| | | | mRNA | |
| DHA | 1599339_at | DOWN | Canis familiaris clone RP81- | NA |
| | | | 117B1, complete sequence | |
| DHA | 1599453_at | DOWN | PREDICTED: Canis familiaris | NA |
| | | | LOC475099 (LOC475099), mRNA | |
| DHA | 1600090_at | UP | PREDICTED: Canis familiaris | AY405366 |
| | | | similar to SEC22 vesicle trafficking | |
| | | | protein-like 2 (LOC478590), mRNA | |
| DHA | 1601347_at | DOWN | Debaryomyces hansenii CBS767, | NA |
| | | | DEHA0D14146g predicted mRNA | |
| DHA | 1602156_at | UP | Mus musculus mRNA for | AL590139 |
| | | | mKIAA4184 protein | |
| DHA | 1602790_at | UP | Homo sapiens aryl hydrocarbon | AC115282 |
| | | | receptor nuclear translocator | |
| | | | (ARNT) gene, complete cds | |
| DHA | 1602966_at | DOWN | Zebrafish DNA sequence from | AL590621 |
| | | | clone DKEYP-75A7 in linkage | |
| | | | group 21, complete sequence | |
| DHA | 1603771_at | DOWN | Canis familiaris clone RP81- | NA |
| | | | 117B1, complete sequence | |
| DHA | 1604372_at | UP | PREDICTED: Canis familiaris | AY411810 |
| | | | LOC475665 (LOC475665), mRNA | |
| DHA | 1605486_at | UP | Homo sapiens pyruvate | AK096428 |
| | | | dehydrogenase kinase 4 mRNA, 3′ | |
| | | | untranslated region, partial | |
| | | | sequence | |
| EPA | 1583329_at | DOWN | Homo sapiens, Similar to secreted | AC018634 |
| | | | frizzled-related protein 4, clone | |
| | | | IMAGE: 4828181, mRNA | |
| EPA | 1583403_at | UP | Sus scrofa carnitine | AK172798 |
| | | | palmitoyltransferase I mRNA, | |
| | | | nuclear gene encoding | |
| | | | mitochondrial protein, complete | |
| | | | cds | |
| EPA | 1584742_at | UP | Human DNA sequence from clone | AL591206 |
| | | | RP11-151J10 on chromosome 9 | |
| | | | Contains the 5′ end of a novel | |
| | | | gene (FLJ20060) (contains | |
| | | | FLJ12902, KIAA1574), the ADFP | |
| | | | gene for adipose differentiation- | |
| | | | related protein (ADRP) | |
| EPA | 1584951_at | UP | PREDICTED: Canis familiaris | CR605429 |
| | | | similar to Adipophilin (Adipose | |
| | | | differentiation-related protein) | |
| | | | (ADRP) (LOC474720), mRNA | |
| EPA | 1585292_at | UP | Homo sapiens methyl CpG binding | AF030876 |
| | | | protein 2 (Rett syndrome) | |
| | | | (MECP2), mRNA | |
| EPA | 1585355_at | UP | PREDICTED: Canis familiaris | CR597463 |
| | | | similar to Adipophilin (Adipose | |
| | | | differentiation-related protein) | |
| | | | (ADRP) (LOC474720), mRNA | |
| EPA | 1586420_at | DOWN | Homo sapiens RAB37, member | BC016615 |
| | | | RAS oncogene family (RAB37), | |
| | | | mRNA | |
| EPA | 1587196_at | UP | PREDICTED: Canis familiaris | NM_147223 |
| | | | LOC475684 (LOC475684), mRNA | |
| EPA | 1587428_at | DOWN | Human DNA sequence from clone | AL589740 |
| | | | RP11-436D23 on chromosome 6 | |
| | | | Contains part of a novel gene, | |
| | | | complete sequence | |
| EPA | 1588088_at | UP | Homo sapiens hypoxia-inducible | BC008573 |
| | | | protein 2, mRNA (cDNA clone | |
| | | | MGC: 17005 IMAGE: 4182067), | |
| | | | complete cds | |
| EPA | 1589797_at | DOWN | Homo sapiens chromosome 15 | AC090651 |
| | | | clone RP11-344A16 map 15q21.3, | |
| | | | complete sequence | |
| EPA | 1589829_s_at | DOWN | PREDICTED: Bos taurus similar to | AC004486 |
| | | | ATP-dependent DNA helicase Q4 | |
| | | | (RecQ protein-like 4) (RecQ4) | |
| | | | (LOC515289), partial mRNA | |
| EPA | 1590407_s_at | UP | Homo sapiens integrin-linked | AJ404847 |
| | | | kinase 1 (ILK) gene, complete cds | |
| EPA | 1591083_at | UP | Homo sapiens clone DNA22780 | AC010323 |
| | | | NL2 (UNQ171) mRNA, complete | |
| | | | cds | |
| EPA | 1592920_at | DOWN | Homo sapiens 12 BAC RP11- | AC090013 |
| | | | 407P2 (Roswell Park Cancer | |
| | | | Institute Human BAC Library) | |
| | | | complete sequence | |
| EPA | 1593146_s_at | UP | Homo sapiens Kruppel-like factor | BC063286 |
| | | | 11 (KLF11), mRNA | |
| EPA | 1593677_at | DOWN | PREDICTED: Canis familiaris | AB070003 |
| | | | similar to hypothetical protein | |
| | | | (LOC475308), mRNA | |
| EPA | 1594091_at | DOWN | PREDICTED: Canis familiaris | NM_024763 |
| | | | similar to FLJ23129 protein isoform | |
| | | | 1 (LOC479538), mRNA | |
| EPA | 1594227_at | UP | Homo sapiens RNA binding motif | AK096015 |
| | | | protein, X-linked (RBMX), mRNA | |
| EPA | 1594231_at | UP | Sus scrofa peptidyl-prolyl cis-trans | NA |
| | | | isomerase A (PPIA), mRNA | |
| EPA | 1594415_at | DOWN | PREDICTED: Bos taurus similar to | AP001675 |
| | | | GTPase, IMAP family member 4 | |
| | | | (Immunity-associated protein 4) | |
| | | | (Immunity-associated nucleotide 1 | |
| | | | protein) (hIAN1) (MSTP062) | |
| | | | (LOC510751), mRNA | |
| EPA | 1594824_at | DOWN | Homo sapiens chromosome 16 | AC130449 |
| | | | clone CTA-233A7, complete | |
| | | | sequence | |
| EPA | 1594939_at | UP | Homo sapiens chromosome 8, | AC090133 |
| | | | clone RP11-813L8, complete | |
| | | | sequence | |
| EPA | 1595021_at | DOWN | Bos taurus mRNA for sodium | NM_000339 |
| | | | chloride cotransporter, partial | |
| EPA | 1595265_at | UP | Yarrowia lipolytica CLIB99, | NG_001333 |
| | | | YALI0C20339g predicted mRNA | |
| EPA | 1595301_at | UP | H. sapiens mRNA for skeletal | AC113382 |
| | | | muscle abundant protein | |
| EPA | 1596553_s_at | DOWN | Homo sapiens chromosome 16 | AK056168 |
| | | | open reading frame 55 (C16orf55), | |
| | | | mRNA | |
| EPA | 1597390_at | DOWN | PREDICTED: Canis familiaris | AY400068 |
| | | | similar to Ataxin-10 | |
| | | | (Spinocerebellar ataxia type 10 | |
| | | | protein) (Brain protein E46 | |
| | | | homolog) (LOC474467), mRNA | |
| EPA | 1597801_at | DOWN | Homo sapiens, clone | AL442128 |
| | | | IMAGE: 4822875, mRNA | |
| EPA | 1597802_at | DOWN | Mus musculus BAC clone RP23- | AL078583 |
| | | | 451I11 from 12, complete | |
| | | | sequence | |
| EPA | 1598585_at | DOWN | Homo sapiens S164 gene, partial | AC011306 |
| | | | cds; PS1 and hypothetical protein | |
| | | | genes, complete cds; and S171 | |
| | | | gene, partial cds | |
| EPA | 1599557_at | DOWN | PREDICTED: Canis familiaris | AY414168 |
| | | | similar to hypothetical protein | |
| | | | MGC12103 (LOC481489), mRNA | |
| EPA | 1599565_at | DOWN | Human DNA sequence from clone | AL139175 |
| | | | RP4-615P17 on chromosome | |
| | | | 1p13-14.3, complete sequence | |
| EPA | 1599601_s_at | DOWN | PREDICTED: Canis familiaris | AY403773 |
| | | | similar to male-enhanced antigen- | |
| | | | bovine (LOC474906), mRNA | |
| EPA | 1600959_at | UP | PREDICTED: Canis familiaris | NA |
| | | | similar to IgA heavy chain constant | |
| | | | region (LOC480452), mRNA | |
| EPA | 1601005_at | DOWN | PREDICTED: Canis familiaris | XM_372592 |
| | | | LOC479025 (LOC479025), mRNA | |
| EPA | 1602471_at | DOWN | Homo sapiens cDNA clone | AC073120 |
| | | | IMAGE: 4797645, partial cds | |
| EPA | 1603225_at | UP | Haemonchus contortus | AC008429 |
| | | | microsatellite Hcms51 sequence | |
| EPA | 1603875_at | DOWN | Homo sapiens cDNA FLJ33460 fis, | AC010092 |
| | | | clone BRAMY2000653, highly | |
| | | | similar to Homo sapiens tousled- | |
| | | | like kinase 1 (TLK1) mRNA | |
| EPA | 1604439_at | DOWN | Homo sapiens mRNA; cDNA | AL137346 |
| | | | DKFZp761M0111 (from clone | |
| | | | DKFZp761M0111) | |
| EPA | 1604600_at | DOWN | Homo sapiens mRNA; cDNA | AC010733 |
| | | | DKFZp686K122 (from clone | |
| | | | DKFZp686K122) | |
| EPA | 1605028_at | DOWN | Canis familiaris secreted B7-1 | NA |
| | | | protein (CD80) gene, alternatively | |
| | | | spliced exon 4 and complete cds | |
| EPA | 1605486_at | UP | Homo sapiens pyruvate | AK096428 |
| | | | dehydrogenase kinase 4 mRNA, 3′ | |
| | | | untranslated region, partial | |
| | | | sequence | |
| EPA | 1605654_at | UP | Mus musculus mbt domain | AK028503 |
| | | | containing 1, mRNA (cDNA clone | |
| | | | MGC: 29000 IMAGE: 2646754), | |
| | | | complete cds | |
| EPA | 1605669_s_at | UP | Homo sapiens cDNA FLJ38323 fis, | AK095642 |
| | | | clone FCBBF3024623, weakly | |
| | | | similar to Homo sapiens C2H2 | |
| | | | (Kruppel-type) zinc finger protein | |
| | | | mRNA | |
| DHA/ | 1582781_at | UP | Canis familiaris L-type Ca channel | AF465484 |
| EPA | | | alpha 1 subunit mRNA, partial cds | |
| DHA/ | 1583031_at | UP | Canis familiaris fibroblast growth | NM_006119 |
| EPA | | | factor-8 (FGF-8) mRNA, partial cds | |
| DHA/ | 1583254_x_at | DOWN | Bos taurus clone IMAGE: 7961516 | X02493 |
| EPA | | | thymosin beta-4-like mRNA, | |
| | | | complete cds | |
| DHA/ | 1583403_at | UP | Sus scrofa carnitine | AK172798 |
| EPA | | | palmitoyltransferase I mRNA, | |
| | | | nuclear gene encoding | |
| | | | mitochondrial protein, complete | |
| | | | cds | |
| DHA/ | 1584742_at | UP | Human DNA sequence from clone | AL591206 |
| EPA | | | RP11-151J10 on chromosome 9 | |
| | | | Contains the 5′ end of a novel | |
| | | | gene (FLJ20060) (contains | |
| | | | FLJ12902, KIAA1574), the ADFP | |
| | | | gene for adipose differentiation- | |
| | | | related protein (ADRP) | |
| DHA/ | 1584951_at | UP | PREDICTED: Canis familiaris | CR605429 |
| EPA | | | similar to Adipophilin (Adipose | |
| | | | differentiation-related protein) | |
| | | | (ADRP) (LOC474720), mRNA | |
| DHA/ | 1585033_at | DOWN | PREDICTED: Canis familiaris | AL121983 |
| EPA | | | similar to KIAA2025 protein | |
| | | | (LOC480065), mRNA | |
| DHA/ | 1585339_at | DOWN | Homo sapiens mRNA for UDP- | AL672237 |
| EPA | | | GalNAc:betaGlcNAc beta 1,3- | |
| | | | galactosaminyltransferase, | |
| | | | polypeptide 2 variant protein | |
| DHA/ | 1585355_at | UP | PREDICTED: Canis familiaris | CR597463 |
| EPA | | | similar to Adipophilin (Adipose | |
| | | | differentiation-related protein) | |
| | | | (ADRP) (LOC474720), mRNA | |
| DHA/ | 1586172_at | DOWN | Homo sapiens chromosome 11, | AC131263 |
| EPA | | | clone RP11-348A11, complete | |
| | | | sequence | |
| DHA/ | 1586287_at | DOWN | Bos taurus mRNA for transcription | AC106818 |
| EPA | | | factor COUP-TFI (COUP-TFI gene) | |
| DHA/ | 1586614_at | DOWN | PREDICTED: Canis familiaris | BC037320 |
| EPA | | | similar to F-box protein SEL10 | |
| | | | (LOC475465), mRNA | |
| DHA/ | 1586695_at | DOWN | Homo sapiens RAD51-like 1 (S. cerevisiae) | BX161515 |
| EPA | | | (RAD51L1), transcript | |
| | | | variant 2, mRNA | |
| DHA/ | 1587254_at | DOWN | PREDICTED: Canis familiaris | AC008785 |
| EPA | | | janus kinase 1 (JAK1), mRNA | |
| DHA/ | 1587413_at | UP | Hirudo medicinalis intermediate | AC005996 |
| EPA | | | filament gliarin mRNA, complete | |
| | | | cds | |
| DHA/ | 1587813_s_at | UP | PREDICTED: Pan troglodytes | AL160175 |
| EPA | | | similar to dJ109F14.3 (novel | |
| | | | putative ring finger protein) | |
| | | | (LOC472236), mRNA | |
| DHA/ | 1589293_at | DOWN | Homo sapiens mRNA for | AB058707 |
| EPA | | | KIAA1804 protein, partial cds | |
| DHA/ | 1589678_s_at | UP | Homo sapiens clone alpha1 mRNA | BK001411 |
| EPA | | | sequence | |
| DHA/ | 1589929_at | DOWN | Homo sapiens solute carrier family | AC145098 |
| EPA | | | 34 (sodium phosphate), member 1, | |
| | | | mRNA (cDNA clone | |
| | | | IMAGE: 5182821), with apparent | |
| | | | retained intron | |
| DHA/ | 1590942_at | DOWN | Human netrin-2 like protein | AC106820 |
| EPA | | | (NTN2L) gene, complete cds | |
| DHA/ | 1591029_at | UP | PREDICTED: Homo sapiens | AC023991 |
| EPA | | | KIAA0146 protein (KIAA0146), | |
| | | | mRNA | |
| DHA/ | 1591083_at | UP | Homo sapiens clone DNA22780 | AC010323 |
| EPA | | | NL2 (UNQ171) mRNA, complete | |
| | | | cds | |
| DHA/ | 1591601_at | DOWN | Human DNA sequence from clone | AL691426 |
| EPA | | | RP11-787B4 on chromosome 9 | |
| | | | Contains the 5′ end of the PAPPA | |
| | | | gene for pregnancy-associated | |
| | | | plasma protein A, a novel gene | |
| | | | and a CpG island, complete | |
| | | | sequence | |
| DHA/ | 1591782_at | UP | PREDICTED: Bos taurus similar to | AC069335 |
| EPA | | | hypothetical protein (LOC514986), | |
| | | | partial mRNA | |
| DHA/ | 1592123_at | DOWN | PREDICTED: Canis familiaris | AY891766 |
| EPA | | | similar to vimentin (LOC477991), | |
| | | | mRNA | |
| DHA/ | 1592160_at | DOWN | PREDICTED: Canis familiaris | BC070246 |
| EPA | | | similar to Fibrinogen alpha/alpha-E | |
| | | | chain precursor (LOC475473), | |
| | | | mRNA | |
| DHA/ | 1592915_s_at | UP | PREDICTED: Canis familiaris | BC004501 |
| EPA | | | similar to hypothetical protein | |
| | | | MGC33867 (LOC478228), mRNA | |
| DHA/ | 1593146_s_at | UP | Homo sapiens Kruppel-like factor | BC063286 |
| EPA | | | 11 (KLF11), mRNA | |
| DHA/ | 1593855_at | DOWN | Felis catus clone RP86-117J4, | AL353710 |
| EPA | | | complete sequence | |
| DHA/ | 1593993_at | DOWN | Pan troglodytes BAC clone RP43- | AC004949 |
| EPA | | | 75I2 from 7, complete sequence | |
| DHA/ | 1594205_at | UP | PREDICTED: Pan troglodytes | DQ048939 |
| EPA | | | similar to putative transcription | |
| | | | factor ZNF131 (LOC461893), | |
| | | | mRNA | |
| DHA/ | 1594291_s_at | DOWN | PREDICTED: Canis familiaris | BC014897 |
| EPA | | | similar to methylcrotonoyl- | |
| | | | Coenzyme A carboxylase 2 (beta) | |
| | | | (LOC478091), mRNA | |
| DHA/ | 1594379_x_at | UP | Felis catus growth arrest and DNA | AL136120 |
| EPA | | | damage-inducible protein 45 | |
| | | | (GADD45), mRNA | |
| DHA/ | 1594413_at | UP | Homo sapiens cytochrome P450, | AC007002 |
| EPA | | | family 26, subfamily B, polypeptide | |
| | | | 1 (CYP26B1), mRNA | |
| DHA/ | 1594564_at | UP | Homo sapiens serine palmitoyl | AF111168 |
| EPA | | | transferase, subunit II gene, | |
| | | | complete cds; and unknown genes | |
| DHA/ | 1594848_at | UP | PREDICTED: Pan troglodytes | AC073263 |
| EPA | | | hypothetical protein XP_513164 | |
| | | | (LOC456583), mRNA | |
| DHA/ | 1594939_at | UP | Homo sapiens chromosome 8, | AC090133 |
| EPA | | | clone RP11-813L8, complete | |
| | | | sequence | |
| DHA/ | 1595083_at | DOWN | PREDICTED: Canis familiaris | AK055530 |
| EPA | | | similar to hypothetical protein | |
| | | | MGC18257 (LOC474943), mRNA | |
| DHA/ | 1595280_at | DOWN | Homo sapiens mRNA; cDNA | AL355298 |
| EPA | | | DKFZp686N1929 (from clone | |
| | | | DKFZp686N1929) | |
| DHA/ | 1595481_at | DOWN | PREDICTED: Canis familiaris | NM_002492 |
| EPA | | | LOC478639 (LOC478639), mRNA | |
| DHA/ | 1595587_at | DOWN | PREDICTED: Canis familiaris | BC048260 |
| EPA | | | similar to copine VIII (LOC477646), | |
| | | | mRNA | |
| DHA/ | 1595673_at | DOWN | PREDICTED: Canis familiaris | BC048351 |
| EPA | | | similar to SDA1 domain containing | |
| | | | 1 (LOC478431), mRNA | |
| DHA/ | 1596041_at | DOWN | Homo sapiens mRNA; cDNA | AL354707 |
| EPA | | | DKFZp686I15205 (from clone | |
| | | | DKFZp686I15205) | |
| DHA/ | 1596238_at | UP | PREDICTED: Canis familiaris | AL110128 |
| EPA | | | similar to palmitoyl-protein | |
| | | | thioesterase 2 isoform a precursor | |
| | | | (LOC474856), mRNA | |
| DHA/ | 1596301_at | DOWN | Mouse DNA sequence from clone | AC000007 |
| EPA | | | RP23-440D4 on chromosome 4, | |
| | | | complete sequence | |
| DHA/ | 1597387_at | UP | PREDICTED: Canis familiaris | BC032398 |
| EPA | | | similar to Alpha-N- | |
| | | | acetylglucosaminidase precursor | |
| | | | (N-acetyl-alpha-glucosaminidase) | |
| | | | (NAG) (LOC490965), mRNA | |
| DHA/ | 1597847_at | UP | PREDICTED: Gallus gallus similar | AC098935 |
| EPA | | | to ubiquitin specific protease 37 | |
| | | | (LOC424217), mRNA | |
| DHA/ | 1599572_at | DOWN | PREDICTED: Canis familiaris | NA |
| EPA | | | similar to ORF2 (LOC475183), | |
| | | | mRNA | |
| DHA/ | 1599950_at | DOWN | PREDICTED: Canis familiaris | AL136304 |
| EPA | | | similar to male-enhanced antigen- | |
| | | | bovine (LOC474906), mRNA | |
| DHA/ | 1600310_at | DOWN | PREDICTED: Canis familiaris | AK223446 |
| EPA | | | similar to piggyBac transposable | |
| | | | element derived 1 (LOC488322), | |
| | | | mRNA | |
| DHA/ | 1600683_at | DOWN | Canis familiaris clone RP81- | NA |
| EPA | | | 391L22, complete sequence | |
| DHA/ | 1601351_at | UP | Canis Familiaris, clone XX-25A1, | NA |
| EPA | | | complete sequence | |
| DHA/ | 1601383_at | UP | PREDICTED: Canis familiaris | BT007509 |
| EPA | | | similar to Putative GTP-binding | |
| | | | protein RAY-like (Rab-like protein | |
| | | | 4) (LOC474517), mRNA | |
| DHA/ | 1601782_at | DOWN | Homo sapiens lactamase, beta 2, | AC022731 |
| EPA | | | mRNA (cDNA clone | |
| | | | IMAGE: 3452575) | |
| DHA/ | 1602033_at | DOWN | PREDICTED: Bos taurus similar to | AL445467 |
| EPA | | | G protein-coupled receptor 23 | |
| | | | (LOC539738), mRNA | |
| DHA/ | 1602162_at | DOWN | Homo sapiens BAC clone RP11- | AC093850 |
| EPA | | | 489P15 from 2, complete | |
| | | | sequence | |
| DHA/ | 1603521_at | DOWN | Homo sapiens cDNA FLJ33134 fis, | BC017798 |
| EPA | | | clone UMVEN2000453, weakly | |
| | | | similar to Mus musculus fetal | |
| | | | globin inducing factor mRNA | |
| DHA/ | 1603534_at | DOWN | PREDICTED: Canis familiaris | AL592064 |
| EPA | | | similar to protein tyrosine | |
| | | | phosphatase, receptor type, Q | |
| | | | isoform 1 precursor (LOC482581), | |
| | | | mRNA | |
| DHA/ | 1603559_s_at | DOWN | PREDICTED: Canis familiaris | AY413985 |
| EPA | | | similar to neural activity-related | |
| | | | ring finger protein (LOC475470), | |
| | | | mRNA | |
| DHA/ | 1603658_s_at | UP | Homo sapiens mRNA; cDNA | AL834247 |
| EPA | | | DKFZp451E012 (from clone | |
| | | | DKFZp451E012); complete cds | |
| DHA/ | 1603674_at | DOWN | Homo sapiens cDNA FLJ13648 fis, | AK023710 |
| EPA | | | clone PLACE1011340, weakly | |
| | | | similar to Homo sapiens IDN3-B | |
| | | | mRNA | |
| DHA/ | 1605317_at | DOWN | Homo sapiens chromosome 16 | AC093509 |
| EPA | | | clone CTD-2337L2, complete | |
| | | | sequence | |
| DHA/ | 1605486_at | UP | Homo sapiens pyruvate | AK096428 |
| EPA | | | dehydrogenase kinase 4 mRNA, 3′ | |
| | | | untranslated region, partial | |
| | | | sequence | |
| DHA/ | 1605832_at | DOWN | Homo sapiens mRNA; cDNA | AK097112 |
| EPA | | | DKFZp451J152 (from clone | |
| | | | DKFZp451J152); complete cds | |
| DHA/ | 1605935_at | DOWN | Mus musculus mRNA for NFI-B | AK024964 |
| EPA | | | protein, complete cds | |
| ALA | 1582455_at | DOWN | Canis familiaris type I collagen pre- | AB209597 |
| | | | pro-alpha1(I) chain (COL1A1) | |
| | | | mRNA, complete cds | |
| ALA | 1584508_at | DOWN | PREDICTED: Pan troglodytes | AK122763 |
| | | | LOC464838 (LOC464838), mRNA | |
| ALA | 1584742_at | UP | Human DNA sequence from clone | AL591206 |
| | | | RP11-151J10 on chromosome 9 | |
| | | | Contains the 5′ end of a novel | |
| | | | gene (FLJ20060) (contains | |
| | | | FLJ12902, KIAA1574), the ADFP | |
| | | | gene for adipose differentiation- | |
| | | | related protein (ADRP) | |
| ALA | 1584951_at | UP | PREDICTED: Canis familiaris | CR605429 |
| | | | similar to Adipophilin (Adipose | |
| | | | differentiation-related protein) | |
| | | | (ADRP) (LOC474720), mRNA | |
| ALA | 1585266_at | DOWN | PREDICTED: Canis familiaris | BC005053 |
| | | | similar to FLJ20859 protein | |
| | | | (LOC475396), mRNA | |
| ALA | 1585355_at | UP | PREDICTED: Canis familiaris | CR597463 |
| | | | similar to Adipophilin (Adipose | |
| | | | differentiation-related protein) | |
| | | | (ADRP) (LOC474720), mRNA | |
| ALA | 1585515_at | UP | PREDICTED: Canis familiaris | AF303134 |
| | | | LOC476210 (LOC476210), mRNA | |
| ALA | 1585553_at | DOWN | PREDICTED: Canis familiaris | BC032361 |
| | | | similar to tenascin-N (LOC490335), | |
| | | | mRNA | |
| ALA | 1586185_at | UP | PREDICTED: Canis familiaris | AC093611 |
| | | | similar to hypothetical protein | |
| | | | LOC90637 (LOC480809), mRNA | |
| ALA | 1587312_at | UP | PREDICTED: Canis familiaris | AC124862 |
| | | | LOC491404 (LOC491404), mRNA | |
| ALA | 1587413_at | UP | Hirudo medicinalis intermediate | AC005996 |
| | | | filament gliarin mRNA, complete | |
| | | | cds | |
| ALA | 1587838_at | DOWN | Homo sapiens fibroblast growth | AL031386 |
| | | | factor 13 (FGF13), transcript | |
| | | | variant 1B, mRNA | |
| ALA | 1588093_at | DOWN | Homo sapiens hypothetical protein | BC039892 |
| | | | FLJ20507, mRNA (cDNA clone | |
| | | | MGC: 47628 IMAGE: 5725347), | |
| | | | complete cds | |
| ALA | 1588502_at | DOWN | Homo sapiens mRNA for cAMP | AB209262 |
| | | | responsive element binding protein | |
| | | | 5 isoform beta variant protein | |
| ALA | 1589017_at | UP | Homo sapiens mRNA for | AB209330 |
| | | | microtubule-associated protein 2 | |
| | | | isoform 2 variant protein | |
| ALA | 1590554_at | UP | PREDICTED: Canis familiaris | AC025842 |
| | | | similar to ATP/GTP binding protein | |
| | | | 1 (LOC479034), mRNA | |
| ALA | 1591083_at | UP | Homo sapiens clone DNA22780 | AC010323 |
| | | | NL2 (UNQ171) mRNA, complete | |
| | | | cds | |
| ALA | 1591749_at | UP | Canis familiaris natural resistance | AY400098 |
| | | | associated macrophage protein | |
| | | | (NRAMP1), mRNA | |
| ALA | 1592201_at | UP | HIV-2 strain A\|G1612 from Ghana | AL929410 |
| | | | gag protein (gag) gene, partial cds | |
| ALA | 1593146_s_at | UP | Homo sapiens Kruppel-like factor | BC063286 |
| | | | 11 (KLF11), mRNA | |
| ALA | 1593222_at | UP | Human DNA sequence from clone | AL139243 |
| | | | RP11-439D8 on chromosome 10 | |
| | | | Contains a novel gene, the HPS1 | |
| | | | gene for Hermansky-Pudlak | |
| | | | syndrome 1, the 3′ end of the | |
| | | | HPSE2 gene for heparanase 2 and | |
| | | | a CpG island, complete | |
| ALA | 1593224_at | UP | PREDICTED: Canis familiaris | AL138842 |
| | | | similar to hemojuvelin isoform a | |
| | | | (LOC475830), mRNA | |
| ALA | 1593710_at | UP | PREDICTED: Bos taurus similar to | AY338490 |
| | | | glutathione reductase | |
| | | | (LOC506406), partial mRNA | |
| ALA | 1593836_at | UP | Canis familiaris clone RP81- | NA |
| | | | 142A6, complete sequence | |
| ALA | 1595172_s_at | UP | PREDICTED: Canis familiaris | NA |
| | | | similar to glyceraldehyde-3- | |
| | | | phosphate dehydrogenase | |
| | | | (LOC479078), mRNA | |
| ALA | 1595533_at | UP | Human DNA sequence from clone | AL355315 |
| | | | RP11-548K23 on chromosome 10 | |
| | | | Contains the ANKRD2 gene for | |
| | | | ankyrin repeat domain 2 (stretch | |
| | | | responsive muscle), six novel | |
| | | | genes, the gene for | |
| | | | phosphatidylinositol 4-kinase | |
| ALA | 1595722_at | UP | Homo sapiens chromosome 17, | AC015920 |
| | | | clone CTD-3022L24, complete | |
| | | | sequence | |
| ALA | 1595801_at | UP | Homo sapiens cDNA FLJ34120 fis, | AK091439 |
| | | | clone FCBBF3009541 | |
| ALA | 1596406_at | UP | Pongo pygmaeus mRNA; cDNA | AC023795 |
| | | | DKFZp459C032 (from clone | |
| | | | DKFZp459C032) | |
| ALA | 1599614_at | UP | PREDICTED: Canis familiaris | AL365364 |
| | | | LOC477772 (LOC477772), mRNA | |
| ALA | 1600037_at | DOWN | Homo sapiens, clone | AC007163 |
| | | | IMAGE: 5294477, mRNA | |
| ALA | 1600155_at | UP | PREDICTED: Canis familiaris | AC011389 |
| | | | LOC479296 (LOC479296), mRNA | |
| ALA | 1600793_at | UP | Drosophila melanogaster | AL157781 |
| | | | CG18408-PA, isoform A (CAP) | |
| | | | mRNA, complete cds | |
| ALA | 1601394_x_at | UP | PREDICTED: Canis familiaris | AC022167 |
| | | | similar to ubiquitin-specific | |
| | | | protease 7 isoform (LOC479854), | |
| | | | mRNA | |
| ALA | 1602423_at | DOWN | PREDICTED: Canis familiaris | AC078880 |
| | | | similar to interferon regulatory | |
| | | | factor 2 binding protein 1 | |
| | | | (LOC484433), mRNA | |
| ALA | 1602589_at | UP | Mustela vison tyrosine | NA |
| | | | aminotransferase gene, complete | |
| | | | cds | |
| ALA | 1603636_at | DOWN | Human DNA sequence from clone | AL031674 |
| | | | RP4-715N11 on chromosome | |
| | | | 20q13.1-13.2 Contains two | |
| | | | putative novel genes, ESTs, STSs | |
| | | | and GSSs, complete sequence | |
| ALA | 1604861_at | DOWN | Homo sapiens chromosome 5 | AC008680 |
| | | | clone CTB-53I9, complete | |
| | | | sequence | |
| ALA | 1605047_at | DOWN | Human DNA sequence from clone | AL713895 |
| | | | RP11-10C13 on chromosome 10 | |
| | | | Contains the 5′ end of the TRIP8 | |
| | | | gene for thyroid hormone receptor | |
| | | | interactor 8 (KIAA1380, | |
| | | | DKFZp761F0118) and the 3′ end | |
| | | | of a novel gene (FLJ1 | |
| ALA | 1605187_at | UP | Human DNA sequence from clone | AL442063 |
| | | | RP11-8N6 on chromosome 9 | |
| | | | Contains the 3′ end of the MELK | |
| | | | gene for maternal embryonic | |
| | | | leucine zipper kinase (KIAA0175), | |
| | | | complete sequence | |
| ALA | 1605429_at | DOWN | Human DNA sequence from clone | AL358073 |
| | | | RP11-458I7 on chromosome 1 | |
| | | | Contains the 5′ end of the ZA20D1 | |
| | | | gene for zinc finger, A20 domain | |
| | | | containing 1, a ribosomal protein | |
| | | | L6 (RPL6) pseudogene, the | |
| | | | VPS45A gene for | |
| ALA | 1605486_at | UP | Homo sapiens pyruvate | AK096428 |
| | | | dehydrogenase kinase 4 mRNA. 3′ | |
| | | | untranslated region, partial | |
| | | | sequence | |
| LA | 1582385_at | DOWN | Canis familiaris Na+-dependent | D26443 |
| | | | glutamate transporter (GLAST), | |
| | | | mRNA | |
| LA | 1582824_at | UP | PREDICTED: Canis familiaris | BC000185 |
| | | | carnitine palmitoyl transferase I | |
| | | | isoform (CPT1), mRNA | |
| LA | 1583273_s_at | DOWN | Homo sapiens mRNA; cDNA | BC008990 |
| | | | DKFZp761G179 (from clone | |
| | | | DKFZp761G179) | |
| LA | 1584258_at | UP | Homo sapiens calsyntenin 2, | BC007943 |
| | | | mRNA (cDNA clone | |
| | | | IMAGE: 4130487), partial cds | |
| LA | 1584677_at | DOWN | PREDICTED: Pan troglodytes | BC024006 |
| | | | similar to cystatin T (LOC469901), | |
| | | | mRNA | |
| LA | 1584742_at | UP | Human DNA sequence from clone | AL591206 |
| | | | RP11-151J10 on chromosome 9 | |
| | | | Contains the 5′ end of a novel | |
| | | | gene (FLJ20060) (contains | |
| | | | FLJ12902, KIAA1574), the ADFP | |
| | | | gene for adipose differentiation- | |
| | | | related protein (ADRP) | |
| LA | 1584951_at | UP | PREDICTED: Canis familiaris | CR605429 |
| | | | similar to Adipophilin (Adipose | |
| | | | differentiation-related protein) | |
| | | | (ADRP) (LOC474720), mRNA | |
| LA | 1585355_at | UP | PREDICTED: Canis familiaris | CR597463 |
| | | | similar to Adipophilin (Adipose | |
| | | | differentiation-related protein) | |
| | | | (ADRP) (LOC474720), mRNA | |
| LA | 1585417_at | UP | Mus musculus microtubule | AC105754 |
| | | | associated monoxygenase, | |
| | | | calponin and LIM domain | |
| | | | containing 3, mRNA (cDNA clone | |
| | | | IMAGE: 30637988), partial cds | |
| LA | 1585604_at | DOWN | Human DNA sequence from clone | AL121927 |
| | | | RP11-175J10 on chromosome 10 | |
| | | | Contains a transforming, acidic | |
| | | | coiled-coil containing protein 1 | |
| | | | (TACC1) pseudogene and a | |
| | | | mitochondrial NADH | |
| | | | dehydrogenase 1 (MTND1) | |
| | | | pseudoge | |
| LA | 1585686_at | UP | PREDICTED: Bos taurus similar to | CR625198 |
| | | | Cold-inducible RNA-binding protein | |
| | | | (Glycine-rich RNA-binding protein | |
| | | | CIRP) (A18 hnRNP) (LOC507120), | |
| | | | mRNA | |
| LA | 1586295_at | DOWN | Homo sapiens downregulated in | BC027860 |
| | | | ovarian cancer 1, mRNA (cDNA | |
| | | | clone MGC: 34368 | |
| | | | IMAGE: 5228947), complete cds | |
| LA | 1588088_at | UP | Homo sapiens hypoxia-inducible | BC008573 |
| | | | protein 2, mRNA (cDNA clone | |
| | | | MGC: 17005 IMAGE: 4182067), | |
| | | | complete cds | |
| LA | 1589569_at | DOWN | PREDICTED: Canis familiaris | BC039825 |
| | | | similar to male germ cell- | |
| | | | associated kinase (LOC478721), | |
| | | | mRNA | |
| LA | 1591083_at | UP | Homo sapiens clone DNA22780 | AC010323 |
| | | | NL2 (UNQ171) mRNA, complete | |
| | | | cds | |
| LA | 1592172_at | UP | Homo sapiens BAC clone CTB- | AC004543 |
| | | | 17C20 from 7, complete sequence | |
| LA | 1594511_s_at | UP | Homo sapiens RGM domain | AK054622 |
| | | | family, member B, mRNA (cDNA | |
| | | | clone IMAGE: 3852164) | |
| LA | 1594801_at | DOWN | Homo sapiens HMGIC fusion | AY309920 |
| | | | partner-like 2 (LHFPL2) mRNA, | |
| | | | complete cds | |
| LA | 1594973_at | UP | PREDICTED: Canis familiaris | AL031387 |
| | | | LOC478197 (LOC478197), mRNA | |
| LA | 1595021_at | DOWN | Bos taurus mRNA for sodium | NM_000339 |
| | | | chloride cotransporter, partial | |
| LA | 1595753_at | DOWN | Homo sapiens CrkRS mRNA, | CR954985 |
| | | | complete cds | |
| LA | 1596117_at | DOWN | Mus musculus piccolo (presynaptic | AP001266 |
| | | | cytomatrix protein) (Pclo), mRNA | |
| LA | 1600646_at | DOWN | Homo sapiens mRNA; cDNA | AC103736 |
| | | | DKFZp547F213 (from clone | |
| | | | DKFZp547F213) | |
| LA | 1600703_at | UP | PREDICTED: Canis familiaris | AC012391 |
| | | | similar to budding uninhibited by | |
| | | | benzimidazoles 3 homolog | |
| | | | (LOC477857), mRNA | |
| LA | 1601942_at | DOWN | PREDICTED: Canis familiaris | AC026358 |
| | | | similar to family with sequence | |
| | | | similarity 20, member A | |
| | | | (LOC480458), mRNA | |
| LA | 1603578_at | DOWN | PREDICTED: Canis familiaris | CR609892 |
| | | | similar to CD63 antigen | |
| | | | (LOC474391), mRNA | |
| LA | 1605486_at | UP | Homo sapiens pyruvate | AK096428 |
| | | | dehydrogenase kinase 4 mRNA, 3′ | |
| | | | untranslated region, partial | |
| | | | sequence | |
| LA | 1605822_at | DOWN | Human dipeptidyl aminopeptidase | M96859 |
| | | | like protein mRNA, complete cds | |
| ARA | 1582824_at | UP | PREDICTED: Canis familiaris | BC000185 |
| | | | carnitine palmitoyl transferase I | |
| | | | isoform (CPT1), mRNA | |
| ARA | 1582851_at | UP | Rattus norvegicus nuclear receptor | BC047875 |
| | | | subfamily 1, group D, member 1, | |
| | | | mRNA (cDNA clone MGC: 72288 | |
| | | | IMAGE: 5698020), complete cds | |
| ARA | 1582999_at | DOWN | Canis familiaris cyclin-dependent | AY399342 |
| | | | kinase inhibitor (WAF1) mRNA, | |
| | | | partial cds | |
| ARA | 1583403_at | UP | Sus scrofa carnitine | AK172798 |
| | | | palmitoyltransferase I mRNA, | |
| | | | nuclear gene encoding | |
| | | | mitochondrial protein, complete | |
| | | | cds | |
| ARA | 1584742_at | UP | Human DNA sequence from clone | AL591206 |
| | | | RP11-151J10 on chromosome 9 | |
| | | | Contains the 5′ end of a novel | |
| | | | gene (FLJ20060) (contains | |
| | | | FLJ12902, KIAA1574), the ADFP | |
| | | | gene for adipose differentiation- | |
| | | | related protein (ADRP) | |
| ARA | 1584951_at | UP | PREDICTED: Canis familiaris | CR605429 |
| | | | similar to Adipophilin (Adipose | |
| | | | differentiation-related protein) | |
| | | | (ADRP) (LOC474720), mRNA | |
| ARA | 1585355_at | UP | PREDICTED: Canis familiaris | CR597463 |
| | | | similar to Adipophilin (Adipose | |
| | | | differentiation-related protein) | |
| | | | (ADRP) (LOC474720), mRNA | |
| ARA | 1586047_s_at | DOWN | Mouse DNA sequence from clone | AL512655 |
| | | | RP23-348N2 on chromosome 11 | |
| | | | Contains the 5′ end of the Ppp3r1 | |
| | | | gene for protein phospatase 3 | |
| | | | regulatory subunit B alpha isoform | |
| | | | (calcineurin 8, type I), a ribosomal | |
| | | | protei | |
| ARA | 1586172_at | DOWN | Homo sapiens chromosome 11, | AC131263 |
| | | | clone RP11-348A11, complete | |
| | | | sequence | |
| ARA | 1586185_at | UP | PREDICTED: Canis familiaris | AC093611 |
| | | | similar to hypothetical protein | |
| | | | LOC90637 (LOC480809), mRNA | |
| ARA | 1586281_{—at} | UP | PREDICTED: Pan troglodytes | BX640828 |
| | | | similar to DEP domain containing | |
| | | | protein 5 (LOC458777), mRNA | |
| ARA | 1587792_at | UP | PREDICTED: Bos taurus similar to | AL049589 |
| | | | phosphoglycerate kinase 1 | |
| | | | (LOC533730), partial mRNA | |
| ARA | 1588088_at | UP | Homo sapiens hypoxia-inducible | BC008573 |
| | | | protein 2, mRNA (cDNA clone | |
| | | | MGC: 17005 IMAGE: 4182067), | |
| | | | complete cds | |
| ARA | 1588903_at | UP | Homo sapiens mRNA; cDNA | U32996 |
| | | | DKFZp686I2148 (from clone | |
| | | | DKFZp686I2148) | |
| ARA | 1590656_at | UP | PREDICTED: Canis familiaris | AY404349 |
| | | | similar to SWI/SNF-related matrix- | |
| | | | associated actin-dependent | |
| | | | regulator of chromatin c1 | |
| | | | (LOC476640), mRNA | |
| ARA | 1590755_at | DOWN | Homo sapiens BAC clone RP11- | AC102953 |
| | | | 1246C19 from 7, complete | |
| | | | sequence | |
| ARA | 1591083_at | UP | Homo sapiens clone DNA22780 | AC010323 |
| | | | NL2 (UNQ171) mRNA, complete | |
| | | | cds | |
| ARA | 1592286_s_at | DOWN | Homo sapiens clone DNA77624 | BC057284 |
| | | | SHATr/JAM3 (UNQ859) mRNA, | |
| | | | complete cds | |
| ARA | 1592610_at | DOWN | Homo sapiens cDNA clone | BC071790 |
| | | | IMAGE: 4611512, partial cds | |
| ARA | 1592947_at | UP | Homo sapiens hypothetical protein | AC016585 |
| | | | FLJ11795 (FLJ11795), mRNA | |
| ARA | 1593146_s_at | UP | Homo sapiens Kruppel-like factor | BC063286 |
| | | | 11 (KLF11), mRNA | |
| ARA | 1593254_at | DOWN | PREDICTED: Canis familiaris | AL020991 |
| | | | similar to 6-phosphofructo-2- | |
| | | | kinase/fructose-2,6-biphosphatase | |
| | | | 1 (6PF-2-K/Fru-2,6-P2ASE liver | |
| | | | isozyme) (LOC491903), mRNA | |
| ARA | 1593907_s_at | DOWN | PREDICTED: Bos taurus similar to | AL034553 |
| | | | 26S proteasome non-ATPase | |
| | | | regulatory subunit 10 (26S | |
| | | | proteasome regulatory subunit | |
| | | | p28) (Gankyrin) (LOC535414), | |
| | | | mRNA | |
| ARA | 1594108_at | UP | Gallus gallus mRNA for | AC100847 |
| | | | hypothetical protein, clone 15k1 | |
| ARA | 1594939_at | UP | Homo sapiens chromosome 8, | AC090133 |
| | | | clone RP11-813L8, complete | |
| | | | sequence | |
| ARA | 1595334_at | DOWN | Homo sapiens mRNA; cDNA | AL031290 |
| | | | DKFZp779M1134 (from clone | |
| | | | DKFZp779M1134) | |
| ARA | 1595495_s_at | UP | Mustela vison NADH | NA |
| | | | dehydrogenase subunit 5 (ND5) | |
| | | | gene, complete cds; mitochondrial | |
| | | | gene for mitochondrial product | |
| ARA | 1595956_at | UP | PREDICTED: Gallus gallus similar | AC025467 |
| | | | to KIAA1389 protein (LOC421523), | |
| | | | mRNA | |
| ARA | 1596476_at | DOWN | Oryza sativa (japonica cultivar- | Z84490 |
| | | | group) chromosome 11 clone | |
| | | | OSJNBb0071K13, complete | |
| | | | sequence | |
| ARA | 1596787_at | DOWN | Homo sapiens CASK interacting | AC100787 |
| | | | protein 2 (CASKIN2), mRNA | |
| ARA | 1597116_at | UP | PREDICTED: Canis familiaris | BX538213 |
| | | | similar to cytoplasmic | |
| | | | polyadenylation element binding | |
| | | | protein 4 (LOC479287), mRNA | |
| ARA | 1598013_at | UP | PREDICTED: Canis familiaris | BC021135 |
| | | | similar to InaD-like protein isoform | |
| | | | 1 (LOC479550), mRNA | |
| ARA | 1598063_at | UP | PREDICTED: Rattus norvegicus | AC112198 |
| | | | similar to proacrosin-binding | |
| | | | protein (LOC500316), mRNA | |
| ARA | 1598902_at | UP | Homo sapiens cDNA clone | BC009735 |
| | | | IMAGE: 3878708, partial cds | |
| ARA | 1599787_at | UP | Homo sapiens, clone | AL035703 |
| | | | IMAGE: 4821877, mRNA, partial | |
| | | | cds | |
| ARA | 1599851_at | UP | PREDICTED: Gallus gallus | AC092040 |
| | | | frizzled-3 (FZ-3), mRNA | |
| ARA | 1601092_at | UP | Homo sapiens TRIAD1 type I | AF099149 |
| | | | mRNA, complete cds | |
| ARA | 1601561_at | DOWN | PREDICTED: Canis familiaris | AL357374 |
| | | | similar to RIKEN cDNA | |
| | | | 2010100O12 (LOC477215), mRNA | |
| ARA | 1601912_at | DOWN | Mus musculus expressed | AL359494 |
| | | | sequence AW538196 | |
| | | | (AW538196), mRNA | |
| ARA | 1602749_at | UP | Homo sapiens BAC clone RP11- | AC108866 |
| | | | 44D21 from 4, complete sequence | |
| ARA | 1603093_at | UP | Homo sapiens genomic DNA, | AP003083 |
| | | | chromosome 11q clone: RP11- | |
| | | | 179B7, complete sequence | |
| ARA | 1603151_at | UP | Rattus norvegicus chromosome | AL033380 |
| | | | 20, major histocompatibility | |
| | | | complex, assembled from 40 | |
| | | | BACs, strain Brown Norway | |
| | | | (BN/ssNHsd), RT1n haplotype; | |
| | | | segment 7/11 | |
| ARA | 1603452_s_at | DOWN | Homo sapiens cDNA clone | AC006211 |
| | | | IMAGE: 4611044, partial cds | |
| ARA | 1603454_at | UP | Bos taurus mRNA for similar to | AL158068 |
| | | | cytochrome c oxidase subunit VIb, | |
| | | | partial cds, clone: ORCS10538 | |
| ARA | 1603839_at | DOWN | PREDICTED: Rattus norvegicus | BX284687 |
| | | | transcription factor EB (predicted) | |
| | | | (Tcfeb_predicted), mRNA | |
| ARA | 1604372_at | UP | PREDICTED: Canis familiaris | AY411810 |
| | | | LOC475665 (LOC475665), mRNA | |
| ARA | 1604969_at | DOWN | Homo sapiens chromosome 17, | AC005332 |
| | | | clone hRPK.147_L_13, complete | |
| | | | sequence | |
| ARA | 1605486_at | UP | Homo sapiens pyruvate | AK096428 |
| | | | dehydrogenase kinase 4 mRNA, 3′ | |
| | | | untranslated region, partial | |
| | | | sequence | |
| SA | Cfa.10737.1.A1_at | DOWN | PREDICTED: Canis familiaris | AL663074 |
| | | | similar to HP1-BP74, transcript | |
| | | | variant 4 (LOC478203), mRNA | |
| SA | Cfa.10872.1.A1_at | UP | Homo sapiens Kruppel-like factor | CR591795 |
| | | | 11, mRNA (cDNA clone | |
| | | | MGC: 71570 IMAGE: 30343877), | |
| | | | complete cds | |
| SA | Cfa.12323.1.A1_at | UP | PREDICTED: Canis familiaris | AC010323 |
| | | | similar to angiopoietin-like 4 protein | |
| | | | (LOC476724), mRNA | |
| SA | Cfa.12533.1.A1_at | UP | PREDICTED: Bos taurus similar to | AC144438 |
| | | | insulin induced gene 1 isoform 1 | |
| | | | (LOC511899), mRNA | |
| SA | Cfa.12594.1.A1_at | UP | Homo sapiens G protein-coupled | AC096920 |
| | | | receptor 17, mRNA (cDNA clone | |
| | | | MGC: 35264 IMAGE: 5174146), | |
| | | | complete cds | |
| SA | Cfa.12839.1.A1_at | DOWN | PREDICTED: Canis familiaris | AC103591 |
| | | | similar to nexilin isoform s | |
| | | | (LOC490202), mRNA | |
| SA | Cfa.17.1.S1_s_at | UP | Canis familiaris organic anion | NM_134431 |
| | | | transporting polypeptide A | |
| | | | (OATPA) mRNA, partial cds | |
| SA | Cfa.17302.1.S1_s_at | DOWN | PREDICTED: Canis familiaris | NM_015549 |
| | | | similar to pleckstrin homology | |
| | | | domain containing, family G, | |
| | | | member 3 (LOC611460), mRNA | |
| SA | Cfa.17415.1.S1_s_at | DOWN | PREDICTED: Canis familiaris | XM_088459 |
| | | | similar to regucalcin gene promotor | |
| | | | region related protein | |
| | | | (LOC607434), mRNA | |
| SA | Cfa.17931.1.S1_s_at | DOWN | PREDICTED: Canis familiaris | NM_022834 |
| | | | similar to von Willebrand factor A | |
| | | | domain-related protein isoform 1 | |
| | | | (LOC607112), mRNA | |
| SA | Cfa.1854.1.A1_at | UP | Homo sapiens fatty acid | AP002380 |
| | | | desaturase 1 (FADS1), mRNA | |
| SA | Cfa.18958.1.S1_at | DOWN | PREDICTED: Canis familiaris | BC003409 |
| | | | similar to OCIA domain containing | |
| | | | 1, transcript variant 3 | |
| | | | (LOC475140), mRNA | |
| SA | Cfa.19447.1.S1_at | DOWN | Homo sapiens lamin B1 (LMNB1), | NM_005573 |
| | | | mRNA | |
| SA | Cfa.19635.1.S1_at | DOWN | Lotus corniculatus var. japonicus | AF165140 |
| | | | gene for hypothetical proteins, | |
| | | | complete and partial cds, | |
| | | | clone: BAC259.12D-1 | |
| SA | Cfa.19704.1.S1_at | DOWN | PREDICTED: Bos taurus similar to | AC006276 |
| | | | immunity-related GTPase family, | |
| | | | Q1 (LOC616834), mRNA | |
| SA | Cfa.20892.1.S1_s_at | UP | PREDICTED: Canis familiaris | CR936765 |
| | | | similar to Ectonucleoside | |
| | | | triphosphate diphosphohydrolase 6 | |
| | | | (NTPDase6) (CD39 antigen-like 2) | |
| | | | (LOC485564), mRNA | |
| SA | Cfa.21023.1.S1_at | UP | PREDICTED: Canis familiaris | AL590762 |
| | | | similar to non-POU domain | |
| | | | containing, octamer-binding, | |
| | | | transcript variant 11 (LOC612773), | |
| | | | mRNA | |
| SA | Cfa.2282.1.S1_at | UP | PREDICTED: Canis familiaris | AK096428 |
| | | | similar to [Pyruvate dehydrogenase | |
| | | | [lipoamide]] kinase isozyme 4, | |
| | | | mitochondrial precursor (Pyruvate | |
| | | | dehydrogenase kinase isoform 4) | |
| | | | (LOC482310), mRNA | |
| SA | Cfa.394.1.A1_x_at | UP | PREDICTED: Canis familiaris | NM_000984 |
| | | | similar to 60S ribosomal protein | |
| | | | L23a (LOC478212), mRNA | |
| SA | Cfa.431.1.A1_at | UP | PREDICTED: Canis familiaris | AL591206 |
| | | | similar to Adipophilin (Adipose | |
| | | | differentiation-related protein) | |
| | | | (ADRP), transcript variant 4 | |
| | | | (LOC474720), mRNA | |
| SA | Cfa.431.2.A1_s_at | UP | PREDICTED: Canis familiaris | NM_001122 |
| | | | similar to Adipophilin (Adipose | |
| | | | differentiation-related protein) | |
| | | | (ADRP), transcript variant 1 | |
| | | | (LOC474720), mRNA | |
| SA | Cfa.5582.1.A1_at | DOWN | Homo sapiens mRNA for dual | AB209010 |
| | | | oxidase 2 precursor variant protein | |
| SA | Cfa.6339.1.A1_at | UP | PREDICTED: Canis familiaris | NM_001122 |
| | | | similar to Adipophilin (Adipose | |
| | | | differentiation-related protein) | |
| | | | (ADRP), transcript variant 3 | |
| | | | (LOC474720), mRNA | |
| SA | Cfa.6361.1.A1_at | DOWN | PREDICTED: Canis familiaris | BX679664 |
| | | | similar to 60S ribosomal protein | |
| | | | L17 (L23), transcript variant 4 | |
| | | | (LOC480221), mRNA | |
| SA | Cfa.6482.1.A1_at | DOWN | PREDICTED: Canis familiaris | AL162191 |
| | | | hypothetical protein LOC612422 | |
| | | | (LOC612422), mRNA | |
| SA | Cfa.6915.1.A1_at | DOWN | Homo sapiens 12 BAC RP11- | AC073655 |
| | | | 1105G2 (Roswell Park Cancer | |
| | | | Institute Human BAC Library) | |
| | | | complete sequence | |
| SA | Cfa.7119.1.A1_s_at | DOWN | PREDICTED: Canis familiaris | AC109357 |
| | | | similar to coilin (LOC480564), | |
| | | | mRNA | |
| SA | Cfa.743.2.S1_a_at | UP | PREDICTED: Bos taurus | BC001282 |
| | | | hypothetical protein LOC614918 | |
| | | | (LOC614918), mRNA | |
| SA | Cfa.7531.1.A1_at | UP | Mouse DNA sequence from clone | AC008732 |
| | | | RP23-287B22 on chromosome 11 | |
| | | | Contains a CpG island, complete | |
| | | | sequence | |
| SA | Cfa.7705.2.A1_s_at | DOWN | PREDICTED: Canis familiaris | NM_007192 |
| | | | similar to chromatin-specific | |
| | | | transcription elongation factor large | |
| | | | subunit, transcript variant 2 | |
| | | | (LOC612874), mRNA | |
| SA | Cfa.791.4.A1_at | UP | PREDICTED: Canis familiaris | NM_000986 |
| | | | similar to ribosomal protein L24, | |
| | | | transcript variant 2 (LOC478547), | |
| | | | mRNA | |
| SA | Cfa.9014.1.A1_at | DOWN | Mus musculus SNF8, ESCRT-II | AC091133 |
| | | | complex subunit, homolog (S. cerevisiae), | |
| | | | mRNA (cDNA clone | |
| | | | IMAGE: 5372918) | |
| SA | Cfa.9506.1.A1_at | UP | Homo sapiens hypoxia-inducible | AF144755 |
| | | | protein 2 (HIG2) mRNA, complete | |
| | | | cds | |
| SA | Cfa.9531.1.A1_at | DOWN | Homo sapiens cyclophilin-related | AC092041 |
| | | | protein mRNA, complete cds | |
| SA | Cfa.9685.2.S1_a_at | UP | PREDICTED: Canis familiaris short | AL138960 |
| | | | tandem repeat locus PEZ20 variant | |
| | | | 19 (LOC476927), mRNA | |
| SA | Cfa.9694.1.A1_at | DOWN | Plasmodium yoelii yoelii str. 17XNL | AL359317 |
| | | | hypothetical protein (PY00634) | |
| | | | mRNA, partial cds | |
| SA | CfaAffx.1102.1.S1_at | DOWN | PREDICTED: Canis familiaris | BC065298 |
| | | | similar to RAB5B, member RAS | |
| | | | oncogene family, transcript variant | |
| | | | 3 (LOC474394), mRNA | |
| SA | CfaAffx.12967.1.S1_at | DOWN | Canis familiaris isolate cOR5D23 | AF399364 |
| | | | olfactory receptor family 5 | |
| | | | subfamily D gene, partial cds | |
| SA | CfaAffx.13599.1.S1_at | DOWN | Nicotiana benthamiana clone 6- | NA |
| | | | 272 unknown mRNA | |
| SA | CfaAffx.14479.1.S1_at | DOWN | PREDICTED: Canis familiaris | AK223603 |
| | | | similar to Protein KIAA0652 | |
| | | | (LOC483632), mRNA | |
| SA | CfaAffx.14595.1.S1_s_at | DOWN | PREDICTED: Canis familiaris | NM_201532 |
| | | | similar to diacylglycerol kinase zeta | |
| | | | (LOC611321), mRNA | |
| SA | CfaAffx.15202.1.S1_s_at | DOWN | PREDICTED: Canis familiaris | AK222695 |
| | | | similar to Syndecan-4 precursor | |
| | | | (Amphiglycan) (SYND4) | |
| | | | (Ryudocan core protein) | |
| | | | (LOC485893), mRNA | |
| SA | CfaAffx.16302.1.S1_x_at | DOWN | PREDICTED: Canis familiaris | AB169501 |
| | | | similar to zinc finger protein 25 | |
| | | | (LOC611218), mRNA | |
| SA | CfaAffx.16493.1.S1_at | DOWN | PREDICTED: Bos taurus similar to | AL079340 |
| | | | Phosphatidylinositol 4-kinase beta | |
| | | | (PtdIns 4-kinase) (PI4Kbeta) | |
| | | | (PI4K-beta) (NPIK) (PI4K92) | |
| | | | (LOC613348), mRNA | |
| SA | CfaAffx.19197.1.S1_at | UP | Homo sapiens amyotrophic lateral | NM_020919 |
| | | | sclerosis 2 (juvenile) (ALS2), | |
| | | | mRNA | |
| SA | CfaAffx.19206.1.S1_at | DOWN | Ipomoea nil Magenta gene for | BX647478 |
| | | | flavonoid 3′-hydroxylase, complete | |
| | | | cds | |
| SA | CfaAffx.197.1.S1_s_at | DOWN | PREDICTED: Canis familiaris bZIP | NM_003204 |
| | | | protein, transcript variant 1 (LCR- | |
| | | | F1), mRNA | |
| SA | CfaAffx.20515.1.S1_s_at | UP | PREDICTED: Canis familiaris | AL110210 |
| | | | similar to protein tyrosine | |
| | | | phosphatase, non-receptor type 23 | |
| | | | (LOC609220), mRNA | |
| SA | CfaAffx.21182.1.S1_s_at | DOWN | PREDICTED: Canis familiaris | BC098376 |
| | | | similar to CG4699-PA, isoform A, | |
| | | | transcript variant 4 (LOC480489), | |
| | | | mRNA | |
| SA | CfaAffx.21280.1.S1_at | DOWN | PREDICTED: Canis familiaris | BC035576 |
| | | | similar to Mitogen-activated protein | |
| | | | kinase kinase kinase 14 (NF-kappa | |
| | | | beta-inducing kinase) | |
| | | | (Serine/threonine-protein kinase | |
| | | | NIK) (HsNIK) (LOC490926), mRNA | |
| SA | CfaAffx.22082.1.S1_s_at | DOWN | PREDICTED: Canis familiaris | XM_370654 |
| | | | similar to zinc finger CCCH type | |
| | | | containing 12A (LOC489416), | |
| | | | mRNA | |
| SA | CfaAffx.22560.1.S1_at | DOWN | PREDICTED: Canis familiaris | BC063306 |
| | | | similar to Cullin-5 (CUL-5) | |
| | | | (Vasopressin-activated calcium- | |
| | | | mobilizing receptor) (VACM-1) | |
| | | | (LOC489422), mRNA | |
| SA | CfaAffx.23320.1.S1_at | DOWN | PREDICTED: Canis familiaris | BC032114 |
| | | | similar to RAD52B (LOC480794), | |
| | | | mRNA | |
| SA | CfaAffx.23784.1.S1_s_at | DOWN | PREDICTED: Canis familiaris | BC023600 |
| | | | similar to aldehyde dehydrogenase | |
| | | | 4A1 precursor (LOC612452), | |
| | | | mRNA | |
| SA | CfaAffx.23872.1.S1_s_at | UP | PREDICTED: Canis familiaris | AL136747 |
| | | | similar to cleavage stimulation | |
| | | | factor, 3 pre-RNA subunit 2, tau | |
| | | | (LOC486459), mRNA | |
| SA | CfaAffx.24040.1.S1_at | UP | PREDICTED: Canis familiaris | NA |
| | | | similar to serine/threonine kinase | |
| | | | 11 interacting protein | |
| | | | (LOC488541), mRNA | |
| SA | CfaAffx.25844.1.S1_at | UP | PREDICTED: Canis familiaris | AK170490 |
| | | | hypothetical LOC22889, transcript | |
| | | | variant 1 (LOC612936), mRNA | |
| SA | CfaAffx.28301.1.S1_s_at | UP | PREDICTED: Canis familiaris | AK222489 |
| | | | similar to angiopoietin-like 4 protein | |
| | | | (LOC476724), mRNA | |
| SA | CfaAffx.2896.1.S1_at | DOWN | PREDICTED: Canis familiaris | NM_006116 |
| | | | similar to Mitogen-activated protein | |
| | | | kinase kinase kinase 7 interacting | |
| | | | protein 1 (TAK1-binding protein 1), | |
| | | | transcript variant 1 (LOC481245), | |
| | | | mRNA | |
| SA | CfaAffx.29858.1.S1_s_at | UP | PREDICTED: Canis familiaris | BC110874 |
| | | | similar to melanoma ubiquitous | |
| | | | mutated protein (LOC612320), | |
| | | | mRNA | |
| SA | CfaAffx.3314.1.S1_at | UP | PREDICTED: Canis familiaris | NM_001122 |
| | | | similar to Adipophilin (Adipose | |
| | | | differentiation-related protein) | |
| | | | (ADRP), transcript variant 4 | |
| | | | (LOC474720), mRNA | |
| SA | CfaAffx.4425.1.S1_at | UP | PREDICTED: Canis familiaris | NM_024620 |
| | | | similar to zinc finger protein 329 | |
| | | | (LOC484234), mRNA | |
| SA | CfaAffx.4438.1.S1_at | UP | PREDICTED: Canis familiaris | AB023184 |
| | | | similar to FERM and PDZ domain | |
| | | | containing 1 (LOC481614), mRNA | |
| SA | CfaAffx.5367.1.S1_at | DOWN | PREDICTED: Canis familiaris | CR614114 |
| | | | similar to claudin 6 (LOC490048), | |
| | | | mRNA | |
| SA | CfaAffx.654.1.S1_at | UP | PREDICTED: Homo sapiens | XM_499342 |
| | | | similar to ribosomal protein S27 | |
| | | | (LOC442598), mRNA | |
| SA | CfaAffx.668.1.S1_at | DOWN | Homo sapiens Kazal type serine | NM_001001325 |
| | | | protease inhibitor 5-like 2 | |
| | | | (SPINK5L2), mRNA | |
| SA | CfaAffx.6703.1.S1_at | DOWN | PREDICTED: Canis familiaris | AK093847 |
| | | | similar to pumilio homolog 2, | |
| | | | transcript variant 6 (LOC607618), | |
| | | | mRNA | |
| SA | CfaAffx.7822.1.S1_s_at | DOWN | PREDICTED: Canis familiaris | BC106940 |
| | | | similar to FYVE-finger-containing | |
| | | | Rab5 effector protein rabenosyn-5 | |
| | | | (LOC484642), mRNA | |
| SA | CfaAffx.7845.1.S1_s_at | UP | Homo sapiens mRNA for TSC-22 | AJ222700 |
| | | | protein | |
| SA | CfaAffx.8861.1.S1_at | UP | Homo sapiens hypothetical | AK095089 |
| | | | LOC387790 (LOC387790), mRNA | |
| SA | CfaAffx.9083.1.S1_at | UP | PREDICTED: Canis familiaris | AK155096 |
| | | | similar to FLJ20859 protein isoform | |
| | | | 2 (LOC475396), mRNA | |
| SA | CfaAffx.9353.1.S1_s_at | DOWN | PREDICTED: Canis familiaris | NM_007192 |
| | | | similar to chromatin-specific | |
| | | | transcription elongation factor large | |
| | | | subunit, transcript variant 1 | |
| | | | (LOC612874), mRNA | |
| SA | CfaAffx.9845.1.S1_s_at | UP | PREDICTED: Canis familiaris | NM_144999 |
| | | | similar to leucine rich repeat | |
| | | | containing 45 (LOC483375), | |
| | | | mRNA | |
| CLA | Cfa.10478.1.A1_at | UP | PREDICTED: Bos taurus similar to | AC005691 |
| | | | Type II inositol-1,4,5-trisphosphate | |
| | | | 5-phosphatase precursor | |
| | | | (Phosphoinositide 5-phosphatase) | |
| | | | (5PTase) (75 kDa inositol | |
| | | | polyphosphate-5-phosphatase) | |
| | | | (LOC538291), partial mRNA | |
| CLA | Cfa.11267.1.A1_at | DOWN | Homo sapiens cDNA clone | BC024645 |
| | | | IMAGE: 4456146, partial cds | |
| CLA | Cfa.11358.1.A1_at | UP | Homo sapiens solute carrier family | AF170802 |
| | | | 20 (phosphate transporter), | |
| | | | member 2 (SLC20A2), mRNA | |
| CLA | Cfa.11413.1.A1_at | DOWN | Homo sapiens BAC clone RP11- | AC016673 |
| | | | 17N4 from 2, complete sequence | |
| CLA | Cfa.11483.1.A1_at | DOWN | Danio rerio POU domain, class 4, | AL138810 |
| | | | transcription factor 1, mRNA | |
| | | | (cDNA clone MGC: 77341 | |
| | | | IMAGE: 6967996), complete cds | |
| CLA | Cfa.11868.1.A1_at | DOWN | PREDICTED: Canis familiaris | AL132640 |
| | | | similar to pleckstrin homology | |
| | | | domain containing, family H (with | |
| | | | MyTH4 domain) member 1 | |
| | | | (LOC480363), mRNA | |
| CLA | Cfa.12323.1.A1_at | UP | PREDICTED: Canis familiaris | AC010323 |
| | | | similar to angiopoietin-like 4 protein | |
| | | | (LOC476724), mRNA | |
| CLA | Cfa.1284.1.S1_at | UP | Homo sapiens mRNA; cDNA | AL133026 |
| | | | DKFZp434C136 (from clone | |
| | | | DKFZp434C136) | |
| CLA | Cfa.13221.1.A1_at | UP | Human DNA sequence from clone | AL137840 |
| | | | RP11-241O12 on chromosome | |
| | | | Xq26.3-27.3 Contains a novel | |
| | | | gene, complete sequence | |
| CLA | Cfa.13649.1.A1_s_at | DOWN | PREDICTED: Canis familiaris | AK074468 |
| | | | similar to Sodium- and chloride- | |
| | | | dependent transporter XTRP2 | |
| | | | (Solute carrier family 6 member 18) | |
| | | | (LOC478631), mRNA | |
| CLA | Cfa.13707.1.A1_at | DOWN | PREDICTED: Bos taurus similar to | BC008070 |
| | | | Ssu72 RNA polymerase II CTD | |
| | | | phosphatase homolog, transcript | |
| | | | variant 2 (LOC614837), mRNA | |
| CLA | Cfa.13930.1.A1_at | DOWN | Aspergillus nidulans FGSC A4 | AL031779 |
| | | | hypothetical protein (AN0430.2), | |
| | | | mRNA | |
| CLA | Cfa.14103.1.A1_at | DOWN | Arabidopsis thaliana clone | AC099053 |
| | | | RAFL15-15-K01 (R20657) putative | |
| | | | cytochrome P450 (At1g13150) | |
| | | | mRNA, complete cds | |
| CLA | Cfa.15679.1.A1_at | UP | PREDICTED: Canis familiaris | BC039170 |
| | | | similar to C10C5.4 (LOC607282), | |
| | | | mRNA | |
| CLA | Cfa.19017.1.S1_at | UP | PREDICTED: Canis familiaris | AL137013 |
| | | | similar to CG5537-PA, transcript | |
| | | | variant 2 (LOC480960), mRNA | |
| CLA | Cfa.1935.1.A1_at | DOWN | PREDICTED: Canis familiaris | AL590440 |
| | | | hypothetical LOC481916 | |
| | | | (LOC481916), mRNA | |
| CLA | Cfa.20000.1.S1_s_at | UP | PREDICTED: Canis familiaris | AC021754 |
| | | | similar to sperm-associated cation | |
| | | | channel 2 isoform 1 (LOC609008), | |
| | | | mRNA | |
| CLA | Cfa.20451.1.S1_at | UP | Mus musculus ubiquitin-like 4, | AC012153 |
| | | | mRNA (cDNA clone MGC: 19132 | |
| | | | IMAGE: 4215699), complete cds | |
| CLA | Cfa.21599.1.S1_s_at | UP | PREDICTED: Canis familiaris | BC040721 |
| | | | similar to smooth muscle myosin | |
| | | | heavy chain 11 isoform SM1-like, | |
| | | | transcript variant 3 (LOC474686), | |
| | | | mRNA | |
| CLA | Cfa.2308.1.A1_at | UP | Mus musculus piwi-like 4 | AC108065 |
| | | | (Drosophila) (Piwil4), mRNA | |
| CLA | Cfa.2586.1.S1_at | UP | Homo sapiens CDC14 cell division | AY675321 |
| | | | cycle 14 homolog B (S. cerevisiae) | |
| | | | (CDC14B) gene, complete cds | |
| CLA | Cfa.3584.1.S1_s_at | UP | Canis familiaris gonadotropin- | NM_000406 |
| | | | releasing hormone receptor | |
| | | | (GNRHR), mRNA | |
| CLA | Cfa.4761.1.S1_at | UP | PREDICTED: Bos taurus similar to | AK125974 |
| | | | GATA zinc finger domain | |
| | | | containing 2A, transcript variant 6 | |
| | | | (LOC508384), mRNA | |
| CLA | Cfa.4817.1.A1_at | DOWN | Mus musculus nephrin NPHS1 | AC024166 |
| | | | (Nphs1) gene, partial cds | |
| CLA | Cfa.5394.1.A1_at | DOWN | Xenopus laevis MGC80410 | AC012618 |
| | | | protein, mRNA (cDNA clone | |
| | | | MGC: 80410 IMAGE: 5155047), | |
| | | | complete cds | |
| CLA | Cfa.5400.1.A1_at | DOWN | Homo sapiens glutathione | AY324826 |
| | | | peroxidase 6 (olfactory) (GPX6), | |
| | | | mRNA | |
| CLA | Cfa.5759.1.A1_at | UP | Homo sapiens fibroblast growth | AC006441 |
| | | | factor 5 (FGF5) gene, complete | |
| | | | cds | |
| CLA | Cfa.5949.1.A1_x_at | UP | Mus musculus RIKEN cDNA | AC009230 |
| | | | 2500001K11 gene | |
| | | | (2500001K11Rik), mRNA | |
| CLA | Cfa.6989.1.A1_at | DOWN | Human mRNA for KIAA0297 gene, | AL589763 |
| | | | partial cds | |
| CLA | Cfa.7584.1.A1_at | DOWN | Canis familiaris forssman | AC091826 |
| | | | synthetase mRNA, complete cds | |
| CLA | Cfa.7855.1.A1_at | UP | PREDICTED: Canis familiaris | AL162595 |
| | | | similar to FKBP12-rapamycin | |
| | | | complex-associated protein | |
| | | | (FK506-binding protein 12- | |
| | | | rapamycin complex-associated | |
| | | | protein 1) (Rapamycin target | |
| | | | protein) (RAPT1) (Mammalian | |
| | | | target of rapamycin) (MTOR), | |
| | | | transcript variant 2 (LOC478232), | |
| | | | mRNA | |
| CLA | Cfa.8008.2.A1_at | UP | PREDICTED: Canis familiaris | AL137818 |
| | | | similar to GTPase activating | |
| | | | Rap/RanGAP domain-like 1 | |
| | | | isoform 1 (LOC490653), mRNA | |
| CLA | Cfa.8798.1.A1_at | UP | Arabidopsis thaliana At1g50920 | AC007269 |
| | | | mRNA sequence | |
| CLA | CfaAffx.10853.1.S1_at | UP | PREDICTED: Canis familiaris | AC090440 |
| | | | cOR2AG1 olfactory receptor family | |
| | | | 2 subfamily AG-like (cOR2AG1), | |
| | | | mRNA | |
| CLA | CfaAffx.1228.1.S1_at | DOWN | Homo sapiens gene for LIM- | AP002762 |
| | | | homeodomain protein Lhx8, partial | |
| | | | cds | |
| CLA | CfaAffx.13210.1.S1_at | UP | Homo sapiens olfactory receptor, | NM_001005493 |
| | | | family 6, subfamily C, member 6 | |
| | | | (OR6C6), mRNA | |
| CLA | CfaAffx.13599.1.S1_at | DOWN | Nicotiana benthamiana clone 6- | NA |
| | | | 272 unknown mRNA | |
| CLA | CfaAffx.13793.1.S1_at | DOWN | PREDICTED: Strongylocentrotus | AL391114 |
| | | | purpuratus similar to | |
| | | | apurinic/apyrimidinic endonuclease | |
| | | | (44.7 kD) (apn-1) (LOC592745), | |
| | | | mRNA | |
| CLA | CfaAffx.17003.1.S1_s_at | UP | PREDICTED: Canis familiaris | CR593118 |
| | | | similar to actinin, alpha 2, transcript | |
| | | | variant 11 (LOC479191), mRNA | |
| CLA | CfaAffx.18214.1.S1_s_at | UP | PREDICTED: Canis familiaris | AK223627 |
| | | | complement component receptor 2 | |
| | | | (CR2), mRNA | |
| CLA | CfaAffx.18414.1.S1_at | DOWN | PREDICTED: Canis familiaris | S67623 |
| | | | similar to Cytochrome P450 24A1, | |
| | | | mitochondrial precursor (P450- | |
| | | | CC24) (Vitamin D(3) 24- | |
| | | | hydroxylase) (1,25- | |
| | | | dihydroxyvitamin D(3) 24- | |
| | | | hydroxylase) (24-OHase) | |
| | | | (LOC485935), mRNA | |
| CLA | CfaAffx.18922.1.S1_at | DOWN | PREDICTED: Canis familiaris | U18799 |
| | | | similar to dystonia 2, torsion | |
| | | | (autosomal recessive) | |
| | | | (LOC488341), mRNA | |
| CLA | CfaAffx.24169.1.S1_at | UP | Mus musculus solute carrier family | U76343 |
| | | | 6 (neurotransmitter transporter, | |
| | | | GABA), member 13, mRNA (cDNA | |
| | | | clone MGC: 19082 | |
| | | | IMAGE: 4195373), complete cds | |
| CLA | CfaAffx.24675.1.S1_x_at | UP | PREDICTED: Canis familiaris | BC000293 |
| | | | similar to expressed in non- | |
| | | | metastatic cells 1, protein (NM23A) | |
| | | | (nucleoside diphosphate kinase) | |
| | | | (LOC611984), mRNA | |
| CLA | CfaAffx.2488.1.S1_at | UP | PREDICTED: Canis familiaris | AL583806 |
| | | | hypothetical protein LOC612694 | |
| | | | (LOC612694), mRNA | |
| CLA | CfaAffx.29768.1.S1_s_at | UP | PREDICTED: Canis familiaris | BX255925 |
| | | | similar to tripartite motif protein 32 | |
| | | | (predicted) (LOC491233), mRNA | |
| CLA | CfaAffx.4438.1.S1_at | UP | PREDICTED: Canis familiaris | AB023184 |
| | | | similar to FERM and PDZ domain | |
| | | | containing 1 (LOC481614), mRNA | |
| CLA | CfaAffx.6670.1.S1_at | UP | PREDICTED: Canis familiaris | BC009972 |
| | | | similar to microtubule associated | |
| | | | monoxygenase, calponin and LIM | |
| | | | domain containing 1 (LOC481958), | |
| | | | mRNA | |
| CLA | CfaAffx.7326.1.S1_s_at | DOWN | PREDICTED: Canis familiaris | NM_145032 |
| | | | similar to F-box and leucine-rich | |
| | | | repeat protein 13 (LOC609997), | |
| | | | mRNA | |

### Example 3

### Genes Differentially Expressed in the Blood of Fat and Lean Animals that can be Used as Class Predictors for Fat and Lean Animals

In order to simplify clinical and scientific analyses and eliminate the need for using solid tissue samples that have to be biopsied from live animals, blood samples from fat and lean dogs may be obtained and used to develop a “class predictor” that can be used to differentiate between fat and lean animals Class prediction is a form of pattern recognition that involves the use of supervised learning algorithms familiar to one of skill in the art (e.g., Weighted Voting, Class Neighbors, K-Nearest Neighbors and Support Vector Machines) to define a group of genes or gene products that can recognize and differentiate between two groups or classes of animals Developing class predictors generally involves the following steps:
A training step:
   In this step two unambiguously defined groups or classes of animals (for example fat and lean animals) are used to train an algorithm to recognize and differentiate between them.
   This step results in the generation of a “class predictor” set of genes Once a “class predictor” group of genes and or gene products is established and validated it can be used to classify new and unknown samples as they become available.
A validation or testing step:
   The ability of the class predictor to make the distinction between the two groups is then tested by using new samples that are different from those used in the training step and allowing the algorithm to use what it had learned in the training step to predict the class to which each new sample belongs.

In our studies with fat and lean animals, Affymetrix Canine-2 GeneChips are used according to methods provided hereinabove to measure the gene expression levels in blood samples taken from animals that are conventionally identified as clinically fat (28 animals with a body condition score of 4 or 5) or lean (12 animals with a body condition score of 2 or 2.5). The GeneChip data is then used to train an algorithm (Support Vector Machines) that is included in the software program GeneSpring (version 7.2, Agilent Technologies) to generate the class predictor. Accordingly, data indicate 65 probes that exhibit differential expression levels between the fat and lean samples with a “p” value of 0.01 (after the application of a false discovery rate correction) (see Table 8). RMA normalized data provided in Table 9 indicates the intensity of the fold change in expression in a fat animal versus lean animal such that a value greater than one indicates that the gene is upregulated in a fat animal, a value of one indicates no change in expression in a fat versus lean animal and a value of less than one indicates that the expression of the gene is greater in a lean animal than a fat animal. Thus, it is contemplated herein that these probes and the genes and gene products that they represent can potentially be used as class predictors to identify fat and lean animals using blood samples without the need to use adipose tissue samples.

TABLE 8
Affymetrix probes representing genes that can be used as class predictors for
fat and lean animals using blood samples instead of adipose tissue samples

| | Affymetrix probe | top-annotation based on BLAST sequence |
|---|---|---|
| | id | similarity |
| 1 | Cfa.10128.1.A1_at | PREDICTED: Canis familiaris similar to alpha-synuclein |
| | | isoform NACP140; transcript variant 3 (LOC478478); |
| | | mRNA |
| 2 | Cfa.10772.1.A1_at | PREDICTED: Canis familiaris similar to ADP-ribosylation |
| | | factor GTPase activating protein 3; transcript variant 5 |
| | | (LOC474477); mRNA |
| 3 | Cfa.11444.1.A1_at | Homo sapiens elk1 oncogene; complete cds |
| 4 | Cfa.1152.1.A1_s_at | PREDICTED: Canis familiaris similar to ubiquitin C- |
| | | terminal hydrolase UCH37 (LOC478958); mRNA |
| 5 | Cfa.11624.1.A1_at | PREDICTED: Canis familiaris similar to retinaldehyde |
| | | binding protein 1 (LOC479039); mRNA |
| 6 | Cfa.13515.1.S1_at | PREDICTED: Canis familiaris similar to Coiled-coil-helix- |
| | | coiled-coil-helix domain containing protein 3; transcript |
| | | variant 5 (LOC607574); mRNA |
| 7 | Cfa.13669.1.A1_at | No available annotation |
| 8 | Cfa.15521.1.A1_at | Pongo pygmaeus mRNA; cDNA DKFZp468H0312 (from |
| | | clone DKFZp468H0312) |
| 9 | Cfa.16699.1.S1_s_at | PREDICTED: Canis familiaris similar to NADH |
| | | dehydrogenase (ubiquinone) 1 alpha subcomplex; 11; |
| | | 14.7 kDa; transcript variant 1 (LOC476735); mRNA |
| 10 | Cfa.17093.1.S1_at | PREDICTED: Canis familiaris similar to ADP-ribosylation |
| | | factor GTPase activating protein 3; transcript variant 2 |
| | | (LOC474477); mRNA |
| 11 | Cfa.18024.1.S1_s_at | PREDICTED: Canis familiaris similar to MAK31-like |
| | | protein (LOC479488); mRNA |
| 12 | Cfa.1945.1.A1_at | No available annotation |
| 13 | Cfa.19577.1.S1_at | PREDICTED: Rattus norvegicus similar to hypothetical |
| | | protein FLJ25439 (LOC502510); mRNA |
| 14 | Cfa.273.3.A1_s_at | PREDICTED: Canis familiaris similar to NADH |
| | | dehydrogenase (ubiquinone) 1 beta subcomplex 8; |
| | | transcript variant 1 (LOC477798); mRNA |
| 15 | Cfa.3698.1.A1_at | Canis familiaris angiotensin II type 2 receptor mRNA; |
| | | partial cds |
| 16 | Cfa.3895.1.A1_s_at | Canis familiaris Sec61 beta subunit (Sec61b); mRNA |
| 17 | Cfa.4245.1.S1_s_at | PREDICTED: Canis familiaris similar to NADH |
| | | dehydrogenase (ubiquinone) Fe—S protein 6; 13 kDa |
| | | (NADH-coenzyme Q reductase) (LOC478629); mRNA |
| 18 | Cfa.4779.1.A1_at | PREDICTED: Bos taurus similar to mal; T-cell |
| | | differentiation protein-like (LOC512289); mRNA |
| 19 | Cfa.5440.1.A1_at | Magnaporthe grisea 70-15 hypothetical protein |
| | | (MG04641.4) partial mRNA |
| 20 | Cfa.5628.1.A1_s_at | PREDICTED: Canis familiaris similar to growth |
| | | differentiation factor 3 precursor (LOC477702); mRNA |
| 21 | Cfa.5672.1.A1_s_at | PREDICTED: Canis familiaris similar to glyceraldehyde-3- |
| | | phosphate dehydrogenase (LOC481027); mRNA |
| 22 | Cfa.583.1.S1_at | Homo sapiens mRNA; cDNA DKFZp761M0111 (from |
| | | clone DKFZp761M0111) |
| 23 | Cfa.6307.1.A1_s_at | PREDICTED: Canis familiaris similar to presenilin |
| | | enhancer 2 (LOC476479); mRNA |
| 24 | Cfa.6307.1.A1_x_at | PREDICTED: Canis familiaris similar to presenilin |
| | | enhancer 2 (LOC476479); mRNA |
| 25 | Cfa.7730.1.A1_at | PREDICTED: Canis familiaris similar to adiponectin |
| | | receptor 2; transcript variant 2 (LOC477732); mRNA |
| 26 | Cfa.8497.1.A1_at | PREDICTED: Canis familiaris similar to Kelch repeat and |
| | | BTB domain containing protein 10 (Kelch-related protein 1) |
| | | (Kel-like protein 23) (Sarcosin); transcript variant 3 |
| | | (LOC478784); mRNA |
| 27 | Cfa.9073.1.A1_s_at | PREDICTED: Canis familiaris similar to MADS box |
| | | transcription enhancer factor 2; polypeptide C (myocyte |
| | | enhancer factor 2C); transcript variant 30 (LOC479155); |
| | | mRNA |
| 28 | Cfa.9519.1.A1_at | full-length cDNA clone CS0DF038YH13 of Fetal brain of |
| | | Homo sapiens (human) |
| 29 | CfaAffx.11304.1.S1_at | PREDICTED: Canis familiaris similar to solute carrier |
| | | family 5 (iodide transporter); member 8 (LOC482626); |
| | | mRNA |
| 30 | CfaAffx.12600.1.S1_s_at | C. familiaris mRNA for TRAM-protein |
| 31 | CfaAffx.12899.1.S1_at | PREDICTED: Bos taurus similar to olfactory receptor |
| | | Olr535 (LOC510433); mRNA |
| 32 | CfaAffx.13068.1.S1_s_at | Canis familiaris carboxypeptidase B1 (tissue) (CPB1); |
| | | mRNA |
| 33 | CfaAffx.13084.1.S1_at | Mus musculus olfactory receptor MOR232-2 gene; |
| | | complete cds |
| 34 | CfaAffx.13369.1.S1_s_at | PREDICTED: Canis familiaris similar to selenoprotein T |
| | | (LOC612992); mRNA |
| 35 | CfaAffx.13927.1.S1_at | PREDICTED: Canis familiaris similar to CG10510-PA |
| | | (LOC477622); mRNA |
| 36 | CfaAffx.13999.1.S1_s_at | PREDICTED: Canis familiaris similar to Transmembrane 9 |
| | | superfamily protein member 3 precursor; transcript variant |
| | | 5 (LOC612786); mRNA |
| 37 | CfaAffx.14593.1.S1_s_at | PREDICTED: Canis familiaris similar to chromodomain |
| | | helicase DNA binding protein 6; transcript variant 1 |
| | | (LOC477230); mRNA |
| 38 | CfaAffx.16220.1.S1_s_at | PREDICTED: Canis familiaris similar to membrane- |
| | | spanning 4-domains; subfamily A; member 6A isoform 2 |
| | | (LOC612553); mRNA |
| 39 | CfaAffx.16368.1.S1_s_at | Canine mRNA for signal recognition particle receptor |
| 40 | CfaAffx.17233.1.S1_s_at | PREDICTED: Canis familiaris similar to ubiquitin- |
| | | conjugating enzyme E2G 2 (LOC611581); mRNA |
| 41 | CfaAffx.18688.1.S1_at | PREDICTED: Canis familiaris hypothetical protein |
| | | LOC609372 (LOC609372); mRNA |
| 42 | CfaAffx.19132.1.S1_s_at | PREDICTED: Canis familiaris similar to uroplakin 2 |
| | | (LOC610673); mRNA |
| 43 | CfaAffx.19769.1.S1_at | PREDICTED: Canis familiaris similar to YTH domain |
| | | protein 1 (Dermatomyositis associated with cancer |
| | | putative autoantigen-1 homolog) (DACA-1 homolog) |
| | | (LOC485968); mRNA |
| 44 | CfaAffx.20665.1.S1_at | PREDICTED: Canis familiaris similar to patched domain |
| | | containing 1; transcript variant 1 (LOC491775); mRNA |
| 45 | CfaAffx.20740.1.S1_s_at | PREDICTED: Canis familiaris similar to a disintegrin and |
| | | metalloproteinase domain 23 preproprotein; transcript |
| | | variant 2 (LOC607871); mRNA |
| 46 | CfaAffx.21676.1.S1_at | PREDICTED: Canis familiaris similar to Ferritin light chain |
| | | (Ferritin L subunit) (LOC491829); mRNA |
| 47 | CfaAffx.2327.1.S1_s_at | PREDICTED: Canis familiaris similar to ADP-ribosylation |
| | | factor GTPase activating protein 3; transcript variant 5 |
| | | (LOC474477); mRNA |
| 48 | CfaAffx.23835.1.S1_at | Homo sapiens protocadherin 15 (PCDH15); mRNA |
| 49 | CfaAffx.24356.1.S1_s_at | PREDICTED: Canis familiaris similar to Growth hormone |
| | | inducible transmembrane protein (Dermal papilla derived |
| | | protein 2); transcript variant 3 (LOC479266); mRNA |
| 50 | CfaAffx.24849.1.S1_at | PREDICTED: Canis familiaris similar to Olfactory receptor |
| | | 7A5 (Olfactory receptor TPCR92) (LOC610545); mRNA |
| 51 | CfaAffx.25142.1.S1_s_at | PREDICTED: Canis familiaris similar to Renal sodium- |
| | | dependent phosphate transport protein 2 |
| | | (Sodium/phosphate cotransporter 2) (Na(+)/Pi |
| | | cotransporter 2) (Renal sodium-phosphate transport |
| | | protein 2) (Renal Na(+)-dependent phosphate |
| | | cotransporter 2); t |
| 52 | CfaAffx.25751.1.S1_at | Macaca fascicularis brain cDNA; clone: QflA-12135; similar |
| | | to human progestin and adipoQ receptor family member VI |
| | | (PAQR6); mRNA; NM_024897.2 |
| 53 | CfaAffx.26483.1.S1_s_at | Canis familiaris non-metastatic cells 2; protein (NM23B) |
| | | expressed in (NME2); mRNA |
| 54 | CfaAffx.28078.1.S1_s_at | PREDICTED: Canis familiaris similar to CD27-binding |
| | | (Siva) protein isoform 1 (LOC612693); mRNA |
| 55 | CfaAffx.28164.1.S1_at | PREDICTED: Canis familiaris similar to Ubiquitin- |
| | | conjugating enzyme E2 A (Ubiquitin-protein ligase A) |
| | | (Ubiquitin carrier protein A) (HR6A) (mHR6A) |
| | | (LOC492095); mRNA |
| 56 | CfaAffx.2860.1.S1_s_at | PREDICTED: Canis familiaris similar to Coiled-coil-helix- |
| | | coiled-coil-helix domain containing protein 3; transcript |
| | | variant 2 (LOC607574); mRNA |
| 57 | CfaAffx.28798.1.S1_at | PREDICTED: Canis familiaris similar to seizure related |
| | | gene 6 (LOC491175); mRNA |
| 58 | CfaAffx.29250.1.S1_s_at | PREDICTED: Canis familiaris similar to CG4646-PA |
| | | (LOC479563); mRNA |
| 59 | CfaAffx.32063.1.S1_at | No available annotation |
| 60 | CfaAffx.360.1.S1_s_at | PREDICTED: Canis familiaris similar to ADAM DEC1 |
| | | precursor (A disintegrin and metalloproteinase domain-like |
| | | protein decysin 1) (ADAM-like protein decysin 1) |
| | | (LOC608742); mRNA |
| 61 | CfaAffx.3860.1.S1_s_at | Homo sapiens mRNA for KIAA1045 protein; partial cds |
| 62 | CfaAffx.604.1.S1_at | PREDICTED: Canis familiaris similar to zinc finger protein |
| | | 91 (HPF7; HTF10) (LOC484590); mRNA |
| 63 | CfaAffx.6669.1.S1_at | PREDICTED: Canis familiaris similar to progesterone |
| | | membrane binding protein (LOC476084); mRNA |
| 64 | CfaAffx.7079.1.S1_at | PREDICTED: Canis familiaris TATA-box binding protein |
| | | (LOC475040); mRNA |
| 65 | CfaAffx.9326.1.S1_s_at | PREDICTED: Canis familiaris similar to mitochondrial |
| | | ribosomal protein L48 isoform 1 (LOC476812); mRNA |

TABLE 9
Class Predictor Gene Set Expression Values Fat vrs Lean

| Gene | Fold Change Fat vrs Lean |
|---|---|
| Cfa.10128.1.A1_at | 1.178 |
| Cfa.10772.1.A1_at | 0.673 |
| Cfa.11444.1.A1_at | 1.167 |
| Cfa.1152.1.A1_s_at | 0.682 |
| Cfa.11624.1.A1_at | 1.128 |
| Cfa.13515.1.S1_at | 0.702 |
| Cfa.13669.1.A1_at | 1.123 |
| Cfa.15521.1.A1_at | 1.103 |
| Cfa.16699.1.S1_s_at | 0.806 |
| Cfa.17093.1.S1_at | 0.688 |
| Cfa.18024.1.S1_s_at | 0.667 |
| Cfa.1945.1.A1_at | 1.141 |
| Cfa.19577.1.S1_at | 1.167 |
| Cfa.273.3.A1_s_at | 0.708 |
| Cfa.3698.1.A1_at | 1.129 |
| Cfa.3895.1.A1_s_at | 0.718 |
| Cfa.4245.1.S1_s_at | 0.696 |
| Cfa.4779.1.A1_at | 1.206 |
| Cfa.5440.1.A1_at | 1.101 |
| Cfa.5628.1.A1_s_at | 1.156 |
| Cfa.5672.1.A1_s_at | 1.105 |
| Cfa.583.1.S1_at | 0.556 |
| Cfa.6307.1.A1_s_at | 0.755 |
| Cfa.6307.1.A1_x_at | 0.837 |
| Cfa.7730.1.A1_at | 1.208 |
| Cfa.8497.1_A1_at | 1.116 |
| Cfa.9073.1.A1_s_at | 0.718 |
| Cfa.9519.1.A1_at | 0.64 |
| CfaAffx.11304.1.S1_at | 1.187 |
| CfaAffx.12600.1.S1_s_at | 0.74 |
| CfaAffx.12899.1.S1_at | 1.32 |
| CfaAffx.13068.1.S1_s_at | 1.298 |
| CfaAffx.13084.1.S1_at | 1.209 |
| CfaAffx.13369.1.S1_s_at | 0.757 |
| CfaAffx.13927.1.S1_at | 1.137 |
| CfaAffx.13999.1.S1_s_at | 0.781 |
| CfaAffx.14593.1.S1_s_at | 1.258 |
| CfaAffx.16220.1.S1_s_at | 1.132 |
| CfaAffx.16368.1.S1_s_at | 0.809 |
| CfaAffx.17233.1.S1_s_at | 0.811 |
| CfaAffx.18688.1.S1_at | 1.193 |
| CfaAffx.19132.1.S1_s_at | 1.164 |
| CfaAffx.19769.1.S1_at | 0.849 |
| CfaAffx.20665.1.S1_at | 1.205 |
| CfaAffx.20740.1.S1_s_at | 1.184 |
| CfaAffx.21676.1.S1_at | 1.156 |
| CfaAffx.2327.1.S1_s_at | 0.657 |
| CfaAffx.23835.1.S1_at | 1.116 |
| CfaAffx.24356.1.S1_s_at | 0.729 |
| CfaAffx.24849.1.S1_at | 1.126 |
| CfaAffx.25142.1.S1_s_at | 1.184 |
| CfaAffx.25751.1.S1_at | 1.102 |
| CfaAffx.26483.1.S1_s_at | 0.807 |
| CfaAffx.28078.1.S1_s_at | 0.883 |
| CfaAffx.28164.1.S1_at | 0.752 |
| CfaAffx.2860.1.S1_s_at | 0.646 |
| CfaAffx.28798.1.S1_at | 1.066 |
| CfaAffx.29250.1.S1_s_at | 0.789 |
| CfaAffx.32063.1.S1_at | 1.16 |
| CfaAffx.360.1.S1_s_at | 1.141 |
| CfaAffx.3860.1.S1_s_at | 1.163 |
| CfaAffx.604.1.S1_at | 1.154 |
| CfaAffx.6669.1.S1_at | 0.816 |
| CfaAffx.7079.1.S1_at | 0.709 |
| CfaAffx.9326.1.S1_s_at | 0.794 |

### Example 4

### Diets Containing Higher Amounts of Long Chain Fatty Acids Promote Weight Loss and can be Used to Re-Program the Gene Expression of the Animal so that it Reflects a Propensity to Become Lean and Potentially Maintain Leanness

The data obtained from in vitro ingredient screens discussed above indicate that some ingredients that are high in long chain fatty acids (see Table 7) may have the potential to affect the expression of genes involved in fat metabolism in a way that would promote leanness of the animal as a whole. This is determined by analyzing data obtained from adipose tissue and from the ingredient assays discussed above using conventional computer algorithm analyses. Code for algorithms useful in this regard are familiar to one of skill in the art and may be developed without undue experimentation. An example of such code is provided below:

SELECT A.PROBE, TO_CHAR ( AVG(DECODE(A.EXPTDAY, ‘D0’,
GENE_NORM_INT, null))/AVG(DECODE(A.EXPTDAY, ‘D14’, GENE_NORM_INT,
null)),‘99999.99999’ ) FATLEAN_FC, STATS_T_TEST_INDEPU( A.EXPTDAY,
GENE_NORM_INT) P_VALUE, B.TOP_HIT_DEF,
COUNT(DISTINCT C.INGREDIENT),
COUNT(DISTINCT D.INGREDIENT)
FROM GERIATRICS_RNRM2 A, TOP_PROBE_ANNOT_2_3 B,
FILT_INDIV_CELLS_2 C, FILT_ACROSS_4_CELLS_2 D
WHERE A.PROBE=B.PROBE AND A.PROBE=C.PROBE (+) AND
A.PROBE=D.PROBE (+) AND
UPPER(A.PROBE) NOT LIKE ‘AFFX%’
GROUP BY A.PROBE, B.TOP_HIT_DEF
HAVING STATS_T_TEST_INDEPU( A.EXPTDAY, GENE_NORM_INT) <= .01 AND
AVG(DECODE(A.EXPTDAY, ‘D0’, GENE_NORM_INT,
null))/AVG(DECODE(A.EXPTDAY, ‘D14’, GENE_NORM_INT, null)) >= 5 AND
SUM(DECODE(PAMCALL, ‘P’, 1, 0)) = 40 ORDER BY PROBE

To confirm that the inclusion of linolenic acid or EPA/DHA (1.5:1) in diets fed to dogs does affect weight loss in dogs, three high protein diets containing either no added long chain fatty acids (Diet A) or added linolenic acid (approximately 1% based on 100% dry matter basis, Diet B) or EPA/DHA (1.5-1, approximately 0.30%:0.20%) (Diet C) were developed for comparison to a high fiber diet that is known to induce weight loss in dogs. In the study, 45 clinically fat dogs are all first fed a nutritionally complete control diet for 30 days prior to the start of the test. After the initial 30 days, the dogs are randomized into 4 groups. Three of the four groups receive one of the test diets and one group is given the high fiber diet as a control for a set period of time, e.g., 4 months. Results indicate that the three experimental foods (Diets A, B and C) have substantially higher digestibility than the higher fiber food. Results also indicate that approximately 38% of the dogs consuming the food containing EPA/DHA reach their weight loss goal at 90 days. Interestingly, dogs consuming the EPA/DHA food also maintain lean muscle mass and bone mineral content. The results also indicate that, at least at the clinical level, diets containing EP/DHA may be as effective as high fiber diets in affecting weight loss.

In order to validate the class predictor probe set and to test its ability to predict fatness or leanness in animals) the class predictor probe set (described in Example 3 above) is applied to gene expression data obtained from the 45 animals participating in the experiment above (expression data not shown). The class predictor analysis confirms that 41 of the 45 animals (approximately 90%) designated “fat” at the beginning of the test are in fact fat (the discrepancy may be due to the subjective nature of the conventional body condition scoring system that is currently used in the clinic). Interestingly, after 14 days of feeding the four diets described above, the class predictor analysis indicates that all animals, regardless of diet, display a “lean” gene expression profile. At the end of the study, it appears that all the animals on the control high fiber diet reflect a “fat” gene expression profile, approximately 25% of the animals on test Diets A and B reflect a biochemically “lean” gene expression profile and approximately 40% of the animals fed on Diet C containing EPA/DHA exhibit a biochemically “lean” gene expression profile (see Table 10).

TABLE 10
Approximate Percentage of Lean Animals
as Predicted by the 65-probe Class Predictor

| | Diet Day 0 | Diet Day 14 | Diet Day 120 |
|---|---|---|---|
| Diet A (n = 12) | 9% | 33% | 25% |
| Diet B (n = 10) | 10% | 40% | 25% |
| Diet C (n = 14) | 7% | 29% | 40% |
| High Fiber Diet (n = | 11% | 30% | 0% |
| 9) | | | |

### Example 5

### Possible Weight Loss Maintenance Experiment

Based on the results of the weight loss experiment discussed above, it is hypothesized that animals fed a diet containing EPA/DHA will not only lose weight but also will maintain the loss for a longer period of time compared to animals fed the other test and control high fiber diets.

In order to characterize the effects of Diets A, B, and C and the high fiber diet on weight loss maintenance, one could perform, for example, the following type of experiment:

Fat animals may be fed the four different diets (as described in Example 4) until they reach an optimum level of “leanness”. They may then be randomized and divided into subgroups that either continue to be fed the same test diet that they were fed previously or are switched to a maintenance diet that is nutritionally balanced but is not designed to induce or maintain weight loss and does not include appreciable amounts of linolenic acid or EPA/DHA, for example.

The animals may then be observed for a set period of time, e.g., up to 3 months, with their weights recorded daily, their body condition scores determined weekly and their percentage body fat determined on a monthly basis using conventional DEXA technologies.

What is claimed is:

1. A combination comprising (a) two or more polynucleotides selected from SEQ ID NOs:1-295 or useful variations thereof or (b) two or more proteins produced by the expression of two or more polynucleotides selected from SEQ ID NOs:1-295 or useful variations thereof.

2. The combination of claim 1 wherein the polynucleotides are selected from SEQ ID NOs:1-70 or useful variations thereof.

3. The combination of claim 1 wherein the polynucleotides are selected from SEQ ID NOs:1-25 or useful variations thereof.

4. The combination of claim 1 wherein the polynucleotides are selected from SEQ ID NOs identified in Table 2 or useful variations thereof.

5. The combination of claim 1 wherein the polynucleotides are selected from SEQ ID NOs identified in Table 3 or useful variations thereof.

6. The combination of claim 1 wherein the polynucleotides are canine polynucleotides.

7. A composition comprising two or more probes suitable for detecting the expression of genes differentially expressed in fat animals compared to lean animals, the probes comprising:
(a) polynucleotides selected from SEQ ID NOs:1-295 or useful variations thereof; or
(b) polypeptides that specifically bind to proteins produced by the expression of one or more polynucleotides selected from SEQ ID NOs:1-295 or useful variations thereof.

8. The composition of claim 7 wherein the polynucleotides are selected from SEQ ID NOs:1-70 or useful variations thereof.

9. The composition of claim 7 wherein the polynucleotides are selected from SEQ ID NOs:1-25 or useful variations thereof.

10. The composition of claim 7 wherein the polynucleotides are selected from SEQ ID NOs identified in Table 2 or useful variations thereof.

11. The composition of claim 7 wherein the polynucleotides are selected from SEQ ID NOs identified in Table 3 or useful variations thereof.

12. The composition of claim 7 wherein the polynucleotides are canine polynucleotides.

13. A device suitable for detecting the expression of a plurality of genes differentially expressed in fat animals compared to lean animals comprising a substrate having a plurality of probes affixed to the substrate at known locations, the probes comprising:
(a) polynucleotides selected from SEQ ID NOs:1-295 or useful variations thereof; or
(b) polypeptides each of which specifically binds to proteins produced by expression of one or more polynucleotides selected from SEQ ID NOs:1-295 or useful variations thereof.

14. The device of claim 13 wherein the polynucleotides are selected from SEQ ID NOs:1-70 or useful variations thereof.

15. The device of claim 13 wherein the polynucleotides are selected from SEQ ID NOs:1-25 or useful variations thereof.

16. The device of claim 13 wherein the polynucleotides are selected from SEQ ID NOs identified in Table 2 or useful variations thereof.

17. The device of claim 13 wherein the polynucleotides are selected from SEQ ID NOs identified in Table 3 or useful variations thereof.

18. The device of claim 13 wherein the polynucleotides are canine polynucleotides.

19. The device of claim 13 wherein the probes are arranged in an array.

20. The device of claim 13 wherein the polypeptide probes are antibodies.

21. The device of claim 20 wherein the antibodies are monoclonal antibodies.

22. A method for detecting the differential expression of one or more genes differentially expressed in fat animals compared to lean animals in a sample comprising:
(a) hybridizing a combination comprising a plurality of polynucleotide probes selected from SEQ ID NOs-1-295 or useful variations thereof with polynucleotides in the sample to form one or more hybridization complexes;
(b) optionally, hybridizing a combination comprising a plurality of polynucleotide probes selected from SEQ ID NOs:1-295 or useful variations thereof with polynucleotides in a standard to form one or more hybridization complexes;
(c) detecting the hybridization complexes from the sample and, optionally, the standard from step (b); and
(d) comparing the hybridization complexes from the sample with the hybridization complexes from a standard, wherein a difference in the amount of hybridization complexes between the standard and sample of 2 fold or more indicate differential expression of genes differentially expressed in fat animals compared to lean animals in the sample.

23. The method of claim 22 wherein the probes are selected from SEQ ID NOs:1-70 or useful variations thereof and the fold difference is 2.5 or more.

24. The method of claim 22 wherein the probes are selected from SEQ ID NOs:1-25 or useful variations thereof and the fold difference is 3 or more.

25. The method of claim 22 wherein the probes are selected from SEQ ID NOs identified in Table 2 or useful variations thereof and the fold difference is 2 or more.

26. The method of claim 22 wherein the probes are selected from SEQ ID NOs identified in Table 3 or useful variations thereof and the fold difference is 2 or more.

27. The method of claim 22 wherein the probes are bound to a substrate.

28. The method of claim 27 wherein the probes are in an array.

29. The method of claim 22 wherein the detecting is performed at intervals for an animal and used to monitor the animal's progress when attempting to modulate the amount of adipose tissue on the animal in response to an adipose tissue modulation program.

30. The method of claim 22 wherein the probes are canine polynucleotides.

31. The method of claim 22 further comprising exposing the sample to a test substance before hybridization, wherein comparison to a standard is indicative of whether the test substance altered the expression of genes differentially expressed in fat animals compared to lean animals in the sample.

32. A method for detecting the differential expression of genes differentially expressed in fat animals compared to lean animals in a sample comprising:
(a) reacting a combination comprising a plurality of polypeptide probes with proteins in the sample under conditions that allow specific binding between the probes and the proteins to occur, wherein the proteins bound by the probes are produced by expression of one or more polynucleotides selected from SEQ ID NOs:1-295 or useful variations thereof;
(b) optionally, reacting a combination comprising a plurality of polypeptide probes with proteins in a standard under conditions that allow specific binding between the probes and the proteins to occur, wherein the proteins bound by the probes are produced by expression of one or more polynucleotides selected from SEQ ID NOs:1-295 or useful variations thereof;
(c) detecting specific binding in the sample and, optionally, the standard from step (b); and
(d) comparing the specific binding in the sample with that of a standard, wherein a difference between the specific binding in the standard and the sample of 2 fold or more indicates differential expression of genes differentially expressed in fat animals compared to lean animals in the sample.

33. The method of claim 32 wherein the probes are selected from SEQ ID NOs:1-70 or useful variations thereof and the fold difference is 2.5 or more.

34. The method of claim 32 wherein the probes are selected from SEQ ID NOs:1-25 or useful variations thereof and the fold difference is 3 or more.

35. The method of claim 32 wherein the probes are selected from SEQ ID NOs identified in Table 2 or useful variations thereof and the fold difference is 2 or more.

36. The method of claim 32 wherein the probes are selected from SEQ ID NOs identified in Table 3 or useful variations thereof and the fold difference is 2 or more.

37. The method of claim 32 wherein the probes are bound to a substrate.

38. The method of claim 37 wherein the probes are in an array.

39. The method of claim 32 further comprising detecting the differential expression of genes differentially expressed in fat animals compared to lean animals for an animal at intervals during an adipose tissue modulation program and determining the effectiveness of the program by observing changes in the expression of the genes.

40. The method of claim 39 wherein a decrease in gene expression indicates a reduction in adipose tissue on the animal.

41. The method of claim 32 wherein the probes are canine polypeptides.

42. The method of claim 32 further comprising exposing the sample to a test substance before reacting the polypeptides with the proteins wherein comparison to a standard is indicative of whether the test substance altered the expression of genes differentially expressed in fat animals compared to lean animals in the sample.

43. A method for measuring the effect of a test substance on the expression of one or more genes differentially expressed in fat animals compared to lean animals and a method for screening a test substance to determine if it is likely to be useful for modulating the amount of adipose tissue on an animal comprising:
(a) determining a first expression profile by measuring the transcription or translation products of one or more polynucleotides selected from SEQ ID NOs:1-295 or useful variations thereof in a test system in the absence of the test substance;
(b) determining a second expression profile by measuring the transcription or translation products of one or more polynucleotides selected from SEQ ID NOs:1-295 or useful variations thereof in a test system in the presence of the test substance; and
(c) comparing the first expression profile to the second expression profile, wherein a change in the second expression profile compared to the first expression profile of 2 fold or more indicates that the test substance effects the expression of polynucleotides differentially expressed in fat animals compared to lean animals and that the test substance is likely to be useful for modulating the amount of adipose tissue on an animal.

44. The method of claim 43 wherein the polynucleotides are selected from SEQ ID NOs:1-70 or useful variations thereof and the fold difference is 2.5 or more.

45. The method of claim 43 wherein the polynucleotides are selected from SEQ ID NOs:1-25 or useful variations thereof and the fold difference is 3 or more.

46. The method of claim 43 wherein the polynucleotides are selected from SEQ ID NOs identified in Table 2 or useful variations thereof and the fold difference is 2 or more.

47. The method of claim 43 wherein the polynucleotides are selected from SEQ ID NOs identified in Table 3 or useful variations thereof and the fold difference is 2 or more.

48. The method of claim 43 wherein determining an expression profile utilizes a plurality of polynucleotides.

49. The method of claim 48 wherein the polynucleotides are bound to a substrate.

50. The method of claim 49 wherein the probes are in an array.

51. The method of claim 43 wherein the standard and test samples are obtained from a canine.

52. A substance identified by the method of claim 42.

53. A method for formulating a prognosis that an animal is likely to become fat or developing a diagnosis that an animal is fat comprising determining if one or more polynucleotides selected from SEQ ID NOs:1-295 or useful variations thereof or one or more polypeptides that specifically bind to proteins produced by expression of one or more polynucleotides selected from SEQ ID NOs:1-295 or useful variations thereof are differentially expressed in the animal compared to one or more lean animals, wherein the animal is determined to be likely to become fat or determined to be fat if the comparison indicates that the polynucleotides or polypeptides are differentially expressed in the animal compared to the lean animals by a fold of 2 or more.

54. The method of claim 53 wherein the polynucleotides are selected from SEQ ID NOs:1-70 or useful variations thereof and the fold difference is 2.5 or more.

55. The method of claim 53 wherein the polynucleotides are selected from SEQ ID NOs:1-25 or useful variations thereof and the fold difference is 3 or more.

56. The method of claim 53 wherein the polynucleotides are selected from SEQ ID NOs identified in Table 2 or useful variations thereof and the fold difference is 2 or more.

57. The method of claim 53 wherein the polynucleotides are selected from SEQ ID NOs identified in Table 3 or useful variations thereof and the fold difference is 2 or more.

58. A method for manipulating the genome of a non-human animal or the expression of the genome of an animal comprising disrupting the expression of one or more genes differentially expressed in fat animals compared to lean animals.

59. The method of claim 58 wherein expression is disrupted by “knocking out” endogenous genes in the animal.

60. The method of claim 58 wherein expression is disrupted by interfering with transcription or translation.

61. The method of claim 58 wherein expression is disrupted using polynucleotides constructed using polynucleotides selected from SEQ ID NOs:1-295 or useful variations thereof.

62. A transgenic animal produced using the method of claim 58.

63. A composition suitable for manipulating the genome of an animal comprising one or more substances that interfere with the expression of one or more genes differentially expressed in fat animals compared to lean animals.

64. The composition of claim 63 wherein the substances are antisense molecules or siRNAs.

65. The composition of claim 63 wherein the substances are polynucleotides constructed using polynucleotides selected from SEQ ID NOs:1-295 or useful variations thereof.

66. A method for modulating the expression of one or more genes differentially expressed in fat animals compared to lean animals or modulating the amount of adipose tissue on an animal comprising administering to the animal a gene expression or tissue modulating amount of a composition comprising one or more of DHA, EPA, EPA and DHA, ALA, LA, ARA, and SA.

67. The method of claim 66 wherein DHA is administered in amounts of from about 1 to about 30; EPA is administered in amounts of from about 1 to about 30; EPA/DHA Combo (1.5:1 ratio) are administered in amounts of from about 4/2 to about 30/45; ALA is administered in amounts of from about 10 to about 100; LA is administered in amounts of from about 30 to about 600; ARA is administered in amounts of from about 5 to about 50; and SA is administered in amounts of from about 3 to about 60, all in mg/kg/day.

68. The method of claim 66 further comprising administering to the animal one or more drugs or other substances that modulate the amount of adipose tissue on an animal.

69. A composition suitable for modulating the expression of one or more genes differentially expressed in fat animals compared to lean animals or modulating the amount of adipose tissue on an animal comprising a gene expression or tissue modulating amount of one or more of DHA, EPA, EPA and DHA, ALA, LA, ARA, and SA.

70. The composition of claim 69 comprising DHA in amounts sufficient to administer to an animal from about 1 to about 30; EPA in amounts sufficient to administer to an animal from about 1 to about 30; EPA/DHA Combo (1.5:1 ratio) in amounts sufficient to administer to an animal from about 4/2 to about 30/45; ALA in amounts sufficient to administer to an animal from about 10 to about 100; LA in amounts sufficient to administer to an animal from about 30 to about 600; ARA in amounts sufficient to administer to an animal from about 5 to about 50; and SA in amounts sufficient to administer to an animal from about 3 to about 60, all in mg/kg/day.

71. The composition of claim 69 further comprising one or more drugs or other substances that modulate the amount of adipose tissue on an animal.

72. A method for selecting an animal for inclusion in one or more groups comprising determining the expression profile of the animal for (a) polynucleotides selected from SEQ ID NOs:1-295 or useful variations thereof or (b) polypeptides each of which specifically binds to proteins produced by expression of one or more polynucleotides selected from SEQ ID NOs:1-295 or useful variations thereof and assigning the animal to a group based upon the expression profile.

73. The method of claim 72 wherein the groups are lean and fat groups and animals are assigned to the fat group based upon a differential expression of 2 fold or more and animals are assigned to the lean group based upon a differential expression of less than 2 fold compared to a standard.

74. A computer system suitable for manipulating data relating to one or more genes differentially expressed in fat animals compared to lean animals comprising a database containing information identifying the expression level of one or more polynucleotides selected from SEQ ID NOs:1-295 or useful variations thereof and/or polypeptides that specifically bind to proteins produced by the expression of one or more polynucleotides selected from SEQ ID NOs:1-295 or useful variations thereof in lean animals and/or fat animals and a user interface to interact with the database.

75. A method for producing an antibody suitable for use in detecting one or more genes differentially expressed in fat animals compared to lean animals comprising:
(a) immunizing an animal with polypeptides produced by expression of one or more polynucleotides selected from SEQ ID NOs:1-295 or useful variations thereof under conditions that elicit an antibody response;
(b) isolating animal antibodies; and
(c) screening the isolated antibodies with the polypeptide, thereby identifying an antibody that specifically binds the polypeptide.

76. An isolated and purified antibody produced using the method of claim 75.

77. A kit suitable for determining the differential expression of one or more genes differentially expressed in fat animals compared to lean animals in a test system comprising in separate containers in a single package or in separate containers in a virtual package, as appropriate for the use and kit component, two or more probes suitable for detecting the expression of genes differentially expressed in fat animals compared to lean animals, the probes comprising:
(a) polynucleotides selected from SEQ ID NOs:1-295 or useful variations thereof; or
(b) polypeptides that specifically bind to proteins produced by the expression of one or more polynucleotides selected from SEQ ID NOs:1-295 or useful variations thereof; and
at least one of (1) instructions for how to use the probes of the present invention; (2) reagents and equipment necessary to use the probes; (3) a composition suitable for modulating the expression of one or more genes differentially expressed in fat animals compared to lean animals; (4) a composition suitable for disrupting the expression of one or more genes differentially expressed in fat animals compared to lean animals; (5) a food composition suitable for modulating the amount of adipose tissue on an animal; and (6) one or more drugs or other substances that that modulate the amount of adipose tissue on an animal.

78. The kit of claim 77 wherein the probes are bound to a substrate.

79. The kit of claim 78 wherein the probes are in an array.

80. The kit of claim 77 wherein the compositions comprise one or more of DHA, EPA, EPA and DHA, ALA, LA, ARA, and SA.

81. A means for communicating information about or instructions for one or more of (1) using the polynucleotides of the present invention for detecting the expression of genes differentially expressed in fat animals compared to lean animals in a sample, (2) using the polynucleotides of the present invention for measuring the effect of a test substance on the expression of one or more genes differentially expressed in fat animals compared to lean animals, (3) using the polynucleotides of the present invention for screening a test substance to determine if it is likely to be useful for modulating the amount of adipose tissue on an animal, (4) using the polynucleotides of the present invention for formulating a prognosis that an animal is likely to become fat or developing a diagnosis that an animal is fat, (5) using the polynucleotides of the present invention for manipulating the genome of a non-human animal or the expression of the genome of an animal, (6) using the polynucleotides of the present invention for modulating the expression of one or more genes differentially expressed in fat animals compared to lean animals or modulating the amount of adipose tissue on an animal, (7) using the polynucleotides of the present invention for selecting an animal for inclusion in one or more groups, (8) using the polynucleotides of the present invention for using computer system to manipulate data relating to genes differentially expressed in fat animals compared to lean animals, (9) administering substances of the present invention to an animal, alone or in combination with the other elements of the present invention, (10) using the substances of the present invention for modulating the amount of adipose tissue on an animal, (11) using the computer system of the present invention, (12) using the kits of the present invention, and (13) using the methods and compositions of the present invention with one or more drugs or other substances that that modulate the amount of adipose tissue on an animal comprising a document, digital storage media, optical storage media, audio presentation, or visual display containing the information or instructions.

82. The means of claim 81 selected from the group consisting of a displayed web site, visual display, kiosk, brochure, product label, package insert, advertisement, handout, public announcement, audiotape, videotape, DVD, CD-ROM, computer readable chip, computer readable card, computer readable disk, computer memory, or combination thereof.

83. Use of the polynucleotide data of Table 1 in the manufacture of a composition for modulating the expression of one or more genes differentially expressed in fat animals compared to lean animals.

84. Use of the class predictor data of Table 8 and Table 9 in the manufacture of a composition for modulating the expression of one or more genes differentially expressed in fat animals compared to lean animals.

85. Use of the polynucleotide data of Table 1 in the manufacture of a kit for diagnosing the body condition score of an animal.

86. Use of the class predictor data of Table 8 and Table 9 in the manufacture of a kit for diagnosing the body condition score of an animal.
